(19) United States
(12) Patent Application Publication
(10) Pub. No.: US 2025/0235527 A1
(43) Pub. Date: Jul. 24, 2025

(54) SYNTHETIC MODIFIED VACCINIA ANKARA (SMVA) BASED CORONAVIRUS VACCINES

(71) Applicant: CITY OF HOPE, Duarte, CA (US)

(72) Inventors: Don J. DIAMOND, Duarte, CA (US); Felix WUSSOW, Duarte, CA (US); Flavia CHIUPPESI, Duarte, CA (US)

(73) Assignee: CITY OF HOPE, Duarte, CA (US)

(21) Appl. No.: 18/711,073

(22) PCT Filed: Nov. 17, 2022
(86) PCT No.: PCT/US22/80065

§ 371 Date: May 16, 2024

Related U.S. Application Data
(60) Provisional application No. 63/280,524, filed on Nov. 17, 2021.

(51) Int. Cl. A61K 39/215; A61K 39/00; A61P 31/14; C12N 15/86

(52) U.S. Cl. CPC A61K 39/215; A61P 31/14; C12N 15/86; A61K 2039/5256; A61K 2039/575; C12N 2710/24143; C12N 2770/20034

(57) ABSTRACT

Disclosed are recombinant synthetic MVA-based vaccines for preventing or treating infections caused by a coronavirus or variants thereof.

### CROSS-REFERENCE TO RELATED APPLICATIONS

This application claims the benefit of U.S. Provisional Patent Application No. 63/280,524, filed on Nov. 17, 2021, the contents of which are incorporated by reference in their entirety.

### SEQUENCE LISTING

This application contains a Sequence Listing, which was submitted in XML format via Patent Center, and is hereby incorporated by reference in its entirety. The XML copy, created on Nov. 15, 2022, is named 0544358219WO00.xml and is 595 KB in size.

### BACKGROUND

Since the emergence of severe acute respiratory syndrome coronavirus 2 (SARS-CoV-2) in China at the end of 2019, SARS-CoV-2 has spread rapidly worldwide, causing a global pandemic with millions of fatalities. Several SARS-CoV-2 vaccines were developed in response to the COVID-19 pandemic at an unprecedented pace and showed 62%-95% efficacy in Phase 3 clinical trials, leading to their emergency use authorization (EUA) in many countries by the end of 2020 or beginning of 2021. The vaccines include those based on mRNA, adenovirus vectors, and nanoparticles that utilize different antigenic forms of the spike (S) protein to induce protective immunity against SARS-CoV-2 primarily through the function of neutralizing antibodies (NAb). The S protein mediates SARS-CoV-2 entry into a host cell through binding to angiotensin-converting enzyme 2 (ACE) and is the major target of NAb. Despite unprecedented mass vaccination and availability of effective COVID-19 vaccines, SARS-CoV-2 remains a threat to human health due to the continuous emergence of variants of concern (VOC) with the ability to evade vaccine-induced immunity.

Following the emergence of Alpha (B.1.1.7), Beta (B.1.351), Gamma (P.1), and Delta (B.1.617), Omicron subvariants have emerged as predominant SARS-CoV-2 VOC, which includes BA.1, BA.2, BA.2 sub-lineages such as BA.2.12.1, BA.4, BA.5, BA.2.75, and more recent Omicron lineage such as BQ.1, BQ.1.1, and XBB. Omicron subvariants have exceptional capacity to evade NAb due to numerous mutations in the S protein that dramatically exceed S mutations of other earlier-occurring SARS-CoV-2 VOC, thereby posing a unique challenge for COVID-19 vaccination. Several studies reported reduced clinical effectiveness against Omicron variants by approved COVID-19 vaccines, which were designed to elicit protective immunity solely based on the S protein of the Wuhan-Hu-1 reference strain. While waning vaccine efficacy against VOC can be counteracted by repeated booster vaccination, COVID-19 booster vaccines with altered or variant-matched antigen design have been developed to specifically enhance the stimulation of cross-protective immunity against emerging SARS-CoV-2 VOC.

Alternative vaccines based on different platforms, or modified epitope or antigen design, could therefore contribute to the establishment of long-term and cross-reactive immunity against SARS-CoV-2 and its emerging VOC. Accordingly, this disclosure provides vaccines using a synthetic modified vaccinia Ankara (sMVA) platform to satisfy an urgent need in the field.

### SUMMARY

Disclosed herein are vaccines and/or other immunogenic compositions, as well as the use thereof in preventing a coronavirus infection, and/or preventing, treating, or reducing the severity of COVID-19 caused by the coronavirus infection in a subject.

In some aspects, provided are vaccines and/or immunogenic compositions for preventing or treating SARS-CoV-2 infection in a subject comprising: (i) a single synthetic DNA fragment comprising the entire genome of a modified vaccinia Ankara (MVA), or two or more synthetic DNA fragments each comprising a partial sequence of the genome of the MVA such that the two or more DNA fragments, when transferred into a host cell, are assembled sequentially and comprise the full-length sequence of the MVA genome, and (ii) one or more DNA sequences encoding the spike (S) protein and the nucleocapsid (N) protein of SARS-CoV-2, subunits, or fragments thereof, inserted into one or more insertion sites of the MVA, wherein the S protein and the N protein are expressed in the host cell upon transfection of the one or more MVA DNA fragments. In some embodiments, the one or more insertion sites comprise Del2, IGR69/70, and Del3.

In some embodiments, the one or more synthetic DNA fragments with inserted antigen sequences are (co-)transfected into permissive cells (e.g., baby hamster kidney (BHK) cells, chicken embryo fibroblasts (CEF)) and subsequently infected with fowl pox virus (FPV) as a helper virus to initiate the reconstitution of recombinant sMVA virus that co-expresses SARS-CoV-2 spike and nucleocapsid antigens.

In some embodiments, the one or more DNA sequences encoding the S protein and the N protein are under the control of an mH5 promoter.

In some embodiments, the DNA sequence encoding the S protein is based on the Wuhan-Hu-1 reference strain or is derived from a variant of concern (VOC). In some embodiments, the DNA sequence encoding the N protein is based on the Wuhan-Hu-1 reference strain or is derived from a VOC. In some embodiments, the VOC is selected from the group consisting of B.1.1.7 (Alpha), B.1.351 (Beta), P.1 (Gamma), B.1.617.2 (Delta), B.1.621 (Mu), C.37 (Lambda), C.1.2, BA.1 (Omicron), BA.2 (Omicron), BA.2.12.1 (Omicron), BA.4 (Omicron), BA.5 (Omicron), BA.2.75 (Omicron), BQ.1 (Omicron), BQ.1.1 (Omicron), and XBB (Omicron).

In some embodiments, the DNA sequence encoding the S protein comprises a nucleotide sequence that is at least 80% identical to any one of SEQ ID NOs: 5, 9, 11, 13, 15, 21, 25, and 29. In some embodiments, the DNA sequence encoding the N protein comprises a nucleotide sequence that is at least 80% identical to any one of SEQ ID NOs: 7, 17, 19, 23, 27, and 31.

In some aspects, provided are vaccines and/or immunogenic compositions for preventing or treating SARS-CoV-2 infection in a subject comprising a synthetic modified vaccinia Ankara (sMVA) vector comprising a nucleotide sequence that is at least 80% identical to SEQ ID NO: 33.

In some embodiments, the vaccine and/or immunogenic composition further comprises a pharmaceutically acceptable carrier, adjuvant, additive, or combination thereof.

In some aspects, provided are methods of preventing or treating SARS-CoV-2 infection in a subject comprising administering to the subject a prophylactically or therapeutically effective amount of the vaccine and/or immunogenic composition according to various embodiments of the present technology.

In some aspects, provided are methods of eliciting an immune response to SARS-CoV-2 in a subject by administering to the subject a prophylactically or therapeutically effective amount of the vaccine and/or immunogenic composition according to various embodiments of the present technology, wherein the subject is infected with SARS-CoV-2 or is at risk of being infected with SARS-CoV-2.

In some embodiments, the SARS-CoV-2 virus comprises the Wuhan-Hu-1 reference strain or a VOC. In some embodiments, the VOC is selected from the group consisting of B.1.1.7 (Alpha), B.1.351 (Beta), P.1 (Gamma), B.1.617.2 (Delta), B.1.621 (Mu), C.37 (Lambda), C.1.2, BA.1 (Omicron), BA.2 (Omicron), BA.2.12.1 (Omicron), BA.4 (Omicron), BA.5 (Omicron), BA.2.75 (Omicron), BQ.1 (Omicron), BQ.1.1 (Omicron), and XBB (Omicron).

In some embodiments, the vaccine and/or immunogenic composition is administered by intramuscular injection, intranasal instillation, intradermal injection, and/or scarification.

In some embodiments, a single dose of the vaccine composition is administered. In some embodiments, two doses of the vaccine composition are administered. In some embodiments, three or more doses of the vaccine composition are administered. In some embodiments, one booster dose of the vaccine and/or immunogenic composition is administered. In some embodiments, two or more booster doses of the vaccine and/or immunogenic composition are administered.

### BRIEF DESCRIPTION OF THE DRAWINGS

This application contains at least one drawing executed in color. Copies of this application with color drawing(s) will be provided to the Office upon request and payment of the necessary fees.

FIGS. 1A-1H show COH04S1 immunogenicity in Syrian hamsters. FIG. 1A shows a design scheme of the COH04S1 construct. COH04S1 is an sMVA vaccine vector that contains full-length SARS-CoV-2 N and S antigens and mH5 promoter sequences inserted into the deletion 2 (Del2) and deletion 3 (Del3) sites as indicated. ITR=inverted terminal repeat. FIG. 1B shows study design. Hamsters were vaccinated twice with COH04S1 by intramuscular (IM) (COH04S1-IM) or intranasal (IN) (COH04S1-IN) route as indicated. Unvaccinated animals and hamsters vaccinated with empty sMVA vector by IM (sMVA-IM) or IN (sMVA-IN) route were used as controls. Blood samples were collected at 0, 28, and 42 days. Hamsters were challenged intranasally at day 42, and body weight changes were recorded daily for 10 days. At endpoint, nasal wash, turbinates, and lung tissue were collected for downstream analyses. FIGS. 1C-1D show IgG endpoint titers. S-, receptor-binding domain (RBD)-, and N-specific binding antibody titers were measured in serum samples of vaccine and control groups at day 28 (d28) post-prime and at day 42 (d42) post-booster vaccination via ELISA. Data are presented as geometric mean values+geometric SD. Dotted lines indicate lower limit of detection. Two-way ANOVA with Tukey's multiple comparison test was used. FIG. 1E shows IgG2-3/IgG1 ratios. S-, RBD-, and N-specific IgG2-3 and IgG1 endpoint titers were measured at day 42 (d42) in serum samples of vaccine and control groups and used to assess IgG2-3/IgG1 ratios. An IgG2-3/IgG1 ratio >1 is indicative of a Th1-biased response. Geometric means are indicated with a line. FIGS. 1F-1G show NAb titers. NAb titers were measured in serum samples of vaccine and control groups post-first (d28) and post-second (d42) vaccination via plaque reduction neutralization titer (PRNT) assay against SARS-CoV-2 infectious virus. Data are presented as geometric mean values+SD. Dotted lines indicate lower limit of detection. Values below the limit of detection (PRNT=20) are indicated as 10. One-way ANOVA with Holm-Sidak's multiple comparison test was used. FIG. 1H shows VOC-specific NAb titers. NAb were measured in pooled serum samples of vaccine and control groups collected at the time of challenge (d42) using pseudoviruses (pv) with S D614G mutation or S sequences based on several VOC, including Alpha (B.1.1.7), Beta (B.1.351), Gamma (P.1), and Delta (B.1.617.2). Titers are expressed as NT50. Fold NT50 reduction in comparison to D614G PV are shown. 0.01<*p<0.05, 0.001<**p<0.01, 0.0001<***p<0.001, ****p<0.0001.

FIGS. 2A-2N show COH04S1 immunogenicity in Syrian hamsters. FIGS. 2A-2F show binding antibody titers. Shown are the binding curves of S, RBD, and N antigen-specific binding antibody titers measured at day 28 (d28) or day 42 (d42) after the first and second vaccinations in serum samples of COH04S1-IM- and COH04S1-IN-vaccinated hamsters and unvaccinated and sMVA vector control animals. Two-way ANOVA with Tukey's multiple comparison test was used. FIGS. 2G-21 show IgG2-3/IgG1 antibody titers. Shown are the IgG endpoint titers of S, RBD, and N antigen-specific IgG2-3 and IgG1 binding antibodies measured in serum samples of day 42 (d42), and ratios are shown. Geometric means are indicated with a line. Dotted lines indicate lower limit of detection. FIGS. 2J-2N show VOC-specific NAb titers. Shown are NAb titers measured at the indicated time points by PV variants with D614G mutation or based on several SARS-CoV-2 VOC in vaccine and control groups.

FIGS. 3A-3H show COH04S1-mediated vaccine protection in hamsters following sub-lethal SARS-CoV-2 challenge. FIG. 3A shows body weight change. Body weight of COH04S1-IM- and COH04S1-IN-vaccinated animals as well as unvaccinated and sMVA-IM and sMVA-IN vector control animals was measured daily for 10 days post-challenge. Weight loss is reported as mean±SD. Two-way ANOVA followed by Tukey's multiple comparison test was used to compare group mean values at each time point. FIG. 3B shows maximal weight loss. Percentage of maximal weight loss is shown in single animals of vaccine and control groups. Lines and bars represent median values and 95% Cl, respectively. A dotted line represents the maximum weight loss allowed before euthanasia. Peaks of weight loss in each group were compared using one-way ANOVA followed by Tukey's multiple comparison test. FIGS. 3C-3D show lung viral loads. SARS-CoV-2 genomic RNA (gRNA) and subgenomic RNA (sgRNA) copies were quantified in lung tissue of vaccine and control groups at day 10 post-challenge by qPCR. Bars show RNA copies geometric mean±geometric SD. Dotted lines represent lower limit of detection. Kruskal-Wallis test followed by Dunn's multiple comparison test was used. FIGS. 3E-3H show histopathological findings. Hematoxylin/eosin-stained lung sections of COH04S1-vaccinated hamsters and control animals at day 10 post-challenge were evaluated by a board-certified pathologist, and microscopic findings were graded based on severity on a scale from 1 to 5 (Table 5). FIG. 3E shows the cumulative pathology score of all histopathologic findings in each group. FIG. 3F shows grading of bronchioalveolar hyperplasia disease severity in each group. One-way ANOVA followed by Holm-Sidak's multiple comparison test was used. FIG. 3G shows the severity of lung inflammatory microscopic findings based on 1-to-5 scaling of four inflammation types as indicated. Bars represent mean values±SD. One-way ANOVA followed by Tukey's multiple comparison test was used. In FIGS. 3B-3F, 0.01<*p<0.05, 0.001<**p<0.01, 0.0001<***p<0.001, ****p<0.0001. FIG. 3H shows representative images of histopathological findings in lung sections of COH04S1-vaccinated animals and control animals. Black arrows indicate moderate and mild bronchioalveolar hyperplasia in lung sections of sMVA-IM and sMVA-IN control animals and COH04S1-IN animals. Black arrows in lung sections of unvaccinated control animals indicate hyperplastic alveolar cells. 10× magnification.

FIGS. 4A-4D show COH04S1-mediated protection of hamsters following sub-lethal SARS-CoV-2 challenge. Shown are SARS-CoV-2 gRNA and sgRNA copies quantified by qPCR in nasal wash and turbinates of COH04S1-IM- and COH04S1-IN-vaccinated hamsters and unvaccinated and sMVA control animals at day 10 post-challenge. Bars show RNA copies geometric mean±geometric SD. Dotted lines represent lower limit of detection of the assay. Kruskal-Wallis test followed by Dunn's multiple comparison test was used. 0.01<*p<0.05, 0.001<**p<0.01.

FIGS. 5A-5B show correlative analysis of immunological and virological parameters in COH04S1-vaccinated hamsters challenged with SARS-CoV-2. Spearman correlation analysis was performed between the indicated pre-challenge immune responses and post-challenge virological assessments of COH04S1-IM- and COH04S1-IN-vaccinated hamsters and unvaccinated and sMVA control animals. FIG. 5A shows that Spearman correlation coefficients were calculated and plotted as a matrix. FIG. 5B shows that two-tailed p values were calculated and indicated as 0.01<*p<0.05, 0.001<**p<0.01, 0.0001<***p<0.001, ****p<0.0001. ns=not significant.

FIGS. 6A-6I show COH04S1 vaccine immunogenicity in nonhuman primates (NHP). FIG. 6A shows study design. African green monkey NHP were vaccinated with COH04S1 vaccine using two-dose (COH04S1-2D; N=6) or single-dose (COH04S1-1D; N=6) vaccination regimen as indicated. Mock-vaccinated NHP and NHP vaccinated with empty sMVA vector using the same dose vaccination regimen were included as controls. Six weeks after vaccination, NHP were challenged by IN/intratracheal (IT) route with SARS-CoV-2 USA-WA1/2020 reference strain. Bronchoalveolar lavages (BAL) and oral and nasal swab samples for viral load analysis were collected at multiple time points post-challenge. A subset of animals in each group were necropsied at days 7 and 21 post-challenge for viral load and histopathology analysis. FIGS. 6B-6D show binding antibody titers. S, RBD, and N antigen-specific binding antibody titers were measured at the indicated time points in serum samples of COH04S1-2D- and COH04S1-1D-vaccinated NHP via ELISA. Data is presented as geometric mean values±geometric SD. Two-way ANOVA followed by Sidak's multiple comparison test was used. FIG. 6E shows VOC-specific antibody titers. S-specific binding antibody titers were measured at day 62 by ELISA using S antigens based on the Wuhan-Hu-1 reference strain or several SARS-CoV-2 VOC, including Beta (B.1.351), Gamma (P.1), and Delta (B.1.617.2). Endpoint titers are presented as geometric mean values±geometric SD. Two-way ANOVA with Sidak's multiple comparison test was used for statistical analysis. FIG. 6F shows BAL antibody titers. S, RBD, and N antigen-specific binding antibody titers were measured by ELISA at day 42 in BAL samples of COH04S1-IM and COH04S1-IN vaccine group. Endpoint titers are presented as geometric mean values±geometric SD. Two-way ANOVA with Sidak's multiple comparison test was used. FIG. 6G shows NAb titers. NAb titers were measured by PRNT assay against SARS-CoV-2 USA-WA1/2020 reference strain in serum samples of vaccinated NHP. Serum dilutions reducing the plaque number by 50% (ID50) are presented as geometric mean values±geometric SD. Two-way ANOVA followed by Sidak's multiple comparison test was used to compare ID50 values. PRNT-assayed NAb titers measured in serum samples of healthcare workers (N=14) vaccinated two times with Pfizer-BioNTech BNT162b2 mRNA vaccine were included. FIGS. 6H-6I show T cell responses. IFNγ-, IL-2-, and IL-4-expressing S and N antigen-specific T cell responses were measured at 2 weeks post-challenge by ELISPOT. Bars represent mean values, and lines represent ±SD. Two-way ANOVA followed by Tukey's multiple comparison test was used. Dotted lines indicate the arbitrary positive threshold of 30 spots/10⁶ cells. 0.01<*p<0.05, 0.001<**p<0.01, 0.0001<***p<0.001, ****p<0.0001.

FIGS. 7A-7J show COH04S1-induced humoral immunity in NHP. FIGS. 7A-7F show binding antibody titers. Shown are S, RBD, and N antigen-specific IgG endpoint titers measured at the indicated time points in COH04S1-2D- and COH04S1-1 D-vaccinated NHP and mock-vaccinated and sMVA vector control animals. Data are presented as geometric mean values±geometric SD. Two-way ANOVA followed by Sidak's multiple comparison test was used. FIGS. 7G-7H show BAL binding antibodies. Shown are endpoint titers of IgG binding antibodies to S, RBD, and N measured in BAL samples in vaccine and control groups at day 42. Endpoint titers are presented as geometric mean values±geometric SD. Two-way ANOVA with Sidak's multiple comparison test was used. FIGS. 7I-7J show VOC-specific binding antibody titers. Shown are endpoint titers of S-specific IgG binding antibodies measured 1 week post-challenge by ELISA with S antigens based on Wuhan-Hu-1 reference strain or several VOC, including Beta (B.1.351), Gamma (P.1), and Delta (B.1.617.2). Endpoint titers are presented as geometric mean values±geometric SD. Two-way ANOVA with Sidak's multiple comparison test was used. 0.01<*p<0.05, 0.001<**p<0.01, 0.0001<***p<0.001, ****p<0.0001.

FIGS. 8A-8F show COH04S1-induced T cell responses in NHP, including IFNγ-, IL-2-, and IL-4-expressing S- and N-specific T cell responses measured 2 weeks pre-challenge by FluoroSpot assay in COH04S1-2D- and COH04S1-1D-vaccinated NHP and mock-vaccinated and sMVA vector control animals. Bars represent mean values, and lines represent ±SD. Two-way ANOVA followed by Tukey's multiple comparison test was used. Dotted lines indicate the arbitrary positive threshold of 30 spots/10⁶ cells. 0.01<*p<0.05.

FIGS. 9A-9I show BAL viral loads in COH04S1-vaccinated NHP following SARS-CoV-2 challenge. SARS-CoV-2 gRNA (FIGS. 9A-9C) and sgRNA (FIGS. 9D-9F) copies and Median Tissue Culture Infectious Dose (TCID50) infectious virus titers (FIGS. 9G-9I) were measured at the indicated days post-challenge in BAL of COH04S1-2D- and COH04S1-1D-vaccinated NHP and mock-vaccinated and sMVA vector control animals. Bars represent geometric means, and lines represent ±geometric SD. Dotted lines represent lower limit of detection. Two-way ANOVA followed by Sidak's multiple comparison test was used. FIGS. 9C, 9F, and 9I show viral loads by area under the curve (AUC). Violin plots show values ranges with median values (dashed line) and quartiles (dotted line). AUC=0 was indicated as 1. One-way ANOVA followed by Tukey's multiple comparison test was used. 0.01<*p<0.05, 0.001<**p<0.01, 0.0001<***p<0.001, ****p<0.0001.

FIGS. 10A-10I show nasal swab viral loads in COH04S1-vaccinated NHP after SARS-CoV-2 challenge. SARS-CoV-2 gRNA (FIGS. 10A-10C) and sgRNA (FIGS. 10D-10F) copies and TCID50 infectious virus titers (FIGS. 10G-10I) were measured at the indicated days post-challenge in nasal swab samples of COH04S1-2D- and COH04S1-1 D-vaccinated NHP and mock-vaccinated and sMVA vector-vaccinated control animals. Lines represent geometric means+geometric SD. Dotted lines represent lower limit of detection. Two-way ANOVA followed by Sidak's multiple comparison test was used. FIGS. 10C, 10F, and 10I show viral loads by AUC. Violin plots show values ranges with median values (dashed line) and quartiles (dotted line). AUC=0 was indicated as 1. One-way ANOVA followed by Tukey's multiple comparison test was used. 0.01<*p<0.05, 0.001<**p<0.01, 0.0001<***p<0.001, ****p<0.0001.

FIGS. 11A-11F show oral swab viral loads in COH04S1-vaccinated NHP following SARS-CoV-2 challenge. SARS-CoV-2 gRNA (FIGS. 11A-11C) and sgRNA (FIGS. 11D-11F) copies were measured by qPCR at the indicated days post-challenge in oral swab samples of COH04S1-2D- and COH04S1-1 D-vaccinated NHP and mock-vaccinated and sMVA vector control animals. Lines represent geometric means±geometric SD. Dotted lines represent assay lower limit of detection. Two-way ANOVA followed by Sidak's multiple comparison test was used. FIGS. 11C and 11F show viral loads by AUC. Violin plots show values ranges with median values (dashed line) and quartiles (dotted line). AUC=0 was indicated as 1. One-way ANOVA followed by Tukey's multiple comparison test was used. 0.01<*p<0.05, 0.001<**p<0.01, 0.0001<***p<0.001, ****p<0.0001.

FIGS. 12A-12L show SARS-CoV-2 viral loads in lungs and other organs of COH04S1-vaccinated NHP following challenge. Given are SARS-CoV-2 gRNA (FIGS. 12A, 12C, and 12E-12H) and sgRNA (FIGS. 12B, 120, and 121-12L) copies measured by qPCR at days 7 and 21 post-challenge in the lungs and several other organs of necropsied COH04S1-2D- and COH04S1-1 D-vaccinated NHP and mock-vaccinated and sMVA vector control animals. Four lung samples were analyzed for each necropsied NHP (see Table 6 for necropsy schedule). Dotted lines represent assay lower limit of detection. Kruskal-Wallis test followed by Dunn's multiple comparison test was used. In FIGS. 12E-12L, statistical evaluation was not performed because of the limited number of samples in the control groups. 0.01<*p<0.05, 0.001<**p<0.01.

FIGS. 13A-13B show correlative analysis of immunological and virological parameters in COH04S1-vaccinated NHP challenged with SARS-CoV-2. Spearman correlation analysis was performed between the indicated pre-challenge and post-challenge immune responses and post-challenge virological assessments of COH04S1-2D- and COH04S1-1D-vaccinated NHP and mock-vaccinated and sMVA control animals. Post-challenge time points are indicated with “+” before the day number. FIG. 13A shows that Spearman correlation coefficients were calculated and plotted as a matrix. FIG. 13B shows that two-tailed p values were calculated and indicated as 0.01<*p<0.05, 0.001<**p<0.01, 0.0001<***p<0.001, ****p<0.0001. ns=not significant.

FIGS. 14A-14L show SARS-CoV-2-specific post-challenge immune responses in COH04S1-vaccinated NHP. SARS-CoV-2-specific humoral and cellular immune responses were measured at days 3, 7, 15, and 21 post-challenge in COH04S1-2D- and COH04S1-1 D-vaccinated NHP and mock-vaccinated and sMVA vector control animals and compared to pre-challenge immunity assessed in these groups. FIGS. 14A-14F show S, RBD, and N antigen-specific binding antibody titers evaluated by ELISA. FIGS. 14G and 14J show NAb titers measured by PRNT assay against SARS-CoV-2 USA-WA1/2020 reference strain. Lines represent geometric means±geometric SD. Dotted lines indicate lower limit of detection. Values below the lower limit of detection are indicated as half the lower limit of detection. FIGS. 14H-141 and 14K-14L show IFNγ-expressing S- and N-specific T cell responses measured by FluoroSpot assay. Bars represent mean values, and lines represent ±SD. Dotted lines indicate the arbitrary positive threshold of 30 spots/10⁶ cells. Two-way ANOVA followed by Tukey's multiple comparison test was used. Time points with <3 samples/group (d+15 and d+21) were excluded from the statistical evaluation. 0.01<*p<0.05, 0.001<**p<0.01, 0.0001<***p<0.001, ****p<0.0001.

FIGS. 15A-15D show SARS-CoV-2-specific post-challenge cellular IL-4-specific responses in COH04S1-vaccinated NHP. SARS-CoV-2-specific cellular immune responses were measured at days 3, 7, 15, and 21 post-challenge in COH04S1-2D- and COH04S1-1 D-vaccinated NHP and mock-vaccinated and sMVA vector control animals and compared to pre-challenge immunity assessed in these groups. IL-4-expressing S- and N-specific T cell responses were measured by FluoroSpot assay. Bars represent mean values, and lines represent ±SD. Dotted lines indicate the arbitrary positive threshold of 30 spots/10⁶ cells. Two-way ANOVA followed by Tukey's multiple comparison test was used. Time points with <3 samples/group (d+15 and d+21) were excluded from the statistical evaluation. ****p<0.0001.

FIGS. 16A-16B show vaccine-mediated protection in hamsters following sub-lethal challenge with SARS-CoV-2 Delta variant (B.1.617.2). FIG. 16A shows body weight change. Syrian hamsters (N=10) were intramuscularly vaccinated two times in a 28-day interval with COH04S1 or COH04S351. COH04S1 expresses S and N antigen sequences based on the original SARS-CoV-2 Wuhan-Hu-1 isolate, while COH04S351 expresses S and N antigen sequences based on SARS-CoV-2 B.1.351 Beta variant, originally isolated in the Republic of South Africa. Control animals were vaccinated with sMVA control vector. All vaccines were administered at 1×10⁸ plaque-forming units (PFU). Two weeks post-boost, hamsters were challenged intranasally with a high dose (6.5×10⁴ TCID50 per hamster) of SARS-CoV-2 B.1.617.2 Delta variant (isolate USA/PHC658/2021). Body weight was measured daily for 10 days post-challenge. Weight loss is reported as mean±SEM. Two-way ANOVA followed by Tukey's multiple comparison test was used to compare group mean values at each time point. FIG. 16B shows maximal weight loss. Percentage of maximal weight loss is shown in single animals within vaccine and control groups (N=5). Only animals that were sacrificed at endpoint were included. Lines and bars represent median values and 95% Cl, respectively. Peaks of weight loss in each group were compared using one-way ANOVA followed by Tukey's multiple comparison test. 0.01<*p<0.05, 0.001<**p<0.01, 0.0001<***p<0.001, ****p<0.0001.

FIGS. 17A-17B illustrate the study design of comparing the immunogenicity of COH04S1 and COH04S351 in mice. Female Balb/c mice (N=6) were intraperitoneally vaccinated two times in a 21-day interval with COH04S351, COH04S1, a combination of COH04S351/COH04S1 in two amounts, or with the two vaccines used in a heterologous prime/boost schedule. Control animals were vaccinated with sMVA control vector (N=6) or PBS (N=5). Single vaccines were administered at 1×10⁷ PFU. The combination of COH04S351/COH04S1 was administered at either 1×10⁷ PFU (mix2×) or 0.5×10⁷ (mix) PFU/each vaccine. Serum samples were collected at baseline, week 2, and endpoint (week 4), when splenocytes were also collected for immunological analyses. p=prime, b=boost.

FIGS. 18A-18C show vaccine-induced SARS-CoV-2-specific binding antibody endpoint titers. S-, RBD-, and N-specific binding endpoint titers were measured in mouse sera collected 2 weeks post-prime and 1 week post-boost vaccination by ELISA using proteins based on the ancestral SARS-CoV-2 virus (Wuhan).

FIG. 19 shows vaccine-induced binding antibody endpoint titers to VOC. S-specific binding endpoint titers were measured in mouse sera collected 1 week post-boost vaccination by ELISA using S proteins based on the ancestral SARS-CoV-2 virus (Wuhan), beta (B.1.351), and gamma (P.1) VOC.

FIG. 20 shows IFNγ cellular responses to ancestral and VOC S and N peptide libraries. S and N peptide libraries based on the sequences from ancestral Wuhan SARS-CoV-2 and beta and gamma VOC were used to stimulate splenocytes of mice vaccinated with COH04S351, COH04S1, and combinations of the two vaccines. ELISPOT was used to quantify the IFNγ-specific cellular response to the antigens.

FIGS. 21A-21B illustrate the study design of comparing the immunogenicity of COH04S1 and C170 in mice. Female Balb/c mice (N=6) were intraperitoneally vaccinated two times in a 21-day interval with C170 (multi-antigenic SARS-CoV-2 sMVA vaccine encoding for S and N sequences from the gamma P.1 VOC originally isolated in Brazil), COH04S1, a combination of C170/COH04S1 in two amounts, or with the two vaccines used in a heterologous prime/boost schedule. Control animals were vaccinated with sMVA control vector or PBS (N=6). Single vaccines were administered at 1×10⁷ PFU. The combination of C170/COH04S1 was administered at either 1×10⁷ PFU (mix2×) or 0.5×10⁷ (mix) PFU/each vaccine. Serum samples were collected at baseline, week 2, and endpoint (week 4), when splenocytes were also collected for immunological analyses. p=prime, b=boost.

FIG. 22 shows vaccine-induced SARS-CoV-2-specific binding antibody endpoint titers. S-, RBD-, and N-specific binding endpoint titers were measured in mouse sera collected 2 weeks post-prime and 1 week post-boost vaccination by ELISA using proteins based on the ancestral SARS-CoV-2 virus (Wuhan).

FIG. 23 shows vaccine-induced binding antibody endpoint titers to VOC. S-specific binding endpoint titers were measured in mouse sera collected 1 week post-boost vaccination by ELISA using S proteins based on the ancestral SARS-CoV-2 virus (Wuhan), beta (B.1.351), and gamma (P.1) VOC.

FIG. 24 shows NAb response to ancestral Wuhan SARS-CoV-2 and VOC. Lentiviral spike pseudoviruses based on the ancestral Wuhan strain with D614G substitution, Alpha, Beta, Gamma, and Delta VOC were used in a neutralization assay to measure SARS-CoV-2-specific NAb in pooled post-boost mouse sera. Shown are the serum dilutions to inhibit 50% of pseudovirus entry into HEK293T-ACE2-susceptible cells (IC50).

FIG. 25 shows IFNγ cellular responses to ancestral Wuhan and VOC S and N peptide libraries. S and N peptide libraries based on the sequences from ancestral Wuhan SARS-CoV-2 and beta and gamma VOC were used to stimulate splenocytes of mice vaccinated with C170, COH04S1, and combinations of the two vaccines. ELISPOT was used to quantify the IFNγ-specific cellular response to the antigens.

FIGS. 26A-26B illustrate the study design of comparing the immunogenicity of COH04S1 and COH04S351 in hamsters. Syrian hamsters (N=30) were intramuscularly vaccinated two times in a 28-day interval with COH04S1 or COH04S351. Control animals (N=30) were vaccinated with sMVA control vector. All vaccines were administered intramuscularly at 1×10⁸ PFU. Two weeks post-boost, hamsters were challenged intranasally with a high dose of SARS-CoV-2 Washington strain (WAS-Calu-3), B.1.351 Beta, or B.1.617.2 Delta variant (isolate USA/PHC658/2021). Body weight was measured daily for 10 days post-challenge. Five hamsters in each group were sacrificed at days 3 and 10 post-challenge for organ collection.

FIGS. 27A-27F show vaccine-induced SARS-CoV-2-specific IgG responses following vaccination with COH04S1 and COH04S351. FIG. 27A shows SARS-CoV-2-specific binding antibody endpoint titers. S-, RBD-, and N-specific IgG titers to ancestral SARS-CoV-2 were measured in serum samples of vaccine and control groups at day 14 (d14) post-prime vaccination via ELISA. FIG. 27B shows variant-specific antibody endpoint titers. Serum IgG binding titers to Beta, Detal, and Omicron-specific S antigens were measured in serum samples of vaccine and control groups at d14 post-prime vaccination via ELISA. FIG. 27C shows Th1/Th2 response. S-, RBD-, and N-specific binding antibodies of the IgG2/3 and IgG1 isotype were measured in serum samples of vaccine and control groups at d14 post-prime vaccination, and IgG2/3/IgG1 ratios were calculated. A ratio >1 is considered a Th1-biased immune response. Dotted lines indicate lower limit of detection.

FIGS. 27D-27F show NAb titers. NAb titers were measured in serum samples of vaccine and control groups at day 42 (d42) post second vaccination via PRNT assay against ancestral SARS-CoV-2 (FIG. 27D) and Beta (FIG. 27E) and Delta (FIG. 27F) variants. Dotted lines indicate lower and upper limit of detection, respectively. Values below the lower limit of detection (ID50=20 in FIGS. 270, 27F, ID50=60 in FIG. 27E) are indicated as half the lower limit of detection, values above the upper limit of detection (ID50=14,580) are indicated as twice the upper limit of detection. Data are presented as geometric mean values±geometric SD. Two-way ANOVA with Tukey's multiple comparison test was used. 0.01<*p<0.05, 0.001<**p<0.01, 0.0001<***p<0.001, ****p<0.0001.

FIGS. 28A-28F show that COH04S1 and COH04S351 protected hamsters from lower respiratory tract infection following challenge with SARS-CoV-2 ancestral virus and Beta and Delta variants. SARS-CoV-2 sgRNA copies were quantified by qPCR in lung tissue (FIGS. 28A-28C) and nasal turbinates (FIGS. 28D-28F) of COH04S1 and COH04S351 vaccine and control groups at days 3 and 10 following challenge with ancestral SARS-CoV-2 (FIGS. 28A, 28D) or SARS-COV-2 Beta (FIGS. 28B, 28E) and Delta (FIGS. 28C, 28F) variants. Lines indicate median sgRNA copies. One-way ANOVA followed by Tukey's multiple comparison test was used. 0.01<*p<0.05, 0.001<**p<0.01, 0.0001<***p<0.001. FIGS. 28G-280 show that COH04S1 and COH04S351 protect hamsters from lung pathology following challenge with SARS-CoV-2 ancestral virus and Beta and Delta variants. Hematoxylin/eosin-stained lung sections of COH04S1- and COH04S351-vaccinated hamsters and control animals at days 3 and 10 following challenge with ancestral SARS-CoV-2 (FIGS. 28G, 28J, 28M) or SARS-CoV-2 Beta (FIGS. 28H, 28K, 28N) and Delta (FIGS. 28I, 28L, 28O) variants were evaluated by a board-certified pathologist and microscopic findings were graded based on severity on a scale from one to five (Table 5). FIGS. 28G-28I show the cumulative pathology score of all histopathologic findings in each group. FIGS. 28J-28L show grading of bronchioalveolar hyperplasia disease severity in each group. FIGS. 28M-28O show the severity of lung inflammatory microscopic findings. Lines indicate median values. Two-way ANOVA followed by Tukey's multiple comparison test was used. 0.01<*p<0.05, 0.001<**p<0.01, 0.0001<***p<0.001, ****p<0.0001.

FIGS. 29A-29F show vaccine-mediated protection in hamsters following sub-lethal challenge with SARS-CoV-2 USA/WA/1, Beta (B.1.351), and Delta variant (B.1.617.2). Syrian hamsters (N=10) were intramuscularly vaccinated two times in a 28-day interval with COH04S1 or COH04S351. Control animals were vaccinated with sMVA control vector. All vaccines were administered at 1×10⁸ PFU. FIGS. 29A, 29C, and 29E show body weight change. Two weeks post-boost, hamsters were challenged intranasally with SARS-CoV-2 Washington strain (FIG. 29A, WAS-Calu-3), B.1.351 Beta (FIG. 29C), or B.1.617.2 Delta variant (FIG. 29E, isolate USA/PHC658/2021). Body weight was measured daily for 10 days post-challenge. Five hamsters in each group were sacrificed at day 5. Weight loss is reported as mean±SEM. Two-way ANOVA followed by Tukey's multiple comparison test was used to compare group mean values at each time point. FIGS. 29B, 29D, and 29F show maximal weight loss. Percentage of maximal weight loss is shown in single animals of vaccine and control groups (N=5). Only animals that were sacrificed at endpoint were included. Lines and bars represent median values and 95% Cl, respectively. Peaks of weight loss in each group were compared using one-way ANOVA followed by Tukey's multiple comparison test. 0.01<*p<0.05, 0.001<**p<0.01, 0.0001<***p<0.001, ****p<0.0001.

FIGS. 30A-30E show that COH04S1 and Omicron BA.1 and Beta sequence-modified vaccines elicit strain-specific antibody responses against SARS-CoV-2 ancestral virus and VOC. FIG. 30A shows that the three vaccine constructs, COH04S1, COH04S529, and COH04S351, are sMVA-vectored COVID-19 vaccines co-expressing S and N antigens based on the Wuhan-Hu-1 reference strain or Omicron BA.1 or Beta variants, respectively. The antigen sequences were inserted into the MVA sites Del2 and Del3 as indicated. FIG. 30B shows study design. Hamsters were vaccinated twice with COH04S1 (n=20), COH04S351 (n=20), or COH04S529 (n=20) by IM route as indicated. Hamsters vaccinated with empty sMVA vector (n=10) or unvaccinated hamsters were used as controls (n=10). Blood samples were collected at days 14 and 42 after the first and second vaccinations. COH04S1-, COH04S529-, and COH04S351-vaccinated hamsters were challenged intranasally at day 42 with Omicron subvariants BA.1 or BA.2.12.1 (n=10/group). sMVA control animals were challenged with BA.1. Unvaccinated controls were challenged with BA.2.12.1. Post-challenge body weight changes were recorded daily for 8 days. Lung tissue and nasal turbinates for viral load measurements and lung histopathology were collected at days 4 and 8 post-challenge (n=5/group/time point). FIGS. 30C-30D show IgG endpoint titers. S-specific (FIG. 30C) and N-specific (FIG. 30D) binding antibody titers to ancestral virus, Beta, and Omicron subvariants BA.1, BA.2, BA.4, and BA.5 were measured in serum samples of vaccine and control groups at day 14 (d14) post-prime vaccination via ELISA. Dotted lines indicate lower limit of detection. FIG. 30E shows NAb titers. NAb titers were measured in serum samples of vaccine and control groups at day 42 (d42) post-second vaccination via PRNT assay against ancestral SARS-CoV-2 (WA/1) and Omicron BA.1. Dotted lines indicate lower and upper limit of detection. Values below the lower limit of detection are indicated as half the lower limit of detection. Data are presented as box plots extending from 25th to 75th percentiles, with lines indicating medians, and whiskers going from minimum to maximum values. Two-way ANOVA with Tukey's multiple comparison test was used in FIGS. 30C-30D. Kruskal-Wallis test was used in e. 0.01<*p<0.05, 0.001<**p<0.01, 0.0001<***p<0.001, ****p<0.0001.

FIGS. 31A-31B show that COH04S1 and Omicron BA.1- and Beta-modified vaccines protect hamsters from weight loss following challenge with Omicron BA.1 or BA.2.12.1. Body weight of COH04S1-, COH04S351-, and COH04S351-vaccinated animals was measured daily for 8 days following challenge with Omicron variants BA.1 (FIG. 31A) and BA.2.12.1 (FIG. 31B). Hamsters vaccinated with empty sMVA vector or unvaccinated hamsters were used as controls. Weight loss compared to day 0 is reported as mean±SEM. Two-way ANOVA followed by Tukey's multiple comparison test was used to compare group mean values at each time point. 0.01<*p<0.05, 0.001<**p<0.01, 0.0001<***p<0.001, ****p<0.0001. Black asterisks indicate time of sacrifice (n=5/group).

FIGS. 32A-32D show that COH04S1 and Omicron BA.1- and Beta-modified vaccines protect hamsters from lower respiratory tract infection following virus challenge with Omicron BA.1 or BA.2.12.1. SARS-CoV-2 gRNA (FIGS. 32A-32B) and sgRNA (FIGS. 32C-32D) copies were quantified by qPCR in lung tissue of COH04S1, COH04S351, and COH04S529 vaccine and control groups at days 4 and 8 following challenge with Omicron subvariants BA.1 (FIGS. 32A, 32C) and BA.2.12.1 (FIGS. 32B, 32D). Lines indicate median RNA copies. Two-way ANOVA followed by Tukey's multiple comparison test was used. **=0.01<p<0.001, ****=p<0.0001.

FIGS. 33A-33D show the efficacy of COH04S1 and Omicron BA.1- and Beta-modified vaccines in protecting hamsters from upper respiratory tract infection following challenge with Omicron BA.1 or BA.2.12.1. SARS-CoV-2 gRNA (FIGS. 33A-33B) and sgRNA (FIGS. 33C-33D) copies were quantified by qPCR in nasal turbinates of COH04S1, COH04S529, and COH04S351 vaccine and control groups at days 4 and 8 following challenge with Omicron subvariants BA.1 (FIGS. 33A, 33C) and BA.2.12.1 (FIGS. 33B, 33D). Lines indicate median RNA copies. One-way ANOVA followed by Tukey's multiple comparison test was used. 0.01<*p<0.05.

FIGS. 34A-34F show that COH04S1 and Omicron BA.1- and Beta-modified vaccines protect hamsters from lung pathology following challenge with Omicron BA.1 or BA.2.12.1. Hematoxylin/eosin-stained lung sections of COH04S1-, COH04S529-, and COH04S351-vaccinated hamsters and control animals at days 4 and 8 following challenge with SARS-CoV-2 BA.1 (FIGS. 34A-34C) or BA.2.12.1 (FIGS. 34D-34F) variants were evaluated by a board-certified pathologist, and microscopic findings were graded based on severity on a scale from 1 to 5 (Table 5). Cumulative pathology score of all histopathologic findings (FIGS. 34A, 34D), grading of bronchioalveolar hyperplasia disease severity (FIGS. 34B, 34E), and severity of lung inflammatory microscopic findings (FIGS. 34C, 34F) are shown. Lines indicate median values. Two-way ANOVA followed by Tukey's multiple comparison test was used. *=0.05<p<0.01, **=0.01<p<0.001, ***=0.001<p<0.0001, ****=p<0.0001.

FIGS. 35A-35B show histopathological findings in lungs of COH04S1-, COH04S529-, and COH04S351-vaccinated hamsters following virus challenge with Omicron BA.1 and BA.2.12.1. Shown are representative images (1.25× and 10× magnification) of histopathological findings in hematoxylin/eosin-stained lung sections of COH04S1-, COH04S529-, and COH04S351-vaccinated hamsters and sMVA or unvaccinated control animals at day 8 post-challenge with SARS-CoV-2 BA.1 (FIG. 35A) or BA.2.12.1 (FIG. 35B) variants. Control animals show extensive areas with deeply basophilic and concentrated findings affecting the majority of the lung. Darker areas in COH04S351- and COH04S529-vaccinated animals (FIG. 35A) are considered insufficiently inflated lungs and not SARS-CoV-2-related. Open airways were observed in COH04S1-, COH04S529-, and COH04S351-vaccinated hamsters. Scale bar=800 μm for 1.25× images. Scale bar=100 μm for 10× images.

FIGS. 36A-36C show sMVA construction. FIG. 36A is a schematic of an MVA genome. The MVA genome is about 178 kbp in length and contains about 9.6 kbp inverted terminal repeat (ITR) sequences. FIG. 36B shows the three sMVA fragments, F1, F2, and F3. The three subgenomic sMVA fragments (F1-F3) comprise about 60 kbp of the left, central, and right parts of the MVA genome as indicated. sMVA F1/F2 and F2/F3 share about 3 kbp overlapping homologous sequences for recombination (dotted crossed lines). Approximate genome positions of commonly used MVA insertion (Del2, IGR69/70, Del3) are indicated. FIG. 36C shows terminal concatemer resolution-hairpin loop-concatemer resolution (CR/HL/CR) sequences. Each of the sMVA fragments contains at both ends a sequence composition comprising a duplex copy of the MVA terminal HL flanked by CR sequences. BAC=bacterial artificial chromosome vector.

FIG. 37 shows the DNA sequence of an sMVA backbone with possible locations of insertion sites Del2, IGR69/70, and Del3, according to some embodiments.

FIG. 38 shows the DNA sequence of an F1 nucleotide fragment that includes a portion of the full MVA backbone sequence flanked by an MVA CR/HL/CR sequence with insertion sites, according to some embodiments.

FIG. 39 shows the DNA sequence of an F2 nucleotide fragment that includes a portion of the full MVA backbone sequence flanked by an MVA CR/HL/CR sequence with insertion sites, according to some embodiments.

FIG. 40 shows the DNA sequence of an F3 nucleotide fragment that includes a portion of the full MVA backbone sequence flanked by an MVA CR/HL/CR sequence with insertion sites, according to some embodiments.

### DETAILED DESCRIPTION

Vaccines and/or Immunogenic Compositions

In some aspects, provided are vaccines and/or immunogenic compositions and the use thereof in preventing a coronavirus infection, or preventing, treating, or reducing the severity of COVID-19 caused by the coronavirus infection in a subject, including, for example, a multi-antigenic sMVA-CoV-2 vaccine using the highly versatile synthetic vaccine platform based on a synthetic modified vaccinia Ankara (sMVA) backbone. MVA is a highly attenuated poxvirus vector and is widely used to develop vaccines for infectious diseases and cancer. There is a long history of safety, efficacy, and long-term protection in humans. Nucleic acid sequences encoding one or more antigens or epitopes of interest, such as the spike (S) protein and/or the nucleocapsid (N) protein of SARS-CoV-2 or fragments thereof, can be cloned into the sMVA backbone to form an sMVA vaccine. In some embodiments, the composition comprises a recombinant sMVA vector comprising, expressing, or capable of expressing one or more heterologous DNA sequences encoding the S protein and the N protein. The sMVA vectors used in accordance with the embodiments disclosed herein include a synthetic MVA genome that serves as a viral backbone (referred to herein as the sMVA backbone or sMVA genome backbone) and one or more heterologous nucleotide sequences that encode (i) a SARS-CoV-2 S protein (or variant, mutant, and/or immunogenic fragment thereof) and (ii) a SARS-CoV-2 N protein (or variant, mutant, and/or immunogenic fragment thereof). The terms “S protein” and “S antigen” are used interchangeably herein, and the terms “N protein” and “N antigen” are used interchangeably herein.

MVA is derived from its parental strain, chorioallantois vaccinia Ankara (CVA), by 570 passages on chicken embryo fibroblasts (CEF). As a result of the attenuation process, MVA has acquired six major genome deletions (Del1-6) as well as multiple shorter deletions, insertions, and point mutations, leading to gene fragmentation, truncation, short internal deletions, and amino acid substitutions. MVA has severely restricted host cell tropism, allowing productive assembly only in avian cells, e.g., CEF and baby hamster kidney (BHK) cells, whereas in human and most other mammalian cells, MVA assembly is abortive due to a late block in virus assembly. Although non-pathogenic and highly attenuated, MVA maintains excellent immunogenicity as demonstrated in various animal models and humans. In the late phase of the smallpox eradication campaign, MVA was used as a priming vector for the replication-competent vaccinia-based vaccine in over 120,000 individuals in Germany and no adverse events were reported. In the past decades, MVA has been developed as a stand-alone smallpox vaccine and is currently pursued by the United States government as a safer alternative to replace the existing vaccinia-based vaccine stocks as a preventative countermeasure in case of a smallpox outbreak. The U.S. Food and Drug Administration (FDA) approved MVA, under the trade name Jynneos (Bavarian Nordic) on Sep. 24, 2019, to prevent both smallpox and monkeypox. Previously, a similar MVA vaccine using the trade name Imvamune was approved in Europe as a smallpox vaccine. Almost all organizations that we are aware of which currently use MVA vectors or derivatives thereof are licensed or owned by academic, commercial, or governmental entities, which greatly restricts their use to commercially develop MVA-based vaccine vectors. A fully synthetic version of MVA from circularized or linear synthetic DNA fragments is produced and disclosed in PCT application No. PCT/US21/16247, the content of which is incorporated by reference in its entirety. The sMVA or the recombinant sMVA can be used as a vaccine for preventing and treating various conditions such as coronavirus infections and associated diseases.

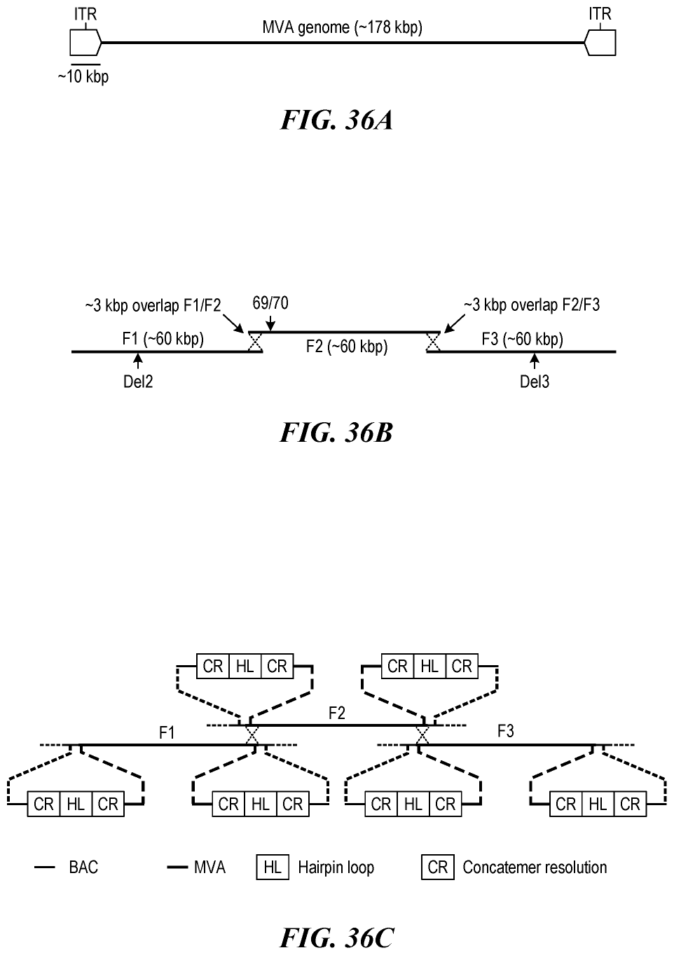

In some embodiments, the sMVA backbone includes an internal unique region (UR) of a parental MVA genome flanked on each side by at least a portion of an inverted terminal repeat (ITR) region of the parental MVA genome (FIG. 36A). The parental genome used to generate the sMVA backbone may be any known MVA strain or variant thereof including, but not limited to, MVA strain Acambis (Accession #AY603355) or MVA strain Antoine (NCBI Accession #U94848). In some embodiments, the sMVA backbone is identical to the corresponding full-length parental MVA genome sequence. In other embodiments, the sMVA backbone is identical or substantially identical to the corresponding full-length parental MVA genome sequence, but includes one or more unintentional mutations originating from chemical synthesis, cloning, propagation, or other errors resulting from the production of the synthetically made virus. For example, in some embodiments, the sMVA backbone has a single T to A nucleotide alteration located in the IGR at three base pairs downstream of open reading frame (ORF) 021 L. In some embodiments, the sMVA backbone has at least 90% sequence identity, at least 91% sequence identity, at least 92% sequence identity, at least 93% sequence identity, at least 94% sequence identity, at least 95% sequence identity, at least 96% sequence identity, at least 97% sequence identity, at least 98% sequence identity, or at least 99% sequence identity to the corresponding full-length parental MVA genome sequence.

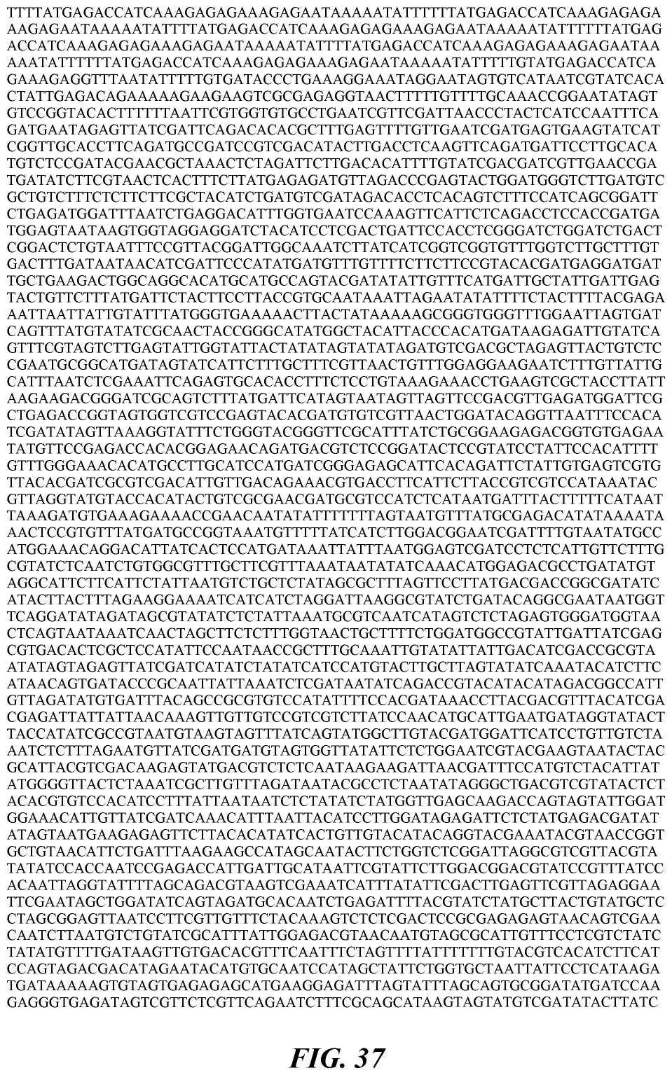

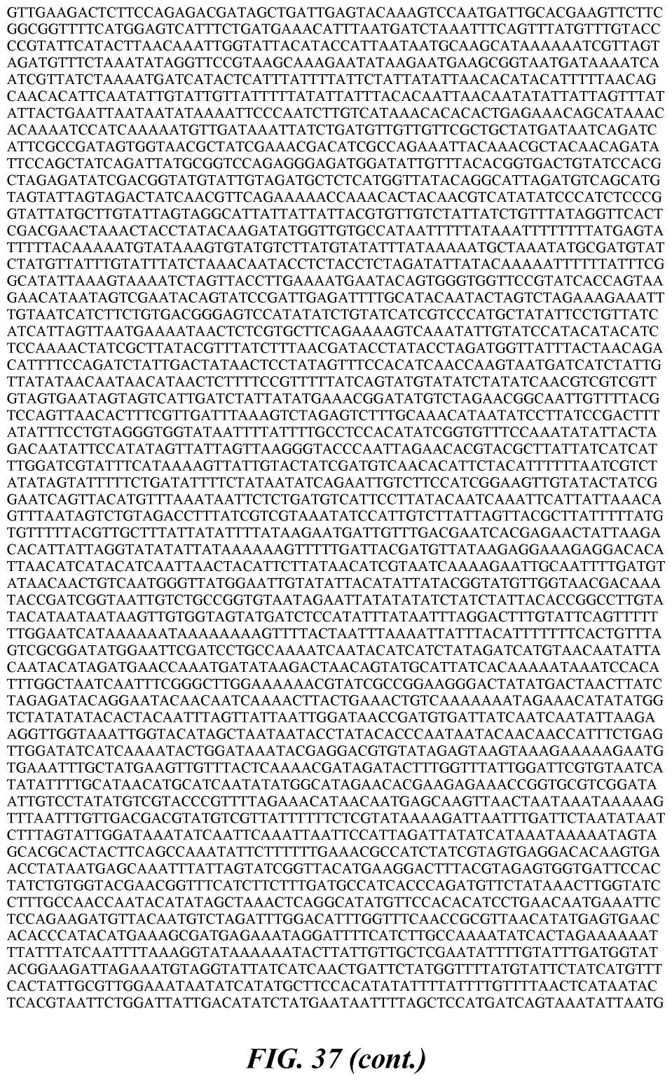

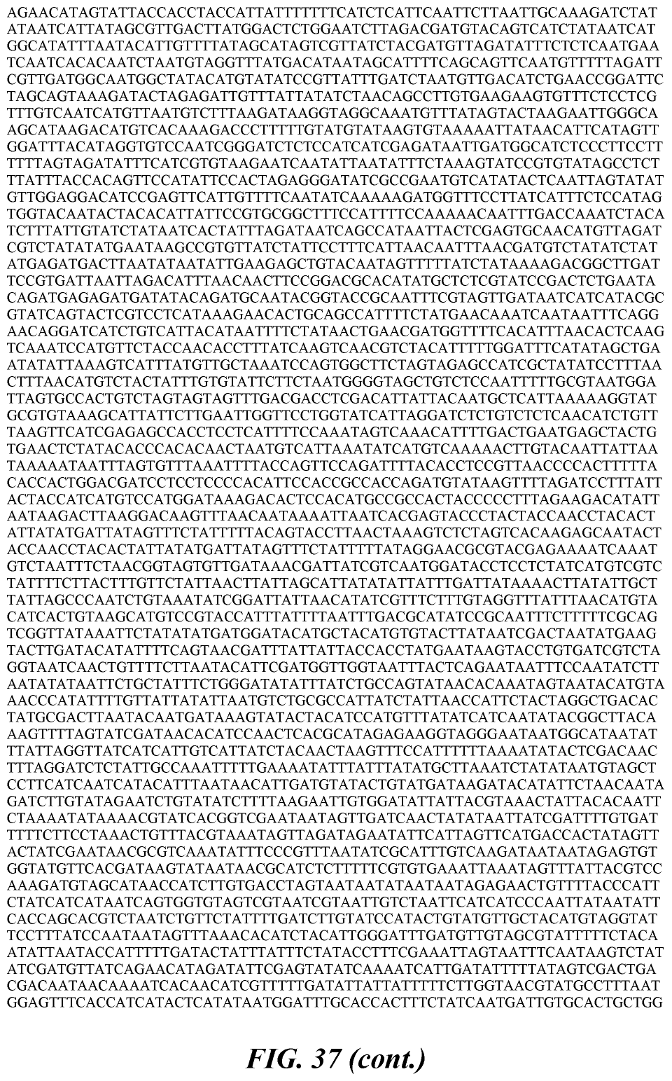

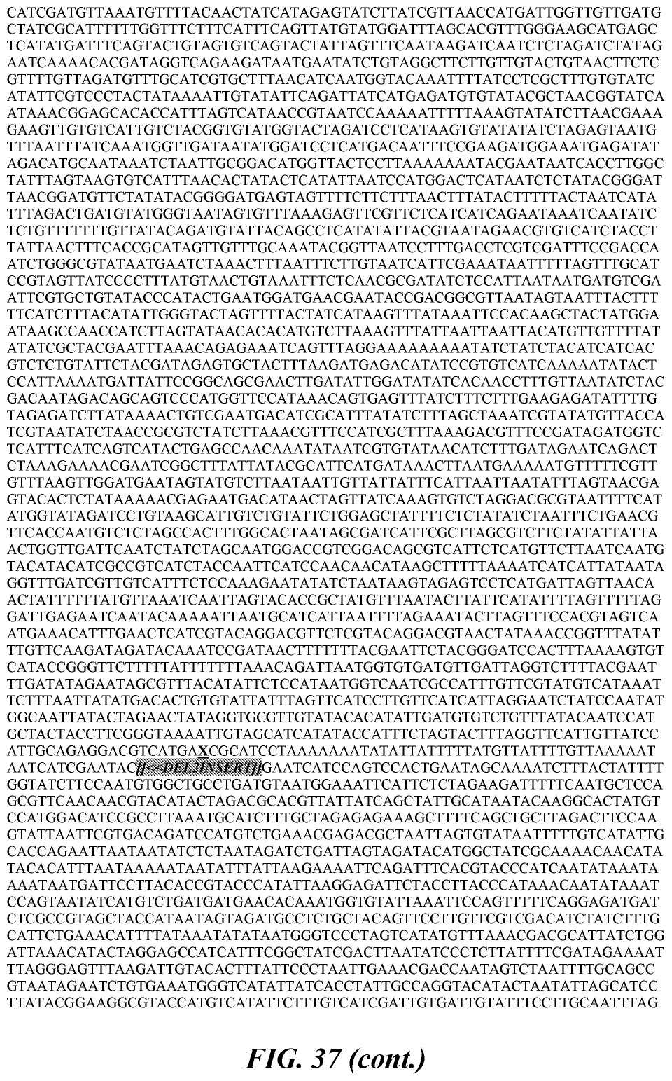

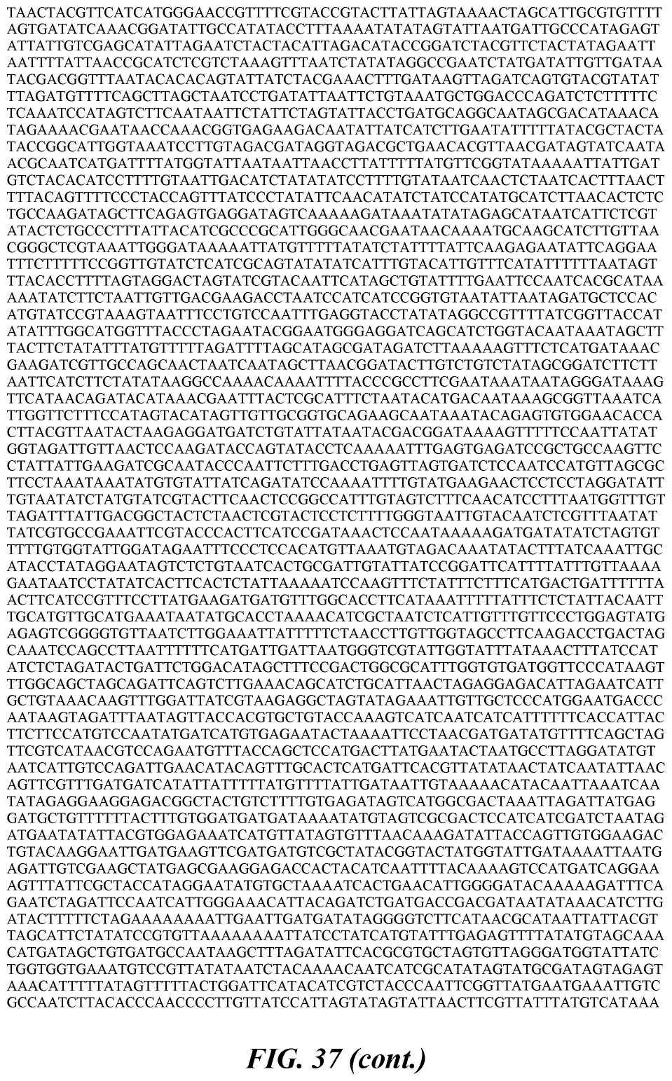

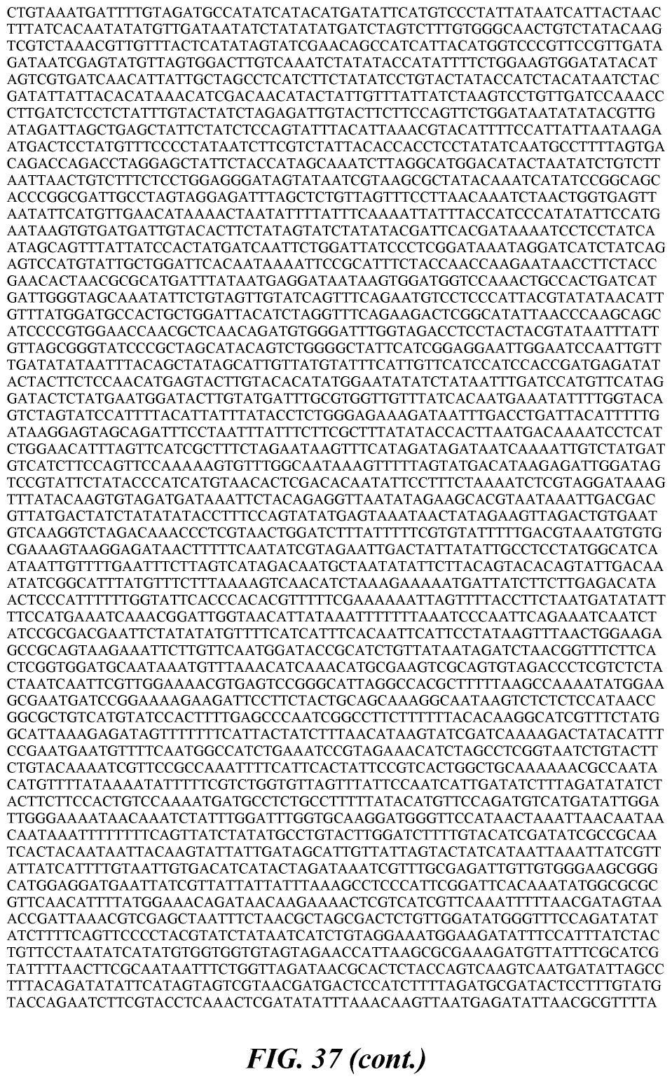

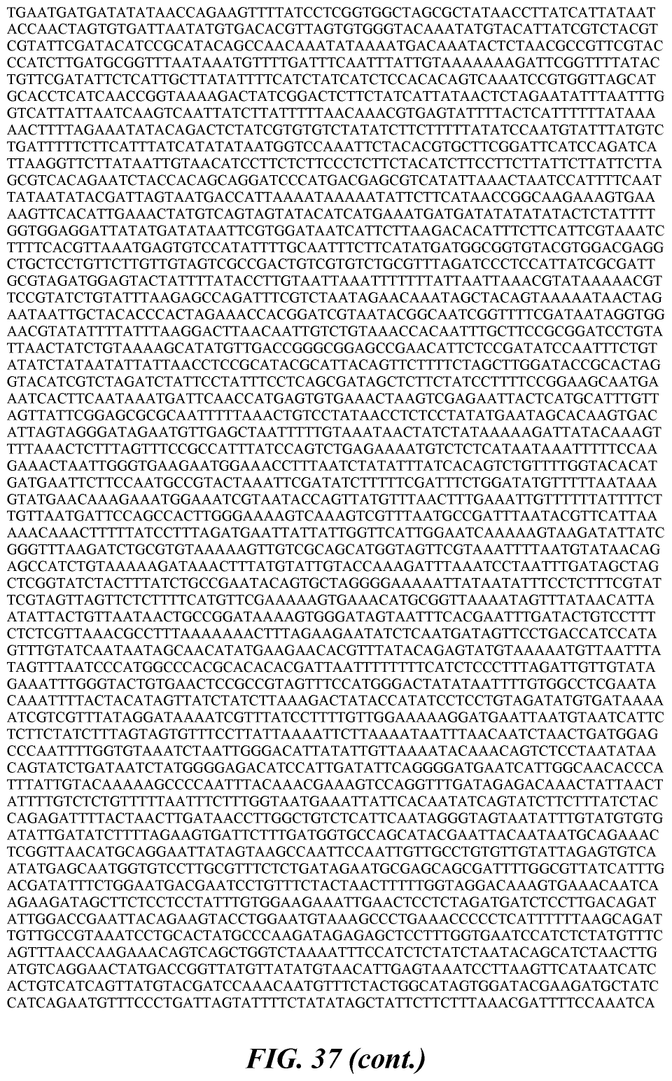

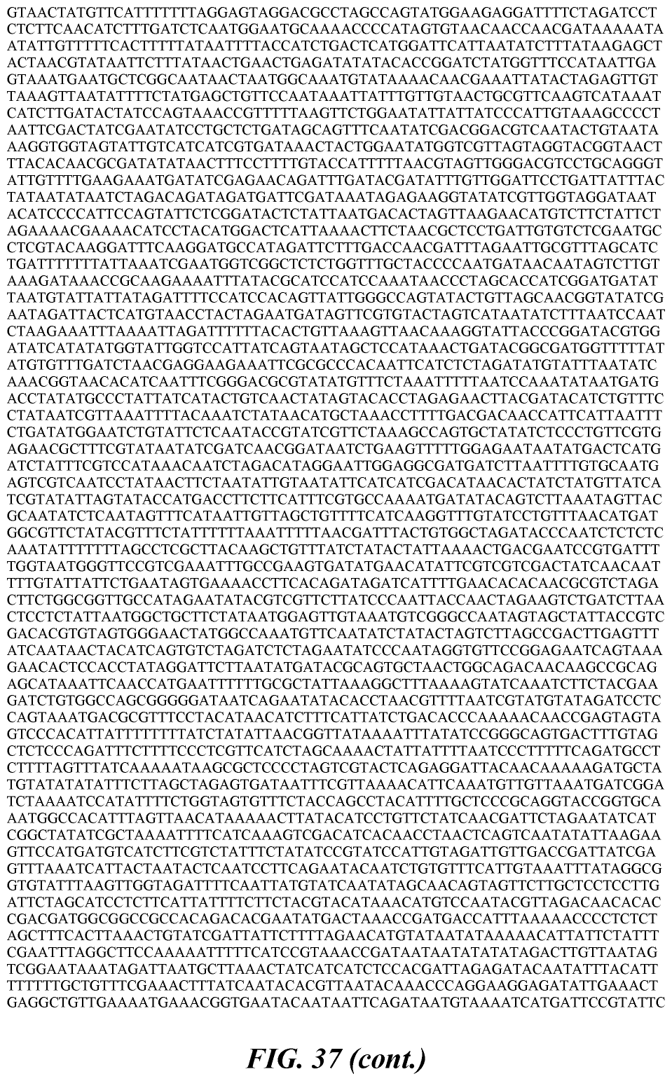

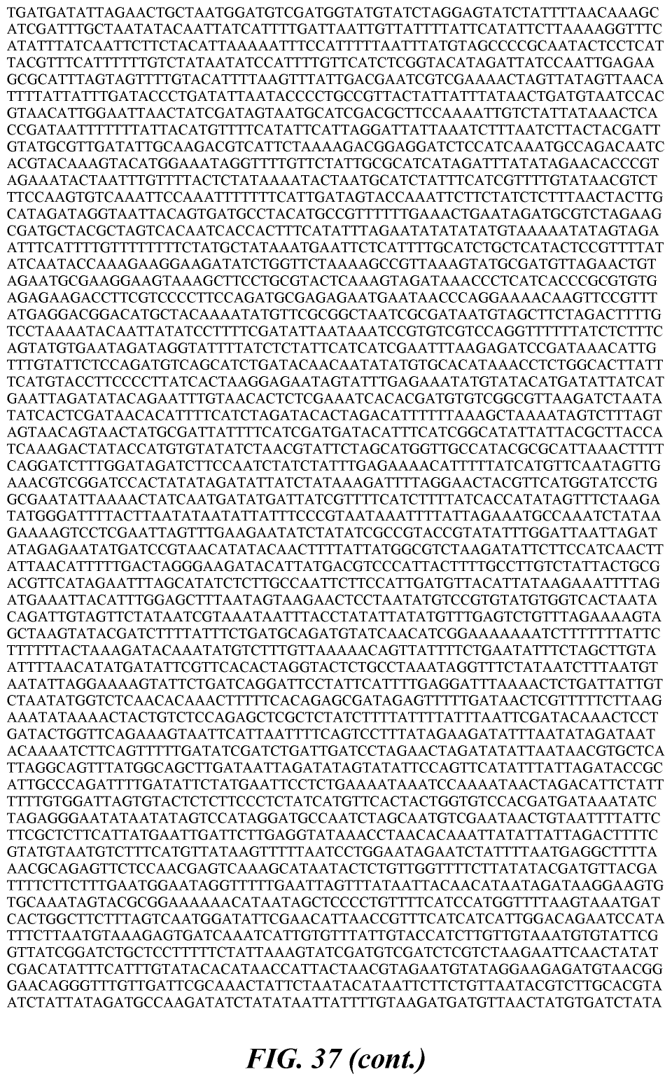

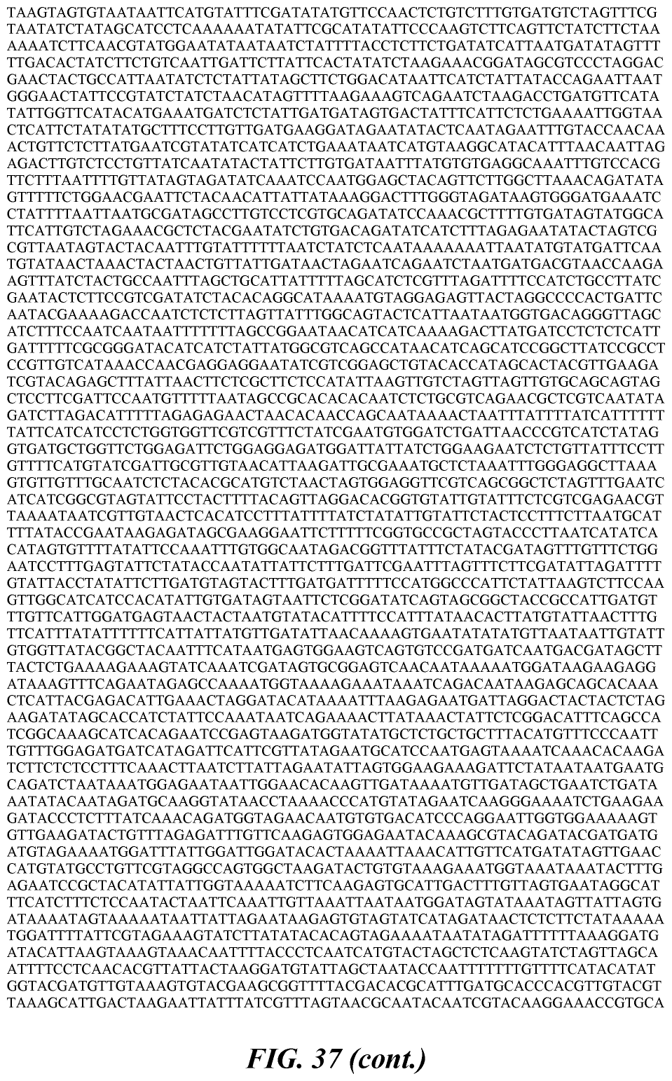

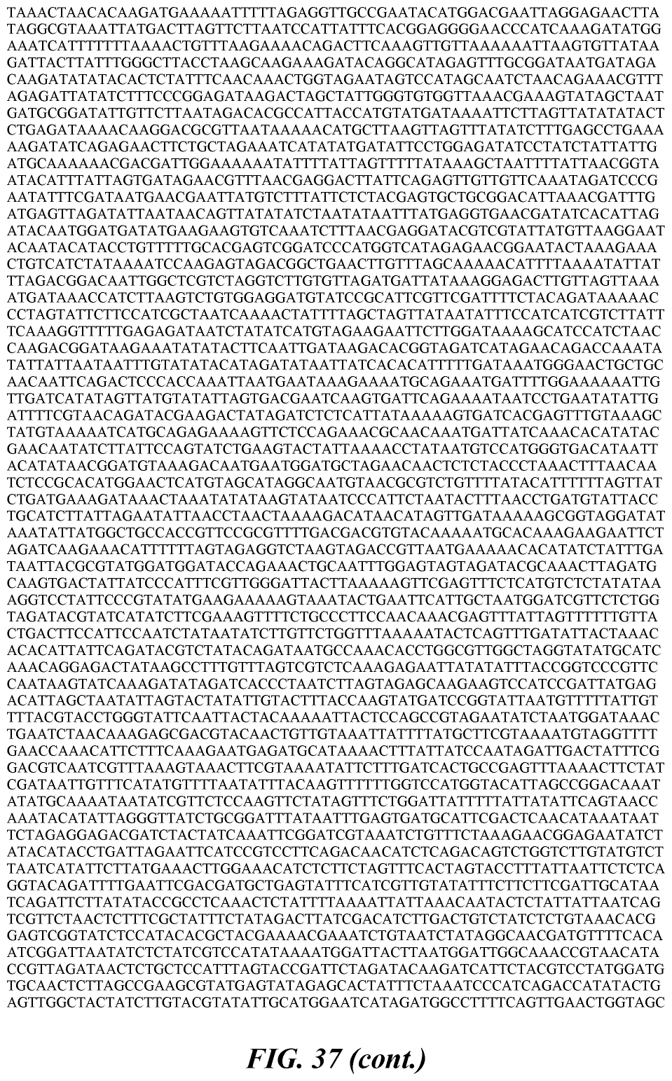

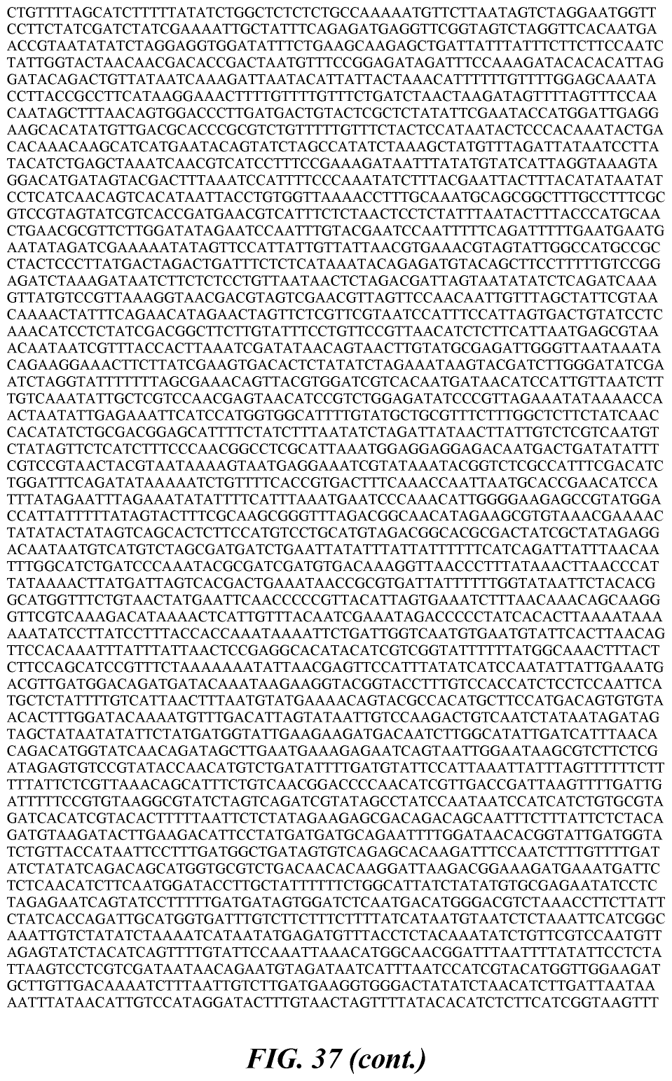

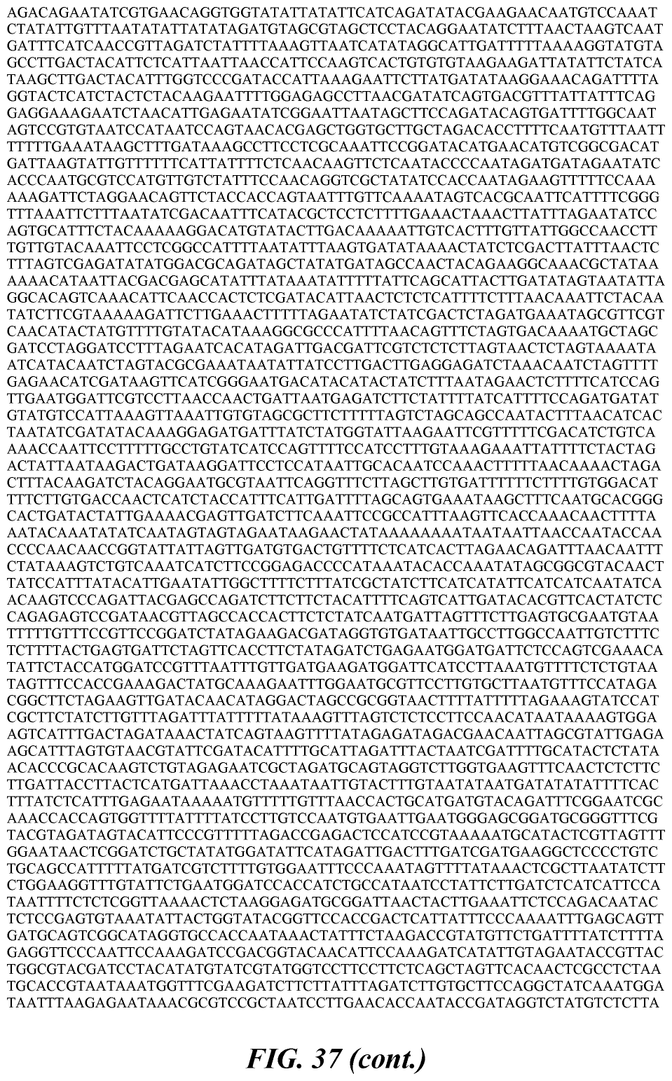

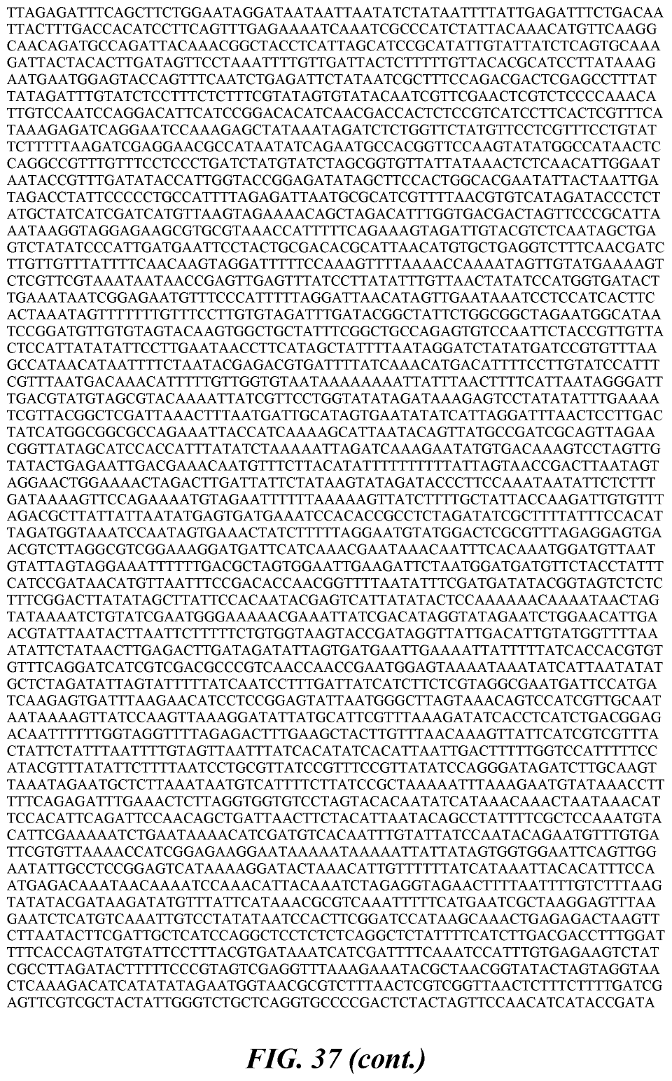

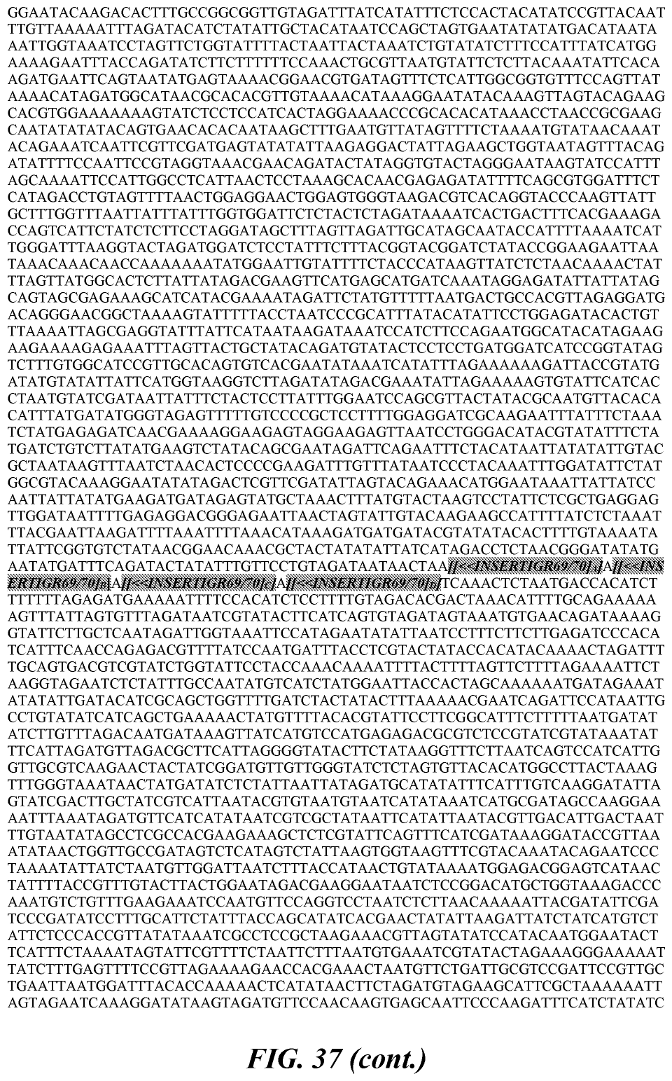

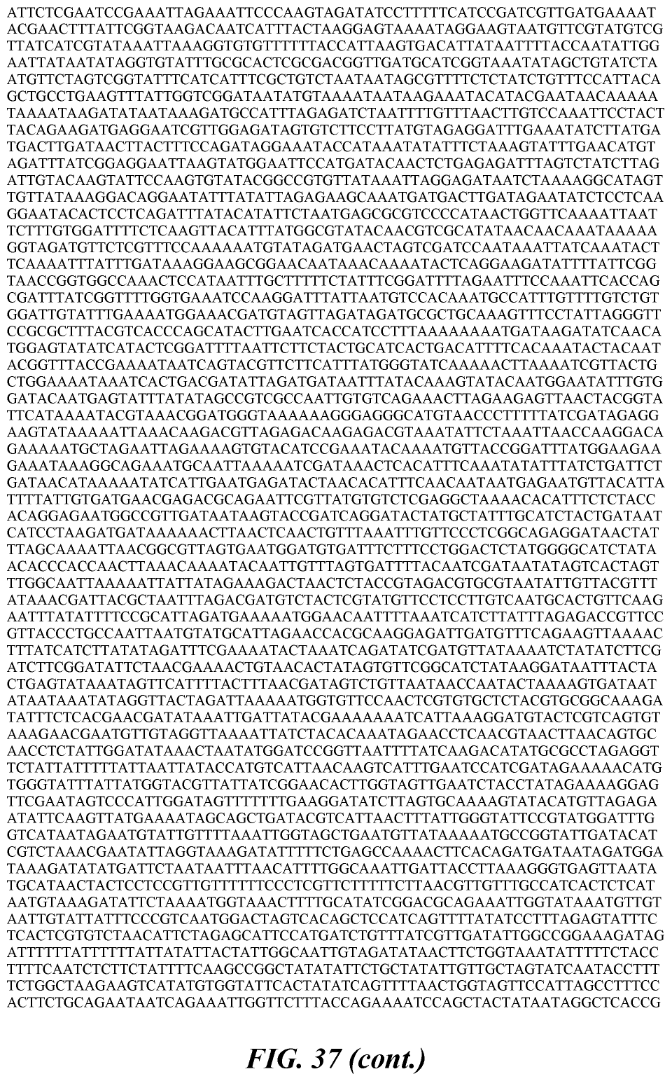

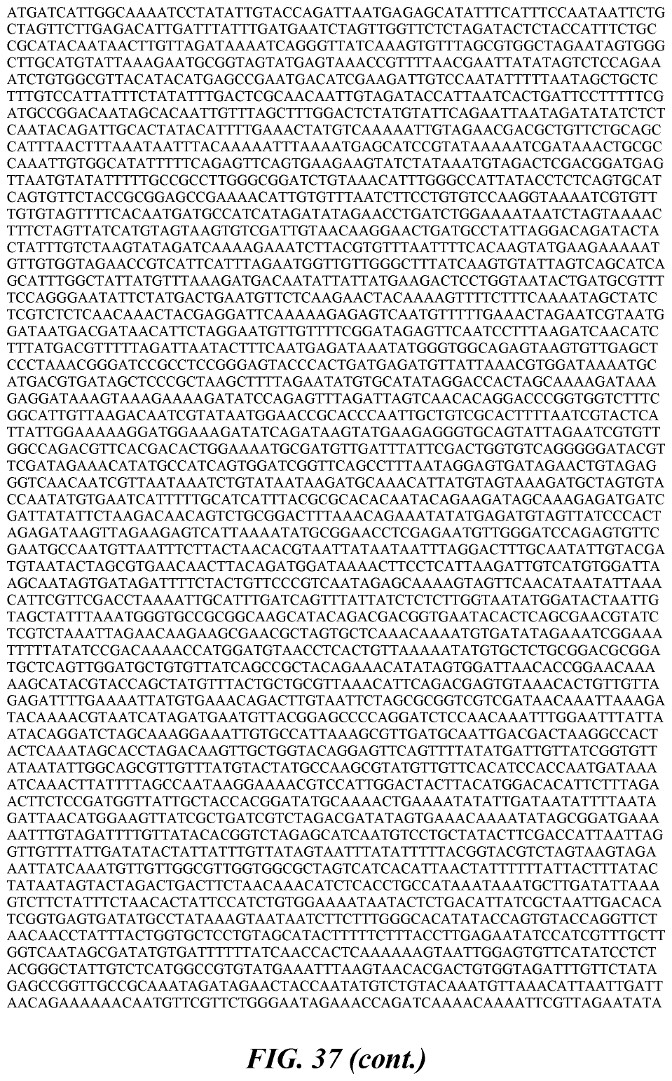

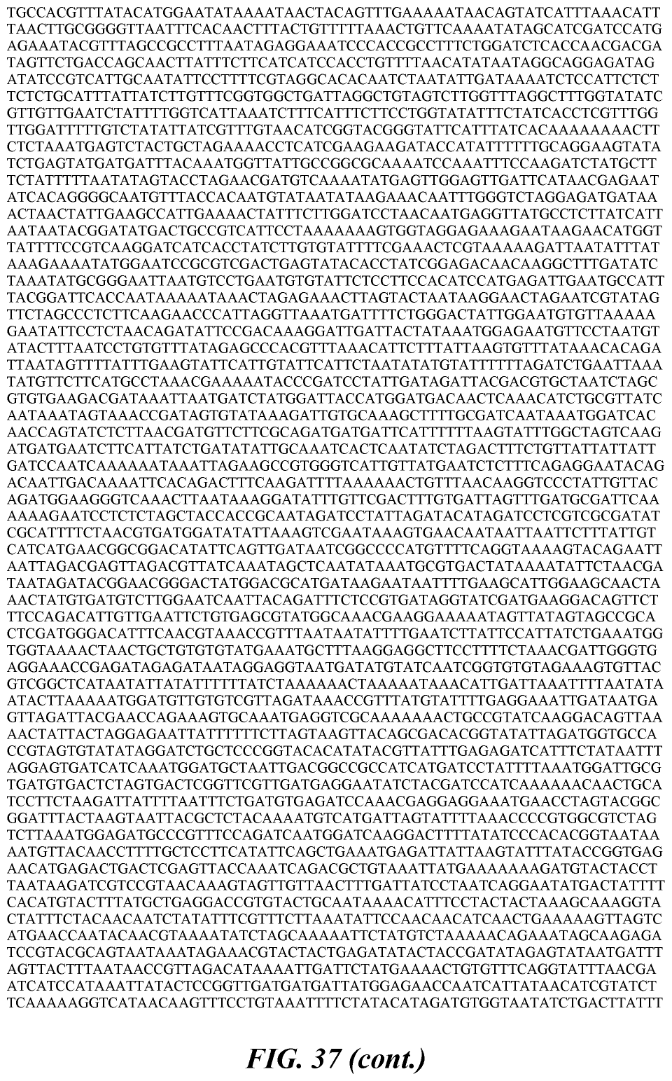

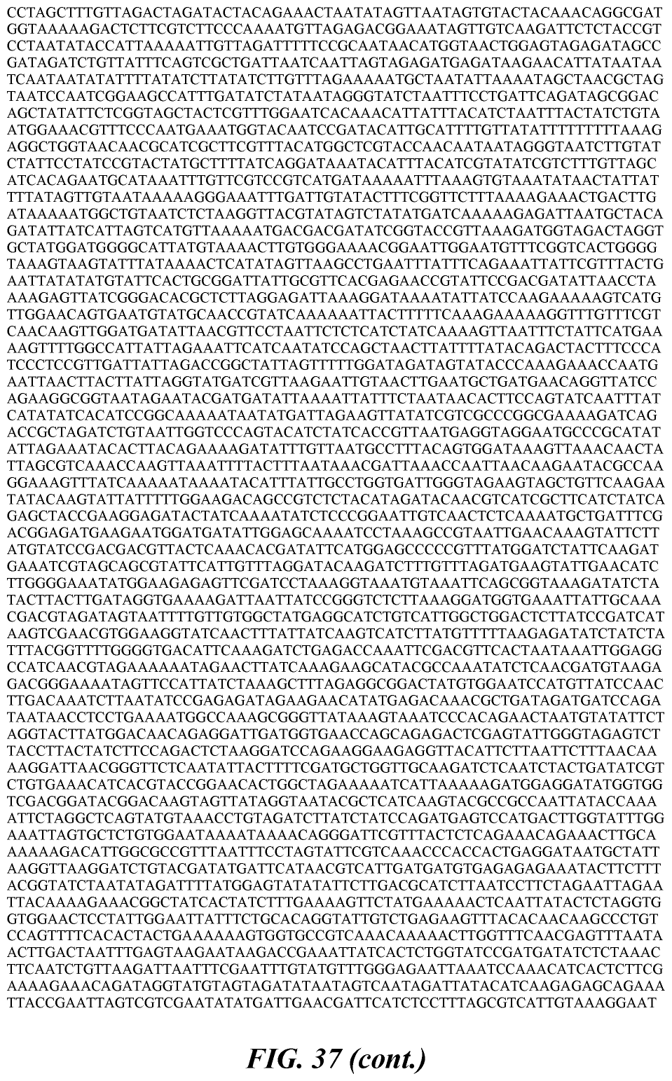

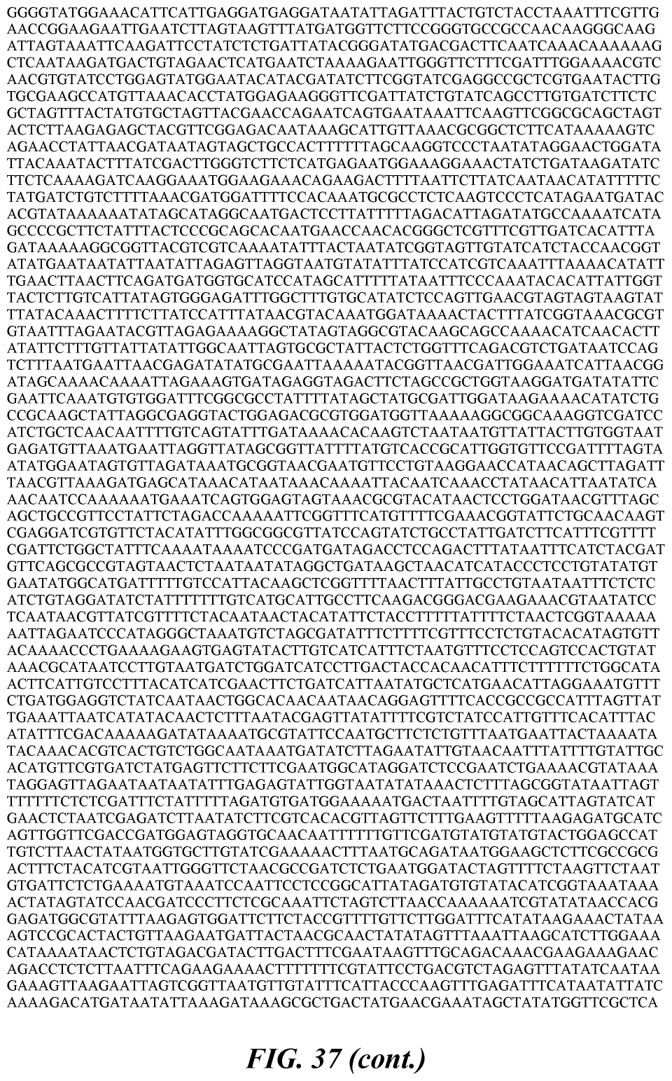

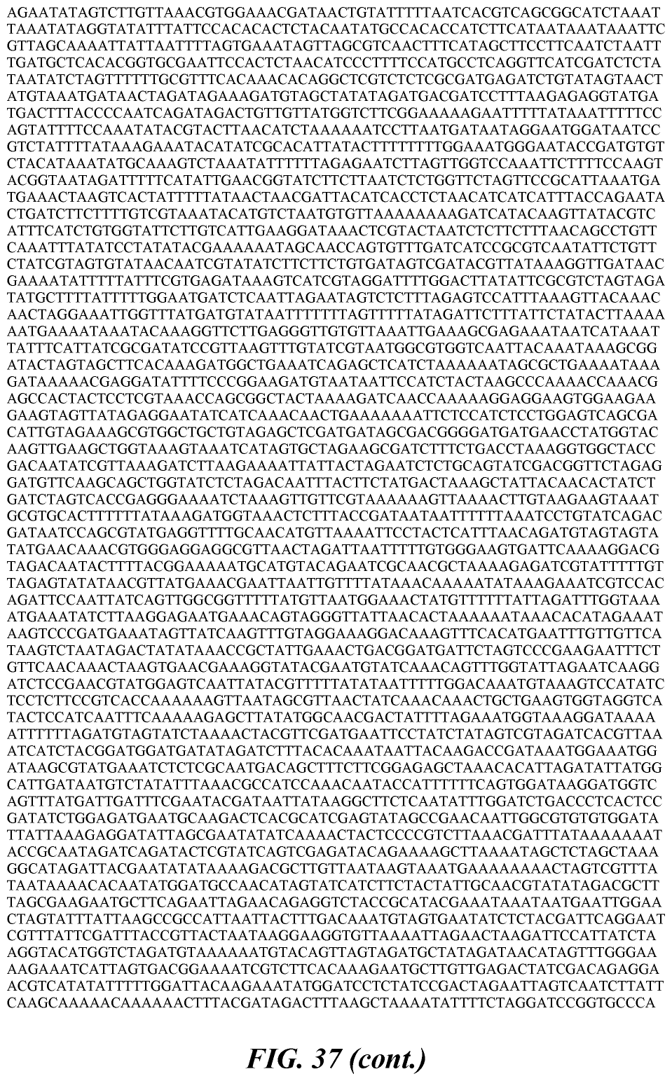

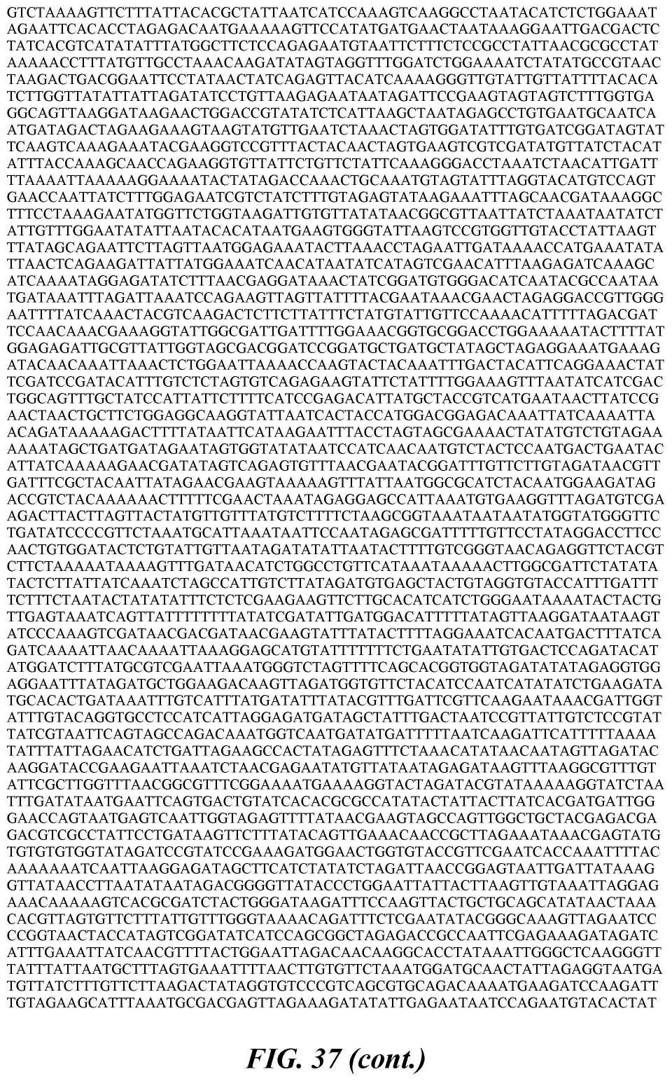

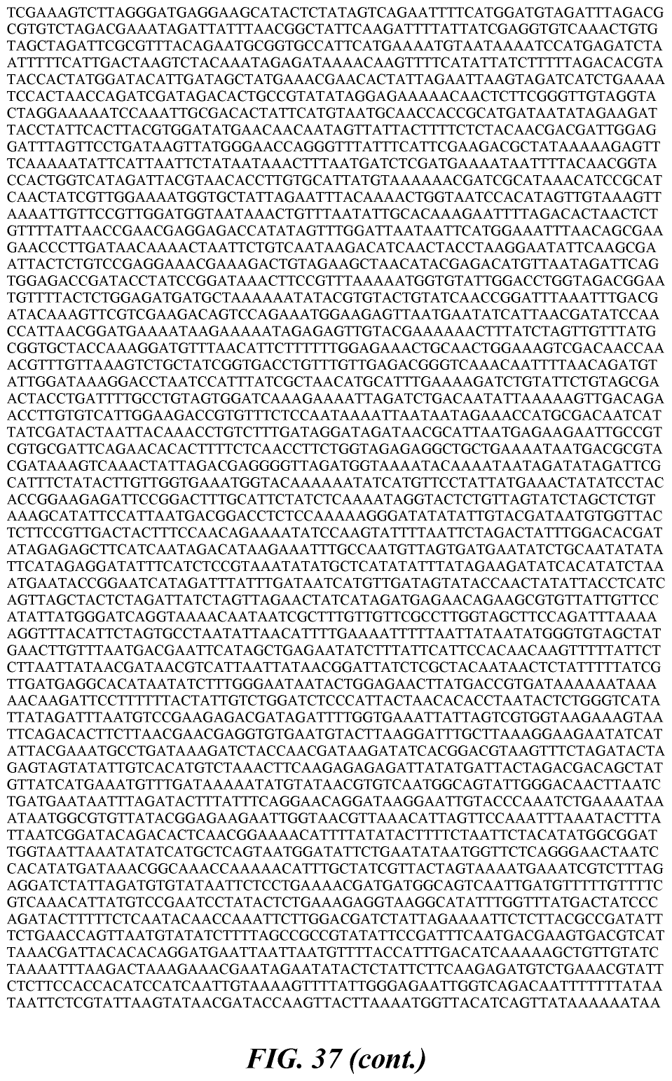

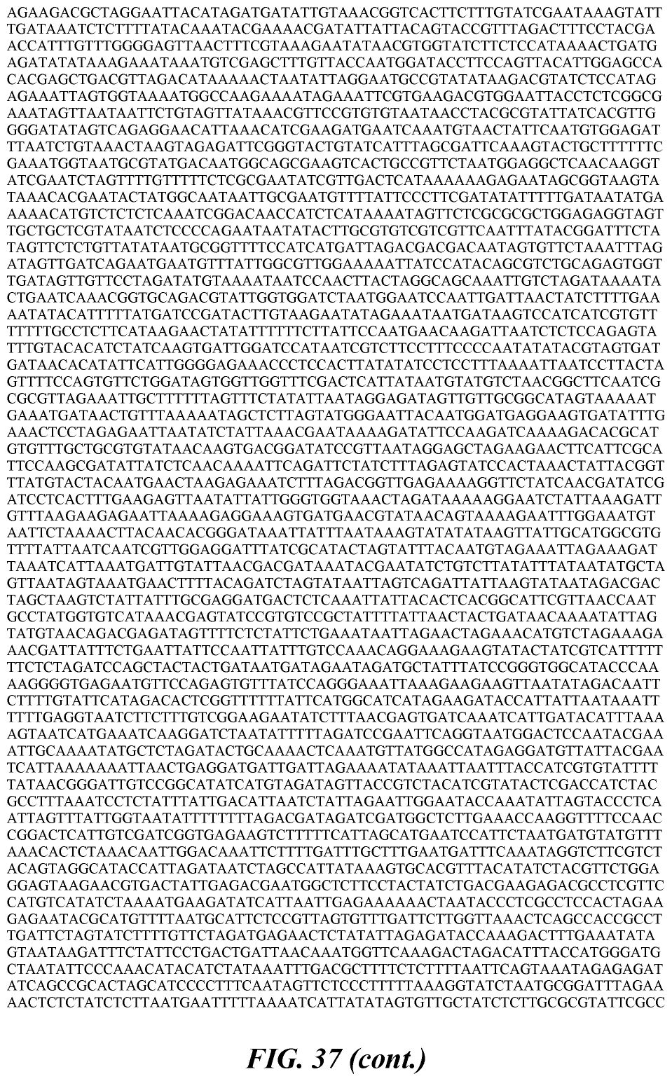

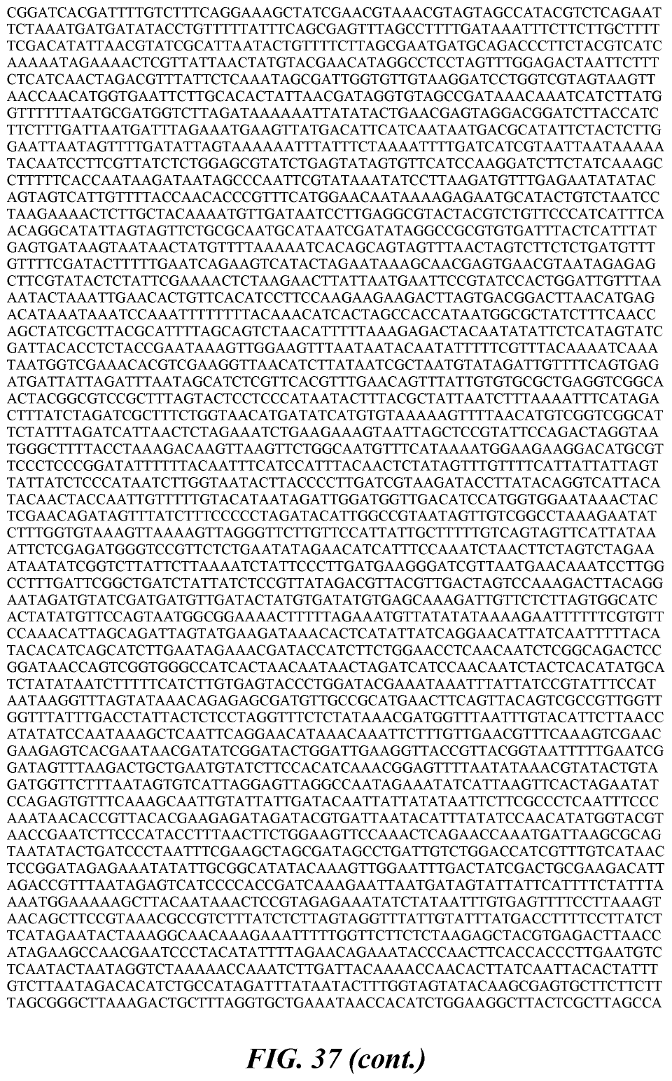

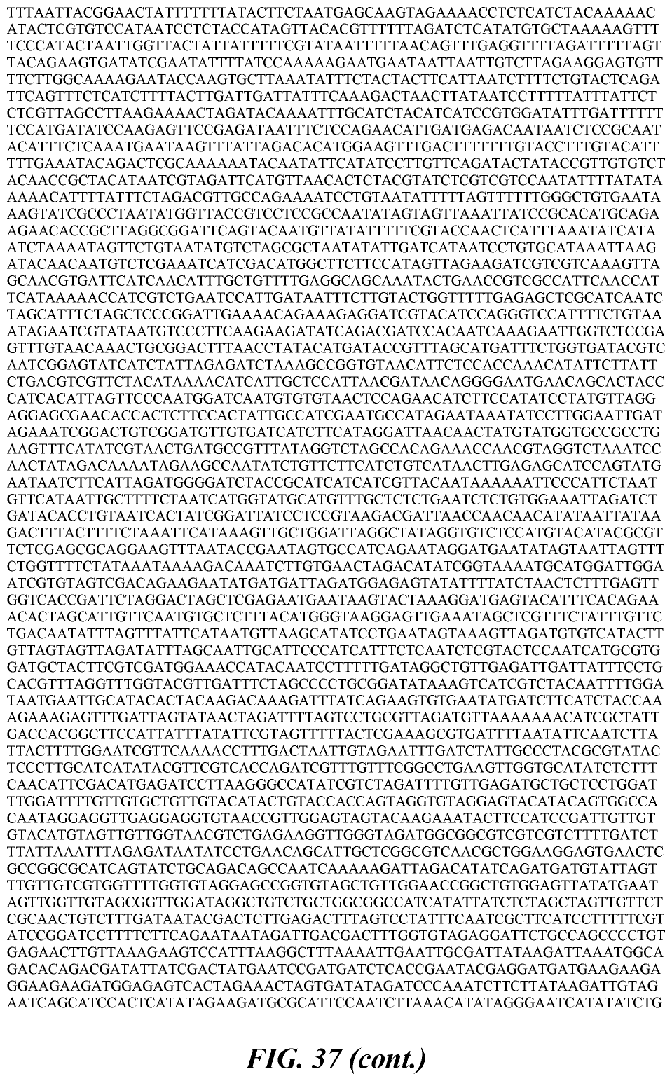

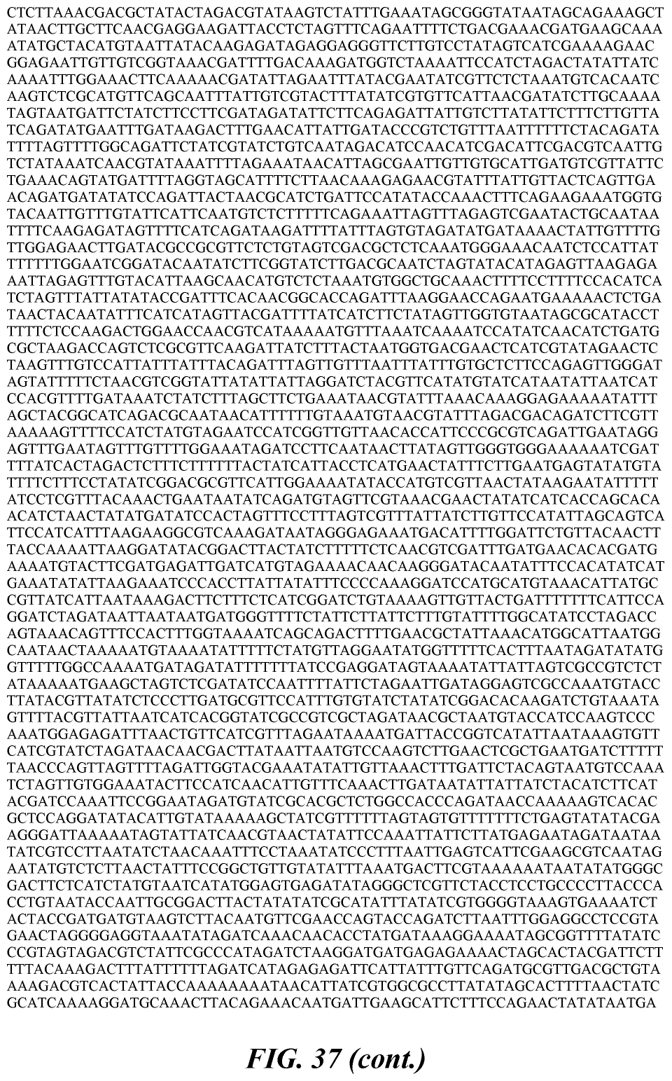

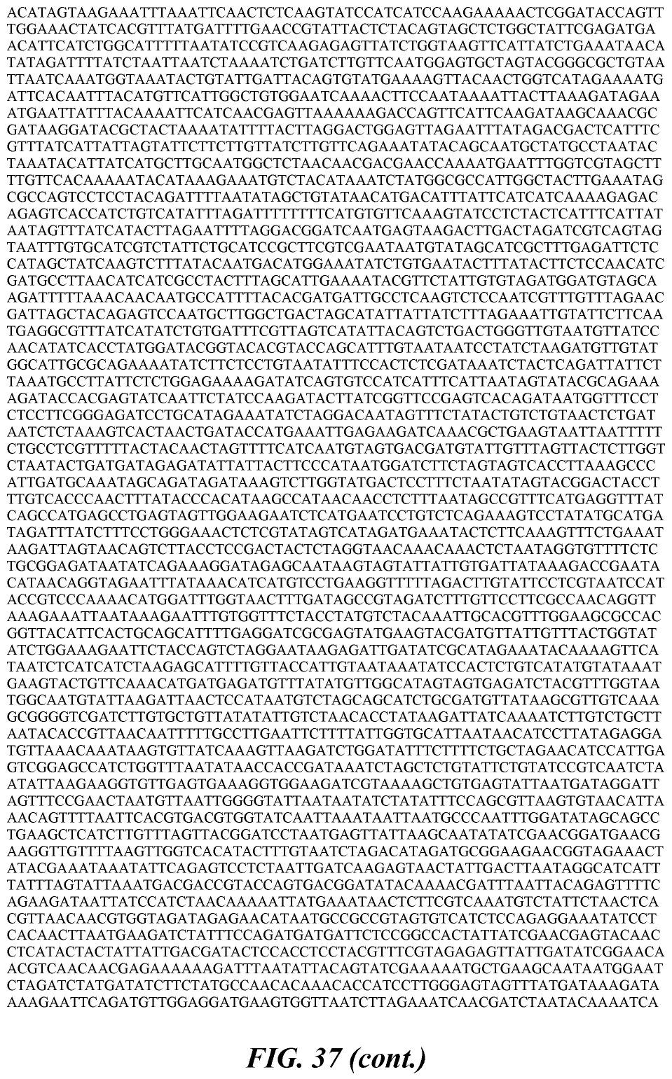

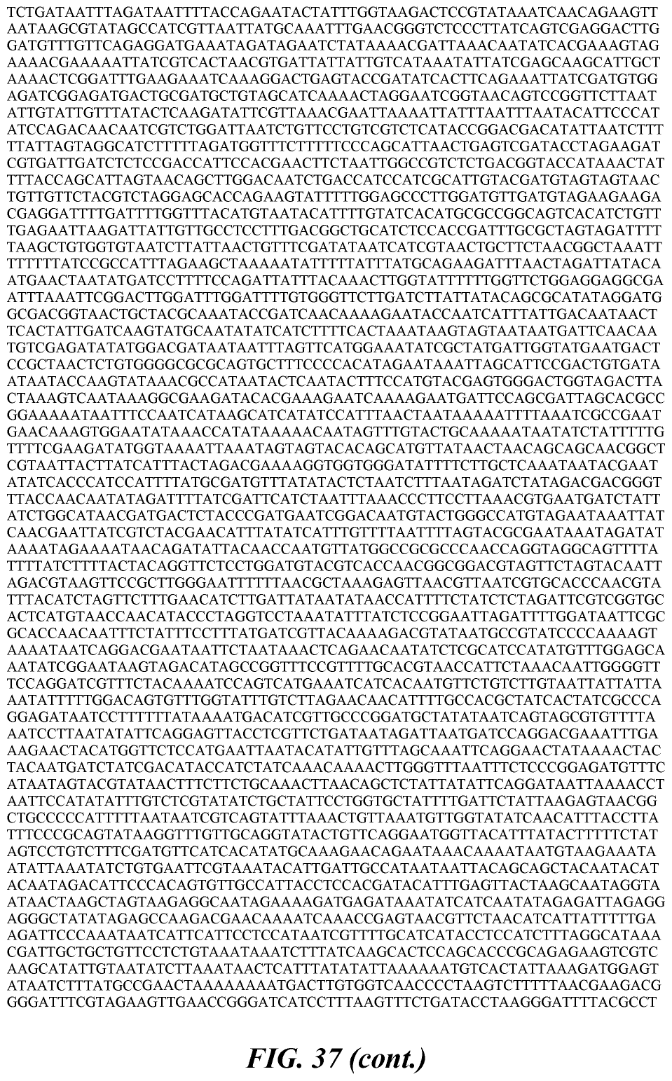

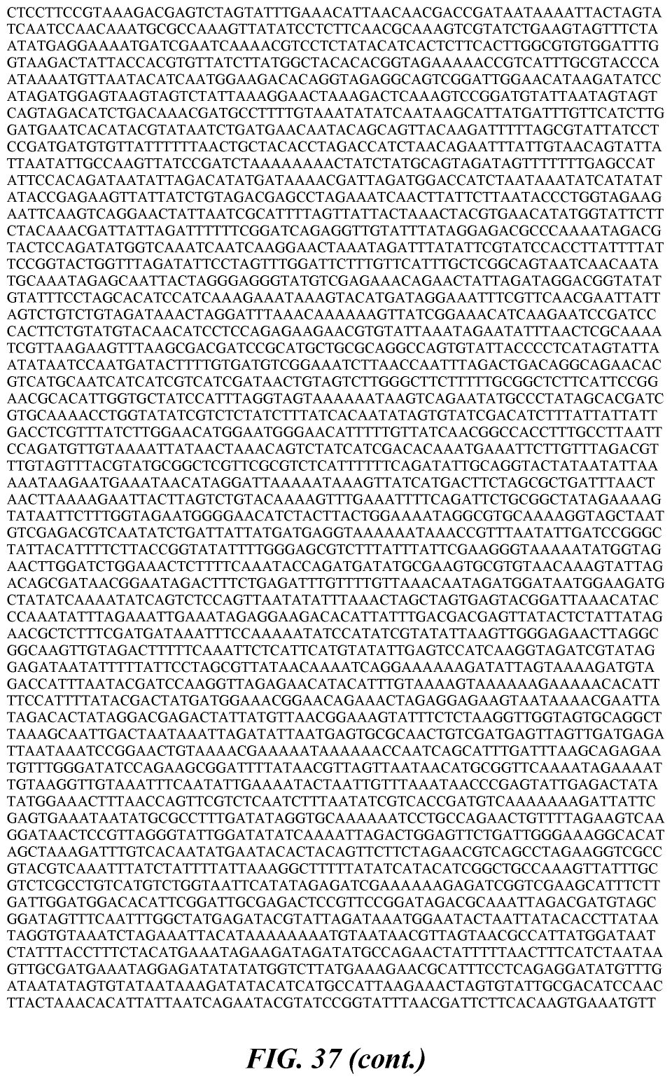

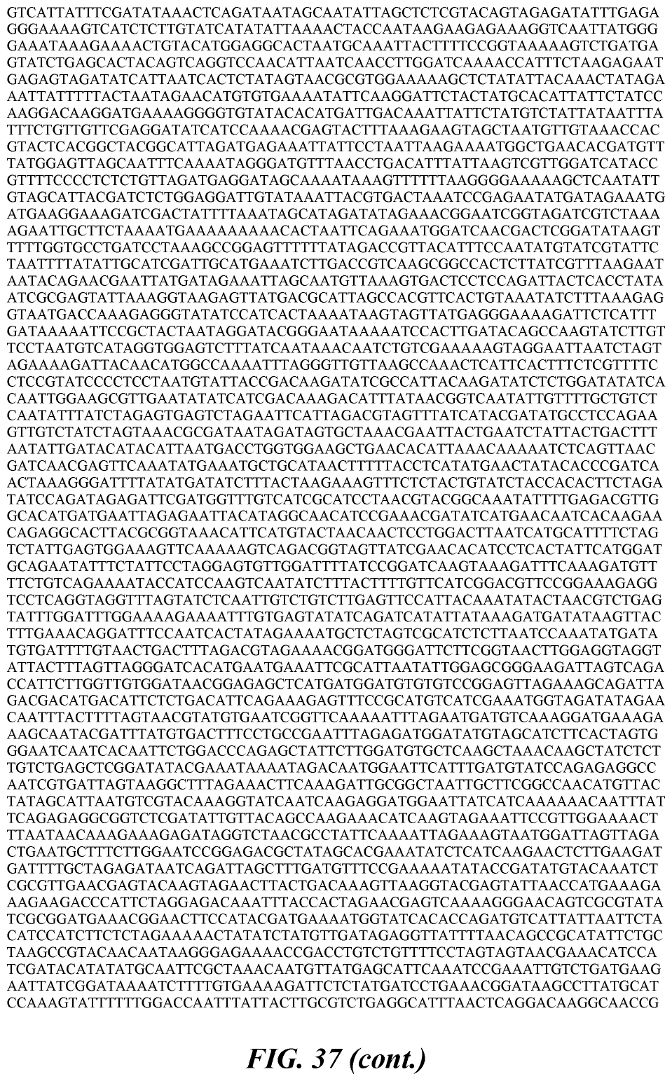

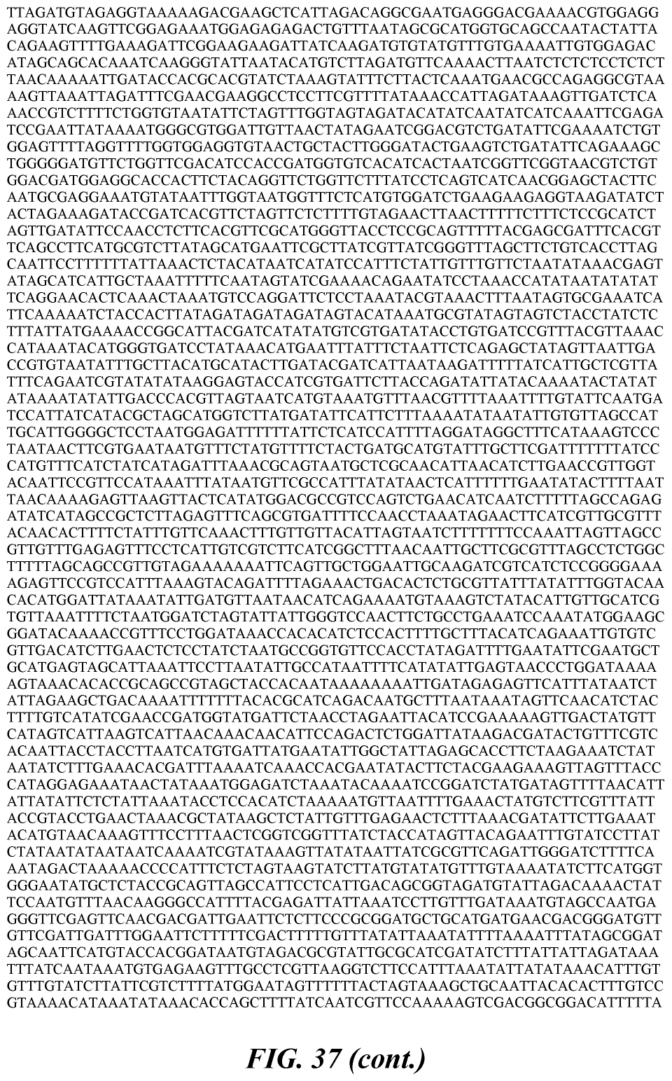

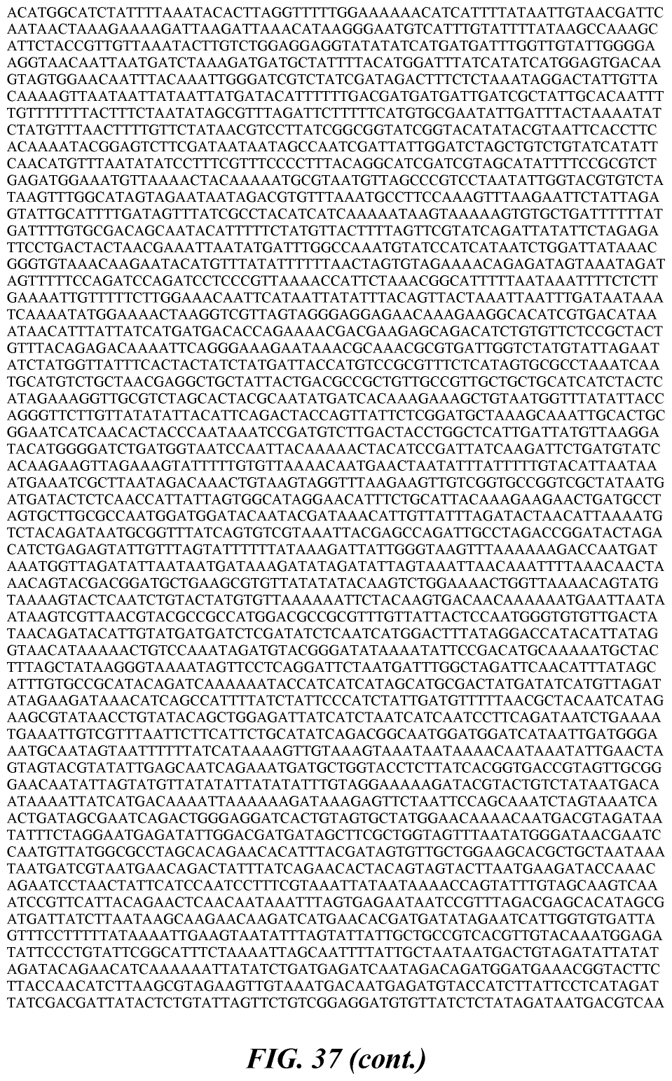

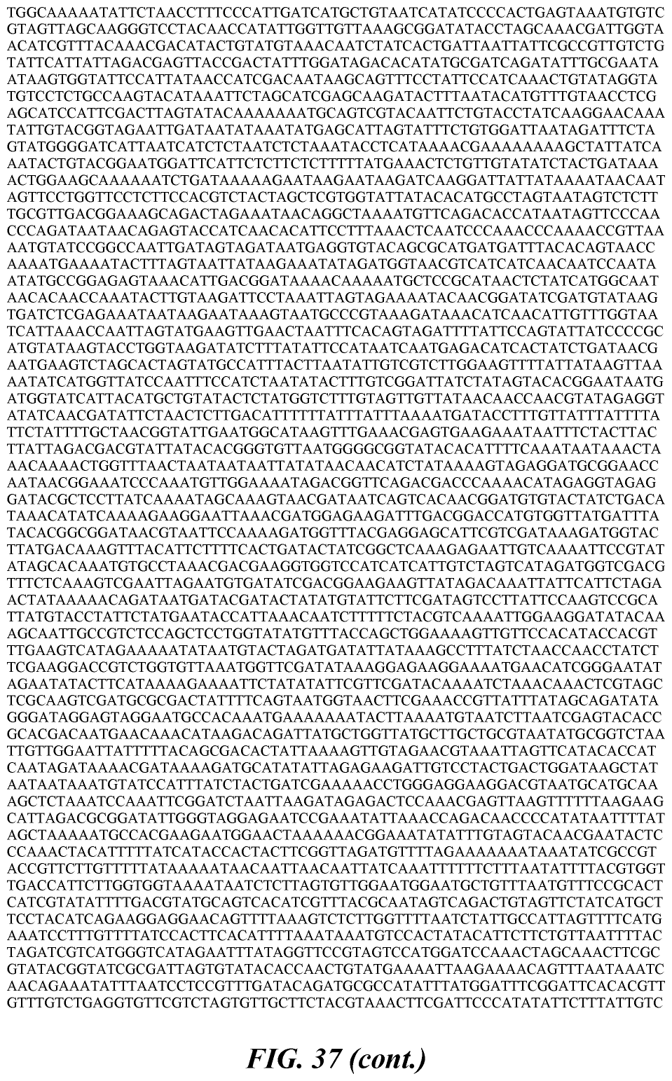

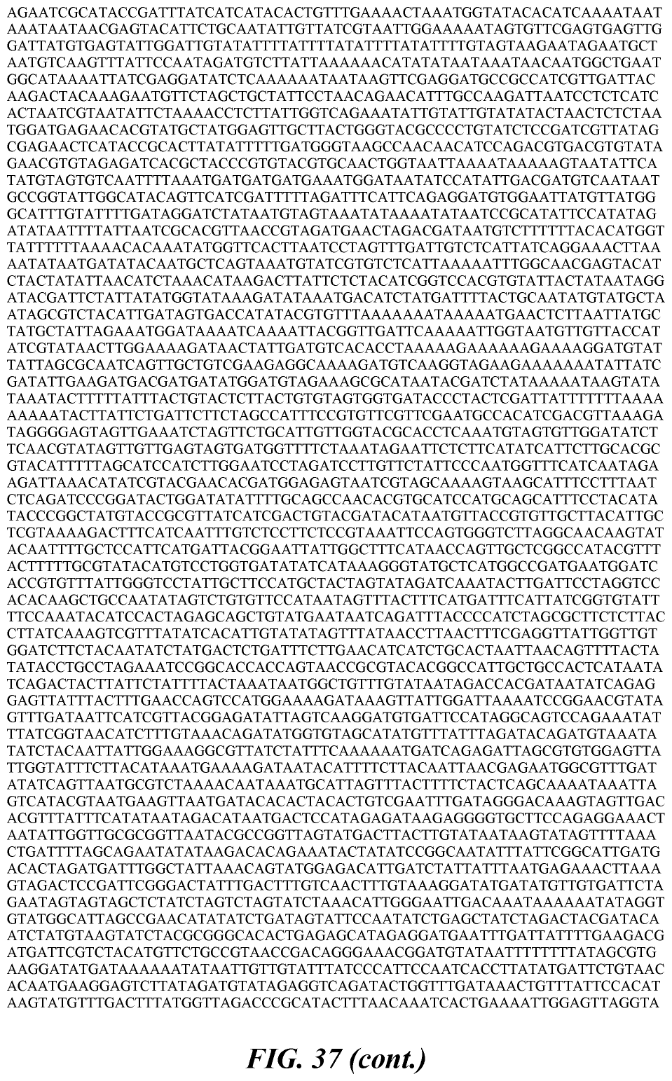

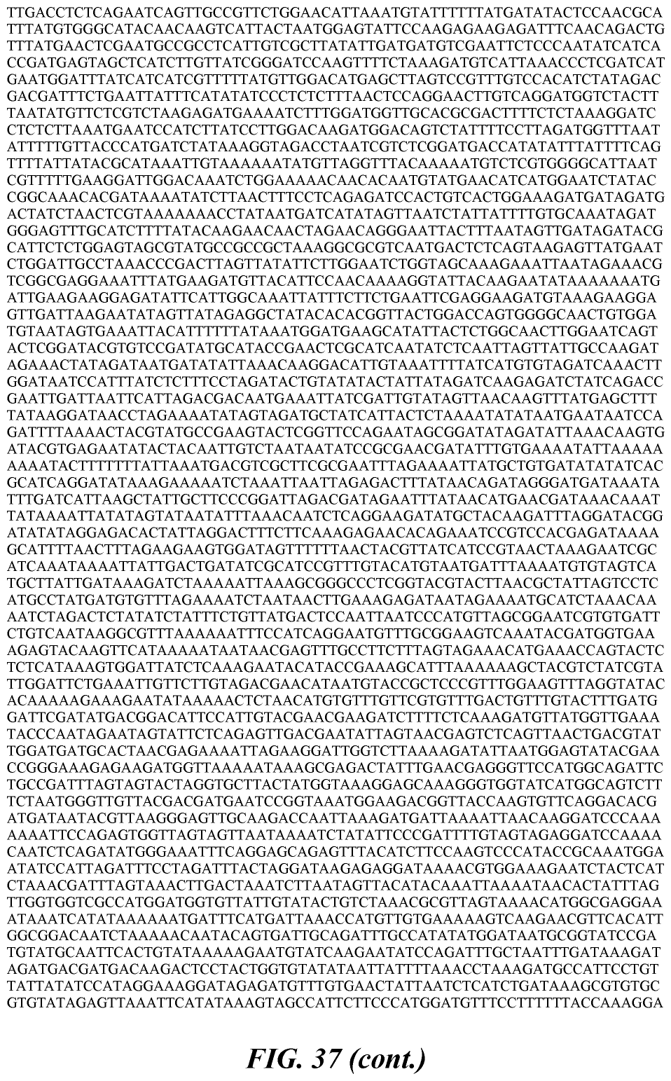

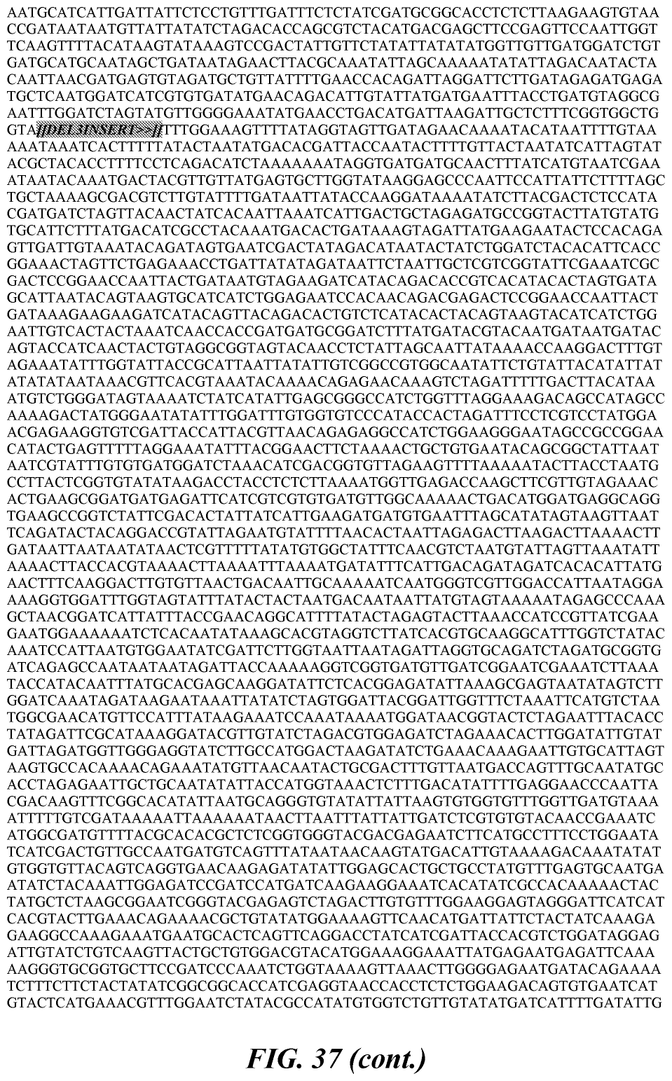

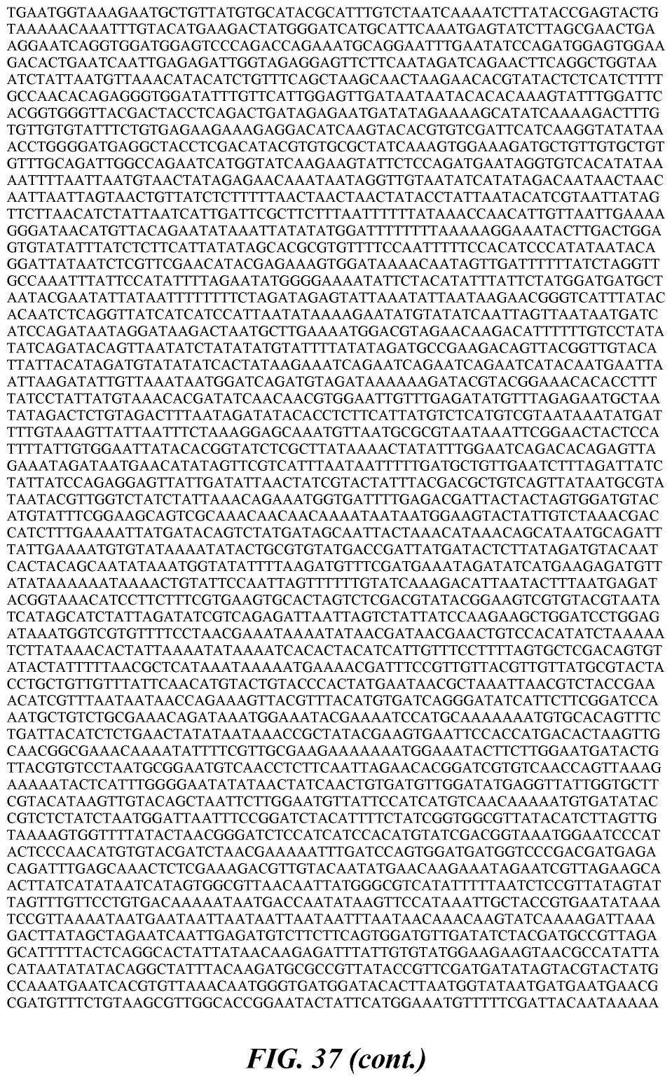

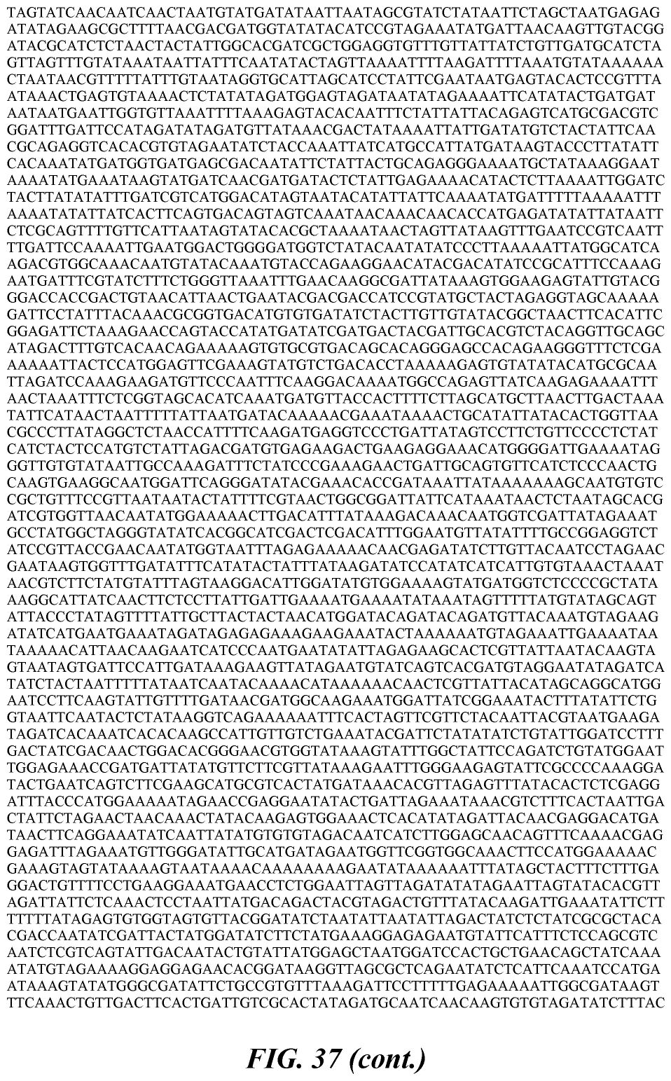

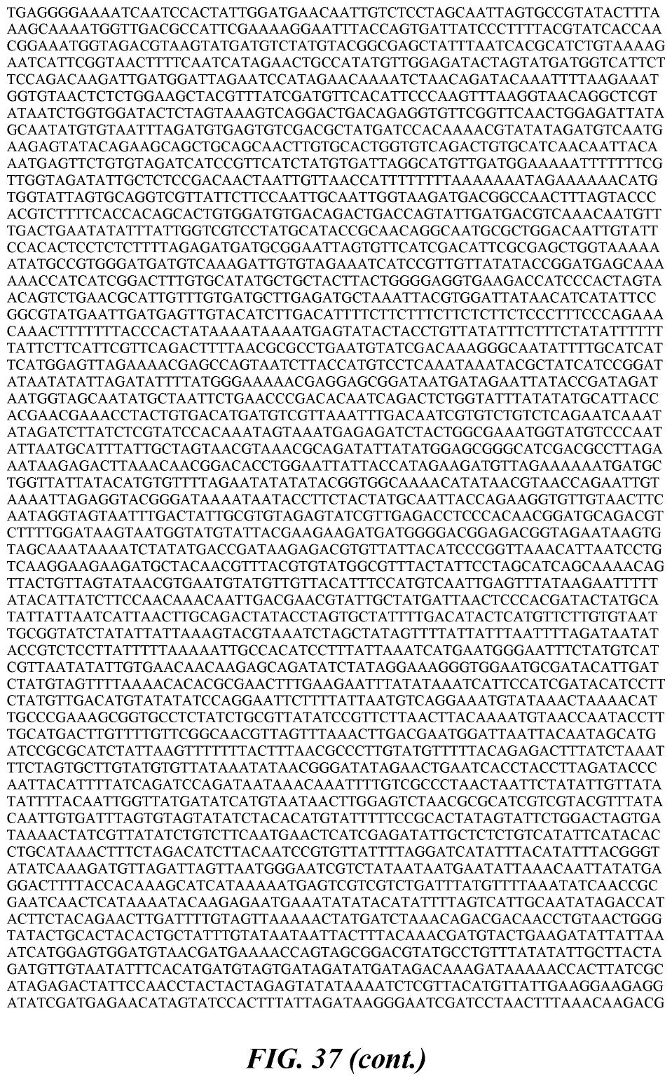

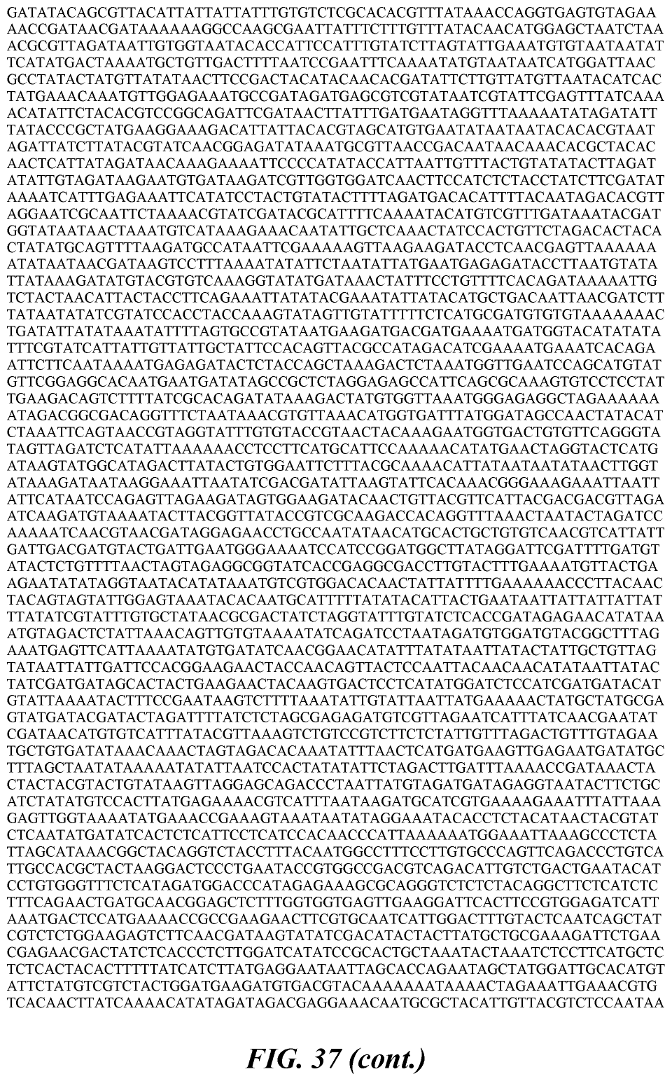

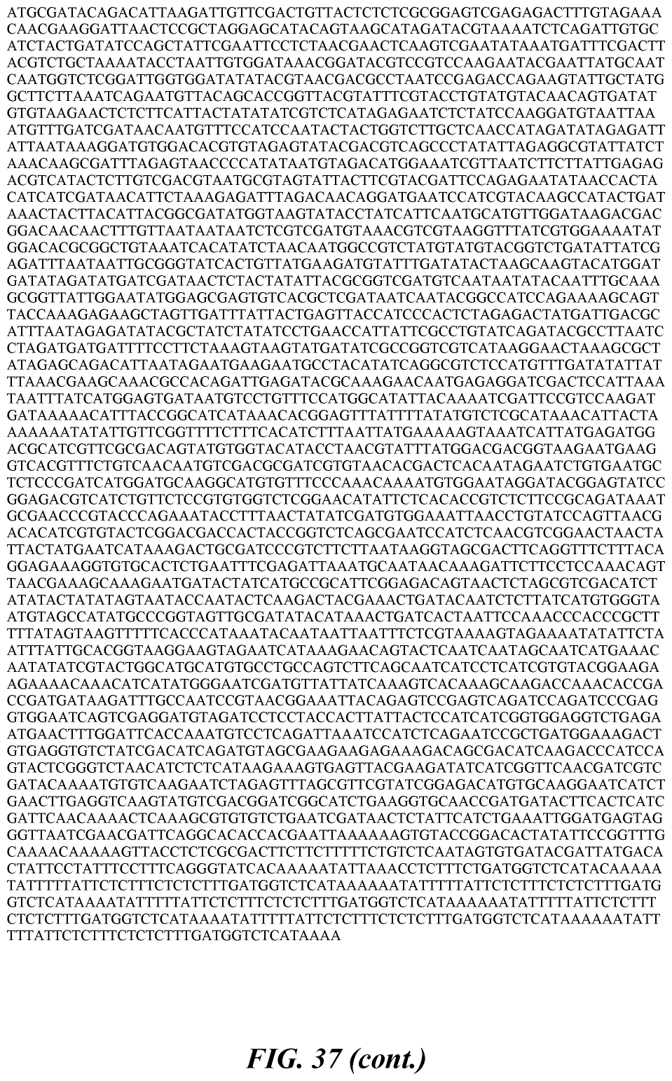

In some embodiments, heterologous nucleotide sequences that encode one or more SARS-CoV-2 proteins (or variants, mutants, and/or immunogenic fragments thereof) are inserted into one or more MVA insertion sites. Non-limiting examples of insertion sites that may be used to insert the heterologous nucleotide sequences include, but are not limited to, Del2, IGR44/45, IGR64/65, IGR69/70, and Del3. For example, FIG. 37 shows a sequence of an sMVA backbone (SEQ ID NO: 1), as well as the possible insertion sites including Del2 (shown as [[<DEL2INSERT]], where “<<” indicates the heterologous nucleotide insert at Del2 is the reverse complement of the SARS-CoV-2 protein sequence (or variants, mutants, and/or immunogenic fragments thereof) sequence); IGR69/70 (shown as [[<INSERTIGR69/70]A], [[<INSERTIGR69/70]B], [[<<NSERTIGR69/70]c], or [[INSERTIGR69/70]D], where “<<” indicates the heterologous nucleotide insert at IGR69/70 is the reverse complement of the SARS-CoV-2 protein sequence (or variants, mutants, and/or immunogenic fragments thereof), and each insertion site is representative of four alternative insertion sites for IGR69/70: A, B, C, or D); and Del3 (shown as [[DEL3INSERT>>]], where “>>” indicates the heterologous nucleotide insert at Del3 is the forward SARS-CoV-2 protein (or variant, mutant, and/or immunogenic fragment thereof) sequence). FIG. 37 also includes an “X” (highlighted and in bold underline) that may be a T or an A according to some embodiments.

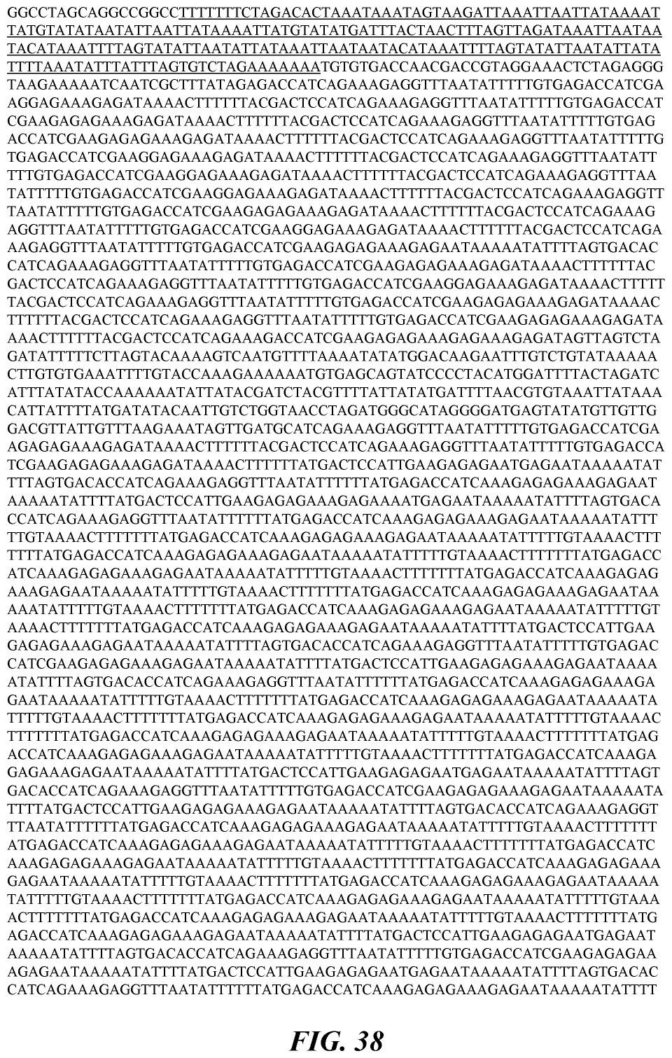

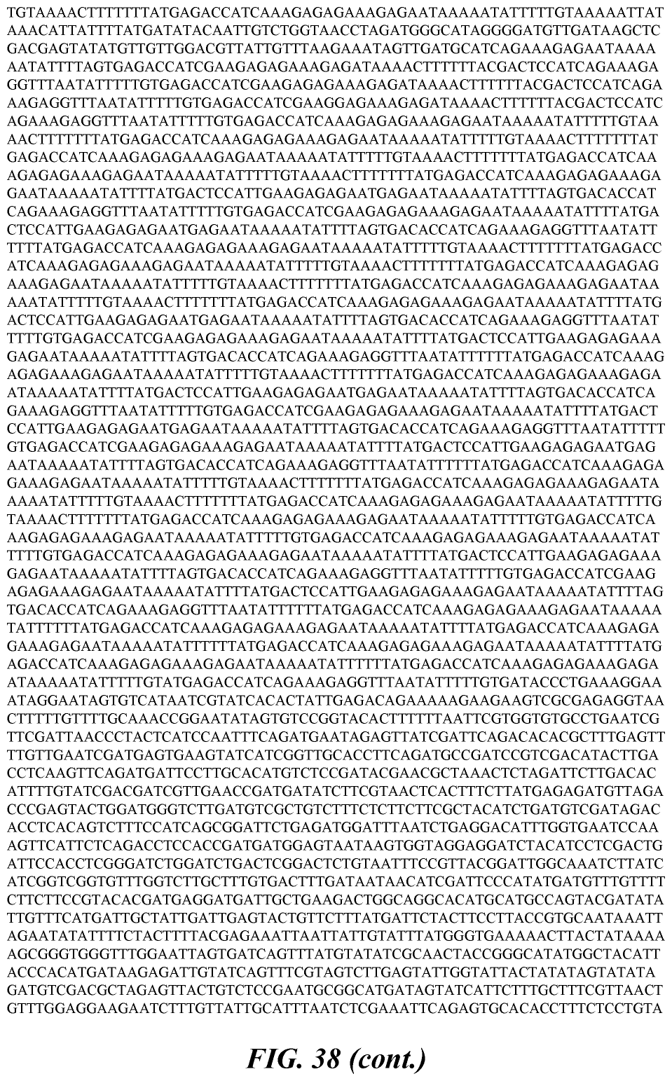

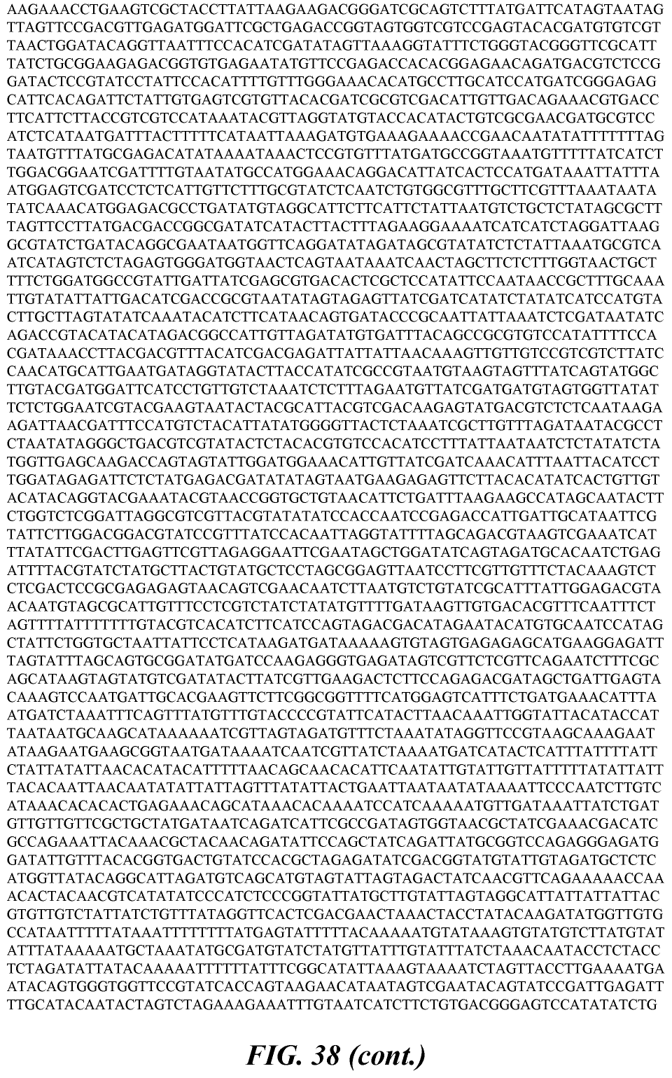

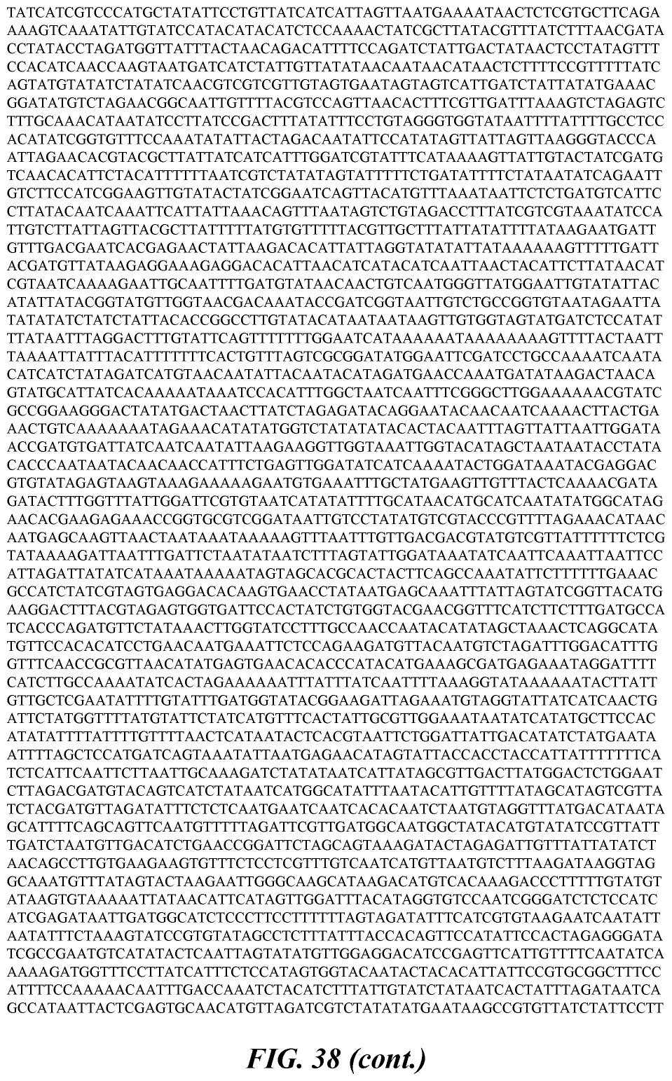

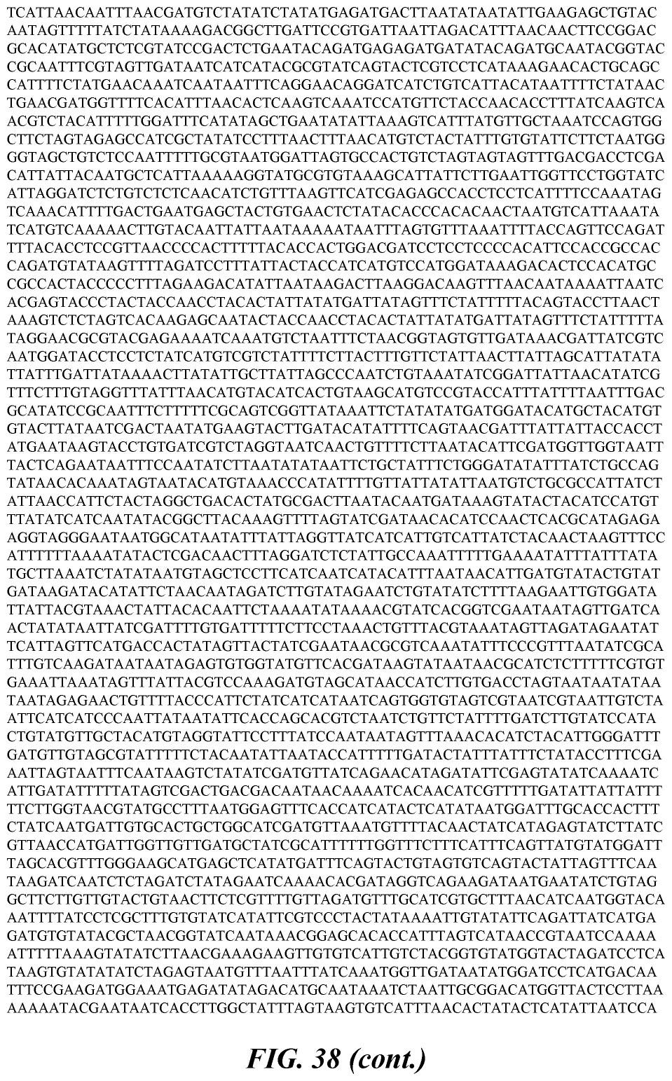

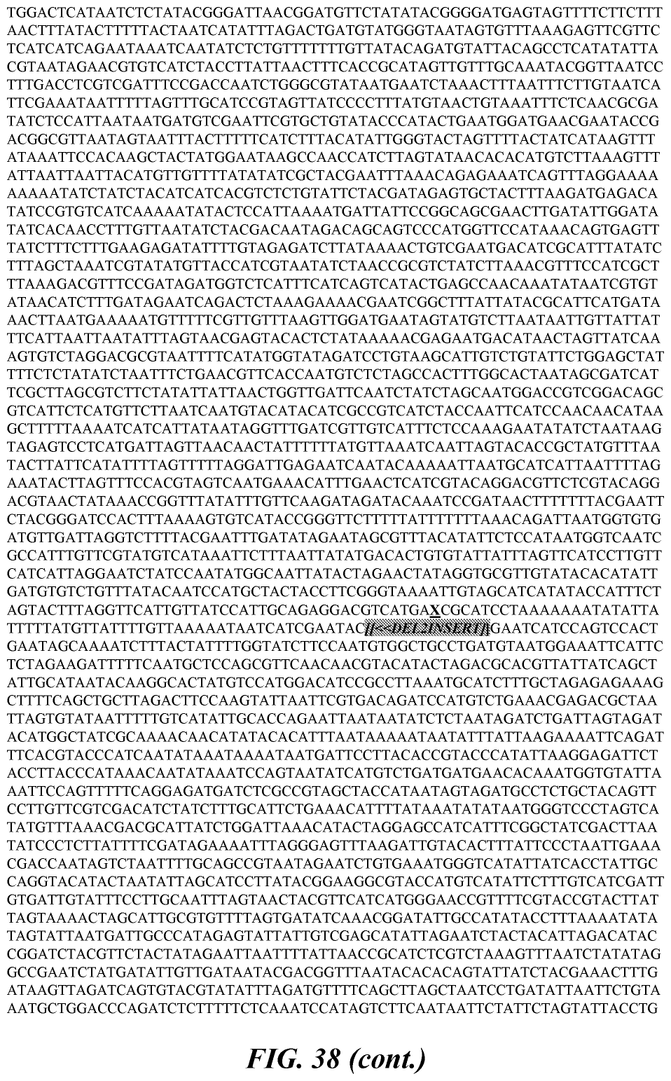

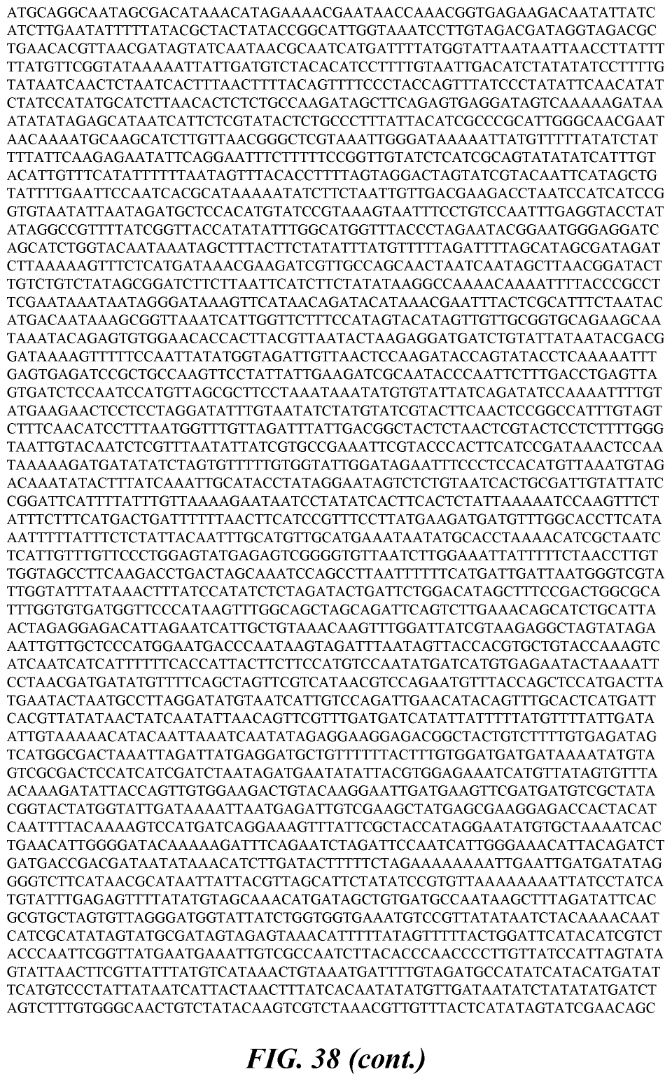

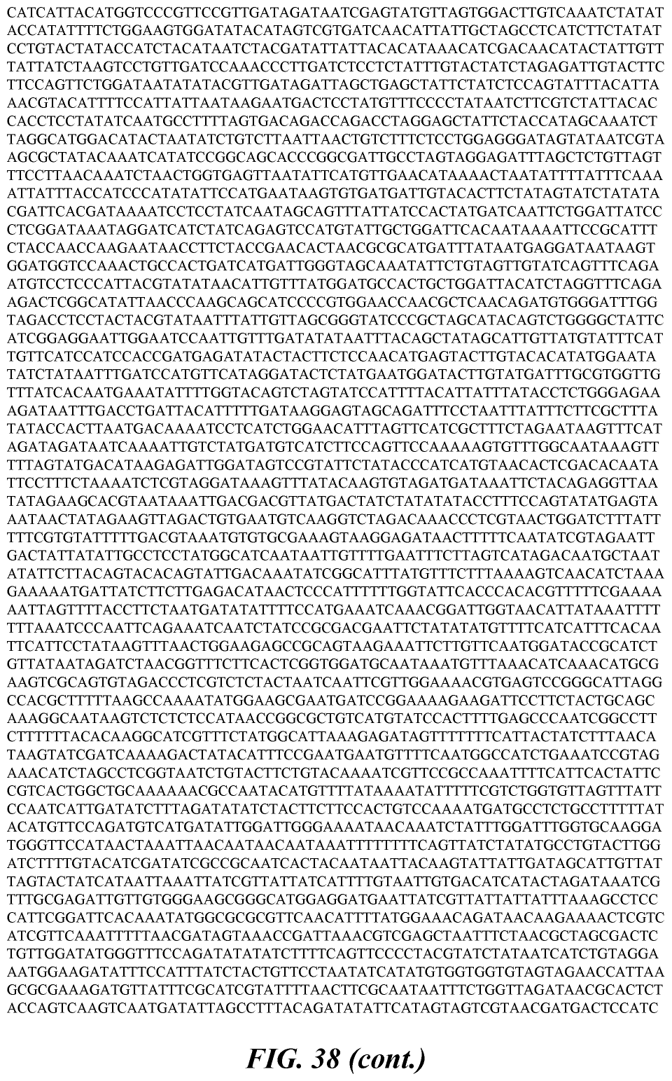

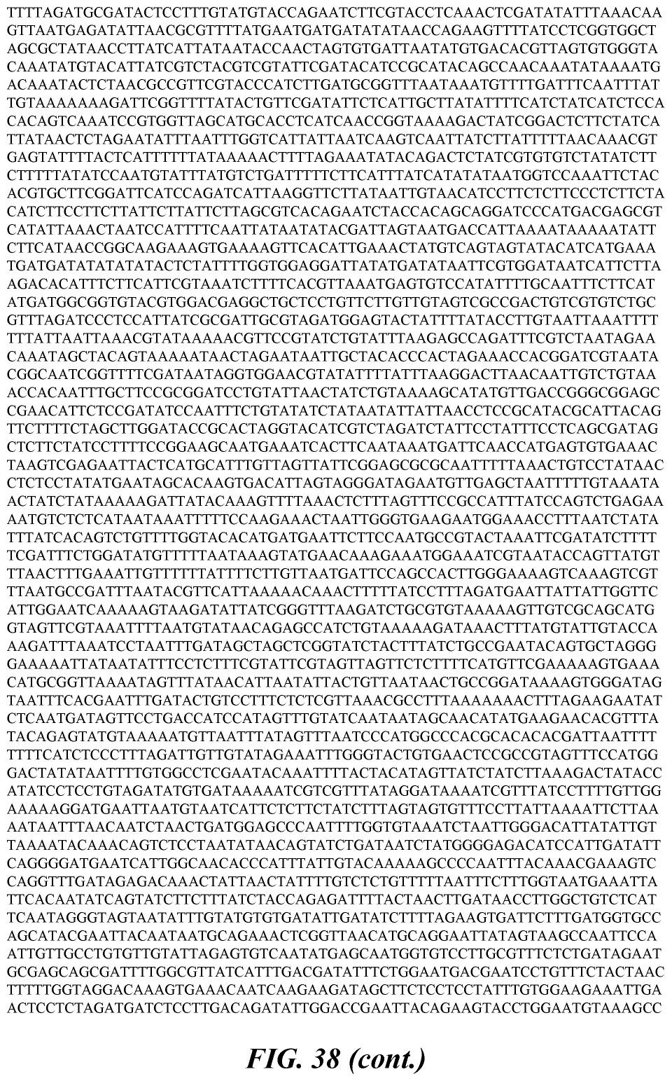

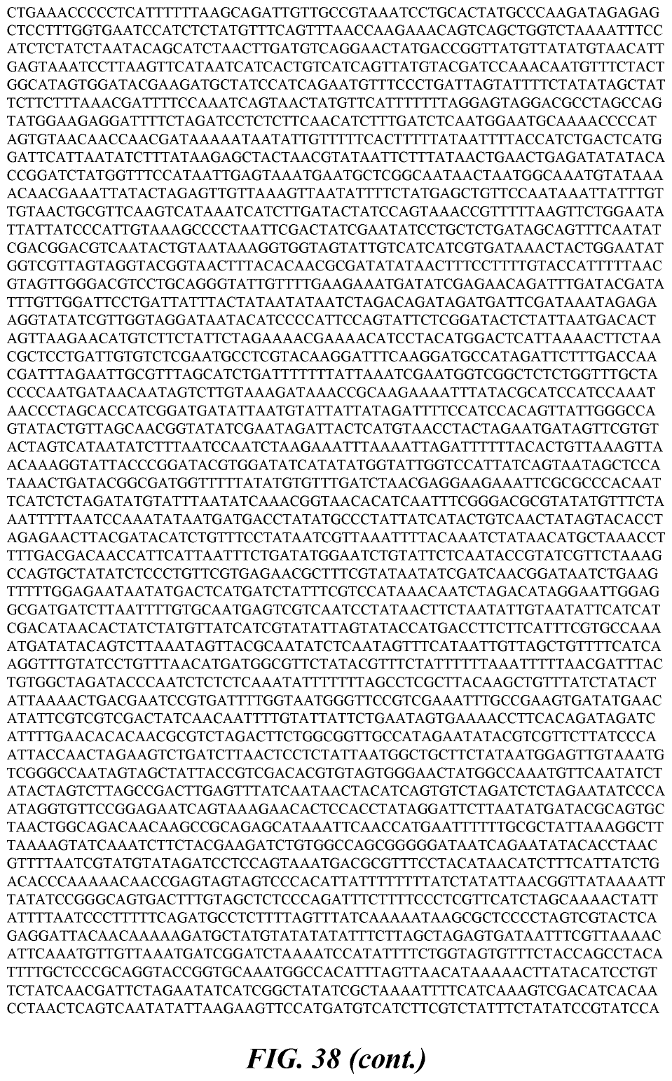

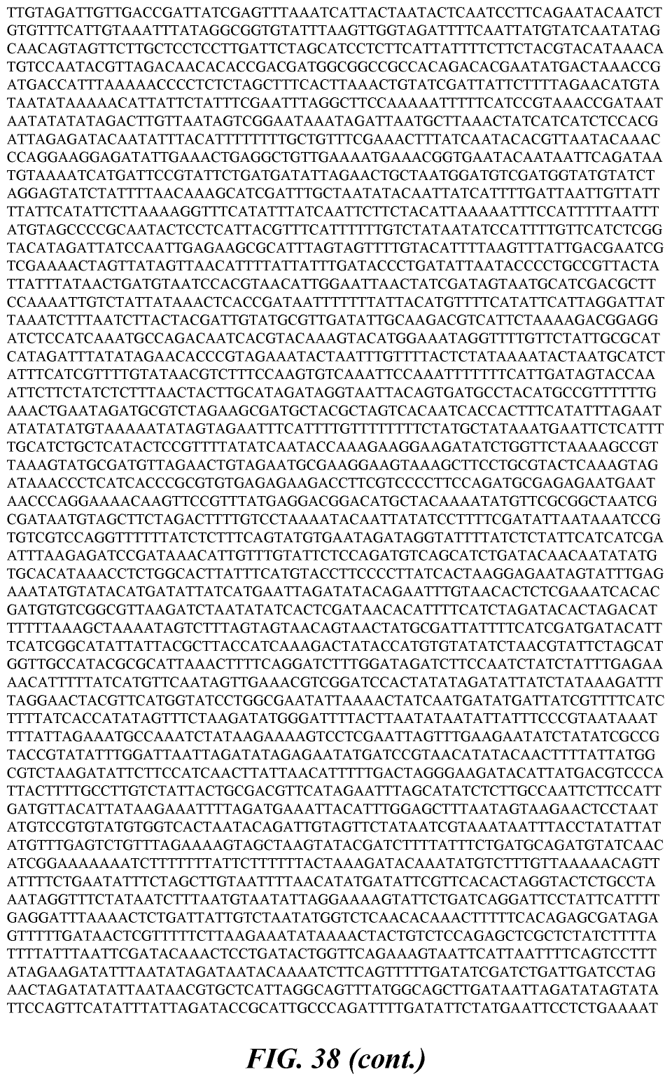

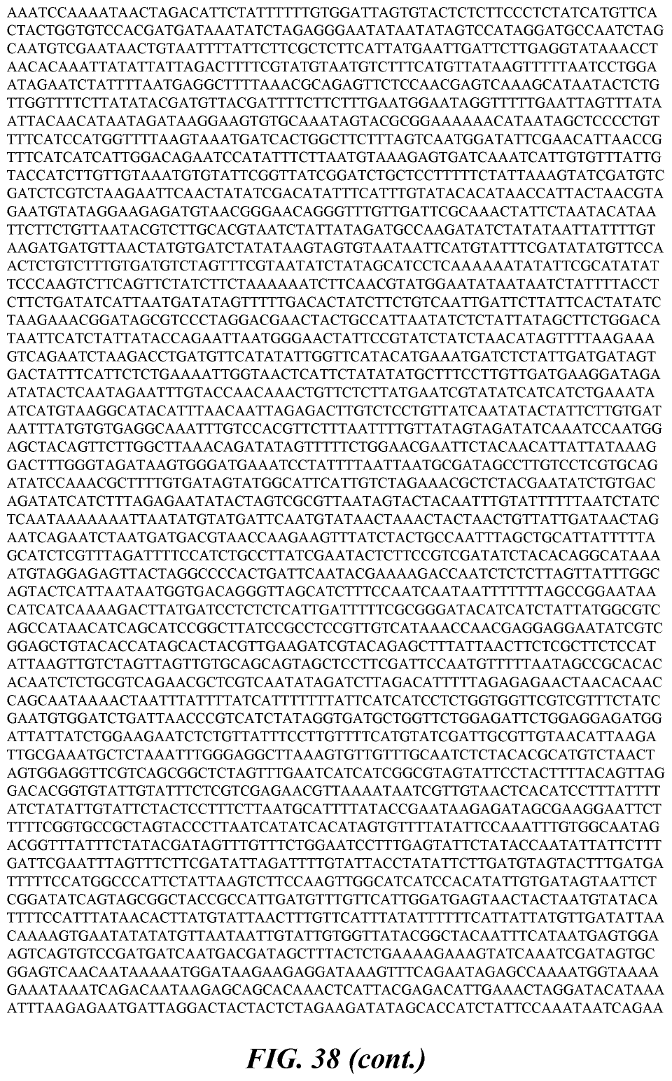

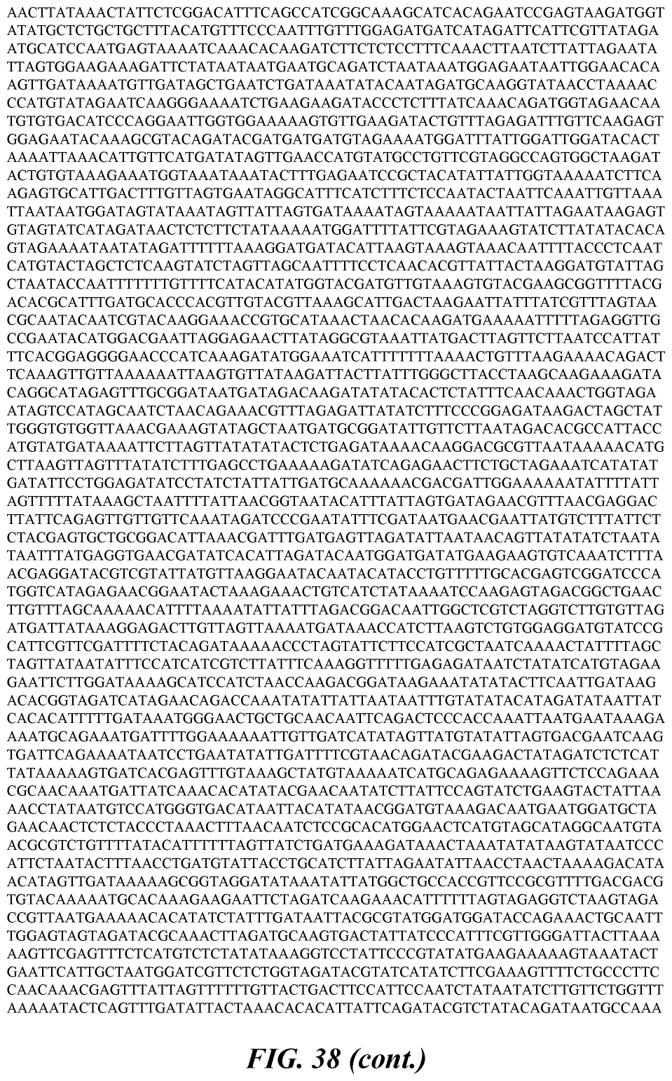

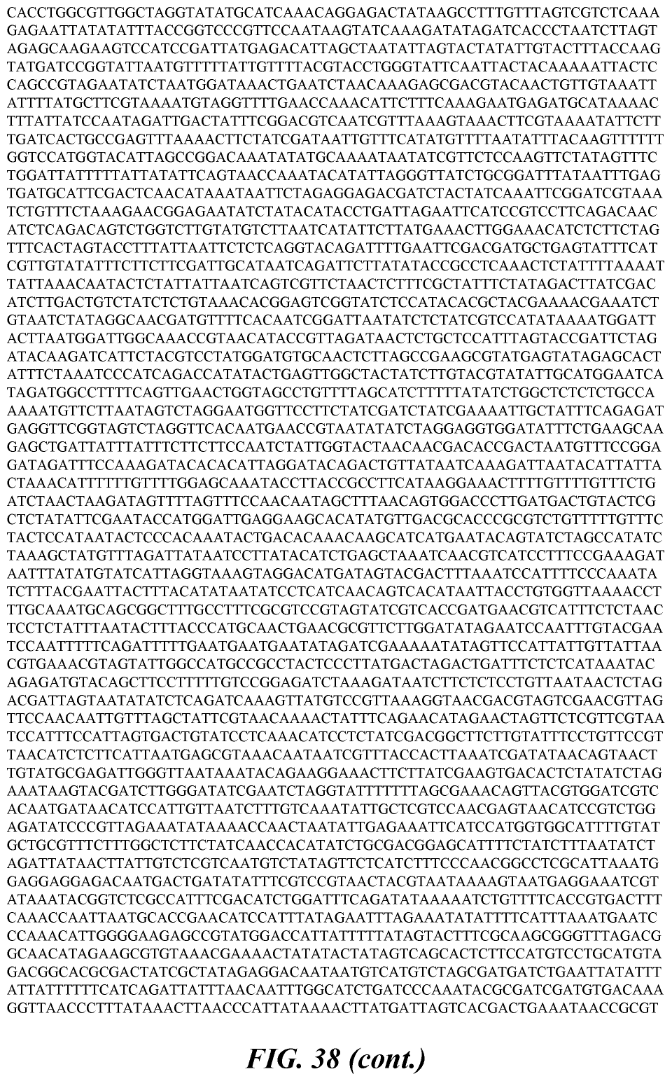

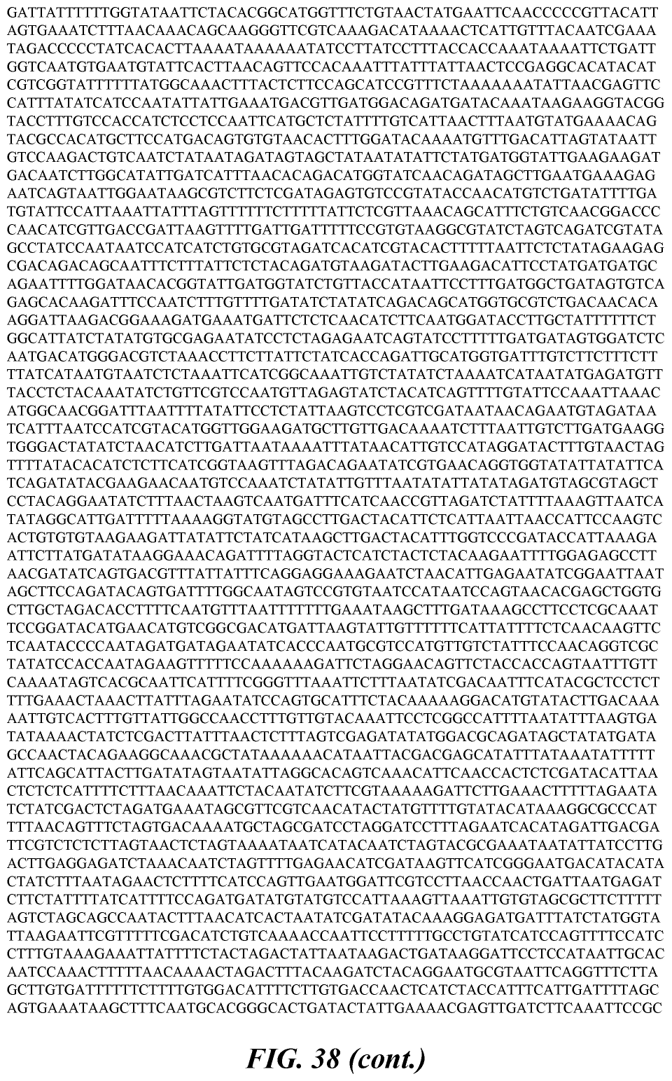

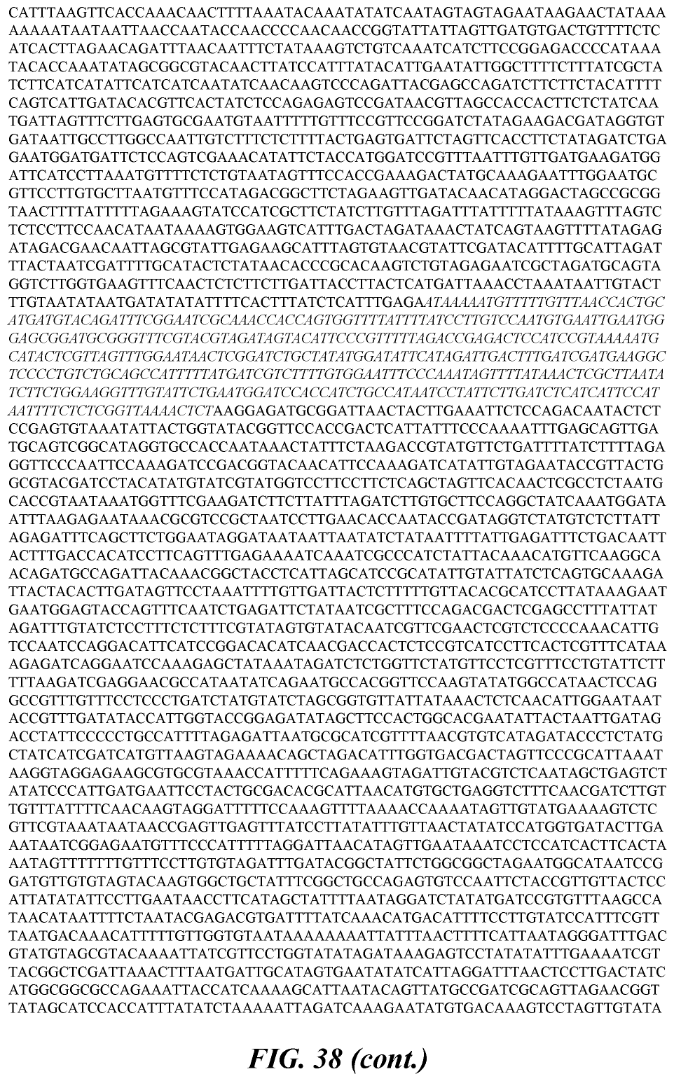

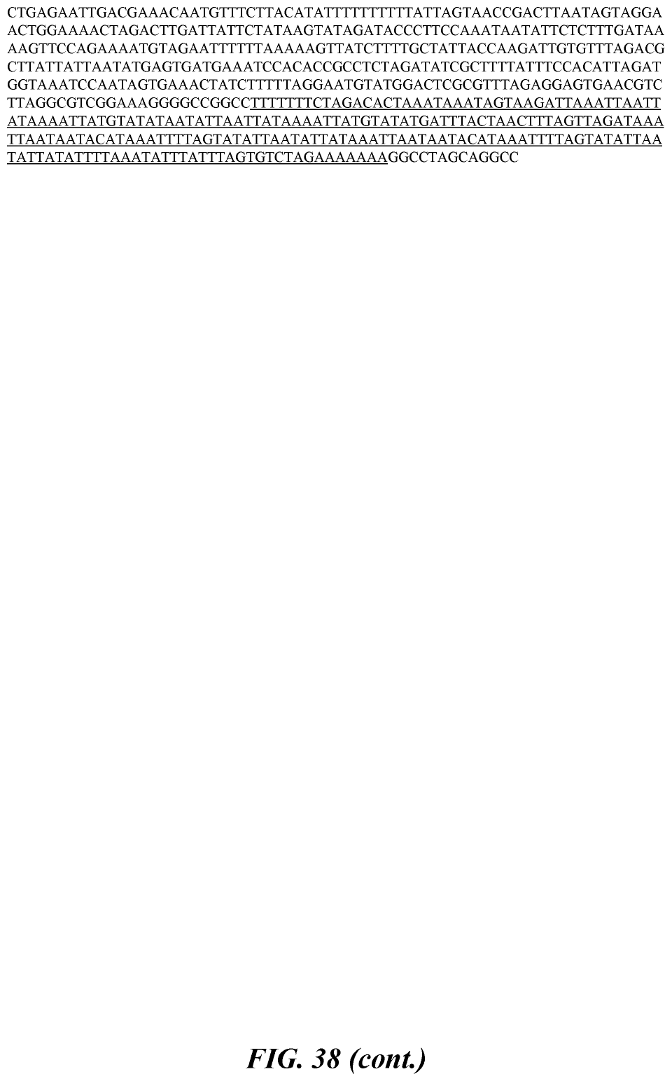

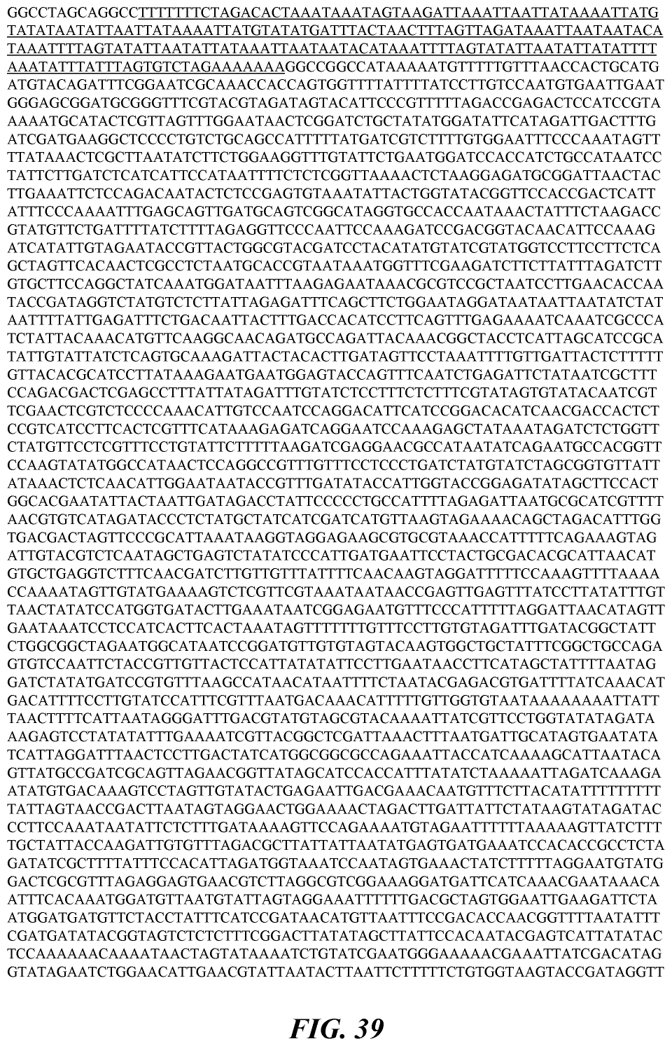

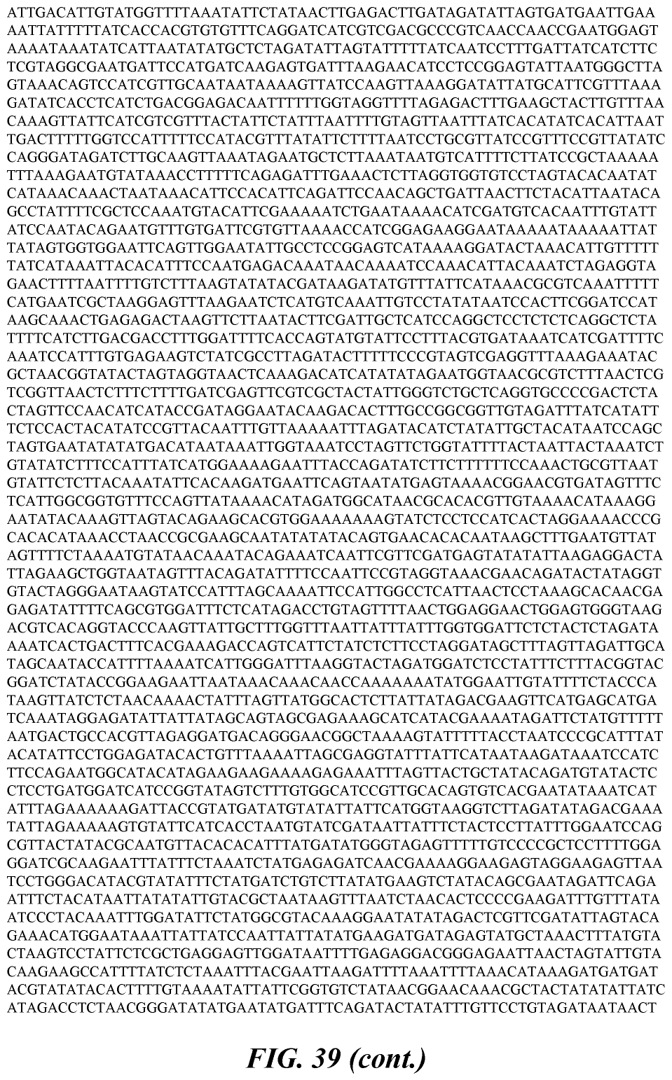

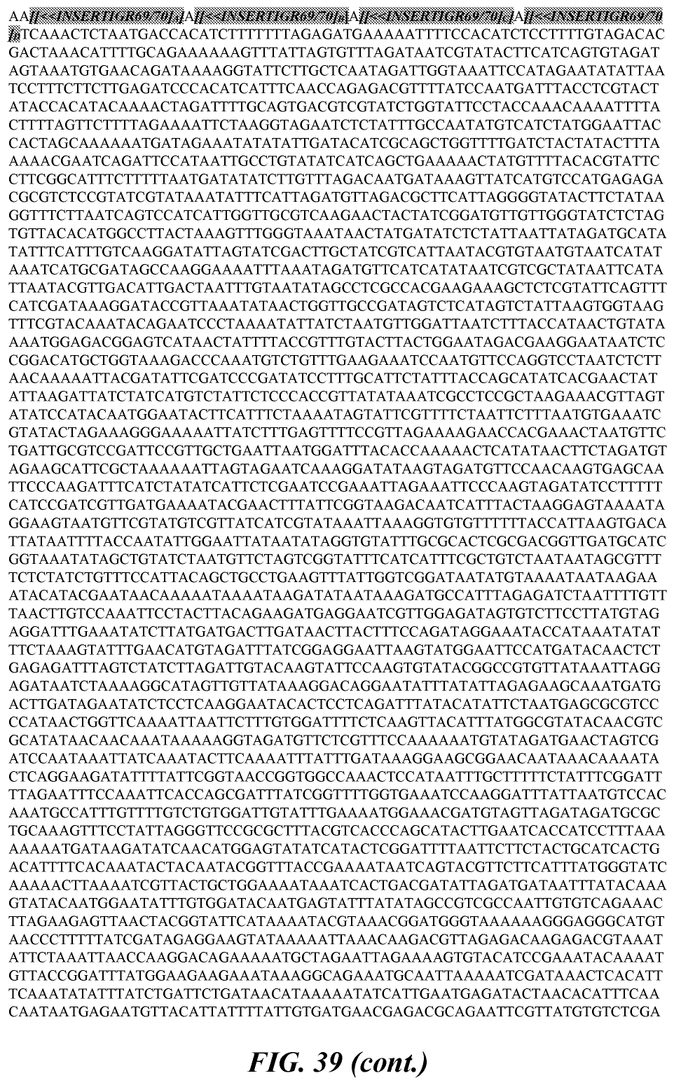

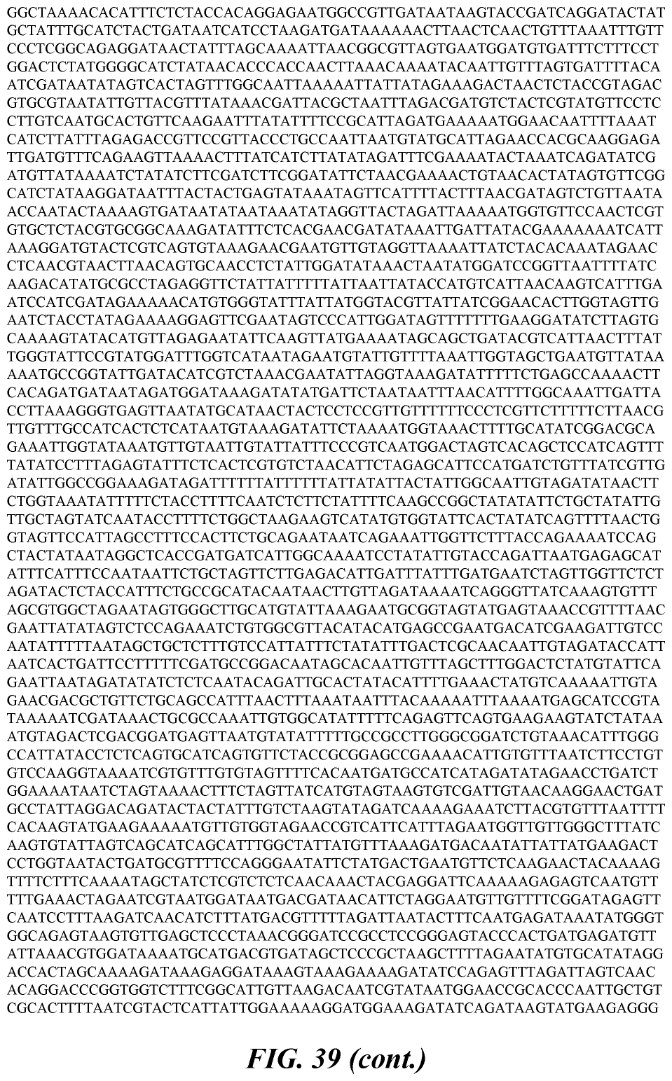

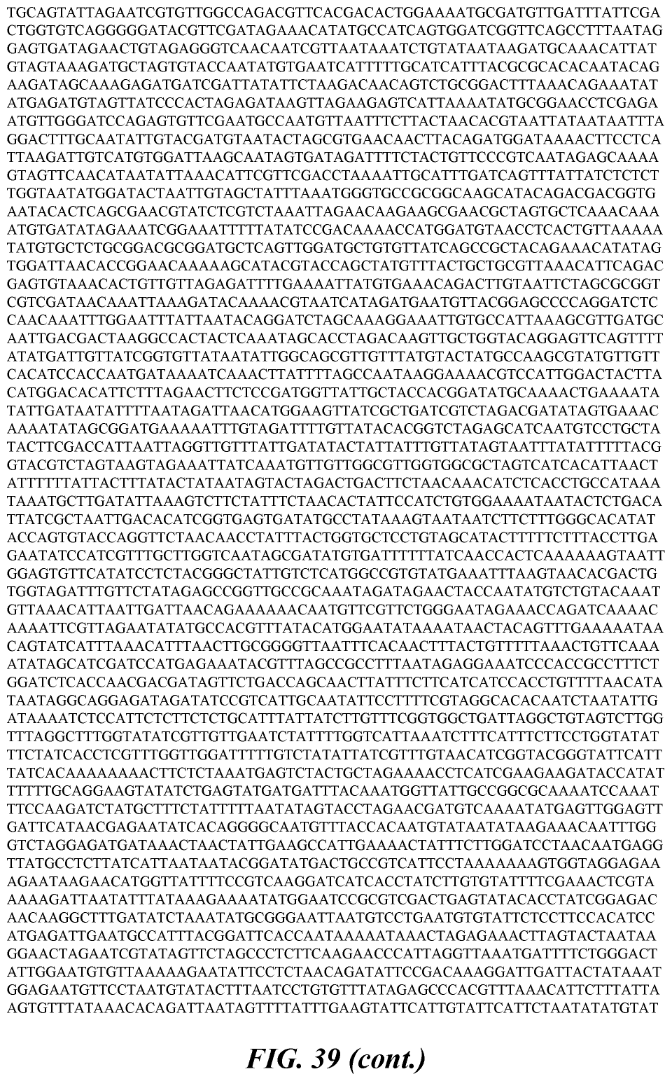

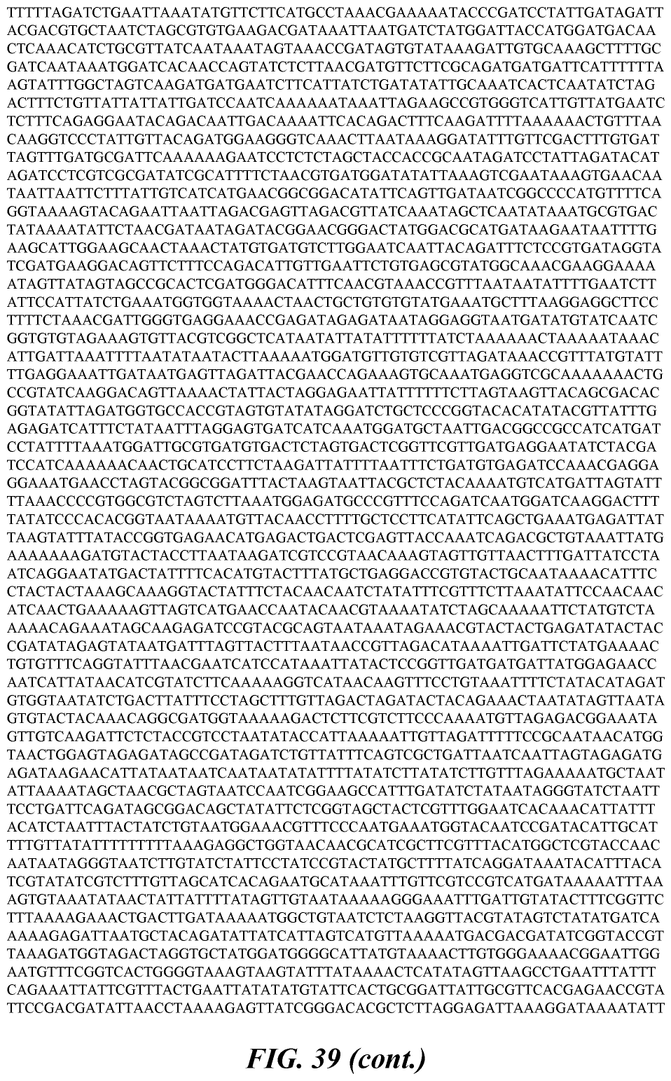

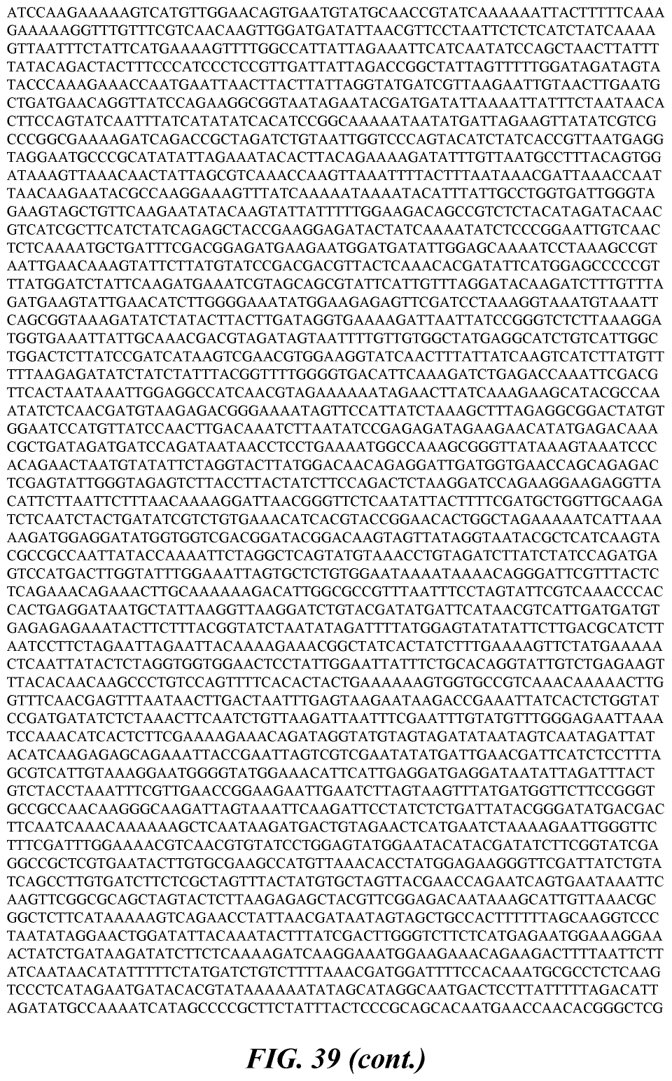

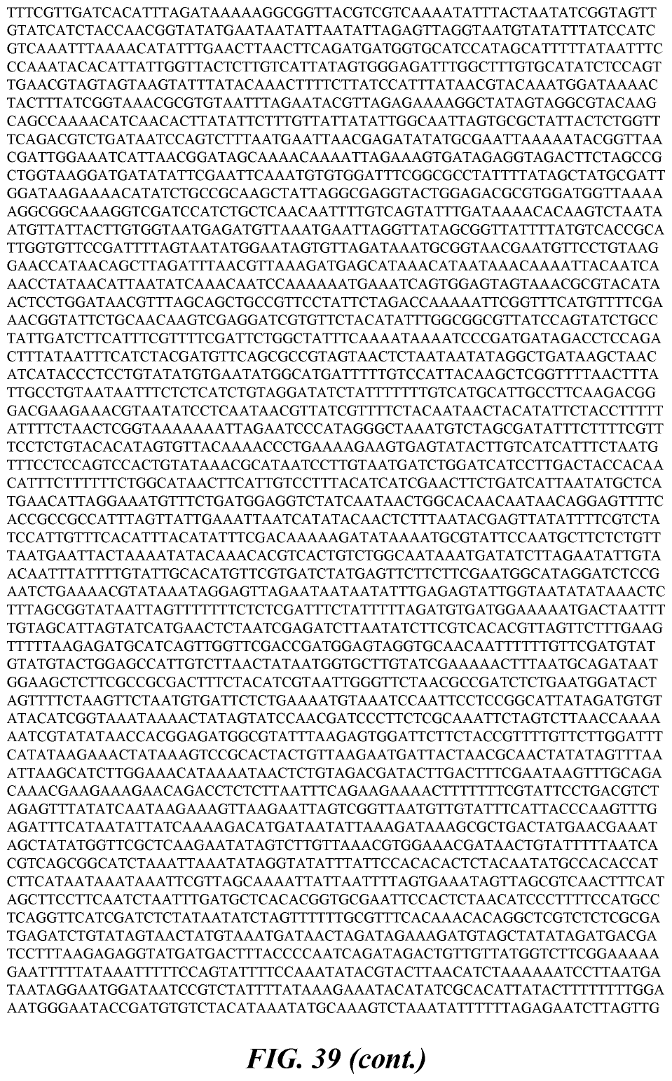

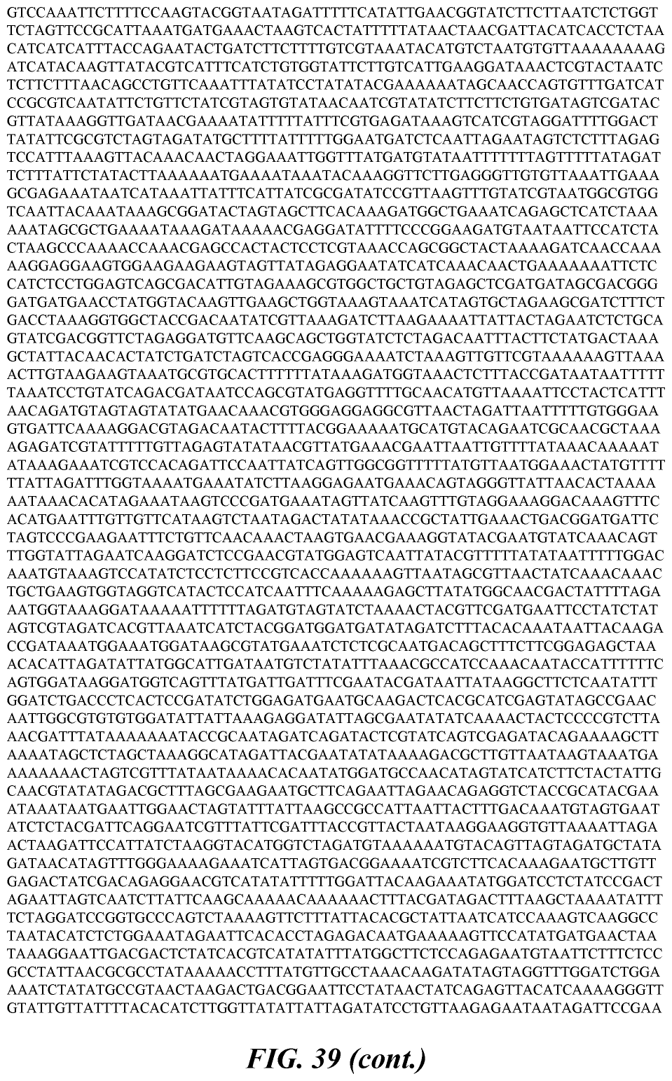

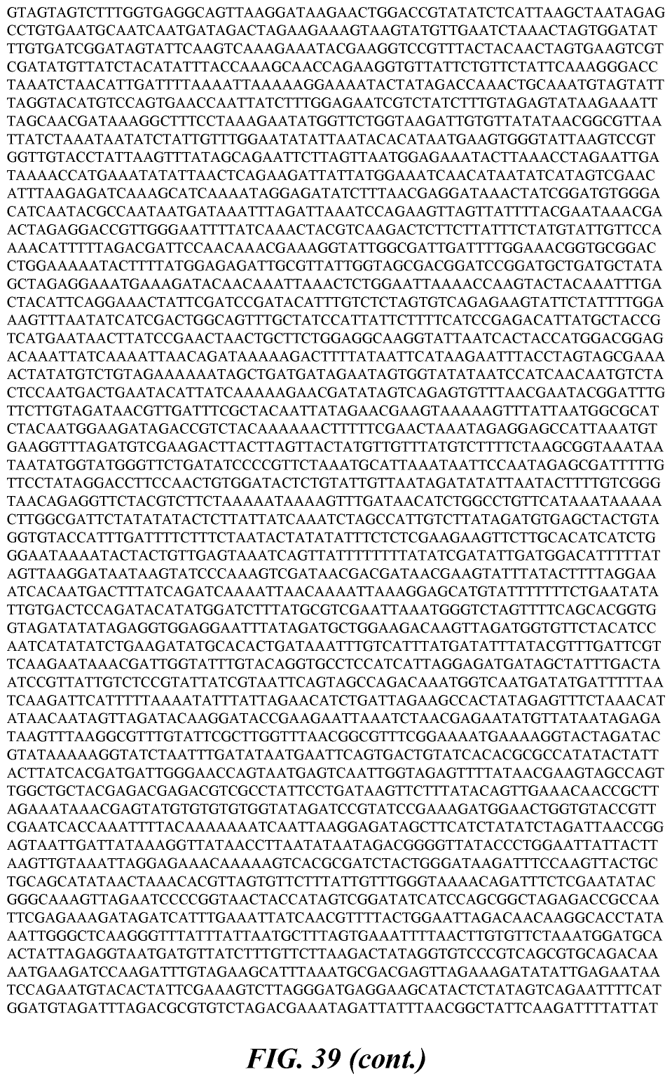

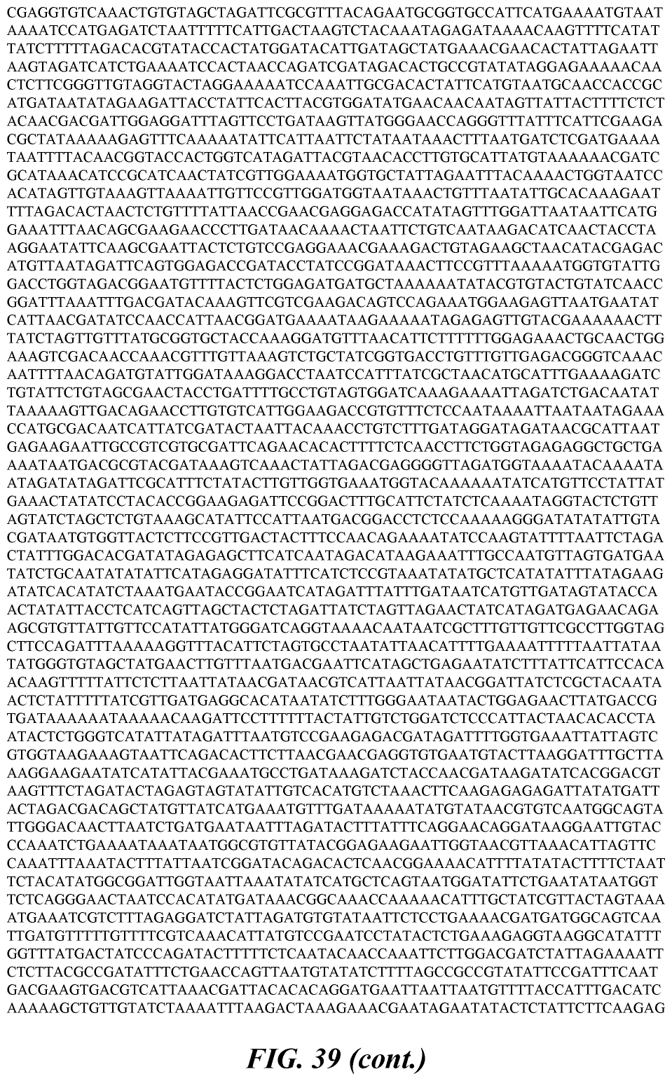

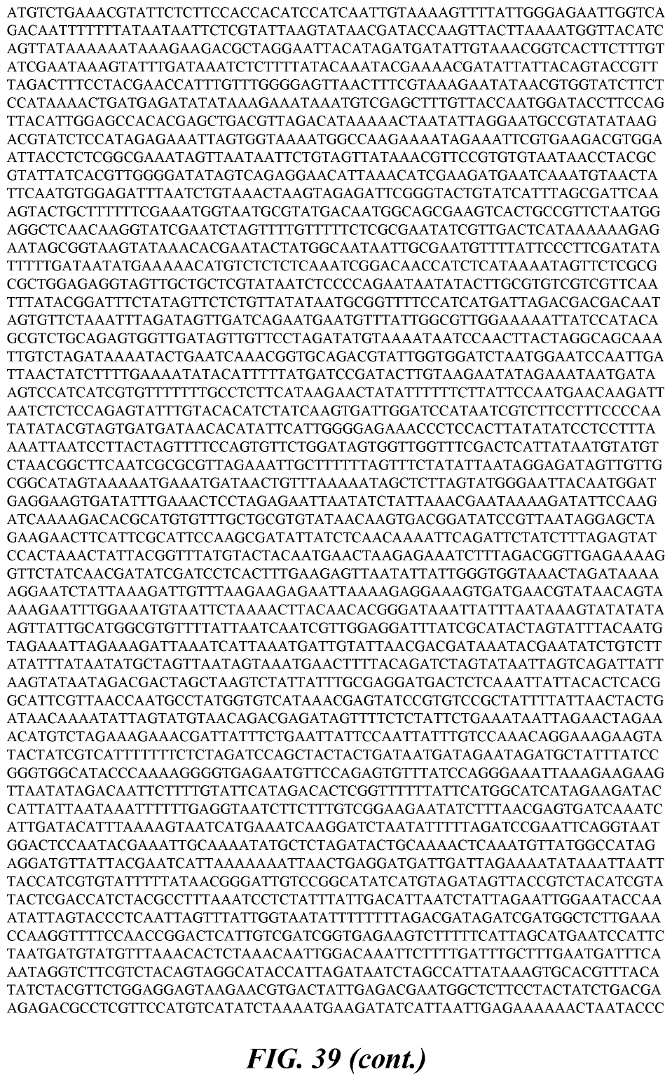

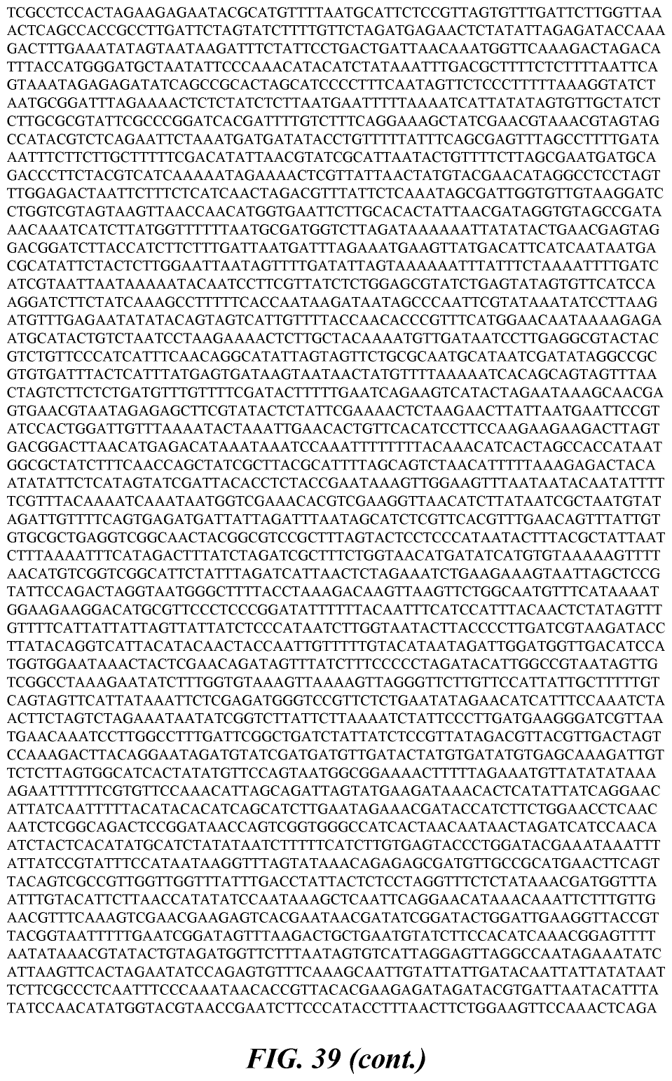

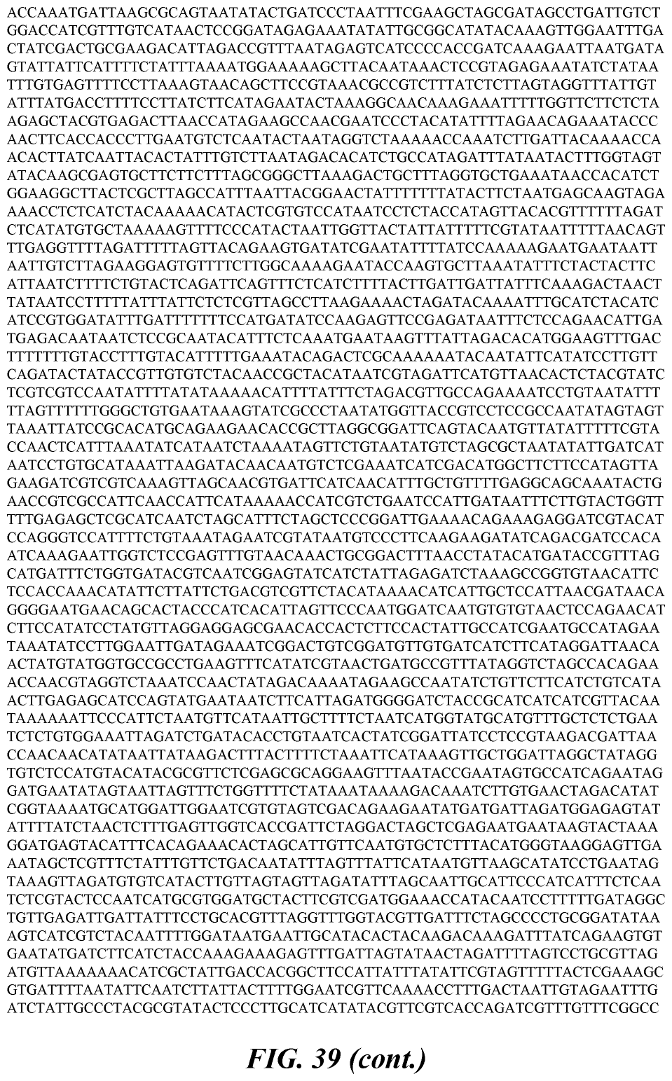

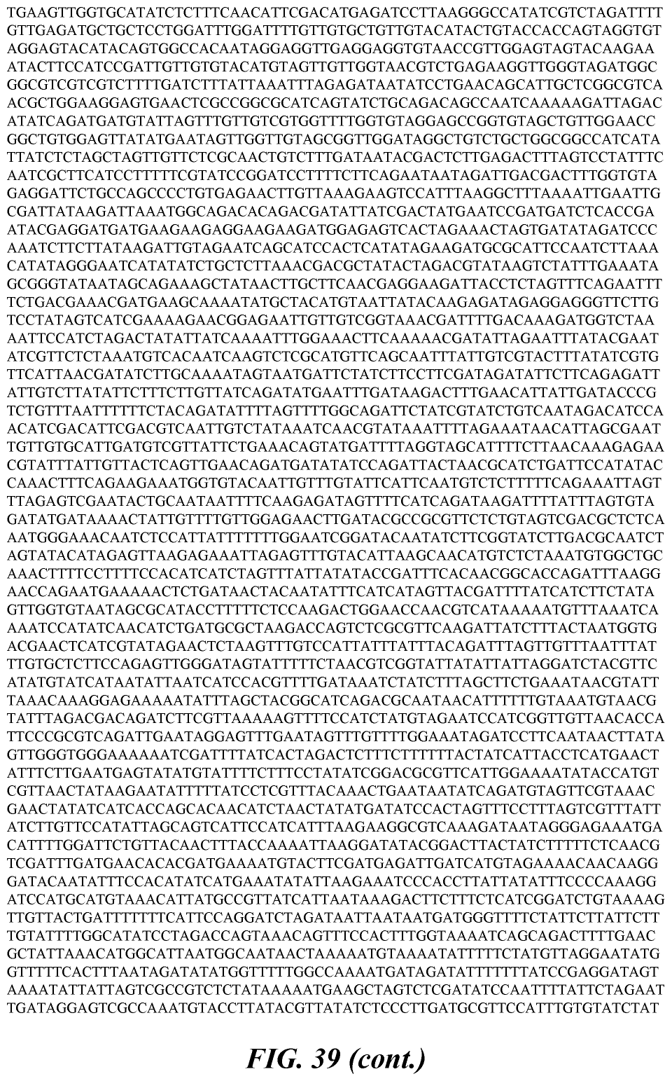

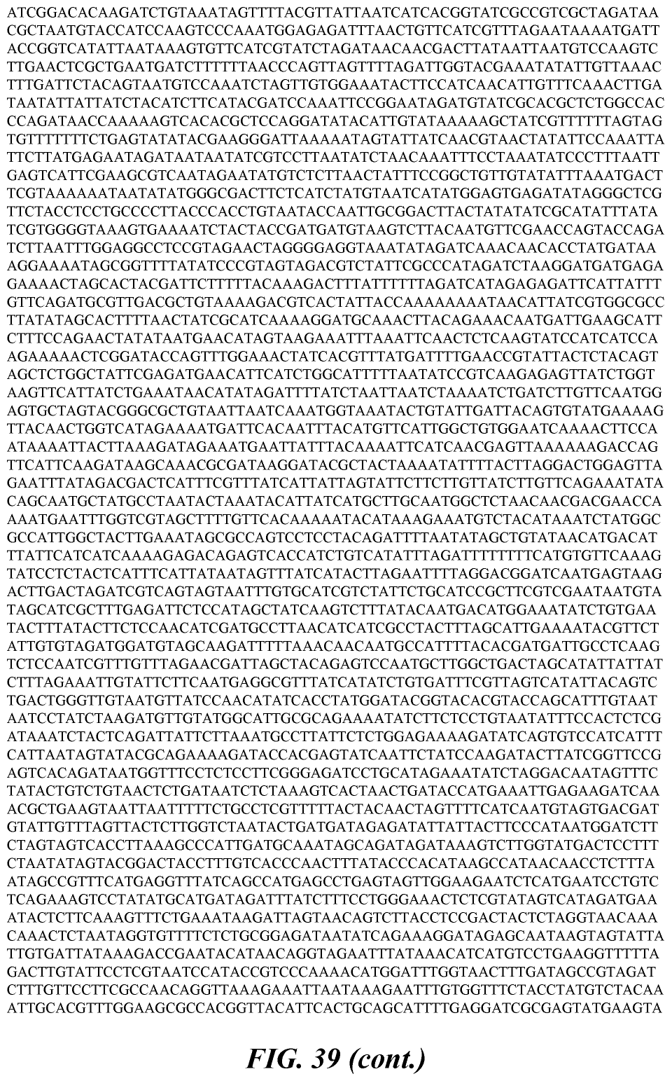

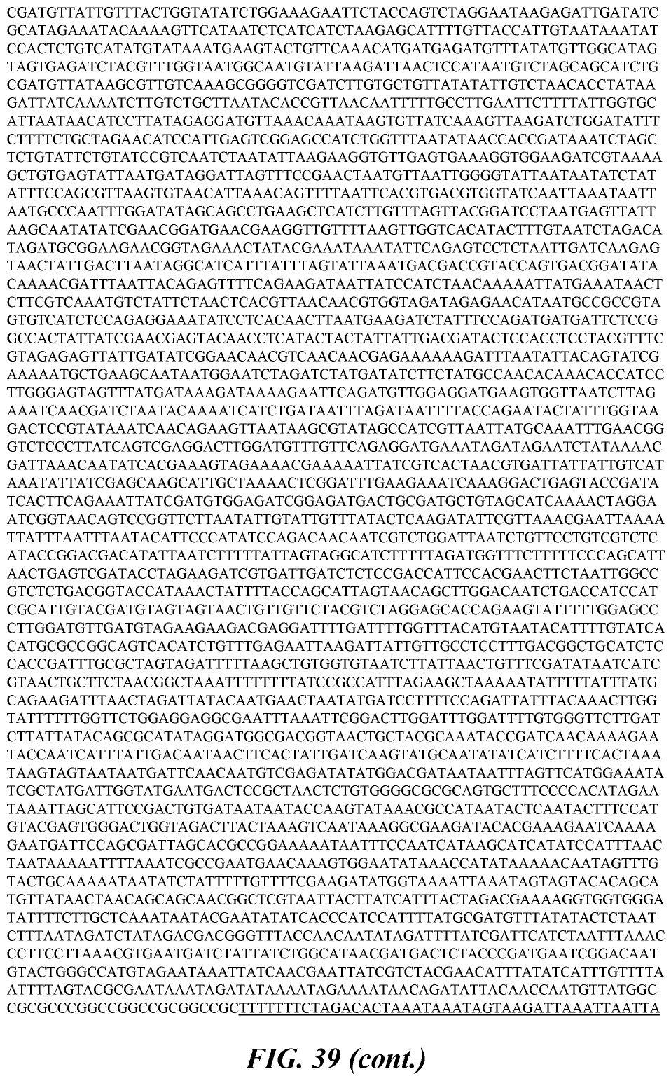

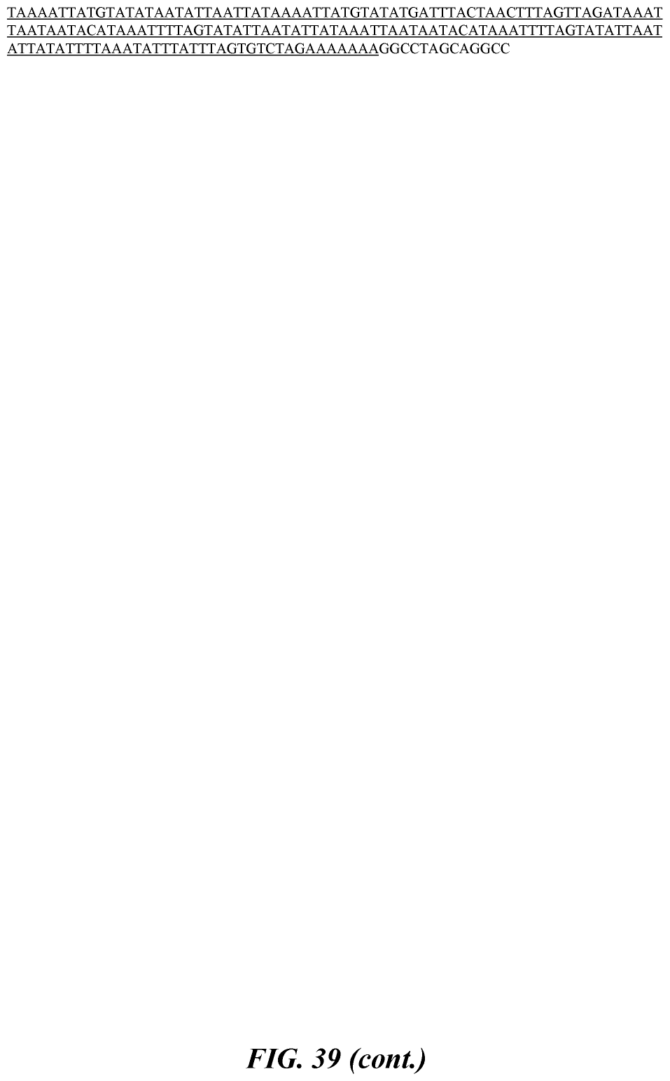

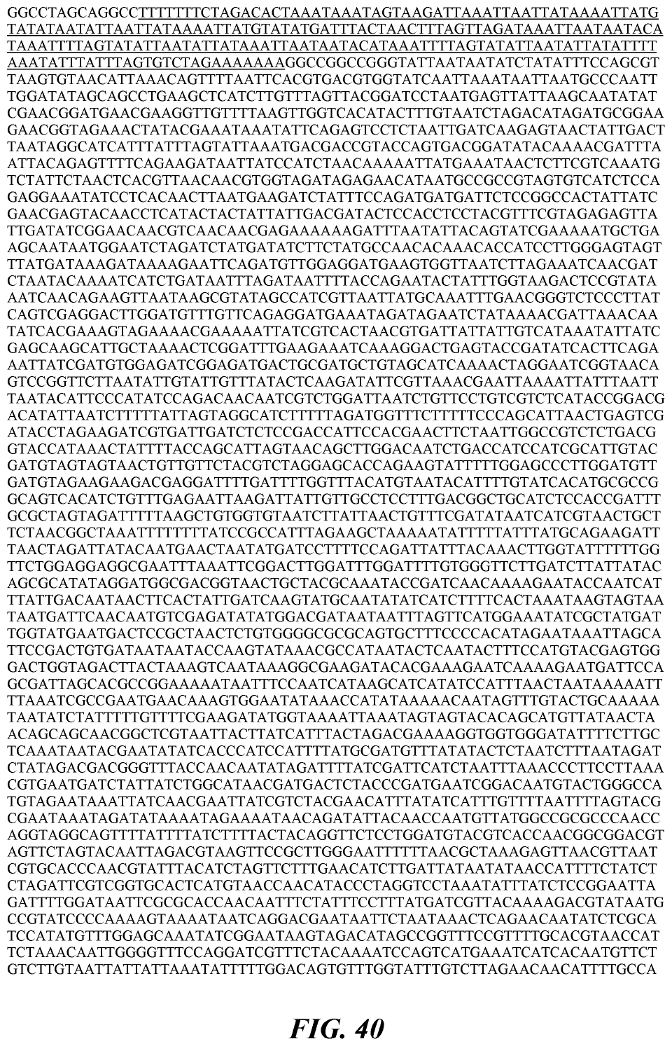

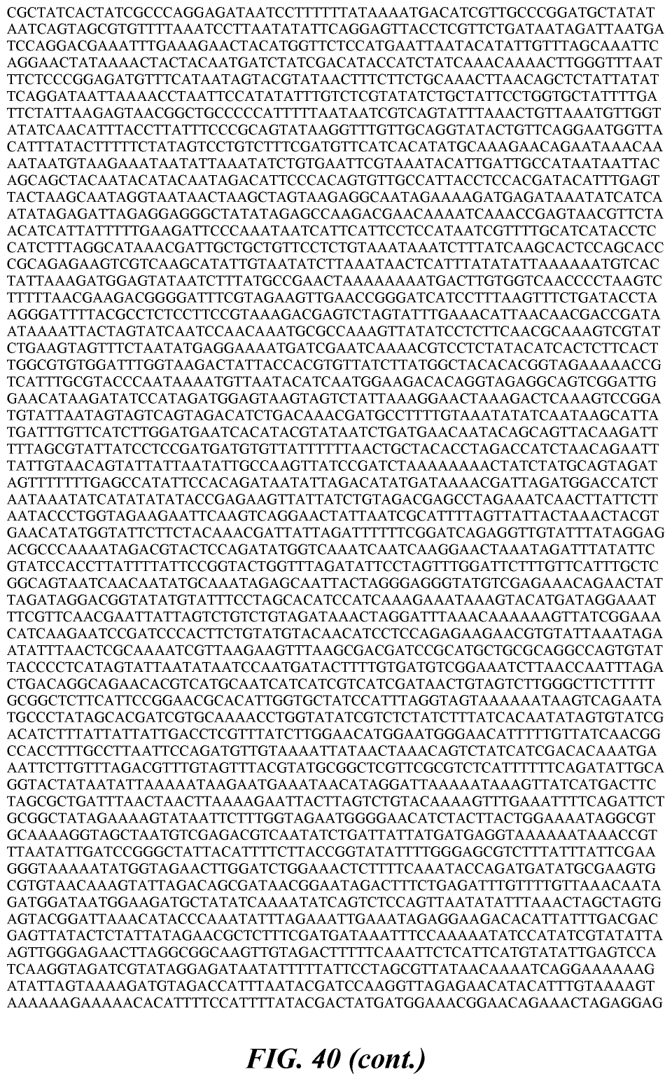

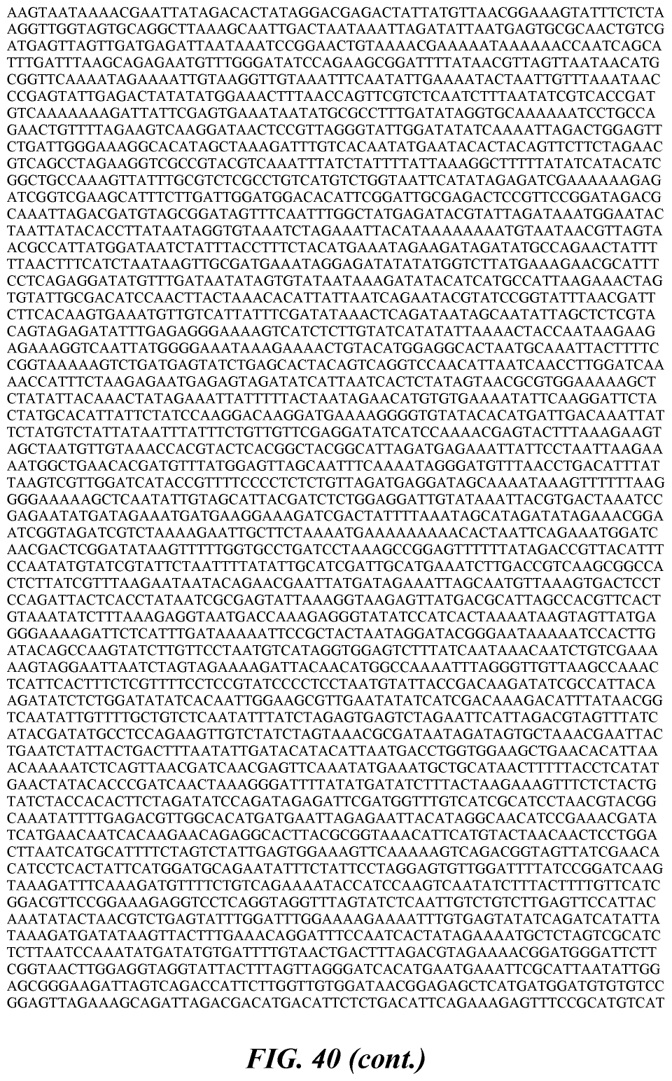

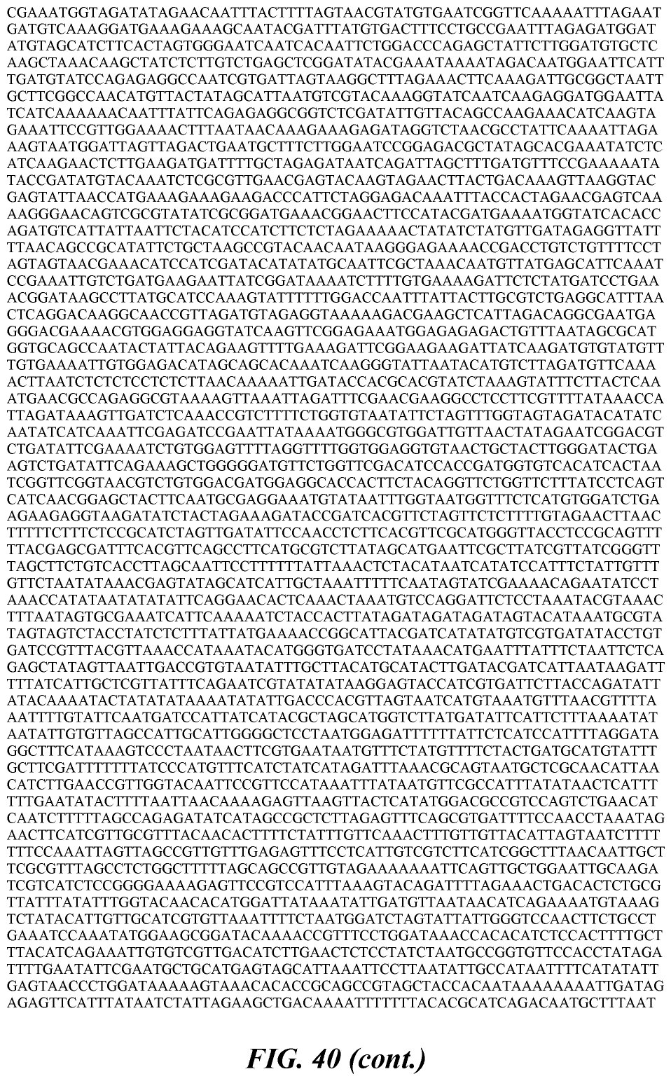

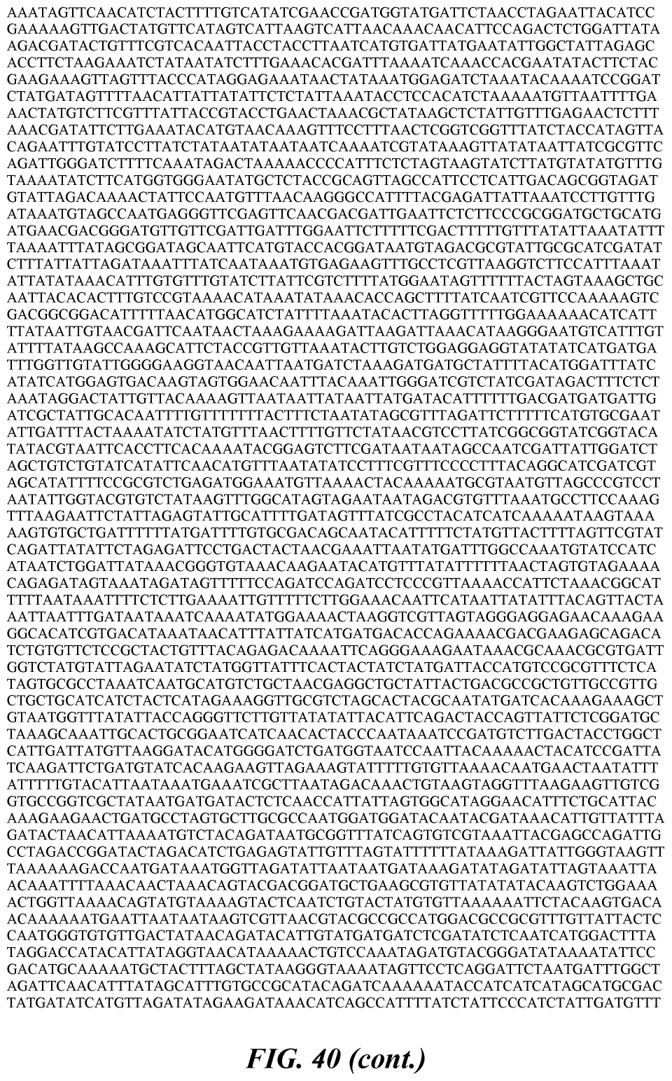

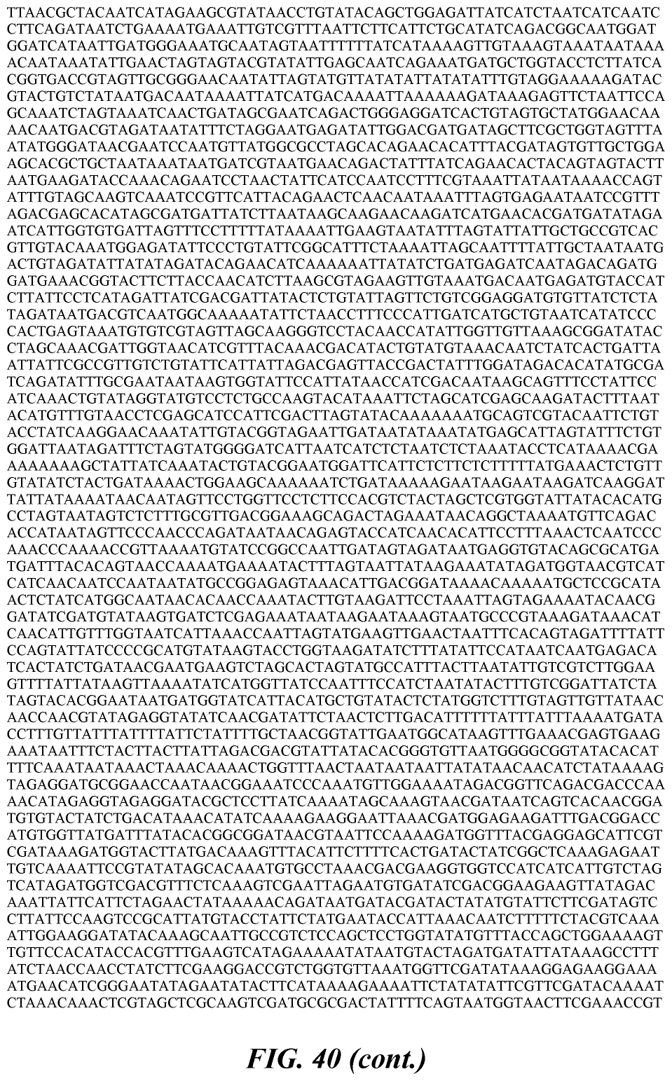

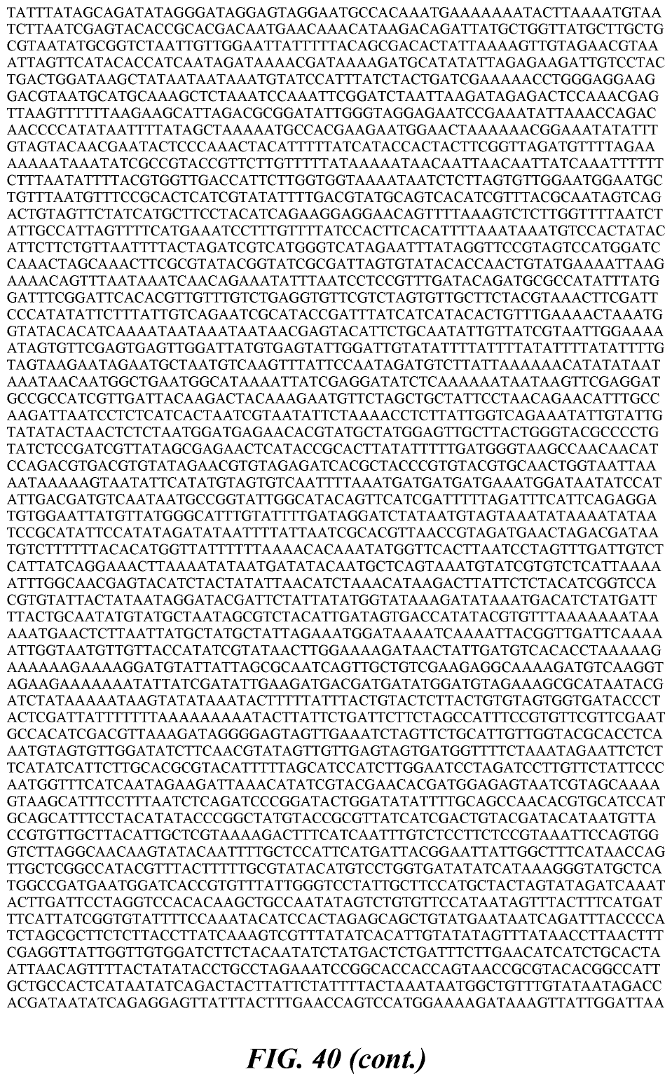

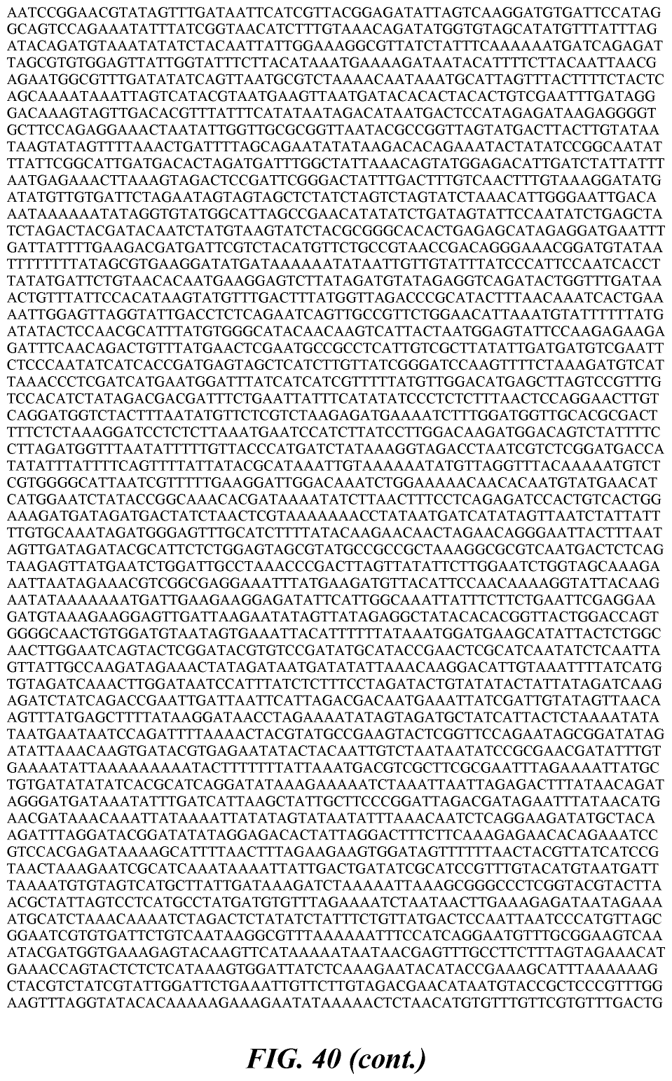

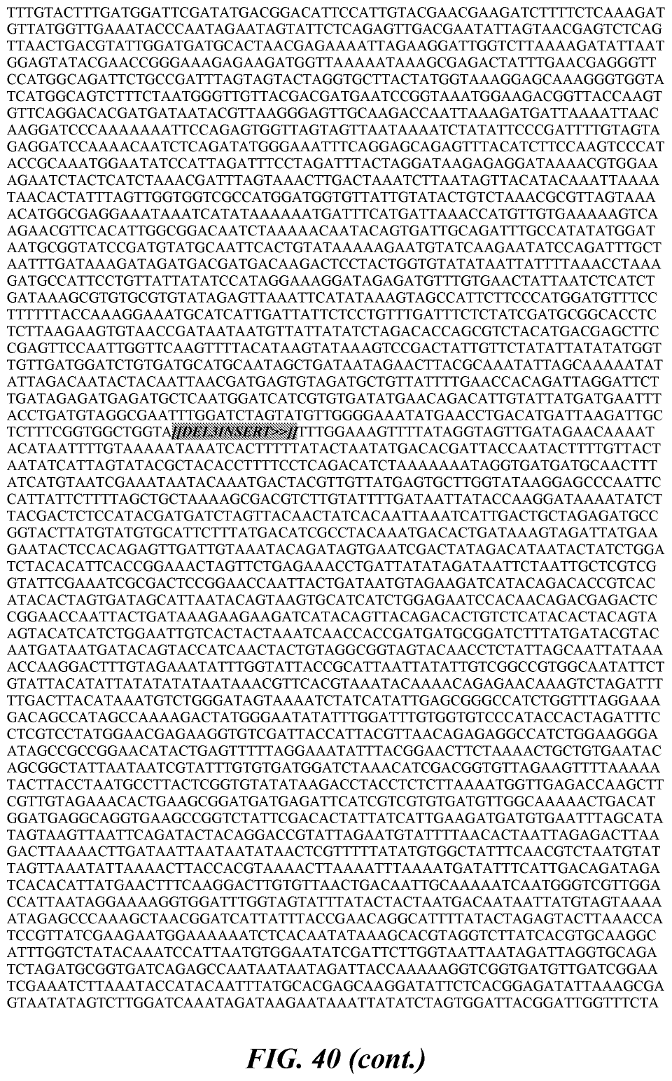

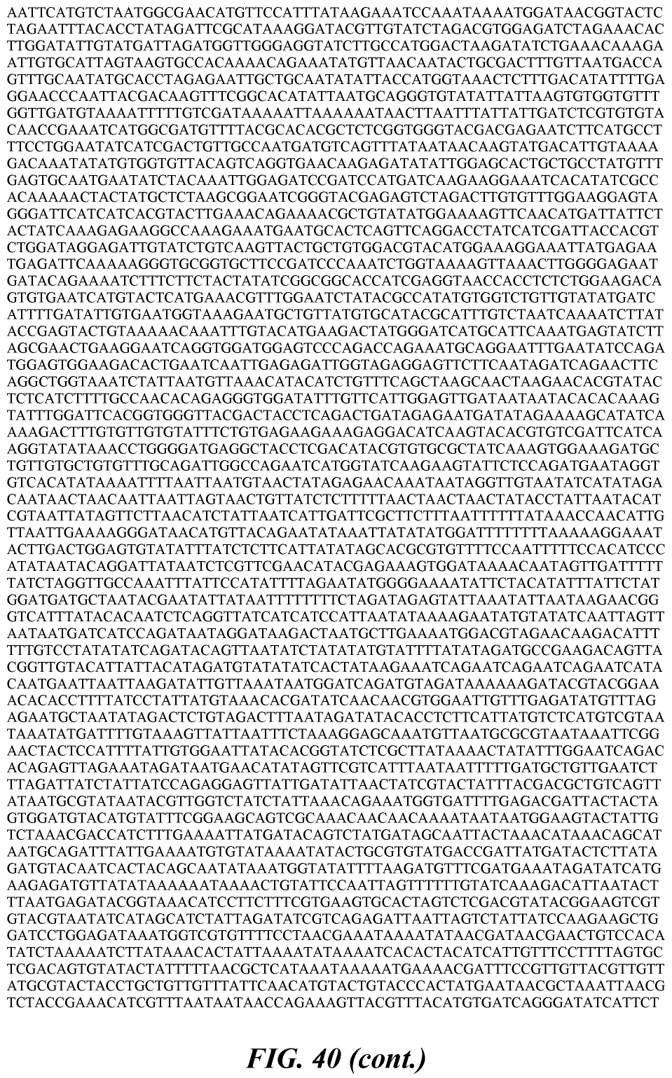

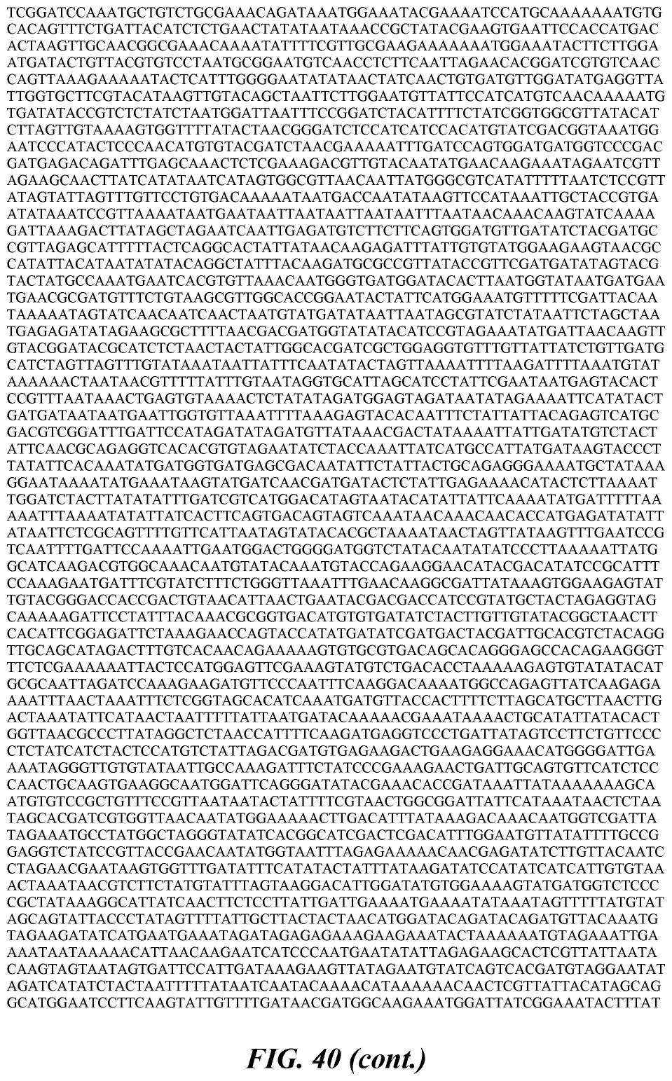

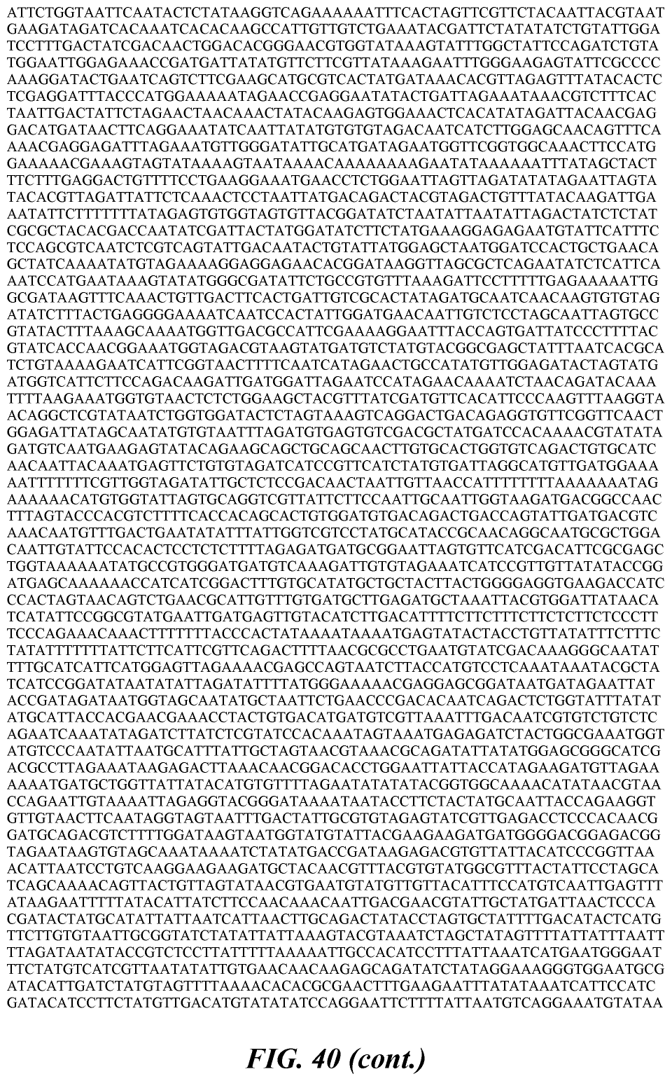

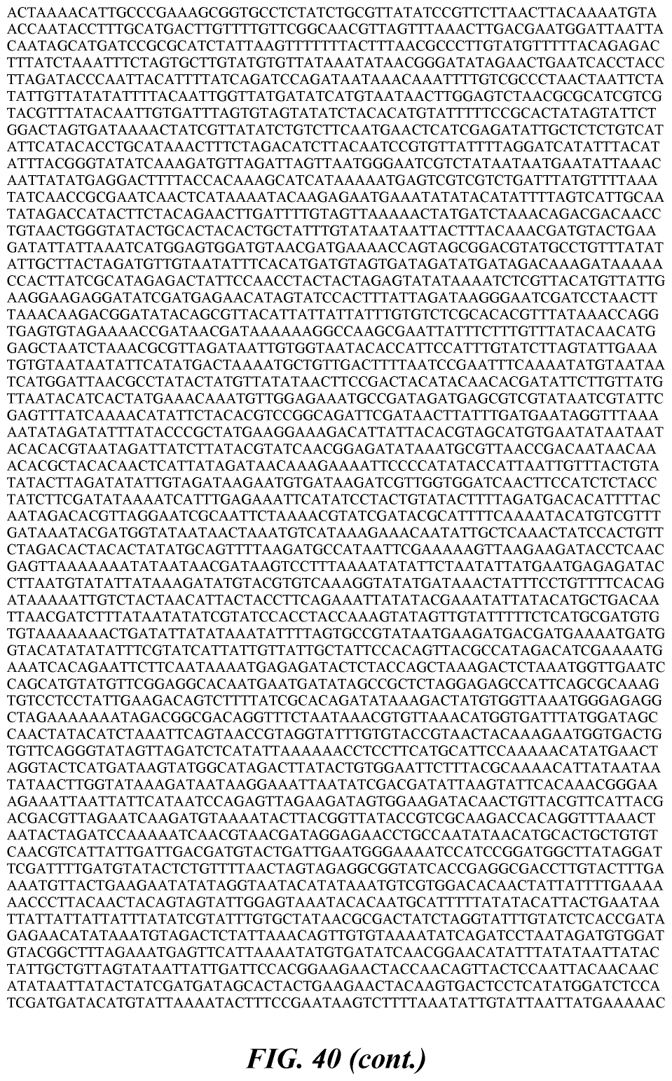

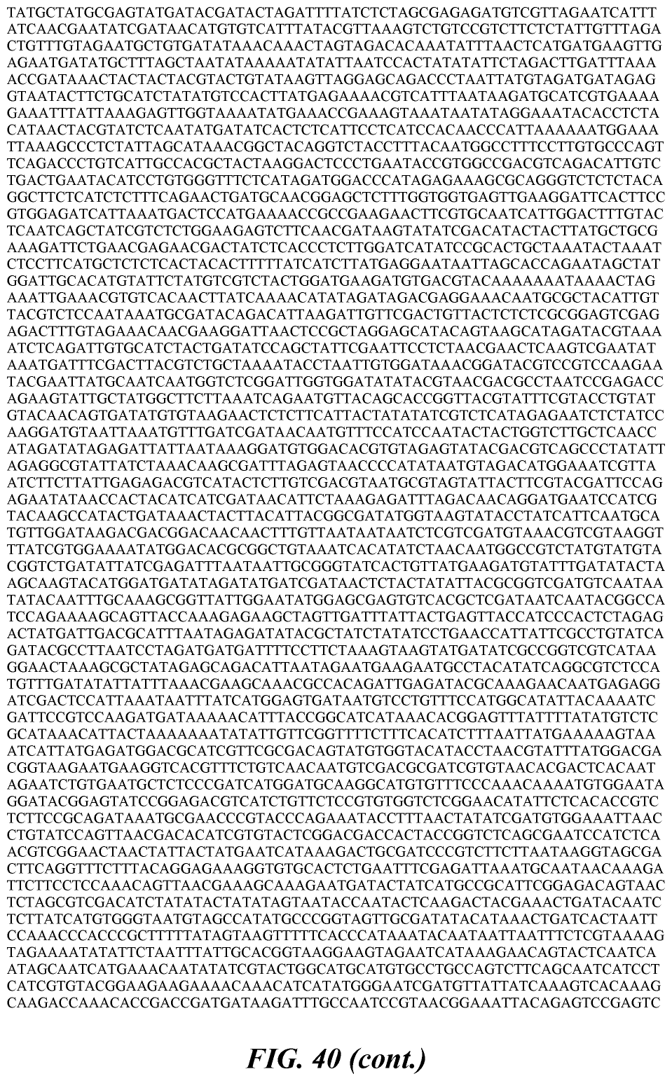

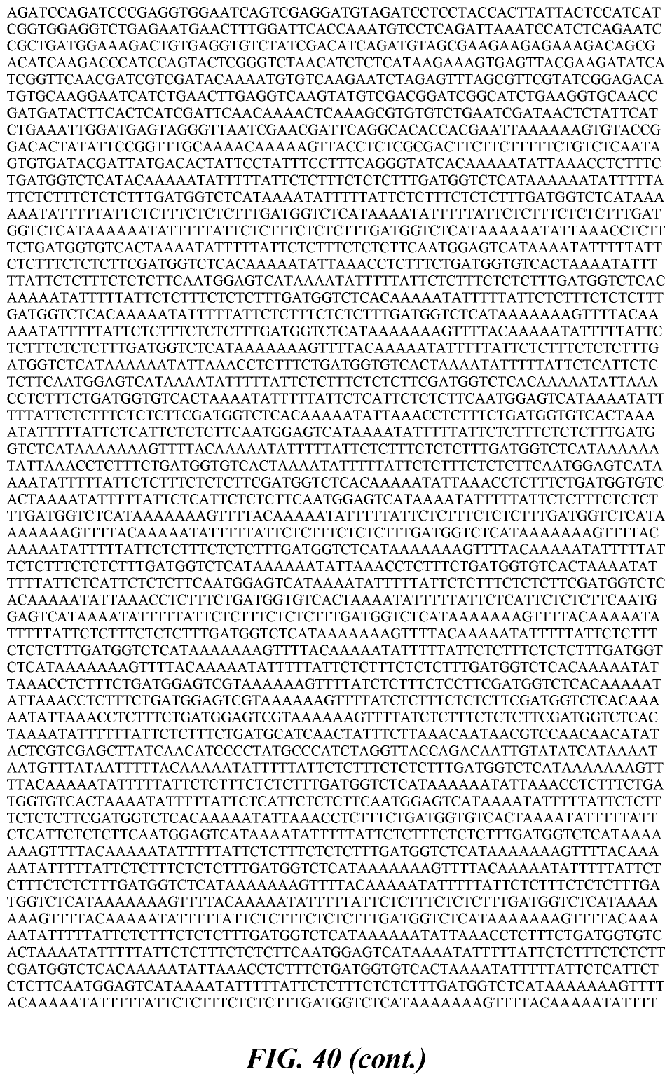

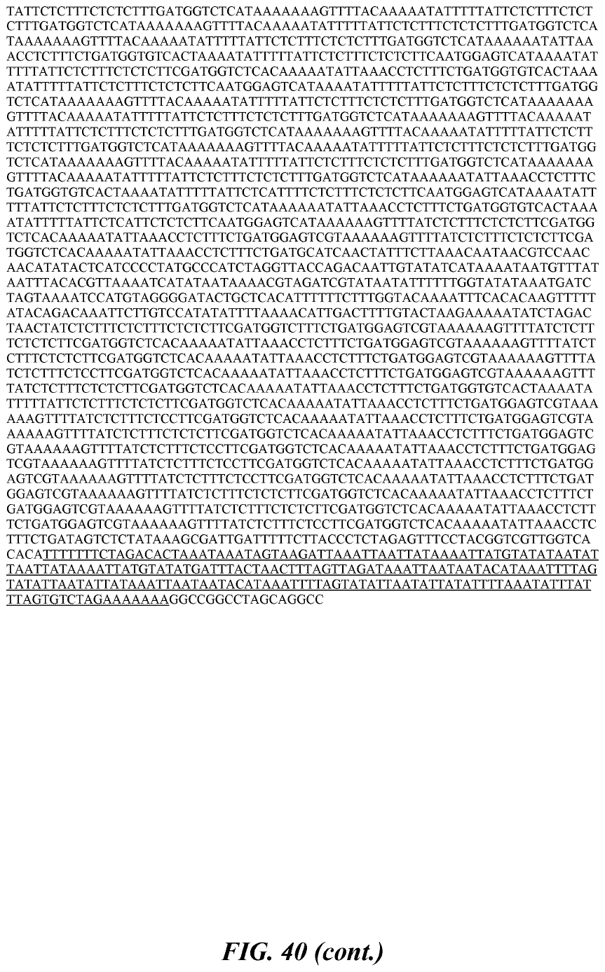

In some embodiments, the sMVA vector that is used in the compositions disclosed herein is generated according to the methods disclosed in International Application Publication No. WO 2021/236550, which is hereby incorporated by reference as if fully disclosed herein and as indicated in the working examples below. In certain of these embodiments, three nucleotide fragments, F1, F2, and F3, are synthesized, each fragment including a portion of the full MVA backbone sequence flanked by an MVA concatemer resolution-hairpin loop-concatemer resolution (CR/HL/CR) sequence (FIGS. 36B-36C). F1 encompasses the left ITR and ˜50 kbp of the left end of the internal UR of the MVA genome; F2 contains ˜60 kbp of the middle part of the internal UR of the MVA genome; and F3 encompasses ˜50 kbp of the right end of the internal UR and the right ITR of the MVA genome. sMVA F1 and F2 as well as sMVA F2 and F3 are designed to share ˜3 kbp overlapping sequences to allow the reconstitution of the complete MVA genome by homologous recombination (FIG. 36B). A duplex copy of the 165-nucleotide-long MVA terminal hairpin loop (HL) flanked by MVA CR sequences is added to both ends of each of the three fragments to promote MVA genome resolution and packaging (FIG. 36C). The three sMVA fragments are cloned and maintained in E. coli(DH10B, EPI300, GS1783) by a yeast-bacterial shuttle vector, termed pCCI-Brick (GeneScript), which contains a bacterial mini-F replicon element that can be used as a bacterial artificial chromosome (BAC) vector to stably propagate the three fragments at a low copy number in bacteria. Next-generation sequencing analysis confirmed the integrity of the MVA genomic sequences of the fragments, with the notable exception of an unknown single-point mutation within sMVA fragment F1 located in a non-coding determining region at 3 bp downstream of 021 L. FIGS. 38-40 (SEQ ID NOs: 2-4) show sequences of F1, F2, and F3, respectively, according to some embodiments. The CR/HL/CR sequences are underlined. The Del2, IGR69/70, and Del3 insertion sites are also indicated in FIGS. 38-40.

In some embodiments, the vaccines and/or other immunogenic compositions comprise a single DNA fragment comprising the entire genome sequence of MVA. The single DNA fragment can be used to transfect a host cell such that the MVA is reconstituted. In other embodiments, the vaccines and/or other immunogenic compositions comprise two or more DNA fragments each comprising a partial sequence of the genome of the MVA and having overlapping sequences at the ends of two adjacent DNA fragments, such that the two or more DNA fragments, when transferred into a host cell, are assembled sequentially and comprise the full-length sequence of the MVA genome. The overlapping sequence may be between about 100 bp and about 5000 bp in length. In certain of these embodiments, the two or more DNA fragments correspond to the F1, F2, and F3 fragments as described. In either of these embodiments, the one or more heterologous nucleotide sequences that encode (i) a SARS-CoV-2 S protein (or variant, mutant, and/or immunogenic fragment thereof) and (ii) a SARS-CoV-2 N protein (or variant, mutant, and/or immunogenic fragment thereof) can be inserted into the insertion sites as described (e.g., Del2, IGR69/70, and Del3). In the embodiments where two or more DNA fragments are used, each fragment may serve to carry a separate SARS-CoV-2 protein sequence (or variant, mutant, and/or immunogenic fragment thereof).

In some embodiments, the vaccines and/or immunogenic compositions comprise a mixture of two or more sMVA vectors which encode two or more different SARS-CoV-2 antigen sequences selected from the Wuhan-Hu-1 reference strain and different VOC. For example, one sMVA vector in the mixture comprises sequences encoding SARS-CoV-2 antigens from the Wuhan-Hu-1 reference strain, and another sMVA vector in the mixture comprises sequences encoding SARS-CoV-2 antigens from a VOC. For another example, one sMVA vector in the mixture comprises sequences encoding SARS-CoV-2 antigens from one VOC, and another sMVA vector in the mixture comprises sequences encoding SARS-CoV-2 antigens from a different VOC.

In some embodiments, the vaccines and/or immunogenic compositions comprise an sMVA vector which encodes two or more different SARS-CoV-2 antigen sequences selected from the Wuhan-Hu-1 reference strain and different VOC. For example, the sMVA vector comprises a sequence encoding a SARS-CoV-2 antigen (e.g., S protein) from the Wuhan-Hu-1 reference strain, and the same sMVA vector further comprises a sequence encoding a different SARS-CoV-2 antigen (e.g., N protein) from a VOC. For another example, the sMVA vector comprises a sequence encoding a SARS-CoV-2 antigen (e.g., S protein) from one VOC, and the same sMVA vector further comprises a sequence encoding another SARS-CoV-2 antigen (e.g., N protein) from a different VOC.

In some embodiments, a nucleotide sequence that encodes a SARS-CoV-2 S protein (or variant, mutant, and/or immunogenic fragment thereof) is inserted into the Del2 site within F1, the IGR69/70 site within F2, or the Del3 site within F3.

In some embodiments, a nucleotide sequence that encodes a SARS-CoV-2 N protein (or variant, mutant, and/or immunogenic fragment thereof) is inserted into the Del2 site within F1, and a nucleotide sequence that encodes a SARS-CoV-2 S protein (or variant, mutant, and/or immunogenic fragment thereof) is inserted into the Del3 site within F3.

In some embodiments, a nucleotide sequence that encodes a SARS-CoV-2 S protein (or variant, mutant, and/or immunogenic fragment thereof) is inserted into the Del2 site within F1, and a nucleotide sequence that encodes a SARS-CoV-2 N protein (or variant, mutant, and/or immunogenic fragment thereof) is inserted into the Del3 site within F3.

In some embodiments, a nucleotide sequence that encodes a SARS-CoV-2 S protein (or variant, mutant, and/or immunogenic fragment thereof) is inserted into the IGR69/70 site within F2, and a nucleotide sequence that encodes a SARS-CoV-2 N protein (or variant, mutant, and/or immunogenic fragment thereof) is inserted into the Del3 site within F3.

In some embodiments, a nucleotide sequence that encodes a SARS-CoV-2 N protein (or variant, mutant, and/or immunogenic fragment thereof) is inserted into the IGR69/70 site within F2, and a nucleotide sequence that encodes a SARS-CoV-2 S protein (or variant, mutant, and/or immunogenic fragment thereof) is inserted into the Del3 site within F3.

In some embodiments, a nucleotide sequence that encodes a SARS-CoV-2 S protein (or variant, mutant, and/or immunogenic fragment thereof) is inserted into the Del2 site within F1, and a nucleotide sequence that encodes a SARS-CoV-2 N protein (or variant, mutant, and/or immunogenic fragment thereof) is inserted into the IGR69/70 site within F2.

In some embodiments, a nucleotide sequence that encodes a SARS-CoV-2 N protein (or variant, mutant, and/or immunogenic fragment thereof) is inserted into the Del2 site within F1, and a nucleotide sequence that encodes a SARS-CoV-2 S protein (or variant, mutant, and/or immunogenic fragment thereof) is inserted into the IGR69/70 site within F2.

In some embodiments, the SARS-CoV-2 S protein (or variant, mutant, and/or immunogenic fragment thereof) and/or the SARS-CoV-2 N protein (or variant, mutant, and/or immunogenic fragment thereof) that are inserted into the F1, F2, and/or F3 fragments as discussed herein are encoded by any SARS-CoV-2 S or N protein, including a reference sequence or any variant or mutants thereof. Exemplary SARS-CoV-2 S or N proteins that are encoded by the heterologous sequences that can be inserted into the F1, F2, and/or F3 fragments as discussed herein (and thus incorporated into the sMVA vector and reconstituted sMVA virus) are found in International Application Publication No. WO 2021/236550, which is hereby incorporated by reference as if fully disclosed herein, as well as additional sequences and mutations discussed below.

In some embodiments, the vaccines and/or immunogenic compositions comprise one or more sMVA vectors, wherein the one or more sMVA vectors comprise one or more heterologous DNA sequences encoding the S protein and/or N protein of the SARS-CoV-2 Wuhan-Hu-1 reference strain. In some embodiments, the one or more sMVA vectors comprise one or more heterologous DNA sequences encoding a mutant S protein and/or N protein such as a mutant S protein and/or N protein based on a VOC, including B.1.1.7 (Alpha), B.1.351 (Beta), P.1 (Gamma), B.1.617.2 (Delta), B.1.621 (Mu), C.37 (Lambda), C.1.2, BA.1 (Omicron), BA.2 (Omicron), BA.2.12.1 (Omicron), BA.4 (Omicron), BA.5 (Omicron), BA.2.75 (Omicron), BQ.1 (Omicron), BQ.1.1 (Omicron), and XBB (Omicron). Exemplary sequences of the variants of the S proteins and N proteins are summarized in Table 1. According to some embodiments, the sMVA vectors are reconstituted sMVA viruses that express or are capable of expressing the one or more S proteins and/or N proteins (or mutants thereof) disclosed herein. In certain of these embodiments, the DNA sequences encoding the S and/or N protein can be codon optimized or comprise silent codon alterations to avoid consecutive nucleotides of the same kind.

TABLE 1
Exemplary sequences of SARS-COV-2 S and N proteins

| SEQ ID | | |
|---|---|---|
| NO: | Sequences | Descriptions |
| 5 | atgtttgtctttcttgtcttattgccactagtctctagtcagtgtgttaatcttacaaccag | Nucleotide |
| | aactcaattaccacctgcatacactaattctttcacacgtggtgtttattaccctgac | sequence/ORF |
| | aaagtattcagatcctcagtattacattcaactcaggacttgttcttacctttcttctcc | (5′ to 3′ end) of |
| | aatgttacttggttccatgctatacatgtctctgggaccaatggtactaagaggtttg | the SARS-CoV- |
| | ataaccctgtcctaccatttaatgatggtgtttatttcgcttccactgagaagtctaac | 2 S antigen |
| | ataataagaggctggatatttggtactactttagattcgaagacccagtccctactt | sequence based |
| | attgttaataacgctactaatgttgttattaaagtctgtgaatttcaattctgtaatgatc | on the Wuhan- |
| | cattcttgggtgtttattaccacaagaacaacaagagttggatggaaagtgagttc | Hu-1 reference |
| | agagtttattctagtgcgaataattgcactttcgaatatgtctctcagcctttccttatg | strain |
| | gaccttgaaggtaaacagggtaatttcaagaatcttagggaatttgtgtttaagaat | (#NC_045512) |
| | attgatggttatttcaagatatattctaagcacacgcctattaatttagtgcgtgatctc | and optimized |
| | cctcagggtttctcggctttagaaccattggtagatttgccaataggtattaacatca | for stability in |
| | ctaggtttcaaactttacttgctttacatagaagttatttgactcctggtgattcttcttca | vaccinia by |
| | ggttggacagctggtgctgcagcttattatgtgggttatcttcaacctaggactttcct | silent codon |
| | attgaaatataatgagaatggaaccattacagatgctgtagactgtgcacttgacc | alteration to |
| | ctctctcagaaacaaagtgtacgttgaaatccttcactgtagagaaaggaatctat | avoid 4 or more |
| | caaacttctaactttagagtccaaccaacagaatctattgttagatttcctaatatta | of the same |
| | caaacttgtgccctttcggtgaagtatttaacgccaccagatttgcatctgtttatgctt | nucleotides in |
| | ggaacaggaagagaatcagcaactgtgttgctgattattctgtcctatataattccg | consecutive |
| | catcattctccacatttaagtgttatggagtgtctcctactaaattaaatgatctctgct | order |
| | ttactaatgtctatgcagattcatttgtaattagaggtgatgaagtcagacaaatcg | |
| | ctccagggcaaactggaaagattgctgattataattataaattaccagatgacttta | |
| | caggctgcgttatagcttggaattctaacaatcttgattctaaggttggtggtaattat | |
| | aattacctgtatagattgtttaggaagtctaatctcaaacctttcgagagagatatttc | |
| | aactgaaatctatcaggccggtagcacaccttgtaatggtgttgaaggatttaattg | |
| | ttactttcctttacaatcatatggtttccaacccactaatggtgttggttaccaaccata | |
| | cagagtagtagtactttcatttgaacttctacatgcaccagcaactgtttgtggacct | |
| | aagaagtctactaatttggttaagaacaaatgtgtcaatttcaacttcaatggtttaa | |
| | caggcacaggtgttcttactgagtctaacaagaagtttctgcctttccaacaatttg | |
| | gcagagacattgctgacactactgatgctgtccgtgatccacagacacttgagatt | |
| | cttgacattacaccatgttcatttggtggtgtcagtgttataacaccaggaacaaat | |
| | acttctaaccaggttgctgttctttatcaggatgttaactgcacagaagtccctgttgc | |
| | tattcatgcagatcaacttactcctacttggcgtgtttattctacaggttctaatgtattt | |
| | caaacacgtgcaggctgtttaataggagctgaacatgtcaacaactcatatgagt | |
| | gtgacatacccattggtgcaggtatatgcgctagttatcagactcagactaattctc | |
| | ctcggcgggcacgtagtgtagctagtcaatccatcattgcctacactatgtcacttg | |
| | gtgcagagaattcagttgcttactctaataactctattgccatacccacaaactttac | |
| | tattagtgttaccacagaaattctaccagtgtctatgaccaagacatcagtagattg | |
| | tacaatgtacatttgtggtgattcaactgaatgcagcaatctattgttgcaatatggc | |
| | agtttctgtacacaattaaaccgtgctttaactggaatagctgttgaacaagacaa | |
| | gaacacccaagaagtatttgcacaagtcaaacaaatttacaagacaccaccaa | |
| | ttaaagatttcggtggatttaatttctcacaaatattaccagatccatctaaaccaag | |
| | caagaggtcatttattgaagatctactattcaacaaagtgacacttgcagatgctg | |
| | gcttcatcaaacaatatggtgattgccttggtgatattgctgctagagacctcatttgt | |
| | gcacagaagtttaacggccttactgtattgccacctttgctcacagatgaaatgatt | |
| | gctcaatacacttctgcactgttagcgggtacaatcacttctggttggacctttggtg | |
| | caggtgctgcattacaaataccatttgctatgcaaatggcttataggtttaatggtatt | |
| | ggagttacacagaatgttctctatgagaaccagaaattgattgccaaccaatttaa | |
| | tagtgctattggcaagattcaagactcactttcttccacagcaagtgcacttggtaa | |
| | acttcaagatgtggtcaaccagaatgcacaagctttaaacacgcttgttaaacaa | |
| | cttagctccaactttggtgcaatttcaagtgtattaaatgatatcctttcacgtcttgac | |
| | aaagttgaggctgaagtgcaaattgataggttgatcacaggcagacttcaaagtt | |
| | tgcagacatatgtgactcaacaattaattagagctgcagaaatcagagcttctgct | |
| | aatcttgctgctactaagatgtcagagtgtgtacttggacaatctaagagagttgat | |
| | ttctgtggaaagggctatcatcttatgtccttccctcagtcagcacctcatggtgtagt | |
| | cttcttgcatgtgacttatgtccctgcacaagagaagaacttcacaactgctcctgc | |
| | catttgtcatgatggtaaagcacactttcctcgtgaaggtgtctttgtttcaaatggca | |
| | cacactggtttgtaacacaaaggaatttctatgaaccacaaatcattactacaga | |
| | caacacatttgtgtctggtaactgtgatgttgtaataggaattgtcaacaacacagtt | |
| | tatgatcctttgcaacctgaattagactcattcaaggaggagttagataaatatttca | |
| | agaatcatacatcaccagatgttgatttaggtgacatctctggcattaatgcttcagt | |
| | tgtaaacattcagaaagaaattgaccgcctcaatgaggttgccaagaatttaaat | |
| | gaatctctcatcgatctccaagaacttggaaagtatgagcagtatatcaaatggc | |
| | catggtacatttggctaggtttcatagctggcttgattgccatagtaatggtgacaatt | |
| | atgctttgctgtatgaccagttgctgtagttgtctcaagggctgttgttcttgtggatcct | |
| | gctgcaaatttgatgaagacgactctgagccagtgctcaaaggagtcaaattac | |
| | attacacataa | |
| 6 | MFVFLVLLPLVSSQCVNLTTRTQLPPAYTNSFTRGVYYPD | Amino acid |
| | KVFRSSVLHSTQDLFLPFFSNVTWFHAIHVSGTNGTKRF | sequence (N- to |
| | DNPVLPFNDGVYFASTEKSNIIRGWIFGTTLDSKTQSLLIV | C-terminus) of |
| | NNATNVVIKVCEFQFCNDPFLGVYYHKNNKSWMESEFR | the SARS-CoV- |
| | VYSSANNCTFEYVSQPFLMDLEGKQGNFKNLREFVFKNI | 2 S antigen |
| | DGYFKIYSKHTPINLVRDLPQGFSALEPLVDLPIGINITRFQ | sequence based |
| | TLLALHRSYLTPGDSSSGWTAGAAAYYVGYLQPRTFLLK | on the Wuhan- |
| | YNENGTITDAVDCALDPLSETKCTLKSFTVEKGIYQTSNF | Hu-1 reference |
| | RVQPTESIVRFPNITNLCPFGEVFNATRFASVYAWNRKRI | strain |
| | SNCVADYSVLYNSASFSTFKCYGVSPTKLNDLCFTNVYA | (#NC_045512) |
| | DSFVIRGDEVRQIAPGQTGKIADYNYKLPDDFTGCVIAWN | |
| | SNNLDSKVGGNYNYLYRLFRKSNLKPFERDISTEIYQAGS | |
| | TPCNGVEGFNCYFPLQSYGFQPTNGVGYQPYRVVVLSF | |
| | ELLHAPATVCGPKKSTNLVKNKCVNFNFNGLTGTGVLTE | |
| | SNKKFLPFQQFGRDIADTTDAVRDPQTLEILDITPCSFGG | |
| | VSVITPGTNTSNQVAVLYQDVNCTEVPVAIHADQLTPTW | |
| | RVYSTGSNVFQTRAGCLIGAEHVNNSYECDIPIGAGICAS | |
| | YQTQTNSPRRARSVASQSIIAYTMSLGAENSVAYSNNSIA | |
| | IPTNFTISVTTEILPVSMTKTSVDCTMYICGDSTECSNLLLQ | |
| | YGSFCTQLNRALTGIAVEQDKNTQEVFAQVKQIYKTPPIK | |
| | DFGGFNFSQILPDPSKPSKRSFIEDLLFNKVTLADAGFIKQ | |
| | YGDCLGDIAARDLICAQKFNGLTVLPPLLTDEMIAQYTSAL | |
| | LAGTITSGWTFGAGAALQIPFAMQMAYRFNGIGVTQNVL | |
| | YENQKLIANQFNSAIGKIQDSLSSTASALGKLQDVVNQNA | |
| | QALNTLVKQLSSNFGAISSVLNDILSRLDKVEAEVQIDRLIT | |
| | GRLQSLQTYVTQQLIRAAEIRASANLAATKMSECVLGQSK | |
| | RVDFCGKGYHLMSFPQSAPHGVVFLHVTYVPAQEKNFT | |
| | TAPAICHDGKAHFPREGVFVSNGTHWFVTQRNFYEPQIIT | |
| | TDNTFVSGNCDVVIGIVNNTVYDPLQPELDSFKEELDKYF | |
| | KNHTSPDVDLGDISGINASVVNIQKEIDRLNEVAKNLNESL | |
| | IDLQELGKYEQYIKWPWYIWLGFIAGLIAIVMVTIMLCCMT | |
| | SCCSCLKGCCSCGSCCKFDEDDSEPVLKGVKLHYT | |
| 7 | atgtctgataacggaccacagaatcagcgaaacgcaccacgcattacgtttggt | Nucleotide |
| | ggaccctcagattcaactggcagtaaccagaatggagaacgcagtggagcgc | sequence/ORF |
| | gatctaaacaacgtcggcctcaaggtttacccaataatactgcgtcttggttcacc | (5′ to 3′ end) of |
| | gctctcactcaacatggcaaggaagaccttaaattccctcgaggacaaggcgtt | the SARS-CoV- |
| | ccaattaacaccaatagcagtccagatgaccaaattggctactaccgaagagct | 2 N antigen |
| | accagacgaattcgtggtggtgacggtaagatgaaagatctcagtccaagatgg | sequence based |
| | tatttctactacctaggaactgggccagaagctggacttccctatggtgctaacaa | on the Wuhan- |
| | agacggcatcatatgggttgcaactgagggagccttgaatacacctaaagatca | Hu-1 reference |
| | cattggcacccgcaatcctgctaacaatgctgcaatcgtgctacaacttcctcaag | strain |
| | gaacaacattgcctaaaggcttctacgcagaagggagcagaggcggcagtca | (#NC_045512) |
| | agcctcttctcgttcctcatcacgtagtcgcaacagttcaagaaattcaactccag | and optimized |
| | gcagcagtagaggaacttctcctgctagaatggctggcaatggcggtgatgctg | for stability in |
| | ctcttgctttgctgctgcttgacagattgaaccagcttgagagcaagatgtctggta | vaccinia by |
| | aaggccaacaacaacaaggccaaactgtcactaagaaatctgctgctgaggct | silent codon |
| | tctaagaagcctcggcagaaacgtactgccactaaagcatacaatgtaacaca | alteration to |
| | agctttcggcagacgtggtccagaacaaacccaaggaaactttggagaccag | avoid 4 or more |
| | gaactaatcagacaaggaactgattacaaacattggccgcaaattgcacaattt | of the same |
| | gctcccagcgcttcagcgttcttcggaatgtcgcgcattggcatggaagtcacac | nucleotides in |
| | cttcgggaacgtggttgacctacacaggtgccatcaaattggatgacaaagatc | consecutive |
| | caaatttcaaagatcaagtcatattgctgaataagcatattgacgcatacaagac | order |
| | attcccaccaacagagcctaagaaggacaagaagaagaaggctgatgaaac | |
| | tcaagccttaccgcagagacagaagaaacagcaaactgtgactcttcttcctgct | |
| | gcagatttggatgatttctccaaacaattgcaacaatccatgagcagtgctgactc | |
| | aactcaggcctaa | |
| 8 | MSDNGPQNQRNAPRITFGGPSDSTGSNQNGERSGARS | Amino acid |
| | KQRRPQGLPNNTASWFTALTQHGKEDLKFPRGQGVPIN | sequence (N- to |
| | TNSSPDDQIGYYRRATRRIRGGDGKMKDLSPRWYFYYL | C-terminus) of |
| | GTGPEAGLPYGANKDGIIWVATEGALNTPKDHIGTRNPA | the SARS-CoV- |
| | NNAAIVLQLPQGTTLPKGFYAEGSRGGSQASSRSSSRSR | 2 N antigen |
| | NSSRNSTPGSSRGTSPARMAGNGGDAALALLLLDRLNQ | sequence based |
| | LESKMSGKGQQQQGQTVTKKSAAEASKKPRQKRTATKA | on the Wuhan- |
| | YNVTQAFGRRGPEQTQGNFGDQELIRQGTDYKHWPQIA | Hu-1 reference |
| | QFAPSASAFFGMSRIGMEVTPSGTWLTYTGAIKLDDKDP | strain |
| | NFKDQVILLNKHIDAYKTFPPTEPKKDKKKKADETQALPQ | (#NC_045512) |
| | RQKKQQTVTLLPAADLDDFSKQLQQSMSSADSTQA | |
| 9 | atgtttgtctttcttgtcttattgccactagtctctagtcagtgtgttaatcttagaaccag | Nucleotide |
| | aactcaattaccacctgcatacactaattctttcacacgtggtgtttattaccctgac | sequence/ORF |
| | aaagtattcagatcctcagtattacattcaactcaggacttgttcttacctttcttctcc | (5′ to 3′ end) of |
| | aatgttacttggttccatgctatacatgtctctgggaccaatggtactaagaggtttg | a codon- |
| | ataaccctgtcctaccatttaatgatggtgtttatttcgcttccactgagaagtctaac | optimized |
| | ataataagaggctggatatttggtactactttagattcgaagacccagtccctactt | SARS-CoV-2 S |
| | attgttaataacgctactaatgttgttattaaagtctgtgaatttcaattctgtaatgatc | antigen |
| | cattcttggatgtttattaccacaagaacaacaagagttggatggaaagtggagtt | sequence based |
| | tattctagtgcgaataattgcactttcgaatatgtctctcagcctttccttatggaccttg | on the Wuhan- |
| | aaggtaaacagggtaatttcaagaatcttagggaatttgtgtttaagaatattgatg | Hu-1 reference |
| | gttatttcaagatatattctaagcacacgcctattaatttagtgcgtgatctccctcag | strain |
| | ggtttctcggctttagaaccattggtagatttgccaataggtattaacatcactaggtt | (#NC_045512) |
| | tcaaactttacttgctttacatagaagttatttgactcctggtgattcttcttcaggttgg | and optimized |
| | acagctggtgctgcagcttattatgtgggttatcttcaacctaggactttcctattgaa | for vaccinia, |
| | atataatgagaatggaaccattacagatgctgtagactgtgcacttgaccctctct | which is further |
| | cagaaacaaagtgtacgttgaaatccttcactgtagagaaaggaatctatcaaa | modified to |
| | cttctaactttagagtccaaccaacagaatctattgttagatttcctaatattacaaa | encode for an S |
| | cttgtgccctttcggtgaagtatttaacgccaccagatttgcatctgtttatgcttgga | antigen that |
| | acaggaagagaatcagcaactgtgttgctgattattctgtcctatataattccgcat | includes |
| | cattctccacatttaagtgttatggagtgtctcctactaaattaaatgatctctgcttta | mutations of the |
| | ctaatgtctatgcagattcatttgtaattagaggtgatgaagtcagacaaatcgctc | B.1.617.2 |
| | cagggcaaactggaaagattgctgattataattataaattaccagatgactttaca | variant lineage |
| | ggctgcgttatagcttggaattctaacaatcttgattctaaggttggtggtaattataa | identified in |
| | ttaccggtatagattgtttaggaagtctaatctcaaacctttcgagagagatatttca | India, including |
| | actgaaatctatcaggccggtagcaaaccttgtaatggtgttgaaggatttaattgt | T19R, G142D, |
| | tactttcctttacaatcatatggtttccaacccactaatggtgttggttaccaaccata | Del156/157, |
| | cagagtagtagtactttcatttgaacttctacatgcaccagcaactgtttgtggacct | R158G, L452R, |
| | aagaagtctactaatttggttaagaacaaatgtgtcaatttcaacttcaatggtttaa | T478K, D614G, |
| | caggcacaggtgttcttactgagtctaacaagaagtttctgcctttccaacaatttg | P681R, and |
| | gcagagacattgctgacactactgatgctgtccgtgatccacagacacttgagatt | D950N |
| | cttgacattacaccatgttcatttggtggtgtcagtgttataacaccaggaacaaat | |
| | acttctaaccaggttgctgttctttatcagggtgttaactgcacagaagtccctgttgc | |
| | tattcatgcagatcaacttactcctacttggcgtgtttattctacaggttctaatgtattt | |
| | caaacacgtgcaggctgtttaataggagctgaacatgtcaacaactcatatgagt | |
| | gtgacatacccattggtgcaggtatatgcgctagttatcagactcagactaattctc | |
| | gtcggcgggcacgtagtgtagctagtcaatccatcattgcctacactatgtcacttg | |
| | gtgcagagaattcagttgcttactctaataactctattgccatacccacaaactttac | |
| | tattagtgttaccacagaaattctaccagtgtctatgaccaagacatcagtagattg | |
| | tacaatgtacatttgtggtgattcaactgaatgcagcaatctattgttgcaatatggc | |
| | agtttctgtacacaattaaaccgtgctttaactggaatagctgttgaacaagacaa | |
| | gaacacccaagaagtatttgcacaagtcaaacaaatttacaagacaccaccaa | |
| | ttaaagatttcggtggatttaatttctcacaaatattaccagatccatctaaaccaag | |
| | caagaggtcatttattgaagatctactattcaacaaagtgacacttgcagatgctg | |
| | gcttcatcaaacaatatggtgattgccttggtgatattgctgctagagacctcatttgt | |
| | gcacagaagtttaacggccttactgtattgccacctttgctcacagatgaaatgatt | |
| | gctcaatacacttctgcactgttagcgggtacaatcacttctggttggacctttggtg | |
| | caggtgctgcattacaaataccatttgctatgcaaatggcttataggtttaatggtatt | |
| | ggagttacacagaatgttctctatgagaaccagaaattgattgccaaccaatttaa | |
| | tagtgctattggcaagattcaagactcactttcttccacagcaagtgcacttggtaa | |
| | acttcagaatgtggtcaaccagaatgcacaagctttaaacacgcttgttaaacaa | |
| | cttagctccaactttggtgcaatttcaagtgtattaaatgatatcctttcacgtcttgac | |
| | aaagttgaggctgaagtgcaaattgataggttgatcacaggcagacttcaaagtt | |
| | tgcagacatatgtgactcaacaattaattagagctgcagaaatcagagcttctgct | |
| | aatcttgctgctactaagatgtcagagtgtgtacttggacaatctaagagagttgat | |
| | ttctgtggaaagggctatcatcttatgtccttccctcagtcagcacctcatggtgtagt | |
| | cttcttgcatgtgacttatgtccctgcacaagagaagaacttcacaactgctcctgc | |
| | catttgtcatgatggtaaagcacactttcctcgtgaaggtgtctttgtttcaaatggca | |
| | cacactggtttgtaacacaaaggaatttctatgaaccacaaatcattactacaga | |
| | caacacatttgtgtctggtaactgtgatgttgtaataggaattgtcaacaacacagtt | |
| | tatgatcctttgcaacctgaattagactcattcaaggaggagttagataaatatttca | |
| | agaatcatacatcaccagatgttgatttaggtgacatctctggcattaatgcttcagt | |
| | tgtaaacattcagaaagaaattgaccgcctcaatgaggttgccaagaatttaaat | |
| | gaatctctcatcgatctccaagaacttggaaagtatgagcagtatatcaaatggc | |
| | catggtacatttggctaggtttcatagctggcttgattgccatagtaatggtgacaatt | |
| | atgctttgctgtatgaccagttgctgtagttgtctcaagggctgttgttcttgtggatcct | |
| | gctgcaaatttgatgaagacgactctgagccagtgctcaaaggagtcaaattac | |
| | attacacataa | |
| 10 | MFVFLVLLPLVSSQCVNLRTRTQLPPAYTNSFTRGVYYP | Amino acid |
| | DKVFRSSVLHSTQDLFLPFFSNVTWFHAIHVSGTNGTKR | sequence (N- to |
| | FDNPVLPFNDGVYFASTEKSNIIRGWIFGTTLDSKTQSLLI | C-terminus) of a |
| | VNNATNVVIKVCEFQFCNDPFLDVYYHKNNKSWMESGV | codon-optimized |
| | YSSANNCTFEYVSQPFLMDLEGKQGNFKNLREFVFKNID | SARS-CoV-2 S |
| | GYFKIYSKHTPINLVRDLPQGFSALEPLVDLPIGINITRFQT | antigen |
| | LLALHRSYLTPGDSSSGWTAGAAAYYVGYLQPRTFLLKY | sequence based |
| | NENGTITDAVDCALDPLSETKCTLKSFTVEKGIYQTSNFR | on the Wuhan- |
| | VQPTESIVRFPNITNLCPFGEVFNATRFASVYAWNRKRIS | Hu-1 reference |
| | NCVADYSVLYNSASFSTFKCYGVSPTKLNDLCFTNVYAD | strain |
| | SFVIRGDEVRQIAPGQTGKIADYNYKLPDDFTGCVIAWNS | (#NC_045512) |
| | NNLDSKVGGNYNYRYRLFRKSNLKPFERDISTEIYQAGSK | and optimized |
| | PCNGVEGFNCYFPLQSYGFQPTNGVGYQPYRVVVLSFE | for vaccinia, |
| | LLHAPATVCGPKKSTNLVKNKCVNFNFNGLTGTGVLTES | which is further |
| | NKKFLPFQQFGRDIADTTDAVRDPQTLEILDITPCSFGGV | modified to |
| | SVITPGTNTSNQVAVLYQGVNCTEVPVAIHADQLTPTWR | encode for an S |
| | VYSTGSNVFQTRAGCLIGAEHVNNSYECDIPIGAGICASY | antigen that |
| | QTQTNSRRRARSVASQSIIAYTMSLGAENSVAYSNNSIAI | includes |
| | PTNFTISVTTEILPVSMTKTSVDCTMYICGDSTECSNLLLQ | mutations of the |
| | YGSFCTQLNRALTGIAVEQDKNTQEVFAQVKQIYKTPPIK | B.1.617.2 |
| | DFGGFNFSQILPDPSKPSKRSFIEDLLFNKVTLADAGFIKQ | variant lineage |
| | YGDCLGDIAARDLICAQKFNGLTVLPPLLTDEMIAQYTSAL | identified in |
| | LAGTITSGWTFGAGAALQIPFAMQMAYRFNGIGVTQNVL | India, including |
| | YENQKLIANQFNSAIGKIQDSLSSTASALGKLQNVVNQNA | T19R, G142D, |
| | QALNTLVKQLSSNFGAISSVLNDILSRLDKVEAEVQIDRLIT | Del156/157, |
| | GRLQSLQTYVTQQLIRAAEIRASANLAATKMSECVLGQSK | R158G, L452R, |
| | RVDFCGKGYHLMSFPQSAPHGVVFLHVTYVPAQEKNFT | T478K, D614G, |
| | TAPAICHDGKAHFPREGVFVSNGTHWFVTQRNFYEPQIIT | P681R, and |
| | TDNTFVSGNCDVVIGIVNNTVYDPLQPELDSFKEELDKYF | D950N |
| | KNHTSPDVDLGDISGINASVVNIQKEIDRLNEVAKNLNESL | |
| | IDLQELGKYEQYIKWPWYIWLGFIAGLIAIVMVTIMLCCMT | |
| | SCCSCLKGCCSCGSCCKFDEDDSEPVLKGVKLHYT | |
| 11 | atgtttgtctttcttgtcttattgccactagtctctagtcagtgtgttaatcttagaaccag | Nucleotide |
| | aactcaattaccacctgcatacactaattctttcacacgtggtgtttattaccctgac | sequence/ORF |
| | aaagtattcagatcctcagtattacattcaactcaggacttgttcttacctttcttctcc | (5′ to 3′ end) of |
| | aatgttacttggttccatgctatacatgtctctgggaccaatggtactaagaggtttg | a codon- |
| | ataaccctgtcctaccatttaatgatggtgtttatttcgcttccactgagaagtctaac | optimized |
| | ataataagaggctggatatttggtactactttagattcgaagacccagtccctactt | SARS-CoV-2 S |
| | attgttaataacgctactaatgttgttattaaagtctgtgaatttcaattctgtaatgatc | antigen |
| | cattcttgggtgtttattaccacaagaacaacaagagttggatggaaagtggtgttt | sequence based |
| | attctagtgcgaataattgcactttcgaatatgtctctcagcctttccttatggaccttg | on the Wuhan- |
| | aaggtaaacagggtaatttcaagaatcttagggaatttgtgtttaagaatattgatg | Hu-1 reference |
| | gttatttcaagatatattctaagcacacgcctattaatttagtgcgtgatctccctcag | strain |
| | ggtttctcggctttagaaccattggtagatttgccaataggtattaacatcactaggtt | (#NC_045512) |
| | tcaaactttacttgctttacatagaagttatttgactcctggtgattctttctcaggttgg | and optimized |
| | acagctggtgctgcagcttattatgtgggttatcttcaacctaggactttcctattgaa | for vaccinia, |
| | atataatgagaatggaaccattacagatgctgtagactgtgcacttgaccctctct | which is further |
| | cagaaacaaagtgtacgttgaaatccttcactgtagagaaaggaatctatcaaa | modified to |
| | cttctaactttagagtccaaccaacagaatctattgttagatttcctaatattacaaa | encode for an S |
| | cttgtgccctttcggtgaagtatttaacgccaccagatttgcatctgtttatgcttgga | antigen that |
| | acaggaagagaatcagcaactgtgttgctgattattctgtcctatataattccgcat | includes |
| | cattctccacatttaagtgttatggagtgtctcctactaaattaaatgatctctgcttta | mutations of the |
| | ctaatgtctatgcagattcatttgtaattagaggtgatgaagtcagacaaatcgctc | B.1.617.2 |
| | cagggcaaactggaaagattgctgattataattataaattaccagatgactttaca | variant lineage |
| | ggctgcgttatagcttggaattctaacaatcttgattctaaggttggtggtaattataa | identified in |
| | ttaccggtatagattgtttaggaagtctaatctcaaacctttcgagagagatatttca | India, including |
| | actgaaatctatcaggccggtagcaaaccttgtaatggtgttgaaggatttaattgt | T19R, E156G, |
| | tactttcctttacaatcatatggtttccaacccactaatggtgttggttaccaaccata | Del157/158, |
| | cagagtagtagtactttcatttgaacttctacatgcaccagcaactgtttgtggacct | S255F, L452R, |
| | aagaagtctactaatttggttaagaacaaatgtgtcaatttcaacttcaatggtttaa | T478K, D614G, |
| | caggcacaggtgttcttactgagtctaacaagaagtttctgcctttccaacaatttg | P681R, and |
| | gcagagacattgctgacactactgatgctgtccgtgatccacagacacttgagatt | D950N |
| | cttgacattacaccatgttcatttggtggtgtcagtgttataacaccaggaacaaat | |
| | acttctaaccaggttgctgttctttatcagggtgttaactgcacagaagtccctgttgc | |
| | tattcatgcagatcaacttactcctacttggcgtgtttattctacaggttctaatgtattt | |
| | caaacacgtgcaggctgtttaataggagctgaacatgtcaacaactcatatgagt | |
| | gtgacatacccattggtgcaggtatatgcgctagttatcagactcagactaattctc | |
| | gtcggcgggcacgtagtgtagctagtcaatccatcattgcctacactatgtcacttg | |
| | gtgcagagaattcagttgcttactctaataactctattgccatacccacaaactttac | |
| | tattagtgttaccacagaaattctaccagtgtctatgaccaagacatcagtagattg | |
| | tacaatgtacatttgtggtgattcaactgaatgcagcaatctattgttgcaatatggc | |
| | agtttctgtacacaattaaaccgtgctttaactggaatagctgttgaacaagacaa | |
| | gaacacccaagaagtatttgcacaagtcaaacaaatttacaagacaccaccaa | |
| | ttaaagatttcggtggatttaatttctcacaaatattaccagatccatctaaaccaag | |
| | caagaggtcatttattgaagatctactattcaacaaagtgacacttgcagatgctg | |
| | gcttcatcaaacaatatggtgattgccttggtgatattgctgctagagacctcatttgt | |
| | gcacagaagtttaacggccttactgtattgccacctttgctcacagatgaaatgatt | |
| | gctcaatacacttctgcactgttagcgggtacaatcacttctggttggacctttggtg | |
| | caggtgctgcattacaaataccatttgctatgcaaatggcttataggtttaatggtatt | |
| | ggagttacacagaatgttctctatgagaaccagaaattgattgccaaccaatttaa | |
| | tagtgctattggcaagattcaagactcactttcttccacagcaagtgcacttggtaa | |
| | acttcagaatgtggtcaaccagaatgcacaagctttaaacacgcttgttaaacaa | |
| | cttagctccaactttggtgcaatttcaagtgtattaaatgatatcctttcacgtcttgac | |
| | aaagttgaggctgaagtgcaaattgataggttgatcacaggcagacttcaaagtt | |
| | tgcagacatatgtgactcaacaattaattagagctgcagaaatcagagcttctgct | |
| | aatcttgctgctactaagatgtcagagtgtgtacttggacaatctaagagagttgat | |
| | ttctgtggaaagggctatcatcttatgtccttccctcagtcagcacctcatggtgtagt | |
| | cttcttgcatgtgacttatgtccctgcacaagagaagaacttcacaactgctcctgc | |
| | catttgtcatgatggtaaagcacactttcctcgtgaaggtgtctttgtttcaaatggca | |
| | cacactggtttgtaacacaaaggaatttctatgaaccacaaatcattactacaga | |
| | caacacatttgtgtctggtaactgtgatgttgtaataggaattgtcaacaacacagtt | |
| | tatgatcctttgcaacctgaattagactcattcaaggaggagttagataaatatttca | |
| | agaatcatacatcaccagatgttgatttaggtgacatctctggcattaatgcttcagt | |
| | tgtaaacattcagaaagaaattgaccgcctcaatgaggttgccaagaatttaaat | |
| | gaatctctcatcgatctccaagaacttggaaagtatgagcagtatatcaaatggc | |
| | catggtacatttggctaggtttcatagctggcttgattgccatagtaatggtgacaatt | |
| | atgctttgctgtatgaccagttgctgtagttgtctcaagggctgttgttcttgtggatcct | |
| | gctgcaaatttgatgaagacgactctgagccagtgctcaaaggagtcaaattac | |
| | attacacataa | |
| 12 | MFVFLVLLPLVSSQCVNLRTRTQLPPAYTNSFTRGVYYP | Amino acid |
| | DKVFRSSVLHSTQDLFLPFFSNVTWFHAIHVSGTNGTKR | sequence (N- to |
| | FDNPVLPFNDGVYFASTEKSNIIRGWIFGTTLDSKTQSLLI | C-terminus) of a |
| | VNNATNVVIKVCEFQFCNDPFLGVYYHKNNKSWMESGV | codon-optimized |
| | YSSANNCTFEYVSQPFLMDLEGKQGNFKNLREFVFKNID | SARS-CoV-2 S |
| | GYFKIYSKHTPINLVRDLPQGFSALEPLVDLPIGINITRFQT | antigen |
| | LLALHRSYLTPGDSFSGWTAGAAAYYVGYLQPRTFLLKY | sequence based |
| | NENGTITDAVDCALDPLSETKCTLKSFTVEKGIYQTSNFR | on the Wuhan- |
| | VQPTESIVRFPNITNLCPFGEVFNATRFASVYAWNRKRIS | Hu-1 reference |
| | NCVADYSVLYNSASFSTFKCYGVSPTKLNDLCFTNVYAD | strain |
| | SFVIRGDEVRQIAPGQTGKIADYNYKLPDDFTGCVIAWNS | (#NC_045512) |
| | NNLDSKVGGNYNYRYRLFRKSNLKPFERDISTEIYQAGSK | and optimized |
| | PCNGVEGFNCYFPLQSYGFQPTNGVGYQPYRVVVLSFE | for vaccinia, |
| | LLHAPATVCGPKKSTNLVKNKCVNFNFNGLTGTGVLTES | which is further |
| | NKKFLPFQQFGRDIADTTDAVRDPQTLEILDITPCSFGGV | modified to |
| | SVITPGTNTSNQVAVLYQGVNCTEVPVAIHADQLTPTWR | encode for an S |
| | VYSTGSNVFQTRAGCLIGAEHVNNSYECDIPIGAGICASY | antigen that |
| | QTQTNSRRRARSVASQSIIAYTMSLGAENSVAYSNNSIAI | includes |
| | PTNFTISVTTEILPVSMTKTSVDCTMYICGDSTECSNLLLQ | mutations of the |
| | YGSFCTQLNRALTGIAVEQDKNTQEVFAQVKQIYKTPPIK | B.1.617.2 |
| | DFGGFNFSQILPDPSKPSKRSFIEDLLFNKVTLADAGFIKQ | variant lineage |
| | YGDCLGDIAARDLICAQKFNGLTVLPPLLTDEMIAQYTSAL | identified in |
| | LAGTITSGWTFGAGAALQIPFAMQMAYRFNGIGVTQNVL | India, including |
| | YENQKLIANQFNSAIGKIQDSLSSTASALGKLQNVVNQNA | T19R, E156G, |
| | QALNTLVKQLSSNFGAISSVLNDILSRLDKVEAEVQIDRLIT | Del157/158, |
| | GRLQSLQTYVTQQLIRAAEIRASANLAATKMSECVLGQSK | S255F, L452R, |
| | RVDFCGKGYHLMSFPQSAPHGVVFLHVTYVPAQEKNFT | T478K, D614G, |
| | TAPAICHDGKAHFPREGVFVSNGTHWFVTQRNFYEPQIIT | P681R, and |
| | TDNTFVSGNCDVVIGIVNNTVYDPLQPELDSFKEELDKYF | D950N |
| | KNHTSPDVDLGDISGINASVVNIQKEIDRLNEVAKNLNESL | |
| | IDLQELGKYEQYIKWPWYIWLGFIAGLIAIVMVTIMLCCMT | |
| | SCCSCLKGCCSCGSCCKFDEDDSEPVLKGVKLHYT | |
| 13 | atgtttgtctttcttgtcttattgccactagtctctagtcagtgtgttaatcttacaaccag | Nucleotide |
| | aactcaattaccacctgcatacactaattctttcacacgtggtgtttattaccctgac | sequence/ORF |
| | aaagtattcagatcctcagtattacattcaactcaggacttgttcttacctttcttctcc | (5′ to 3′ end) of |
| | aatgttacttggttccatgctatacatgtctctgggaccaatggtactaagaggtttg | a codon- |
| | ataaccctgtcctaccatttaatgatggtgtttatttcgcttccattgagaagtctaac | optimized |
| | ataataagaggctggatatttggtactactttagattcgaagacccagtccctactt | SARS-CoV-2 S |
| | attgttaataacgctactaatgttgttattaaagtctgtgaatttcaattctgtaatgatc | antigen |
| | cattcttgggtgtttctaaccacaagaacaacaagagttggatggaaagtgagttc | sequence based |
| | agagtttattctagtgcgaataattgcactttcgaatatgtctctcagcctttccttatg | on the Wuhan- |
| | gaccttgaaggtaaacagggtaatttcaagaatcttagggaatttgtgtttaagaat | Hu-1 reference |
| | attgatggttatttcaagatatattctaagcacacgcctattaatttagtgcgtgatctc | strain |
| | cctcagggtttctcggctttagaaccattggtagatttgccaataggtattaacatca | (#NC_045512) |
| | ctaggtttcaaactttacttgctttacatagaagttatttgactcctggtgattcttcttca | and optimized |
| | ggttggacagctggtgctgcagcttattatgtgggttatcttcaacctaggactttcct | for vaccinia, |
| | attgaaatataatgagaatggaaccattacagatgctgtagactgtgcacttgacc | which is further |
| | ctctctcagaaacaaagtgtacgttgaaatccttcactgtagagaaaggaatctat | modified to |
| | caaacttctaactttagagtccaaccaacagaatctattgttagatttcctaatatta | encode for an S |
| | caaacttgtgccctttcggtgaagtatttaacgccaccaaatttgcatctgtttatgctt | antigen that |
| | ggaacaggaagagaatcagcaactgtgttgctgattattctgtcctatataattccg | includes |
| | catcattctccacatttaagtgttatggagtgtctcctactaaattaaatgatctctgct | mutations of the |
| | ttactaatgtctatgcagattcatttgtaattagaggtgatgaagtcagacaaatcg | B.1.621 variant |
| | ctccagggcaaactggaaagattgctgattataattataaattaccagatgacttta | lineage, |
| | caggctgcgttatagcttggaattctaacaatcttgattctaaggttggtggtaattat | including T95N, |
| | aattacctgtatagattgtttaggaagtctaatctcaaacctttcgagagagatatttc | Y144S, Y145N, |
| | aactgaaatctatcaggccggtagcacaccttgtaatggtgttaaaggatttaattg | R346K, E484K, |
| | ttactttcctttacaatcatatggtttccaacccacttatggtgttggttaccaaccata | N501Y, D614G, |
| | cagagtagtagtactttcatttgaacttctacatgcaccagcaactgtttgtggacct | P681H, and |
| | aagaagtctactaatttggttaagaacaaatgtgtcaatttcaacttcaatggtttaa | D950N |
| | caggcacaggtgttcttactgagtctaacaagaagtttctgcctttccaacaatttg | |
| | gcagagacattgctgacactactgatgctgtccgtgatccacagacacttgagatt | |
| | cttgacattacaccatgttcatttggtggtgtcagtgttataacaccaggaacaaat | |
| | acttctaaccaggttgctgttctttatcagggtgttaactgcacagaagtccctgttgc | |
| | tattcatgcagatcaacttactcctacttggcgtgtttattctacaggttctaatgtattt | |
| | caaacacgtgcaggctgtttaataggagctgaacatgtcaacaactcatatgagt | |
| | gtgacatacccattggtgcaggtatatgcgctagttatcagactcagactaattctc | |
| | atcggcgggcacgtagtgtagctagtcaatccatcattgcctacactatgtcacttg | |
| | gtgcagagaattcagttgcttactctaataactctattgccatacccacaaactttac | |
| | tattagtgttaccacagaaattctaccagtgtctatgaccaagacatcagtagattg | |
| | tacaatgtacatttgtggtgattcaactgaatgcagcaatctattgttgcaatatggc | |
| | agtttctgtacacaattaaaccgtgctttaactggaatagctgttgaacaagacaa | |
| | gaacacccaagaagtatttgcacaagtcaaacaaatttacaagacaccaccaa | |
| | ttaaagatttcggtggatttaatttctcacaaatattaccagatccatctaaaccaag | |
| | caagaggtcatttattgaagatctactattcaacaaagtgacacttgcagatgctg | |
| | gcttcatcaaacaatatggtgattgccttggtgatattgctgctagagacctcatttgt | |
| | gcacagaagtttaacggccttactgtattgccacctttgctcacagatgaaatgatt | |
| | gctcaatacacttctgcactgttagcgggtacaatcacttctggttggacctttggtg | |
| | caggtgctgcattacaaataccatttgctatgcaaatggcttataggtttaatggtatt | |
| | ggagttacacagaatgttctctatgagaaccagaaattgattgccaaccaatttaa | |
| | tagtgctattggcaagattcaagactcactttcttccacagcaagtgcacttggtaa | |
| | acttcagaatgtggtcaaccagaatgcacaagctttaaacacgcttgttaaacaa | |
| | cttagctccaactttggtgcaatttcaagtgtattaaatgatatcctttcacgtcttgac | |
| | aaagttgaggctgaagtgcaaattgataggttgatcacaggcagacttcaaagtt | |
| | tgcagacatatgtgactcaacaattaattagagctgcagaaatcagagcttctgct | |
| | aatcttgctgctactaagatgtcagagtgtgtacttggacaatctaagagagttgat | |
| | ttctgtggaaagggctatcatcttatgtccttccctcagtcagcacctcatggtgtagt | |
| | cttcttgcatgtgacttatgtccctgcacaagagaagaacttcacaactgctcctgc | |
| | catttgtcatgatggtaaagcacactttcctcgtgaaggtgtctttgtttcaaatggca | |
| | cacactggtttgtaacacaaaggaatttctatgaaccacaaatcattactacaga | |
| | caacacatttgtgtctggtaactgtgatgttgtaataggaattgtcaacaacacagtt | |
| | tatgatcctttgcaacctgaattagactcattcaaggaggagttagataaatatttca | |
| | agaatcatacatcaccagatgttgatttaggtgacatctctggcattaatgcttcagt | |
| | tgtaaacattcagaaagaaattgaccgcctcaatgaggttgccaagaatttaaat | |
| | gaatctctcatcgatctccaagaacttggaaagtatgagcagtatatcaaatggc | |
| | catggtacatttggctaggtttcatagctggcttgattgccatagtaatggtgacaatt | |
| | atgctttgctgtatgaccagttgctgtagttgtctcaagggctgttgttcttgtggatcct | |
| | gctgcaaatttgatgaagacgactctgagccagtgctcaaaggagtcaaattac | |
| | attacacataa | |
| 14 | MFVFLVLLPLVSSQCVNLTTRTQLPPAYTNSFTRGVYYPD | Amino acid |
| | KVFRSSVLHSTQDLFLPFFSNVTWFHAIHVSGTNGTKRF | sequence (N- to |
| | DNPVLPFNDGVYFASIEKSNIIRGWIFGTTLDSKTQSLLIVN | C-terminus) of a |
| | NATNVVIKVCEFQFCNDPFLGVSNHKNNKSWMESEFRV | codon-optimized |
| | YSSANNCTFEYVSQPFLMDLEGKQGNFKNLREFVFKNID | SARS-CoV-2 S |
| | GYFKIYSKHTPINLVRDLPQGFSALEPLVDLPIGINITRFQT | antigen |
| | LLALHRSYLTPGDSSSGWTAGAAAYYVGYLQPRTFLLKY | sequence based |
| | NENGTITDAVDCALDPLSETKCTLKSFTVEKGIYQTSNFR | on the Wuhan- |
| | VQPTESIVRFPNITNLCPFGEVFNATKFASVYAWNRKRIS | Hu-1 reference |
| | NCVADYSVLYNSASFSTFKCYGVSPTKLNDLCFTNVYAD | strain |
| | SFVIRGDEVRQIAPGQTGKIADYNYKLPDDFTGCVIAWNS | (#NC_045512) |
| | NNLDSKVGGNYNYLYRLFRKSNLKPFERDISTEIYQAGST | and optimized |
| | PCNGVKGFNCYFPLQSYGFQPTYGVGYQPYRVVVLSFE | for vaccinia, |
| | LLHAPATVCGPKKSTNLVKNKCVNFNFNGLTGTGVLTES | which is further |
| | NKKFLPFQQFGRDIADTTDAVRDPQTLEILDITPCSFGGV | modified to |
| | SVITPGTNTSNQVAVLYQGVNCTEVPVAIHADQLTPTWR | encode for an S |
| | VYSTGSNVFQTRAGCLIGAEHVNNSYECDIPIGAGICASY | antigen that |
| | QTQTNSHRRARSVASQSIIAYTMSLGAENSVAYSNNSIAI | includes |
| | PTNFTISVTTEILPVSMTKTSVDCTMYICGDSTECSNLLLQ | mutations of the |
| | YGSFCTQLNRALTGIAVEQDKNTQEVFAQVKQIYKTPPIK | B.1.621 variant |
| | DFGGFNFSQILPDPSKPSKRSFIEDLLFNKVTLADAGFIKQ | lineage, |
| | YGDCLGDIAARDLICAQKFNGLTVLPPLLTDEMIAQYTSAL | including T95N, |
| | LAGTITSGWTFGAGAALQIPFAMQMAYRFNGIGVTQNVL | Y144S, Y145N, |
| | YENQKLIANQFNSAIGKIQDSLSSTASALGKLQNVVNQNA | R346K, E484K, |
| | QALNTLVKQLSSNFGAISSVLNDILSRLDKVEAEVQIDRLIT | N501Y, D614G, |
| | GRLQSLQTYVTQQLIRAAEIRASANLAATKMSECVLGQSK | P681H, and |
| | RVDFCGKGYHLMSFPQSAPHGVVFLHVTYVPAQEKNFT | D950N |
| | TAPAICHDGKAHFPREGVFVSNGTHWFVTQRNFYEPQIIT | |
| | TDNTFVSGNCDVVIGIVNNTVYDPLQPELDSFKEELDKYF | |
| | KNHTSPDVDLGDISGINASVVNIQKEIDRLNEVAKNLNESL | |
| | IDLQELGKYEQYIKWPWYIWLGFIAGLIAIVMVTIMLCCMT | |
| | SCCSCLKGCCSCGSCCKFDEDDSEPVLKGVKLHYT | |
| 15 | atgtttgtctttcttgtcttattgctactagtctctagtcagtgtgttaatcttacaaccag | Nucleotide |
| | aactcaattaccacctgcatacactaattctttcacacgtggtgtttattaccctgac | sequence/ORF |
| | aaagtattcagatcctcagtattacattcaactcaggacttgttcttacctttcttctcc | (5′ to 3′ end) of |
| | aatgttacttggttccatgctatacatgtctctgggaccaatggtactaagaggtttg | a codon- |
| | ataaccctgtcctaccatttaatgatggtgtttatttcgcttccactgagaagtctaac | optimized |
| | ataataagaggctggatatttggtactactttagattcgaagacccagtccctactt | SARS-CoV-2 S |
| | attgttaataacgctactaatgttgttattaaagtctgtgaatttcaattctttaatgatc | antigen |
| | cattcttgggtgtttaccacaagaacaacaagagttggatggaaagtgagttcag | sequence based |
| | agtttattctagtgcgaataattgcactttcgaatatgtctctcagcctttccttatgga | on the Wuhan- |
| | ccttgaaggtaaacagggtaatttcaagaatcttagcgaatttgtgtttaagaatatt | Hu-1 reference |
| | gatggttatttcaagatatattctaagcacacgcctattaatttagtgcgtggtctccc | strain |
| | tcagggtttctcggctttagaaccattggtagatttgccaataggtattaacatcact | (#NC_045512) |
| | aggtttcaaactttattacatagaagttatttgactcctggtgattcttcttcaggttgga | and optimized |
| | cagctggtgctgcagcttattatgtgggttatcttcaacctaggactttcctattgaaa | for vaccinia, |
| | tataatgagaatggaaccattacagatgctgtagactgtgcacttgaccctctctc | which is further |
| | agaaacaaagtgtacgttgaaatccttcactgtagagaaaggaatctatcaaact | modified to |
| | tctaactttagagtccaaccaacagaatctattgttagatttcctaatattacaaactt | encode for an S |
| | gtgccctttcggtgaagtatttaacgccaccagatttgcatctgtttatgcttggaac | antigen that |
| | aggaagagaatcagcaactgtgttgctgattattctgtcctatataattccgcatcat | includes |
| | tctccacatttaagtgttatggagtgtctcctactaaattaaatgatctctgctttacta | mutations of the |
| | atgtctatgcagattcatttgtaattagaggtgatgaagtcagacaaatcgctccag | C.1.2 variant |
| | ggcaaactggaaagattgctgattataattataaattaccagatgactttacaggc | lineage, |
| | tgcgttatagcttggaattctaacaatcttgattctaaggttggtggtaatcataatta | including P9L, |
| | cctgtatagattgtttaggaagtctaatctcaaacctttcgagagagatatttcaact | C136F, R190S, |
| | gaaatctatcaggccggtagcacaccttgtaatggtgttaaaggatttaattgttact | D215G, |
| | ttcctttacaatcatatggtttccaacccacttatggtgttggttaccaaccatacaga | Del242/243, |
| | gtagtagtactttcatttgaacttctacatgcaccagcaactgtttgtggacctaaga | Y449Y, E484K, |
| | agtctactaatttggttaagaacaaatgtgtcaatttcaacttcaatggtttaacagg | N501Y, D614G, |
| | cacaggtgttcttactgagtctaacaagaagtttctgcctttccaacaatttggcag | H655Y, N679K, |
| | agacattgctgacactactgatgctgtccgtgatccacagacacttgagattcttga | T7161, and |
| | cattacaccatgttcatttggtggtgtcagtgttataacaccaggaacaaatacttct | T859N |
| | aaccaggttgctgttctttatcagggtgttaactgcacagaagtccctgttgctattc | |
| | atgcagatcaacttactcctacttggcgtgtttattctacaggttctaatgtatttcaaa | |
| | cacgtgcaggctgtttaataggagctgaatatgtcaacaactcatatgagtgtgac | |
| | atacccattggtgcaggtatatgcgctagttatcagactcagactaagtctcctcgg | |
| | cgggcacgtagtgtagctagtcaatccatcattgcctacactatgtcacttggtgca | |
| | gagaattcagttgcttactctaataactctattgccatacccataaactttactattag | |
| | tgttaccacagaaattctaccagtgtctatgaccaagacatcagtagattgtacaa | |
| | tgtacatttgtggtgattcaactgaatgcagcaatctattgttgcaatatggcagtttct | |
| | gtacacaattaaaccgtgctttaactggaatagctgttgaacaagacaagaaca | |
| | cccaagaagtatttgcacaagtcaaacaaatttacaagacaccaccaattaaa | |
| | gatttcggtggatttaatttctcacaaatattaccagatccatctaaaccaagcaag | |
| | aggtcatttattgaagatctactattcaacaaagtgacacttgcagatgctggcttc | |
| | atcaaacaatatggtgattgccttggtgatattgctgctagagacctcatttgtgcac | |
| | agaagtttaacggccttaatgtattgccacctttgctcacagatgaaatgattgctc | |
| | aatacacttctgcactgttagcgggtacaatcacttctggttggacctttggtgcag | |
| | gtgctgcattacaaataccatttgctatgcaaatggcttataggtttaatggtattgg | |
| | agttacacagaatgttctctatgagaaccagaaattgattgccaaccaatttaata | |
| | gtgctattggcaagattcaagactcactttcttccacagcaagtgcacttggtaaac | |
| | ttcaagatgtggtcaaccagaatgcacaagctttaaacacgcttgttaaacaactt | |
| | agctccaactttggtgcaatttcaagtgtattaaatgatatcctttcacgtcttgacaa | |
| | agttgaggctgaagtgcaaattgataggttgatcacaggcagacttcaaagtttg | |
| | cagacatatgtgactcaacaattaattagagctgcagaaatcagagcttctgcta | |
| | atcttgctgctactaagatgtcagagtgtgtacttggacaatctaagagagttgattt | |
| | ctgtggaaagggctatcatcttatgtccttccctcagtcagcacctcatggtgtagtc | |
| | ttcttgcatgtgacttatgtccctgcacaagagaagaacttcacaactgctcctgcc | |
| | atttgtcatgatggtaaagcacactttcctcgtgaaggtgtctttgtttcaaatggcac | |
| | acactggtttgtaacacaaaggaatttctatgaaccacaaatcattactacagac | |
| | aacacatttgtgtctggtaactgtgatgttgtaataggaattgtcaacaacacagttt | |
| | atgatcctttgcaacctgaattagactcattcaaggaggagttagataaatatttca | |
| | agaatcatacatcaccagatgttgatttaggtgacatctctggcattaatgcttcagt | |
| | tgtaaacattcagaaagaaattgaccgcctcaatgaggttgccaagaatttaaat | |
| | gaatctctcatcgatctccaagaacttggaaagtatgagcagtatatcaaatggc | |
| | catggtacatttggctaggtttcatagctggcttgattgccatagtaatggtgacaatt | |
| | atgctttgctgtatgaccagttgctgtagttgtctcaagggctgttgttcttgtggatcct | |
| | gctgcaaatttgatgaagacgactctgagccagtgctcaaaggagtcaaattac | |
| | attacacataa | |
| 16 | MFVFLVLLLLVSSQCVNLTTRTQLPPAYTNSFTRGVYYPD | Amino acid |
| | KVFRSSVLHSTQDLFLPFFSNVTWFHAIHVSGTNGTKRF | sequence (N- to |
| | DNPVLPFNDGVYFASTEKSNIIRGWIFGTTLDSKTQSLLIV | C-terminus) of a |
| | NNATNVVIKVCEFQFFNDPFLGVYHKNNKSWMESEFRVY | codon-optimized |
| | SSANNCTFEYVSQPFLMDLEGKQGNFKNLSEFVFKNIDG | SARS-CoV-2 S |
| | YFKIYSKHTPINLVRGLPQGFSALEPLVDLPIGINITRFQTL | antigen |
| | LHRSYLTPGDSSSGWTAGAAAYYVGYLQPRTFLLKYNEN | sequence based |
| | GTITDAVDCALDPLSETKCTLKSFTVEKGIYQTSNFRVQP | on the Wuhan- |
| | TESIVRFPNITNLCPFGEVFNATRFASVYAWNRKRISNCV | Hu-1 reference |
| | ADYSVLYNSASFSTFKCYGVSPTKLNDLCFTNVYADSFVI | strain |
| | RGDEVRQIAPGQTGKIADYNYKLPDDFTGCVIAWNSNNL | (#NC_045512) |
| | DSKVGGNHNYLYRLFRKSNLKPFERDISTEIYQAGSTPCN | and optimized |
| | GVKGFNCYFPLQSYGFQPTYGVGYQPYRVVVLSFELLHA | for vaccinia, |
| | PATVCGPKKSTNLVKNKCVNFNFNGLTGTGVLTESNKKF | which is further |
| | LPFQQFGRDIADTTDAVRDPQTLEILDITPCSFGGVSVITP | modified to |
| | GTNTSNQVAVLYQGVNCTEVPVAIHADQLTPTWRVYSTG | encode for an S |
| | SNVFQTRAGCLIGAEYVNNSYECDIPIGAGICASYQTQTK | antigen that |
| | SPRRARSVASQSIIAYTMSLGAENSVAYSNNSIAIPINFTIS | includes |
| | VTTEILPVSMTKTSVDCTMYICGDSTECSNLLLQYGSFCT | mutations of the |
| | QLNRALTGIAVEQDKNTQEVFAQVKQIYKTPPIKDFGGFN | C.1.2 variant |
| | FSQILPDPSKPSKRSFIEDLLFNKVTLADAGFIKQYGDCLG | lineage, |
| | DIAARDLICAQKFNGLNVLPPLLTDEMIAQYTSALLAGTITS | including P9L, |
| | GWTFGAGAALQIPFAMQMAYRFNGIGVTQNVLYENQKLI | C136F, R190S, |
| | ANQFNSAIGKIQDSLSSTASALGKLQDVVNQNAQALNTLV | D215G, |
| | KQLSSNFGAISSVLNDILSRLDKVEAEVQIDRLITGRLQSL | Del242/243, |
| | QTYVTQQLIRAAEIRASANLAATKMSECVLGQSKRVDFC | Y449Y, E484K, |
| | GKGYHLMSFPQSAPHGVVFLHVTYVPAQEKNFTTAPAIC | N501Y, D614G, |
| | HDGKAHFPREGVFVSNGTHWFVTQRNFYEPQIITTDNTF | H655Y, N679K, |
| | VSGNCDVVIGIVNNTVYDPLQPELDSFKEELDKYFKNHTS | T716, and |
| | PDVDLGDISGINASVVNIQKEIDRLNEVAKNLNESLIDLQEL | T859N |
| | GKYEQYIKWPWYIWLGFIAGLIAIVMVTIMLCCMTSCCSCL | |
| | KGCCSCGSCCKFDEDDSEPVLKGVKLHYT | |
| 17 | atgtctgataacggaccacagaatcagcgaaacgcaccacgcattacgtttggt | Nucleotide |
| | ggaccctcagattcaactggcagtaaccagaatggagaacgcagtggagcgc | sequence/ORF |
| | gatctaaacaacgtcggcctcaaggtttacccaataatactgcgtcttggttcacc | (5′ to 3′ end) of |
| | gctctcactcaacatggcaaggaaggccttaaattccctcgaggacaaggcgtt | a codon- |
| | ccaattaacaccaatagcagtccagatgaccaaattggctactaccgaagagct | optimized |
| | accagacgaattcgtggtggtgacggtaagatgaaagatctcagtccaagatgg | SARS-CoV-2 N |
| | tatttctactacctaggaactgggccagaagctggacttccctatggtgctaacaa | antigen |
| | agacggcatcatatgggttgcaactgagggagccttgaatacacctaaagatca | sequence based |
| | cattggcacccgcaatcctgctaacaatgctgcaatcgtgctacaacttcctcaag | on the Wuhan- |
| | gaacaacattgcctaaaggcttctacgcagaagggagcagaggcggcagtca | Hu-1 reference |
| | agcctcttctcgttcctcatcacgtagtcgcaacagttcaagaaattcaactccag | strain |
| | gcagcagtatgggaacttctcctgctagaatggctggcaatggctgtgatgctgct | (#NC_045512) |
| | cttgctttgctgctgcttgacagattgaaccagcttgagagcaagatgtctggtaaa | and optimized |
| | ggccaacaacaacaaggccaaactgtcactaagaaatctgctgctgaggcttct | for vaccinia, |
| | aagaagcctcggcagaaacgtactgccactaaagcatacaatgtaacacaag | which is further |
| | ctttcggcagacgtggtccagaacaaacccaaggaaactttggagaccagga | modified to |
| | actaatcagacaaggaactgattacaaacattggccgcaaattgcacaatttgct | encode for an N |
| | cccagcgcttcagcgttcttcggaatgtcgcgcattggcatggaagtcacaccttc | antigen that |
| | gggaacgtggttgacctacacaggtgccatcaaattggatgacaaagatccaa | includes |
| | atttcaaagatcaagtcatattgctgaataagcatattgacgcatacaagacattc | mutations of the |
| | ccaccaacagagcctaagaaggacaagaagaagaaggcttatgaaactcaa | B.1.617.2 |
| | gccttaccgcagagacagaagaaacagcaaactgtgactcttcttcctgctgca | variant lineage |
| | gatttggatgatttctccaaacaattgcaacaatccatgagcagtgctgactcaac | identified in |
| | tcaggcctaa | India, including |
| | | D63G, R203M, |
| | | G215C, and |
| | | D377Y |
| 18 | MSDNGPQNQRNAPRITFGGPSDSTGSNQNGERSGARS | Amino acid |
| | KQRRPQGLPNNTASWFTALTQHGKEGLKFPRGQGVPIN | sequence (N- to |
| | TNSSPDDQIGYYRRATRRIRGGDGKMKDLSPRWYFYYL | C-terminus) of a |
| | GTGPEAGLPYGANKDGIIWVATEGALNTPKDHIGTRNPA | codon-optimized |
| | NNAAIVLQLPQGTTLPKGFYAEGSRGGSQASSRSSSRSR | SARS-CoV-2 N |
| | NSSRNSTPGSSMGTSPARMAGNGCDAALALLLLDRLNQ | antigen |
| | LESKMSGKGQQQQGQTVTKKSAAEASKKPRQKRTATKA | sequence based |
| | YNVTQAFGRRGPEQTQGNFGDQELIRQGTDYKHWPQIA | on the Wuhan- |
| | QFAPSASAFFGMSRIGMEVTPSGTWLTYTGAIKLDDKDP | Hu-1 reference |
| | NFKDQVILLNKHIDAYKTFPPTEPKKDKKKKAYETQALPQ | strain |
| | RQKKQQTVTLLPAADLDDFSKQLQQSMSSADSTQA | (#NC_045512) |
| | | and optimized |
| | | for vaccinia, |
| | | which is further |
| | | modified to |
| | | encode for an N |
| | | antigen that |
| | | includes |
| | | mutations of the |
| | | B.1.617.2 |
| | | variant lineage |
| | | identified in |
| | | India, including |
| | | D63G, R203M, |
| | | G215C, and |
| | | D377Y |
| 19 | atgtctgataacggaccacagaatcagcgaaacgcactacgcattacgtttggt | Nucleotide |
| | ggaccctcagattcaactggcagtaaccagaatggagaacgcagtggagcgc | sequence/ORF |
| | gatctaaacaacgtcggcctcaaggtttacccaataatactgcgtcttggttcacc | (5′ to 3′ end) of |
| | gctctcactcaacatggcaaggaagaccttaaattccctcgaggacaaggcgtt | a codon- |
| | ccaattaacaccaatagcagtccagatgaccaaattggctactaccgaagagct | optimized |
| | accagacgaattcgtggtggtgacggtaagatgaaagatctcagtccaagatgg | SARS-CoV-2 N |
| | tatttctactacctaggaactgggccagaagctggacttccctatggtgctaacaa | antigen |
| | agacggcatcatatgggttgcaactgagggagccttgaatacacctaaagatca | sequence based |
| | cattggcacccgcaatcctgctaacaatgctgcaatcgtgctacaacttcctcaag | on the Wuhan- |
| | gaacaacattgcctaaaggcttctacgcagaagggagcagaggcggcagtca | Hu-1 reference |
| | agcctcttctcgttcctcatcacgtagtcgcaacagttcaagaaattcaactccag | strain |
| | gcagcagtaaacgaacttctcctgctagaatggctggcaatggcggtgatgctg | (#NC_045512) |
| | ctcttgctttgctgctgcttgacagattgaaccagcttgagagcaagatgtctggta | and optimized |
| | aaggccaacaacaacaaggccaaactgtcactaagaaatctgctgctgaggct | for vaccinia, |
| | tctaagaagcctcggcagaaacgtactgccactaaagcatacaatgtaacaca | which is further |
| | agctttcggcagacgtggtccagaacaaacccaaggaaactttggagaccag | modified to |
| | gaactaatcagacaaggaactgattacaaacattggccgcaaattgcacaattt | encode for an N |
| | gctcccagcgcttcagcgttcttcggaatgtcgcgcattggcatggaagtcacac | antigen that |
| | cttcgggaacgtggttgacctacacaggtgccatcaaattggatgacaaagatc | includes |
| | caaatttcaaagatcaagtcatattgctgaataagcatattgacgcatacaagac | mutations of the |
| | attcccaccaacagagcctaagaaggacaagaagaagaaggctgatgaaac | C.1.2 variant |
| | tcaagccttaccgcacagacagaagaaacagcaaactgtgactcttcttcctgct | lineage, |
| | gcagatttggatgatttctccaaacaattgcaacaatccatgagcagtgctgactc | including P13L, |
| | aactcaggcctaa | R203K, G204R, |
| | | and Q384H |
| 20 | MSDNGPQNQRNALRITFGGPSDSTGSNQNGERSGARSK | Amino acid |
| | QRRPQGLPNNTASWFTALTQHGKEDLKFPRGQGVPINT | sequence (N- to |
| | NSSPDDQIGYYRRATRRIRGGDGKMKDLSPRWYFYYLG | C-terminus) of a |
| | TGPEAGLPYGANKDGIIWVATEGALNTPKDHIGTRNPAN | codon-optimized |
| | NAAIVLQLPQGTTLPKGFYAEGSRGGSQASSRSSSRSRN | SARS-CoV-2 N |
| | SSRNSTPGSSKRTSPARMAGNGGDAALALLLLDRLNQLE | antigen |
| | SKMSGKGQQQQGQTVTKKSAAEASKKPRQKRTATKAYN | sequence based |
| | VTQAFGRRGPEQTQGNFGDQELIRQGTDYKHWPQIAQF | on the Wuhan- |
| | APSASAFFGMSRIGMEVTPSGTWLTYTGAIKLDDKDPNF | Hu-1 reference |
| | KDQVILLNKHIDAYKTFPPTEPKKDKKKKADETQALPHRQ | strain |
| | KKQQTVTLLPAADLDDFSKQLQQSMSSADSTQA | (#NC_045512) |
| | | and optimized |
| | | for vaccinia, |
| | | which is further |
| | | modified to |
| | | encode for an N |
| | | antigen that |
| | | includes |
| | | mutations of the |
| | | C.1.2 variant |
| | | lineage, |
| | | including P13L, |
| | | R203K, G204R, |
| | | and Q384H |
| 21 | atgtttgtctttcttgtcttattgccactagtctctagtcagtgtgttaactttacaaccag | Nucleotide |
| | aactcaattaccacctgcatacactaattctttcacacgtggtgtttattaccctgac | sequence/ORF |
| | aaagtattcagatcctcagtattacattcaactcaggacttgttcttacctttcttctcc | (5′ to 3′ end) of |
| | aatgttacttggttccatgctatacatgtctctgggaccaatggtactaagaggtttg | a codon- |
| | ctaaccctgtcctaccatttaatgatggtgtttatttcgcttccactgagaagtctaac | optimized |
| | ataataagaggctggatatttggtactactttagattcgaagacccagtccctactt | SARS-CoV-2 S |
| | attgttaataacgctactaatgttgttattaaagtctgtgaatttcaattctgtaatgatc | antigen |
| | cattcttgggtgtttattaccacaagaacaacaagagttggatggaaagtgagttc | sequence based |
| | agagtttattctagtgcgaataattgcactttcgaatatgtctctcagcctttccttatg | on the Wuhan- |
| | gaccttgaaggtaaacagggtaatttcaagaatcttagggaatttgtgtttaagaat | Hu-1 reference |
| | attgatggttatttcaagatatattctaagcacacgcctattaatttagtgcgtggtctc | strain |
| | cctcagggtttctcggctttagaaccattggtagatttgccaataggtattaacatca | (#NC_045512) |
| | ctaggtttcaaactttacatataagttatttgactcctggtgattcttcttcaggttggac | and optimized |
| | agctggtgctgcagcttattatgtgggttatcttcaacctaggactttcctattgaaat | for vaccinia, |
| | ataatgagaatggaaccattacagatgctgtagactgtgcacttgaccctctctca | which is further |
| | gaaacaaagtgtacgttgaaatccttcactgtagagaaaggaatctatcaaactt | modified to |
| | ctaactttagagtccaaccaacagaatctattgttagatttcctaatattacaaactt | encode for an S |
| | gtgccctttcggtgaagtatttaacgccaccagatttgcatctgtttatgcttggaac | antigen that |
| | aggaagagaatcagcaactgtgttgctgattattctgtcctatataattccgcatcat | includes |
| | tctccacatttaagtgttatggagtgtctcctactaaattaaatgatctctgctttacta | mutations of the |
| | atgtctatgcagattcatttgtaattagaggtgatgaagtcagacaaatcgctccag | B.1.351 variant |
| | ggcaaactggaaatattgctgattataattataaattaccagatgactttacaggct | lineage, |
| | gcgttatagcttggaattctaacaatcttgattctaaggttggtggtaattataattac | including L18F, |
| | ctgtatagattgtttaggaagtctaatctcaaacctttcgagagagatatttcaactg | D80A, D215G, |
| | aaatctatcaggccggtagcacaccttgtaatggtgttaaaggatttaattgttacttt | Del242-244, |
| | cctttacaatcatatggtttccaacccacttatggtgttggttaccaaccatacagag | R246I, N501Y, |
| | tagtagtactttcatttgaacttctacatgcaccagcaactgtttgtggacctaagaa | E484K, K417N, |
| | gtctactaatttggttaagaacaaatgtgtcaatttcaacttcaatggtttaacaggc | D614G, and |
| | acaggtgttcttactgagtctaacaagaagtttctgcctttccaacaatttggcaga | A701V |
| | gacattgctgacactactgatgctgtccgtgatccacagacacttgagattcttgac | |
| | attacaccatgttcatttggtggtgtcagtgttataacaccaggaacaaatacttcta | |
| | accaggttgctgttctttatcagggtgttaactgcacagaagtccctgttgctattcat | |
| | gcagatcaacttactcctacttggcgtgtttattctacaggttctaatgtatttcaaac | |
| | acgtgcaggctgtttaataggagctgaacatgtcaacaactcatatgagtgtgac | |
| | atacccattggtgcaggtatatgcgctagttatcagactcagactaattctcctcgg | |
| | cgggcacgtagtgtagctagtcaatccatcattgcctacactatgtcacttggtgta | |
| | gagaattcagttgcttactctaataactctattgccatacccacaaactttactattag | |
| | tgttaccacagaaattctaccagtgtctatgaccaagacatcagtagattgtacaa | |
| | tgtacatttgtggtgattcaactgaatgcagcaatctattgttgcaatatggcagtttct | |
| | gtacacaattaaaccgtgctttaactggaatagctgttgaacaagacaagaaca | |
| | cccaagaagtatttgcacaagtcaaacaaatttacaagacaccaccaattaaa | |
| | gatttcggtggatttaatttctcacaaatattaccagatccatctaaaccaagcaag | |
| | aggtcatttattgaagatctactattcaacaaagtgacacttgcagatgctggcttc | |
| | atcaaacaatatggtgattgccttggtgatattgctgctagagacctcatttgtgcac | |
| | agaagtttaacggccttactgtattgccacctttgctcacagatgaaatgattgctc | |
| | aatacacttctgcactgttagcgggtacaatcacttctggttggacctttggtgcag | |
| | gtgctgcattacaaataccatttgctatgcaaatggcttataggtttaatggtattgg | |
| | agttacacagaatgttctctatgagaaccagaaattgattgccaaccaatttaata | |
| | gtgctattggcaagattcaagactcactttcttccacagcaagtgcacttggtaaac | |
| | ttcaagatgtggtcaaccagaatgcacaagctttaaacacgcttgttaaacaactt | |
| | agctccaactttggtgcaatttcaagtgtattaaatgatatcctttcacgtcttgacaa | |
| | agttgaggctgaagtgcaaattgataggttgatcacaggcagacttcaaagtttg | |
| | cagacatatgtgactcaacaattaattagagctgcagaaatcagagcttctgcta | |
| | atcttgctgctactaagatgtcagagtgtgtacttggacaatctaagagagttgattt | |
| | ctgtggaaagggctatcatcttatgtccttccctcagtcagcacctcatggtgtagtc | |
| | ttcttgcatgtgacttatgtccctgcacaagagaagaacttcacaactgctcctgcc | |
| | atttgtcatgatggtaaagcacactttcctcgtgaaggtgtctttgtttcaaatggcac | |
| | acactggtttgtaacacaaaggaatttctatgaaccacaaatcattactacagac | |
| | aacacatttgtgtctggtaactgtgatgttgtaataggaattgtcaacaacacagttt | |
| | atgatcctttgcaacctgaattagactcattcaaggaggagttagataaatatttca | |
| | agaatcatacatcaccagatgttgatttaggtgacatctctggcattaatgcttcagt | |
| | tgtaaacattcagaaagaaattgaccgcctcaatgaggttgccaagaatttaaat | |
| | gaatctctcatcgatctccaagaacttggaaagtatgagcagtatatcaaatggc | |
| | catggtacatttggctaggtttcatagctggcttgattgccatagtaatggtgacaatt | |
| | atgctttgctgtatgaccagttgctgtagttgtctcaagggctgttgttcttgtggatcct | |
| | gctgcaaatttgatgaagacgactctgagccagtgctcaaaggagtcaaattac | |
| | attacacataa | |
| 22 | MFVFLVLLPLVSSQCVNFTTRTQLPPAYTNSFTRGVYYPD | Amino acid |
| | KVFRSSVLHSTQDLFLPFFSNVTWFHAIHVSGTNGTKRFA | sequence (N- to |
| | NPVLPFNDGVYFASTEKSNIIRGWIFGTTLDSKTQSLLIVN | C-terminus) of a |
| | NATNVVIKVCEFQFQNDPFLGVYYHKNNKSWMESEFRVY | codon-optimized |
| | SSANNCTFEYVSQPFLMDLEGKQGNFKNLREFVFKNIDG | SARS-CoV-2 S |
| | YFKIYSKHTPINLVRGLPQGFSALEPLVDLPIGINITRFQTL | antigen |
| | HISYLTPGDSSSGWTAGAAAYYVGYLQPRTFLLKYNENG | sequence based |
| | TITDAVDCALDPLSETKCTLKSFTVEKGIYQTSNFRVQPTE | on the Wuhan- |
| | SIVRFPNITNLCPFGEVFNATRFASVYAWNRKRISNCVAD | Hu-1 reference |
| | YSVLYNSASFSTFKCYGVSPTKLNDLCFTNVYADSFVIRG | strain |
| | DEVRQIAPGQTGNIADYNYKLPDDFTGCVIAWNSNNLDS | (#NC_045512) |
| | KVGGNYNYLYRLFRKSNLKPFERDISTEIYQAGSTPCNGV | and optimized |
| | KGFNCYFPLQSYGFQPTYGVGYQPYRVVVLSFELLHAPA | for vaccinia, |
| | TVCGPKKSTNLVKNKCVNFNFNGLTGTGVLTESNKKFLP | which is further |
| | FQQFGRDIADTTDAVRDPQTLEILDITPCSFGGVSVITPGT | modified to |
| | NTSNQVAVLYQGVNCTEVPVAIHADQLTPTWRVYSTGSN | encode for an S |
| | VFQTRAGCLIGAEHVNNSYECDIPIGAGICASYQTQTNSP | antigen that |
| | RRARSVASQSIIAYTMSLGVENSVAYSNNSIAIPTNFTISVT | includes |
| | TEILPVSMTKTSVDCTMYICGDSTECSNLLLQYGSFCTQL | mutations of the |
| | NRALTGIAVEQDKNTQEVFAQVKQIYKTPPIKDFGGFNFS | B.1.351 variant |
| | QILPDPSKPSKRSFIEDLLFNKVTLADAGFIKQYGDCLGDI | lineage, |
| | AARDLICAQKFNGLTVLPPLLTDEMIAQYTSALLAGTITSG | including L18F, |
| | WTFGAGAALQIPFAMQMAYRFNGIGVTQNVLYENQKLIA | D80A, D215G, |
| | NQFNSAIGKIQDSLSSTASALGKLQDVVNQNAQALNTLVK | Del242-244, |
| | QLSSNFGAISSVLNDILSRLDKVEAEVQIDRLITGRLQSLQ | R2461, N501Y, |
| | TYVTQQLIRAAEIRASANLAATKMSECVLGQSKRVDFCGK | E484K, K417N, |
| | GYHLMSFPQSAPHGVVFLHVTYVPAQEKNFTTAPAICHD | D614G, and |
| | GKAHFPREGVFVSNGTHWFVTQRNFYEPQIITTDNTFVS | A701V |
| | GNCDVVIGIVNNTVYDPLQPELDSFKEELDKYFKNHTSPD | |
| | VDLGDISGINASVVNIQKEIDRLNEVAKNLNESLIDLQELG | |
| | KYEQYIKWPWYIWLGFIAGLIAIVMVTIMLCCMTSCCSCLK | |
| | GCCSCGSCCKFDEDDSEPVLKGVKLHYT | |
| 23 | atgtctgataacggaccacagaatcagcgaaacgcaccacgcattacgtttggt | Nucleotide |
| | ggaccctcagattcaactggcagtaaccagaatggagaacgcagtggagcgc | sequence/ORF |
| | gatctaaacaacgtcggcctcaaggtttacccaataatactgcgtcttggttcacc | (5′ to 3′ end) of |
| | gctctcactcaacatggcaaggaagaccttaaattccctcgaggacaaggcgtt | a codon- |
| | ccaattaacaccaatagcagtccagatgaccaaattggctactaccgaagagct | optimized |
| | accagacgaattcgtggtggtgacggtaagatgaaagatctcagtccaagatgg | SARS-CoV-2 N |
| | tatttctactacctaggaactgggccagaagctggacttccctatggtgctaacaa | antigen |
| | agacggcatcatatgggttgcaactgagggagccttgaatacacctaaagatca | sequence based |
| | cattggcacccgcaatcctgctaacaatgctgcaatcgtgctacaacttcctcaag | on the Wuhan- |
| | gaacaacattgcctaaaggcttctacgcagaagggagcagaggcggcagtca | Hu-1 reference |
| | agcctcttctcgttcctcatcacgtagtcgcaacagttcaagaaattcaactccag | strain |
| | gcagcagtagaggaatctctcctgctagaatggctggcaatggcggtgatgctg | (#NC_045512) |
| | ctcttgctttgctgctgcttgacagattgaaccagcttgagagcaagatgtctggta | and optimized |
| | aaggccaacaacaacaaggccaaactgtcactaagaaatctgctgctgaggct | for vaccinia, |
| | tctaagaagcctcggcagaaacgtactgccactaaagcatacaatgtaacaca | which is further |
| | agctttcggcagacgtggtccagaacaaacccaaggaaactttggagaccag | modified to |
| | gaactaatcagacaaggaactgattacaaacattggccgcaaattgcacaattt | encode for an N |
| | gctcccagcgcttcagcgttcttcggaatgtcgcgcattggcatggaagtcacac | antigen that |
| | cttcgggaacgtggttgacctacacaggtgccatcaaattggatgacaaagatc | includes |
| | caaatttcaaagatcaagtcatattgctgaataagcatattgacgcatacaagac | mutations of the |
| | attcccaccaacagagcctaagaaggacaagaagaagaaggctgatgaaac | B.1.351 variant |
| | tcaagccttaccgcagagacagaagaaacagcaaactgtgactcttcttcctgct | lineage, |
| | gcagatttggatgatttctccaaacaattgcaacaatccatgagcagtgctgactc | including T205I |
| | aactcaggcctaa | |
| 24 | MSDNGPQNQRNAPRITFGGPSDSTGSNQNGERSGARS | Amino acid |
| | KQRRPQGLPNNTASWFTALTQHGKEDLKFPRGQGVPIN | sequence (N- to |
| | TNSSPDDQIGYYRRATRRIRGGDGKMKDLSPRWYFYYL | C-terminus) of a |
| | GTGPEAGLPYGANKDGIIWVATEGALNTPKDHIGTRNPA | codon-optimized |
| | NNAAIVLQLPQGTTLPKGFYAEGSRGGSQASSRSSSRSR | SARS-CoV-2 N |
| | NSSRNSTPGSSRGISPARMAGNGGDAALALLLLDRLNQL | antigen |
| | ESKMSGKGQQQQGQTVTKKSAAEASKKPRQKRTATKAY | sequence based |
| | NVTQAFGRRGPEQTQGNFGDQELIRQGTDYKHWPQIAQ | on the Wuhan- |
| | FAPSASAFFGMSRIGMEVTPSGTWLTYTGAIKLDDKDPN | Hu-1 reference |
| | FKDQVILLNKHIDAYKTFPPTEPKKDKKKKADETQALPQR | strain |
| | QKKQQTVTLLPAADLDDFSKQLQQSMSSADSTQA | (#NC_045512) |
| | | and optimized |
| | | for vaccinia, |
| | | which is further |
| | | modified to |
| | | encode for an N |
| | | antigen that |
| | | includes |
| | | mutations of the |
| | | B.1.351 variant |
| | | lineage, |
| | | including T205I |
| 25 | atgtttgtctttcttgtcttattgccactagtctctagtcagtgtgttaatcttacaaccag | Nucleotide |
| | aactcaattaccacctgcatacactaattctttcacacgtggtgtttattaccctgac | sequence/ORF |
| | aaagtattcagatcctcagtattacattcaactcaggacttgttcttacctttcttctcc | (5′ to 3′ end) of |
| | aatgttacttggttccatgttatatctgggaccaatggtactaagaggtttgataacc | a codon- |
| | ctgtcctaccatttaatgatggtgtttatttcgcttccattgagaagtctaacataataa | optimized |
| | gaggctggatatttggtactactttagattcgaagacccagtccctacttattgttaat | SARS-CoV-2 S |
| | aacgctactaatgttgttattaaagtctgtgaatttcaattctgtaatgatccattcttgg | antigen |
| | atcacaagaacaacaagagttggatggaaagtgagttcagagtttattctagtgc | sequence based |
| | gaataattgcactttcgaatatgtctctcagcctttccttatggaccttgaaggtaaa | on the Wuhan- |
| | cagggtaatttcaagaatcttagggaatttgtgtttaagaatattgatggttatttcaa | Hu-1 reference |
| | gatatattctaagcacacgcctattatagtgcgtgaacctgaagatctccctcagg | strain |
| | gtttctcggctttagaaccattggtagatttgccaataggtattaacatcactaggttt | (#NC_045512) |
| | caaactttacttgctttacatagaagttatttgactcctggtgattcttcttcaggttgga | and optimized |
| | cagctggtgctgcagcttattatgtgggttatcttcaacctaggactttcctattgaaa | for vaccinia, |
| | tataatgagaatggaaccattacagatgctgtagactgtgcacttgaccctctctc | which is further |
| | agaaacaaagtgtacgttgaaatccttcactgtagagaaaggaatctatcaaact | modified to |
| | tctaactttagagtccaaccaacagaatctattgttagatttcctaatattacaaactt | encode for an S |
| | gtgccctttcgacgaagtatttaacgccaccagatttgcatctgtttatgcttggaac | antigen that |
| | aggaagagaatcagcaactgtgttgctgattattctgtcctatataatctcgcacca | includes |
| | ttcttcacatttaagtgttatggagtgtctcctactaaattaaatgatctctgctttacta | mutations of the |
| | atgtctatgcagattcatttgtaattagaggtgatgaagtcagacaaatcgctccag | B.1.1.529/BA.1 |
| | ggcaaactggaaatattgctgattataattataaattaccagatgactttacaggct | variant lineage, |
| | gcgttatagcttggaattctaacaaacttgattctaaggttagtggtaattataattac | including A67V, |
| | ctgtatagattgtttaggaagtctaatctcaaacctttcgagagagatatttcaactg | Del69-70 (HV), |
| | aaatctatcaggccggtaacaaaccttgtaatggtgttgcaggatttaattgttacttt | T95I, G142D, |
| | cctttacgatcatatagtttccgacccacttatggtgttggtcaccaaccatacaga | Del143-145 |
| | gtagtagtactttcatttgaacttctacatgcaccagcaactgtttgtggacctaaga | (VYY), Del211 |
| | agtctactaatttggttaagaacaaatgtgtcaatttcaacttcaatggtttgaaagg | (N), L212I, |
| | cacaggtgttcttactgagtctaacaagaagtttctgcctttccaacaatttggcag | Ins214 (EPE), |
| | agacattgctgacactactgatgctgtccgtgatccacagacacttgagattcttga | G339D, S371L, |
| | cattacaccatgttcatttggtggtgtcagtgttataacaccaggaacaaatacttct | S373P, S375F, |
| | aaccaggttgctgttctttatcagggtgttaactgcacagaagtccctgttgctattc | K417N, N440K, |
| | atgcagatcaacttactcctacttggcgtgtttattctacaggttctaatgtatttcaaa | G446S, S477N, |
| | cacgtgcaggctgtttaataggagctgaatatgtcaacaactcatatgagtgtgac | T478K, E484A, |
| | atacccattggtgcaggtatatgcgctagttatcagactcagactaagtctcatcg | Q493R, G496S, |
| | gcgggcacgtagtgtagctagtcaatccatcattgcctacactatgtcacttggtgc | Q498R, N501Y, |
| | agagaattcagttgcttactctaataactctattgccatacccacaaactttactatta | Y505H, T547K, |
| | gtgttaccacagaaattctaccagtgtctatgaccaagacatcagtagattgtaca | D614G, H655Y, |
| | atgtacatttgtggtgattcaactgaatgcagcaatctattgttgcaatatggcagttt | N679K, P681H, |
| | ctgtacacaattaaagcgtgctttaactggaatagctgttgaacaagacaagaac | N764K, D796Y, |
| | acccaagaagtatttgcacaagtcaaacaaatttacaagacaccaccaattaa | N856K, Q954H, |
| | gtatttcggtggatttaatttctcacaaatattaccagatccatctaaaccaagcaa | N969K, and |
| | gaggtcatttattgaagatctactattcaacaaagtgacacttgcagatgctggctt | L981F |
| | catcaaacaatatggtgattgccttggtgatattgctgctagagacctcatttgtgca | |
| | cagaagtttaagggccttactgtattgccacctttgctcacagatgaaatgattgct | |
| | caatacacttctgcactgttagcgggtacaatcacttctggttggacctttggtgcag | |
| | gtgctgcattacaaataccatttgctatgcaaatggcttataggtttaatggtattgg | |
| | agttacacagaatgttctctatgagaaccagaaattgattgccaaccaatttaata | |
| | gtgctattggcaagattcaagactcactttcttccacagcaagtgcacttggtaaac | |
| | ttcaagatgtggtcaaccacaatgcacaagctttaaacacgcttgttaaacaactt | |
| | agctccaagtttggtgcaatttcaagtgtattaaatgatatcttctcacgtcttgacaa | |
| | agttgaggctgaagtgcaaattgataggttgatcacaggcagacttcaaagtttg | |
| | cagacatatgtgactcaacaattaattagagctgcagaaatcagagcttctgcta | |
| | atcttgctgctactaagatgtcagagtgtgtacttggacaatctaagagagttgattt | |
| | ctgtggaaagggctatcatcttatgtccttccctcagtcagcacctcatggtgtagtc | |
| | ttcttgcatgtgacttatgtccctgcacaagagaagaacttcacaactgctcctgcc | |
| | atttgtcatgatggtaaagcacactttcctcgtgaaggtgtctttgtttcaaatggcac | |
| | acactggtttgtaacacaaaggaatttctatgaaccacaaatcattactacagac | |
| | aacacatttgtgtctggtaactgtgatgttgtaataggaattgtcaacaacacagttt | |
| | atgatcctttgcaacctgaattagactcattcaaggaggagttagataaatatttca | |
| | agaatcatacatcaccagatgttgatttaggtgacatctctggcattaatgcttcagt | |
| | tgtaaacattcagaaagaaattgaccgcctcaatgaggttgccaagaatttaaat | |
| | gaatctctcatcgatctccaagaacttggaaagtatgagcagtatatcaaatggc | |
| | catggtacatttggctaggtttcatagctggcttgattgccatagtaatggtgacaatt | |
| | atgctttgctgtatgaccagttgctgtagttgtctcaagggctgttgttcttgtggatcct | |
| | gctgcaaatttgatgaagacgactctgagccagtgctcaaaggagtcaaattac | |
| | attacacataa | |
| 26 | MFVFLVLLPLVSSQCVNLTTRTQLPPAYTNSFTRGVYYPD | Amino acid |
| | KVFRSSVLHSTQDLFLPFFSNVTWFHVISGTNGTKRFDN | sequence (N- to |
| | PVLPFNDGVYFASIEKSNIIRGWIFGTTLDSKTQSLLIVNNA | C-terminus) of a |
| | TNVVIKVCEFQFCNDPFLDHKNNKSWMESEFRVYSSANN | codon-optimized |
| | CTFEYVSQPFLMDLEGKQGNFKNLREFVFKNIDGYFKIYS | SARS-CoV-2 S |
| | KHTPIIVREPEDLPQGFSALEPLVDLPIGINITRFQTLLALH | antigen |
| | RSYLTPGDSSSGWTAGAAAYYVGYLQPRTFLLKYNENG | sequence based |
| | TITDAVDCALDPLSETKCTLKSFTVEKGIYQTSNFRVQPTE | on the Wuhan- |
| | SIVRFPNITNLCPFDEVFNATRFASVYAWNRKRISNCVAD | Hu-1 reference |
| | YSVLYNLAPFFTFKCYGVSPTKLNDLCFTNVYADSFVIRG | strain |
| | DEVRQIAPGQTGNIADYNYKLPDDFTGCVIAWNSNKLDS | (#NC_045512) |
| | KVSGNYNYLYRLFRKSNLKPFERDISTEIYQAGNKPCNGV | and optimized |
| | AGFNCYFPLRSYSFRPTYGVGHQPYRVVVLSFELLHAPA | for vaccinia, |
| | TVCGPKKSTNLVKNKCVNFNFNGLKGTGVLTESNKKFLP | which is further |
| | FQQFGRDIADTTDAVRDPQTLEILDITPCSFGGVSVITPGT | modified to |
| | NTSNQVAVLYQGVNCTEVPVAIHADQLTPTWRVYSTGSN | encode for an S |
| | VFQTRAGCLIGAEYVNNSYECDIPIGAGICASYQTQTKSH | antigen that |
| | RRARSVASQSIIAYTMSLGAENSVAYSNNSIAIPTNFTISVT | includes |
| | TEILPVSMTKTSVDCTMYICGDSTECSNLLLQYGSFCTQL | mutations of the |
| | KRALTGIAVEQDKNTQEVFAQVKQIYKTPPIKYFGGFNFS | B.1.1.529/BA.1 |
| | QILPDPSKPSKRSFIEDLLFNKVTLADAGFIKQYGDCLGDI | variant lineage, |
| | AARDLICAQKFKGLTVLPPLLTDEMIAQYTSALLAGTITSG | including A67V, |
| | WTFGAGAALQIPFAMQMAYRFNGIGVTQNVLYENQKLIA | Del69-70 (HV), |
| | NQFNSAIGKIQDSLSSTASALGKLQDVVNHNAQALNTLVK | T95I, G142D, |
| | QLSSKFGAISSVLNDIFSRLDKVEAEVQIDRLITGRLQSLQ | Del143-145 |
| | TYVTQQLIRAAEIRASANLAATKMSECVLGQSKRVDFCGK | (VYY), Del211 |
| | GYHLMSFPQSAPHGVVFLHVTYVPAQEKNFTTAPAICHD | (N), L212I, |
| | GKAHFPREGVFVSNGTHWFVTQRNFYEPQIITTDNTFVS | Ins214 (EPE), |
| | GNCDVVIGIVNNTVYDPLQPELDSFKEELDKYFKNHTSPD | G339D, S371L, |
| | VDLGDISGINASVVNIQKEIDRLNEVAKNLNESLIDLQELG | S373P, S375F, |
| | KYEQYIKWPWYIWLGFIAGLIAIVMVTIMLCCMTSCCSCLK | K417N, N440K, |
| | GCCSCGSCCKFDEDDSEPVLKGVKLHYT | G446S, S477N, |
| | | T478K, E484A, |
| | | Q493R, G496S, |
| | | Q498R, N501Y, |
| | | Y505H, T547K, |
| | | D614G, H655Y, |
| | | N679K, P681H, |
| | | N764K, D796Y, |
| | | N856K, Q954H, |
| | | N969K, and |
| | | L981F |
| 27 | atgtctgataacggaccacagaatcagcgaaacgcactacgcattacgtttggt | Nucleotide |
| | ggaccctcagattcaactggcagtaaccagaatggaggagcgcgatctaaac | sequence/ORF |
| | aacgtcggcctcaaggtttacccaataatactgcgtcttggttcaccgctctcactc | (5′ to 3′ end) of |
| | aacatggcaaggaagaccttaaattccctcgaggacaaggcgttccaattaac | a codon- |
| | accaatagcagtccagatgaccaaattggctactaccgaagagctaccagacg | optimized |
| | aattcgtggtggtgacggtaagatgaaagatctcagtccaagatggtatttctact | SARS-CoV-2 N |
| | acctaggaactgggccagaagctggacttccctatggtgctaacaaagacggc | antigen |
| | atcatatgggttgcaactgagggagccttgaatacacctaaagatcacattggca | sequence based |
| | cccgcaatcctgctaacaatgctgcaatcgtgctacaacttcctcaaggaacaac | on the Wuhan- |
| | attgcctaaaggcttctacgcagaagggagcagaggcggcagtcaagcctcttc | Hu-1 reference |
| | tcgttcctcatcacgtagtcgcaacagttcaagaaattcaactccaggcagcagt | strain |
| | aaacgaacttctcctgctagaatggctggcaatggcggtgatgctgctcttgctttg | (#NC_045512) |
| | ctgctgcttgacagattgaaccagcttgagagcaagatgtctggtaaaggccaa | and optimized |
| | caacaacaaggccaaactgtcactaagaaatctgctgctgaggcttctaagaa | for vaccinia, |
| | gcctcggcagaaacgtactgccactaaagcatacaatgtaacacaagctttcgg | which is further |
| | cagacgtggtccagaacaaacccaaggaaactttggagaccaggaactaatc | modified to |
| | agacaaggaactgattacaaacattggccgcaaattgcacaatttgctcccagc | encode for an N |
| | gcttcagcgttcttcggaatgtcgcgcattggcatggaagtcacaccttcgggaac | antigen that |
| | gtggttgacctacacaggtgccatcaaattggatgacaaagatccaaatttcaaa | includes |
| | gatcaagtcatattgctgaataagcatattgacgcatacaagacattcccaccaa | mutations of the |
| | cagagcctaagaaggacaagaagaagaaggctgatgaaactcaagccttac | B.1.1.529/BA.1 |
| | cgcagagacagaagaaacagcaaactgtgactcttcttcctgctgcagatttgg | variant lineage, |
| | atgatttctccaaacaattgcaacaatccatgagcagtgctgactcaactcaggc | including P13L, |
| | ctaa | Del31-33 (ERS), |
| | | R203K, G204R |
| 28 | MSDNGPQNQRNALRITFGGPSDSTGSNQNGGARSKQR | Amino acid |
| | RPQGLPNNTASWFTALTQHGKEDLKFPRGQGVPINTNSS | sequence (N- to |
| | PDDQIGYYRRATRRIRGGDGKMKDLSPRWYFYYLGTGP | C-terminus) of a |
| | EAGLPYGANKDGIIWVATEGALNTPKDHIGTRNPANNAAI | codon-optimized |
| | VLQLPQGTTLPKGFYAEGSRGGSQASSRSSSRSRNSSR | SARS-CoV-2 N |
| | NSTPGSSKRTSPARMAGNGGDAALALLLLDRLNQLESKM | antigen |
| | SGKGQQQQGQTVTKKSAAEASKKPRQKRTATKAYNVTQ | sequence based |
| | AFGRRGPEQTQGNFGDQELIRQGTDYKHWPQIAQFAPS | on the Wuhan- |
| | ASAFFGMSRIGMEVTPSGTWLTYTGAIKLDDKDPNFKDQ | Hu-1 reference |
| | VILLNKHIDAYKTFPPTEPKKDKKKKADETQALPQRQKKQ | strain |
| | QTVTLLPAADLDDFSKQLQQSMSSADSTQA | (#NC_045512) |
| | | and optimized |
| | | for vaccinia, |
| | | which is further |
| | | modified to |
| | | encode for an N |
| | | antigen that |
| | | includes |
| | | mutations of the |
| | | B.1.1.529/BA.1 |
| | | variant lineage, |
| | | including P13L, |
| | | Del31-33 (ERS), |
| | | R203K, G204R |
| 29 | atgtttgtctttcttgtcttattgccactagtctctagtcagtgtgttaactttacaaacag | Nucleotide |
| | aactcaattaccatctgcatacactaattctttcacacgtggtgtttattaccctgaca | sequence/ORF |
| | aagtattcagatcctcagtattacattcaactcaggacttgttcttacctttcttctcca | (5′ to 3′ end) of |
| | atgttacttggttccatgctatacatgtctctgggaccaatggtactaagaggtttgat | a codon- |
| | aaccctgtcctaccatttaatgatggtgtttatttcgcttccactgagaagtctaacat | optimized |
| | aataagaggctggatatttggtactactttagattcgaagacccagtccctacttatt | SARS-CoV-2 S |
| | gttaataacgctactaatgttgttattaaagtctgtgaatttcaattctgtaattatccat | antigen |
| | tcttgggtgtttattaccacaagaacaacaagagttggatggaaagtgagttcag | sequence based |
| | agtttattctagtgcgaataattgcactttcgaatatgtctctcagcctttccttatgga | on the Wuhan- |
| | ccttgaaggtaaacagggtaatttcaagaatcttagcgaatttgtgtttaagaatatt | Hu-1 reference |
| | gatggttatttcaagatatattctaagcacacgcctattaatttagtgcgtgatctccc | strain |
| | tcagggtttctcggctttagaaccattggtagatttgccaataggtattaacatcact | (#NC_045512) |
| | aggtttcaaactttacttgctttacatagaagttatttgactcctggtgattcttcttcag | and optimized |
| | gttggacagctggtgctgcagcttattatgtgggttatcttcaacctaggactttccta | for vaccinia, |
| | ttgaaatataatgagaatggaaccattacagatgctgtagactgtgcacttgaccc | which is further |
| | tctctcagaaacaaagtgtacgttgaaatccttcactgtagagaaaggaatctatc | modified to |
| | aaacttctaactttagagtccaaccaacagaatctattgttagatttcctaatattac | encode for an S |
| | aaacttgtgccctttcggtgaagtatttaacgccaccagatttgcatctgtttatgctt | antigen that |
| | ggaacaggaagagaatcagcaactgtgttgctgattattctgtcctatataattccg | includes |
| | catcattctccacatttaagtgttatggagtgtctcctactaaattaaatgatctctgct | mutations of the |
| | ttactaatgtctatgcagattcatttgtaattagaggtgatgaagtcagacaaatcg | P.1 variant |
| | ctccagggcaaactggaacgattgctgattataattataaattaccagatgacttta | lineage, |
| | caggctgcgttatagcttggaattctaacaatcttgattctaaggttggtggtaattat | including L18F, |
| | aattacctgtatagattgtttaggaagtctaatctcaaacctttcgagagagatatttc | T20N, P26S, |
| | aactgaaatctatcaggccggtagcacaccttgtaatggtgttaaaggatttaattg | D138Y, R190S, |
| | ttactttcctttacaatcatatggtttccaacccacttatggtgttggttaccaaccata | K417T, E484K, |
| | cagagtagtagtactttcatttgaacttctacatgcaccagcaactgtttgtggacct | N501Y, D614G, |
| | aagaagtctactaatttggttaagaacaaatgtgtcaatttcaacttcaatggtttaa | H655Y, T1027I, |
| | caggcacaggtgttcttactgagtctaacaagaagtttctgcctttccaacaatttg | and V1167F |
| | gcagagacattgctgacactactgatgctgtccgtgatccacagacacttgagatt | |
| | cttgacattacaccatgttcatttggtggtgtcagtgttataacaccaggaacaaat | |
| | acttctaaccaggttgctgttctttatcagggtgttaactgcacagaagtccctgttgc | |
| | tattcatgcagatcaacttactcctacttggcgtgtttattctacaggttctaatgtattt | |
| | caaacacgtgcaggctgtttaataggagctgaatatgtcaacaactcatatgagt | |
| | gtgacatacccattggtgcaggtatatgcgctagttatcagactcagactaattctc | |
| | ctcggcgggcacgtagtgtagctagtcaatccatcattgcctacactatgtcacttg | |
| | gtgcagagaattcagttgcttactctaataactctattgccatacccacaaactttac | |
| | tattagtgttaccacagaaattctaccagtgtctatgaccaagacatcagtagattg | |
| | tacaatgtacatttgtggtgattcaactgaatgcagcaatctattgttgcaatatggc | |
| | agtttctgtacacaattaaaccgtgctttaactggaatagctgttgaacaagacaa | |
| | gaacacccaagaagtatttgcacaagtcaaacaaatttacaagacaccaccaa | |
| | ttaaagatttcggtggatttaatttctcacaaatattaccagatccatctaaaccaag | |
| | caagaggtcatttattgaagatctactattcaacaaagtgacacttgcagatgctg | |
| | gcttcatcaaacaatatggtgattgccttggtgatattgctgctagagacctcatttgt | |
| | gcacagaagtttaacggccttactgtattgccacctttgctcacagatgaaatgatt | |
| | gctcaatacacttctgcactgttagcgggtacaatcacttctggttggacctttggtg | |
| | caggtgctgcattacaaataccatttgctatgcaaatggcttataggtttaatggtatt | |
| | ggagttacacagaatgttctctatgagaaccagaaattgattgccaaccaatttaa | |
| | tagtgctattggcaagattcaagactcactttcttccacagcaagtgcacttggtaa | |
| | acttcaagatgtggtcaaccagaatgcacaagctttaaacacgcttgttaaacaa | |
| | cttagctccaactttggtgcaatttcaagtgtattaaatgatatcctttcacgtcttgac | |
| | aaagttgaggctgaagtgcaaattgataggttgatcacaggcagacttcaaagtt | |
| | tgcagacatatgtgactcaacaattaattagagctgcagaaatcagagcttctgct | |
| | aatcttgctgctattaagatgtcagagtgtgtacttggacaatctaagagagttgatt | |
| | tctgtggaaagggctatcatcttatgtccttccctcagtcagcacctcatggtgtagt | |
| | cttcttgcatgtgacttatgtccctgcacaagagaagaacttcacaactgctcctgc | |
| | catttgtcatgatggtaaagcacactttcctcgtgaaggtgtctttgtttcaaatggca | |
| | cacactggtttgtaacacaaaggaatttctatgaaccacaaatcattactacaga | |
| | caacacatttgtgtctggtaactgtgatgttgtaataggaattgtcaacaacacagtt | |
| | tatgatcctttgcaacctgaattagactcattcaaggaggagttagataaatatttca | |
| | agaatcatacatcaccagatgttgatttaggtgacatctctggcattaatgcttcattt | |
| | gtaaacattcagaaagaaattgaccgcctcaatgaggttgccaagaatttaaatg | |
| | aatctctcatcgatctccaagaacttggaaagtatgagcagtatatcaaatggcc | |
| | atggtacatttggctaggtttcatagctggcttgattgccatagtaatggtgacaatt | |
| | atgctttgctgtatgaccagttgctgtagttgtctcaagggctgttgttcttgtggatcct | |
| | gctgcaaatttgatgaagacgactctgagccagtgctcaaaggagtcaaattac | |
| | attacacataa | |
| 30 | MFVFLVLLPLVSSQCVNFTNRTQLPSAYTNSFTRGVYYP | Amino acid |
| | DKVFRSSVLHSTQDLFLPFFSNVTWFHAIHVSGTNGTKR | sequence (N- to |
| | FDNPVLPFNDGVYFASTEKSNIIRGWIFGTTLDSKTQSLLI | C-terminus) of a |
| | VNNATNVVIKVCEFQFCNYPFLGVYYHKNNKSWMESEFR | codon-optimized |
| | VYSSANNCTFEYVSQPFLMDLEGKQGNFKNLSEFVFKNI | SARS-CoV-2 S |
| | DGYFKIYSKHTPINLVRDLPQGFSALEPLVDLPIGINITRFQ | antigen |
| | TLLALHRSYLTPGDSSSGWTAGAAAYYVGYLQPRTFLLK | sequence based |
| | YNENGTITDAVDCALDPLSETKCTLKSFTVEKGIYQTSNF | on the Wuhan- |
| | RVQPTESIVRFPNITNLCPFGEVFNATRFASVYAWNRKRI | Hu-1 reference |
| | SNCVADYSVLYNSASFSTFKCYGVSPTKLNDLCFTNVYA | strain |
| | DSFVIRGDEVRQIAPGQTGTIADYNYKLPDDFTGCVIAWN | (#NC_045512) |
| | SNNLDSKVGGNYNYLYRLFRKSNLKPFERDISTEIYQAGS | and optimized |
| | TPCNGVKGFNCYFPLQSYGFQPTYGVGYQPYRVVVLSF | for vaccinia, |
| | ELLHAPATVCGPKKSTNLVKNKCVNFNFNGLTGTGVLTE | which is further |
| | SNKKFLPFQQFGRDIADTTDAVRDPQTLEILDITPCSFGG | modified to |
| | VSVITPGTNTSNQVAVLYQGVNCTEVPVAIHADQLTPTW | encode for an S |
| | RVYSTGSNVFQTRAGCLIGAEYVNNSYECDIPIGAGICAS | antigen that |
| | YQTQTNSPRRARSVASQSIIAYTMSLGAENSVAYSNNSIA | includes |
| | IPTNFTISVTTEILPVSMTKTSVDCTMYICGDSTECSNLLLQ | mutations of the |
| | YGSFCTQLNRALTGIAVEQDKNTQEVFAQVKQIYKTPPIK | P.1 variant |
| | DFGGFNFSQILPDPSKPSKRSFIEDLLFNKVTLADAGFIKQ | lineage, |
| | YGDCLGDIAARDLICAQKFNGLTVLPPLLTDEMIAQYTSAL | including L18F, |
| | LAGTITSGWTFGAGAALQIPFAMQMAYRFNGIGVTQNVL | T20N, P26S, |
| | YENQKLIANQFNSAIGKIQDSLSSTASALGKLQDVVNQNA | D138Y, R190S, |
| | QALNTLVKQLSSNFGAISSVLNDILSRLDKVEAEVQIDRLIT | K417T, E484K, |
| | GRLQSLQTYVTQQLIRAAEIRASANLAAIKMSECVLGQSK | N501Y, D614G, |
| | RVDFCGKGYHLMSFPQSAPHGVVFLHVTYVPAQEKNFT | H655Y, T1027I, |
| | TAPAICHDGKAHFPREGVFVSNGTHWFVTQRNFYEPQIIT | and V1167F |
| | TDNTFVSGNCDVVIGIVNNTVYDPLQPELDSFKEELDKYF | |
| | KNHTSPDVDLGDISGINASFVNIQKEIDRLNEVAKNLNESL | |
| | IDLQELGKYEQYIKWPWYIWLGFIAGLIAIVMVTIMLCCMT | |
| | SCCSCLKGCCSCGSCCKFDEDDSEPVLKGVKLHYT | |
| 31 | atgtctgataacggaccacagaatcagcgaaacgcaccacgcattacgtttggt | Nucleotide |
| | ggaccctcagattcaactggcagtaaccagaatggagaacgcagtggagcgc | sequence/ORF |
| | gatctaaacaacgtcggcctcaaggtttacccaataatactgcgtcttggttcacc | (5′ to 3′ end) of |
| | gctctcactcaacatggcaaggaagaccttaaattccctcgaggacaaggcgtt | a codon- |
| | ccaattaacaccaatagcagtcgagatgaccaaattggctactaccgaagagct | optimized |
| | accagacgaattcgtggtggtgacggtaagatgaaagatctcagtccaagatgg | SARS-CoV-2 N |
| | tatttctactacctaggaactgggccagaagctggacttccctatggtgctaacaa | antigen |
| | agacggcatcatatgggttgcaactgagggagccttgaatacacctaaagatca | sequence based |
| | cattggcacccgcaatcctgctaacaatgctgcaatcgtgctacaacttcctcaag | on the Wuhan- |
| | gaacaacattgcctaaaggcttctacgcagaagggagcagaggcggcagtca | Hu-1 reference |
| | agcctcttctcgttcctcatcacgtagtcgcaacagttcaagaaattcaactccag | strain |
| | gcagcagtaaacgaacttctcctgctagaatggctggcaatggcggtgatgctg | (#NC_045512) |
| | ctcttgctttgctgctgcttgacagattgaaccagcttgagagcaagatgtctggta | and optimized |
| | aaggccaacaacaacaaggccaaactgtcactaagaaatctgctgctgaggct | for vaccinia, |
| | tctaagaagcctcggcagaaacgtactgccactaaagcatacaatgtaacaca | which is further |
| | agctttcggcagacgtggtccagaacaaacccaaggaaactttggagaccag | modified to |
| | gaactaatcagacaaggaactgattacaaacattggccgcaaattgcacaattt | encode for an N |
| | gctcccagcgcttcagcgttcttcggaatgtcgcgcattggcatggaagtcacac | antigen that |
| | cttcgggaacgtggttgacctacacaggtgccatcaaattggatgacaaagatc | includes |
| | caaatttcaaagatcaagtcatattgctgaataagcatattgacgcatacaagac | mutations of the |
| | attcccaccaacagagcctaagaaggacaagaagaagaaggctgatgaaac | P.1 variant |
| | tcaagccttaccgcagagacagaagaaacagcaaactgtgactcttcttcctgct | lineage, |
| | gcagatttggatgatttctccaaacaattgcaacaatccatgagcagtgctgactc | including P80R, |
| | aactcaggcctaa | R203K, and |
| | | G204K |
| 32 | MSDNGPQNQRNAPRITFGGPSDSTGSNQNGERSGARS | Amino acid |
| | KQRRPQGLPNNTASWFTALTQHGKEDLKFPRGQGVPIN | sequence (N- to |
| | TNSSRDDQIGYYRRATRRIRGGDGKMKDLSPRWYFYYL | C-terminus) of a |
| | GTGPEAGLPYGANKDGIIWVATEGALNTPKDHIGTRNPA | codon-optimized |
| | NNAAIVLQLPQGTTLPKGFYAEGSRGGSQASSRSSSRSR | SARS-CoV-2 N |
| | NSSRNSTPGSSKRTSPARMAGNGGDAALALLLLDRLNQL | antigen |
| | ESKMSGKGQQQQGQTVTKKSAAEASKKPRQKRTATKAY | sequence based |
| | NVTQAFGRRGPEQTQGNFGDQELIRQGTDYKHWPQIAQ | on the Wuhan- |
| | FAPSASAFFGMSRIGMEVTPSGTWLTYTGAIKLDDKDPN | Hu-1 reference |
| | FKDQVILLNKHIDAYKTFPPTEPKKDKKKKADETQALPQR | strain |
| | QKKQQTVTLLPAADLDDFSKQLQQSMSSADSTQA | (#NC_045512) |
| | | and optimized |
| | | for vaccinia, |
| | | which is further |
| | | modified to |
| | | encode for an N |
| | | antigen that |
| | | includes |
| | | mutations of the |
| | | P.1 variant |
| | | lineage, |
| | | including P80R, |
| | | R203K, and |
| | | G204K |

In some embodiments, the vaccines and/or immunogenic compositions comprise one or more sMVA vectors, wherein the one or more sMVA vectors comprise one or more heterologous DNA sequences each comprising or consisting of a nucleotide sequence set forth in any one of SEQ ID NOs: 5, 9, 11, 13, 15, 21, 25, and 29, or a nucleotide sequence that is at least about 80% identical (e.g., at least about 80%, at least about 85%, at least about 90%, at least about 95%, at least about 96%, at least about 97%, at least about 98%, at least about 99%, or about 100% identical) to the nucleotide sequence set forth in any one of SEQ ID NOs: 5, 9, 11, 13, 15, 21, 25, and 29. In some embodiments, the vaccines and/or immunogenic compositions comprise one or more sMVA vectors, wherein the one or more sMVA vectors comprise one or more heterologous DNA sequences encoding a SARS-CoV-2 antigen (e.g., S protein) comprising or consisting of an amino acid sequence set forth in any one of SEQ ID NOs: 6, 10, 12, 14, 16, 22, 26, and 30, or an amino acid sequence that is at least about 80% identical (e.g., at least about 80%, at least about 85%, at least about 90%, at least about 95%, at least about 96%, at least about 97%, at least about 98%, at least about 99%, or about 100% identical) to the amino acid sequence set forth in any one of SEQ ID NOs: 6, 10, 12, 14, 16, 22, 26, and 30.

In some embodiments, the vaccines and/or immunogenic compositions comprise one or more sMVA vectors, wherein the one or more sMVA vectors comprise one or more heterologous DNA sequences each comprising or consisting of a nucleotide sequence set forth in any one of SEQ ID NOs: 7, 17, 19, 23, 27, and 31, or a nucleotide sequence that is at least about 80% identical (e.g., at least about 80%, at least about 85%, at least about 90%, at least about 95%, at least about 96%, at least about 97%, at least about 98%, at least about 99%, or about 100% identical) to the nucleotide sequence set forth in any one of SEQ ID NOs: 7, 17, 19, 23, 27, and 31. In some embodiments, the vaccines and/or immunogenic compositions comprise one or more sMVA vectors, wherein the one or more sMVA vectors comprise one or more heterologous DNA sequences encoding a SARS-CoV-2 antigen (e.g., N protein) comprising or consisting of an amino acid sequence set forth in any one of SEQ ID NOs: 8, 18, 20, 24, 28, and 32, or an amino acid sequence that is at least about 80% identical (e.g., at least about 80%, at least about 85%, at least about 90%, at least about 95%, at least about 96%, at least about 97%, at least about 98%, at least about 99%, or about 100% identical) to the amino acid sequence set forth in any one of SEQ ID NOs: 8, 18, 20, 24, 28, and 32.

In some embodiments, the vaccines and/or immunogenic compositions comprise one or more sMVA vectors, wherein the one or more sMVA vectors comprise one or more heterologous DNA sequences encoding an S protein and an N protein based on the ancestral Wuhan-Hu-1 reference strain. In certain of these embodiments, the heterologous DNA sequences comprise or consist of nucleotide sequences set forth in SEQ ID NOs: 5 and 7, respectively, or nucleotide sequences that are at least about 80% identical (e.g., at least about 80%, at least about 85%, at least about 90%, at least about 95%, at least about 96%, at least about 97%, at least about 98%, at least about 99%, or about 100% identical) to the nucleotide sequences set forth in SEQ ID NOs: 5 and 7, respectively. The corresponding S protein and N protein encoded by the heterologous DNA sequences comprise or consist of amino acid sequences set forth in SEQ ID NOs: 6 and 8, respectively, or amino acid sequences that are at least about 80% identical (e.g., at least about 80%, at least about 85%, at least about 90%, at least about 95%, at least about 96%, at least about 97%, at least about 98%, at least about 99%, or about 100% identical) to the amino acid sequences set forth in SEQ ID NOs: 6 and 8, respectively.

In some embodiments, the vaccines and/or immunogenic compositions comprise one or more sMVA vectors, wherein the one or more sMVA vectors comprise one or more heterologous DNA sequences encoding an S protein and an N protein based on the B.1.351 (Beta) strain. In certain of these embodiments, the heterologous DNA sequences comprise or consist of nucleotide sequences set forth in SEQ ID NOs: 21 and 23, respectively, or nucleotide sequences that are at least about 80% identical (e.g., at least about 80%, at least about 85%, at least about 90%, at least about 95%, at least about 96%, at least about 97%, at least about 98%, at least about 99%, or about 100% identical) to the nucleotide sequences set forth in SEQ ID NOs: 21 and 23, respectively. The corresponding S protein and N protein encoded by the heterologous DNA sequences comprise or consist of amino acid sequences set forth in SEQ ID NOs: 22 and 24, respectively, or amino acid sequences that are at least about 80% identical (e.g., at least about 80%, at least about 85%, at least about 90%, at least about 95%, at least about 96%, at least about 97%, at least about 98%, at least about 99%, or about 100% identical) to the amino acid sequences set forth in SEQ ID NOs: 22 and 24, respectively.

In some embodiments, the vaccines and/or immunogenic compositions comprise one or more sMVA vectors, wherein the one or more sMVA vectors comprise one or more heterologous DNA sequences encoding an S protein and an N protein based on the P.1 (Gamma) strain. In certain of these embodiments, the heterologous DNA sequences comprise or consist of nucleotide sequences set forth in SEQ ID NOs: 29 and 3′, respectively, or nucleotide sequences that are at least about 80% identical (e.g., at least about 80%, at least about 85%, at least about 90%, at least about 95%, at least about 96%, at least about 97%, at least about 98%, at least about 99%, or about 100% identical) to the nucleotide sequences set forth in SEQ ID NOs: 29 and 31, respectively. The corresponding S protein and N protein encoded by the heterologous DNA sequences comprise or consist of amino acid sequences set forth in SEQ ID NOs: 30 and 32, respectively, or amino acid sequences that are at least about 80% identical (e.g., at least about 80%, at least about 85%, at least about 90%, at least about 95%, at least about 96%, at least about 97%, at least about 98%, at least about 99%, or about 100% identical) to the amino acid sequences set forth in SEQ ID NOs: 30 and 32, respectively.

In some embodiments, the vaccines and/or immunogenic compositions comprise one or more sMVA vectors, wherein the one or more sMVA vectors comprise one or more heterologous DNA sequences encoding an S protein and an N protein based on the B.1.617.2 (Delta) strain. In certain of these embodiments, the heterologous DNA sequences comprise or consist of nucleotide sequences set forth in SEQ ID NOs: 9 and 17, respectively, or nucleotide sequences that are at least about 80% identical (e.g., at least about 80%, at least about 85%, at least about 90%, at least about 95%, at least about 96%, at least about 97%, at least about 98%, at least about 99%, or about 100% identical) to the nucleotide sequences set forth in SEQ ID NOs: 9 and 17, respectively. The corresponding S protein and N protein encoded by the heterologous DNA sequences comprise or consist of amino acid sequences set forth in SEQ ID NOs: 10 and 18, respectively, or amino acid sequences that are at least about 80% identical (e.g., at least about 80%, at least about 85%, at least about 90%, at least about 95%, at least about 96%, at least about 97%, at least about 98%, at least about 99%, or about 100% identical) to the amino acid sequences set forth in SEQ ID NOs: 10 and 18, respectively.

In some embodiments, the vaccines and/or immunogenic compositions comprise one or more sMVA vectors, wherein the one or more sMVA vectors comprise one or more heterologous DNA sequences encoding an S protein and an N protein based on the B.1.617.2 (Delta) strain. In certain of these embodiments, the heterologous DNA sequences comprise or consist of nucleotide sequences set forth in SEQ ID NOs: 11 and 17, respectively, or nucleotide sequences that are at least about 80% identical (e.g., at least about 80%, at least about 85%, at least about 90%, at least about 95%, at least about 96%, at least about 97%, at least about 98%, at least about 99%, or about 100% identical) to the nucleotide sequences set forth in SEQ ID NOs: 11 and 17, respectively. The corresponding S protein and N protein encoded by the heterologous DNA sequences comprise or consist of amino acid sequences set forth in SEQ ID NOs: 12 and 18, respectively, or amino acid sequences that are at least about 80% identical (e.g., at least about 80%, at least about 85%, at least about 90%, at least about 95%, at least about 96%, at least about 97%, at least about 98%, at least about 99%, or about 100% identical) to the amino acid sequences set forth in SEQ ID NOs: 12 and 18, respectively.

In some embodiments, the vaccines and/or immunogenic compositions comprise one or more sMVA vectors, wherein the one or more sMVA vectors comprise one or more heterologous DNA sequences encoding an S protein and an N protein based on the C.1.2 strain. In certain of these embodiments, the heterologous DNA sequences comprise or consist of nucleotide sequences set forth in SEQ ID NOs: 15 and 19, respectively, or nucleotide sequences that are at least about 80% identical (e.g., at least about 80%, at least about 85%, at least about 90%, at least about 95%, at least about 96%, at least about 97%, at least about 98%, at least about 99%, or about 100% identical) to the nucleotide sequences set forth in SEQ ID NOs: 15 and 19, respectively. The corresponding S protein and N protein encoded by the heterologous DNA sequences comprise or consist of amino acid sequences set forth in SEQ ID NOs: 16 and 20, respectively, or amino acid sequences that are at least about 80% identical (e.g., at least about 80%, at least about 85%, at least about 90%, at least about 95%, at least about 96%, at least about 97%, at least about 98%, at least about 99%, or about 100% identical) to the amino acid sequences set forth in SEQ ID NOs: 16 and 20, respectively.

In some embodiments, the vaccines and/or immunogenic compositions comprise one or more sMVA vectors, wherein the one or more sMVA vectors comprise one or more heterologous DNA sequences encoding an S protein and an N protein based on the B.1.1.529/BA.1 (Omicron) strain. In certain of these embodiments, the heterologous DNA sequences comprise or consist of nucleotide sequences set forth in SEQ ID NOs: 25 and 27, respectively, or nucleotide sequences that are at least about 80% identical (e.g., at least about 80%, at least about 85%, at least about 90%, at least about 95%, at least about 96%, at least about 97%, at least about 98%, at least about 99%, or about 100% identical) to the nucleotide sequences set forth in SEQ ID NOs: 25 and 27, respectively. The corresponding S protein and N protein encoded by the heterologous DNA sequences comprise or consist of amino acid sequences set forth in SEQ ID NOs: 26 and 28, respectively, or amino acid sequences that are at least about 80% identical (e.g., at least about 80%, at least about 85%, at least about 90%, at least about 95%, at least about 96%, at least about 97%, at least about 98%, at least about 99%, or about 100% identical) to the amino acid sequences set forth in SEQ ID NOs: 26 and 28, respectively.

In some embodiments, the vaccines and/or immunogenic compositions comprise one or more sMVA vectors, wherein the one or more sMVA vectors comprise one or more heterologous DNA sequences encoding a SARS-CoV-2 antigen (e.g., S protein, N protein) that has one or more mutations compared to the ancestral Wuhan-Hu-1 reference strain.

In some embodiments, the encoded mutant S protein based on the B.1.617.2 lineage comprises one or more of the following mutations: T19R, E156G, Del157/158, S255F, L452R, T478K, D614G, P681R, D950N, G142D, Del156/157, R158G, A222, L5F, R21T, T51I, H66Y, K77T, D80Y, T95I, G181V, R214H, P251L, D253A, V289I, V308L, A411S, G446V, T547I, A570S, T572I, Q613H, S640F, E661D, Q675H, T719I, P809S, A845S, 1850L, A879S, D979E, A1078S, H1101Y, D1127G, L1141W, G1167V, K1191N, G1291V, and V1264L. Other mutations such as K417T may also be included.

In some embodiments, the encoded mutant S protein based on VOC lineage B.1.351 (Beta) comprises one or more of the following mutations: L18F, D80A, D215G, Del242-244, R246I, N501Y, E484K, K417N, D614G, and A701V.

In some embodiments, the encoded mutant S protein based on VOC lineage P.1 (Gamma) comprises one or more of the following mutations: L18F, T20N, P26S, D138Y, R190S, K417T, E484K, N501Y, D614G, H655Y, T1027I, and V1167F.

In some embodiments, the encoded mutant S protein based on VOC lineage B.1.617.2 (Delta) comprises one or more of the following mutations: T19R, G142D, Del156-157, R158G, L452R, T478K, D614G, P681R, and D950N.

In some embodiments, the encoded mutant S protein based on VOC lineage B.1.617.2 (Delta) comprises one or more of the following mutations: T19R, K77T, Del157-158, L452R, T478K, D614G, P681R, and D950N.

In some embodiments, the encoded mutant S protein based on VOC lineage B.1.617.2 (Delta) comprises one or more of the following mutations: T19R, E156G, Del157-158, S255F, L452R, T478K, D614G, P681R, and D950N.

In some embodiments, the encoded mutant S protein based on VOC lineage B.1.617.2 (Delta) comprises one or more of the following mutations: T19R, L452R, T478K, D614G, P681R, and D950N and additionally one or more mutations comprising G142D, R158G, A222V, S255F, E156G, T95I, K77T or deletions Del156-157 or Del157-158.

In some embodiments, the encoded mutant S protein based on VOC lineage B.1.1.529/BA.1 (Omicron) comprises one or more of the following mutations: A67V, Del69-70 (HV), T95I, G142D, Del143-145 (VYY), Del211 (N), L212I, Ins214 (EPE), G339D, S371 L, S373P, S375F, K417N, N440K, G446S, S477N, T478K, E484A, Q493R, G496S, Q498R, N501Y, Y505H, T547K, D614G, H655Y, N679K, P681H, N764K, D796Y, N856K, Q954H, N969K, and L981F.

In some embodiments, the encoded mutant S protein based on VOC lineage BQ.1 (Omicron) comprises one or more of the following mutations: T191, Del24-26 (LPS), A27S, Del69-70 (HV), G142D, V213G, G339D, S371F, S373P, S375F, T376A, D405N, R408S, K417N, N440K, K444T, L452R, N460K, S477N, T478K, E484A, F486V, Q493Q, 0498R, N501Y, Y505H, D614G, H655Y, N679K, P681H, N764K, D796Y, 0954H, and N969K.

In some embodiments, the encoded mutant S protein based on VOC lineage BQ.1.1 (Omicron) comprises one or more of the following mutations: T191, Del24-26 (LPS), A27S, Del69-70 (HV), G142D, V213G, G339D, R346T, S371F, S373P, S375F, T376A, D405N, R408S, K417N, N440K, K444T, L452R, N460K, S477N, T478K, E484A, F486V, Q493Q, Q498R, N501Y, Y505H, D614G, H655Y, N679K, P681H, N764K, D796Y, Q954H, and N969K.

In some embodiments, the encoded mutant S protein based on VOC lineage XBB (Omicron) comprises one or more of the following mutations: T191, Del24-26 (LPS), A27S, V83A, G142D, Del144 (Y), H146Q, Q183E, V213E, G339H, R346T, L368I, S371F, S373P, S375F, T376A, D405N, R408S, K417N, N440K, V445P, G446S, N460K, S477N, T478K, E484A, F486S, F490S, R4930, 0498R, N501Y, Y505H, D614G, H655Y, N679K, P681H, N764K, D796Y, 0954H, and N969K.

In some embodiments, the encoded mutant N protein based on VOC lineage B.1.351 (Beta) comprises a T2051 mutation.

In some embodiments, the encoded mutant N protein based on VOC lineage P.1 (Gamma) comprises one or more of the following mutations: P80R, R203K, and G204R.

In some embodiments, the encoded mutant N protein based on VOC lineage P.1 (Gamma) comprises one or more of the following mutations: P80R, R203K, and G204K.

In some embodiments, the encoded mutant N protein based on VOC lineage B.1.617.2 (Delta) comprises one or more of the following mutations: D63G, R203M, G215C, and D377Y.

In some embodiments, the encoded mutant N protein based on VOC lineage B.1.617.2 (Delta) comprises one or more of the following mutations: D63G, R203M, and D377Y.

In some embodiments, the encoded mutant N protein based on VOC lineage B.1.617.2 (Delta) comprises one or more of the following mutations: D63G, R203M, D377Y, and R385K.

In some embodiments, the encoded mutant N protein based on VOC lineage B.1.1.529/BA.1 (Omicron) comprises one or more of the following mutations: P13L, Del31-33 (ERS), R203K, G204R.

In some embodiments, the encoded mutant N protein based on VOC lineage BQ.1 (Omicron) comprises one or more of the following mutations: P13L, Del31-33 (ERS), E136D, R203K, G204R, and S413R.

In some embodiments, the encoded mutant N protein based on VOC lineage BQ.1.1 (Omicron) comprises one or more of the following mutations: P13L, Del31-33 (ERS), E136D, R203K, G204R, and S413R.

In some embodiments, the encoded mutant N protein based on VOC lineage XBB (Omicron) comprises one or more of the following mutations: P13L, Del31-33 (ERS), R203K, G204R, and S413R.

In some embodiments, the heterologous DNA sequences encoding a SARS-CoV-2 antigen (e.g., S protein, N protein) further comprise a promoter to drive expression of the SARS-CoV-2 antigen. In some embodiments, the promotor is an mH5 promoter. Other frequently used promoters include, for example, elongation factor 1 alpha (EF1α) promoter, cytomegalovirus (CMV) immediate-early promoter (Greenaway et al., Gene 18: 355-360 (1982)), simian vacuolating virus 40 (SV40) early promoter (Fiers et al., Nature 273:113-120 (1978)), spleen focus-forming virus (SFFV) promoter, phosphoglycerate kinase (PGK) promoter (Adra et al., Gene 60(1):65-74 (1987)), human beta actin promoter, polyubiquitin C gene (UBC) promoter, and CAG promoter (Nitoshi et al., Gene 108:193-199 (1991)).

In some embodiments, the vaccines and/or immunogenic compositions may further comprise one or more pharmaceutically acceptable carriers, adjuvents, additives, excipients, preservatives, or a combination thereof. A “pharmaceutically acceptable carrier or excipient” refers to a pharmaceutically acceptable material, composition, or vehicle that is involved in carrying or transporting a compound of interest from one tissue, organ, or portion of the body to another tissue, organ, or portion of the body. For example, the carrier or excipient may be a liquid or solid filler, diluent, excipient, solvent, or encapsulating material, or some combination thereof. Each component of the carrier or excipient must be “pharmaceutically acceptable,” in that it must be compatible with the other ingredients of the formulation. It also must be suitable for contact with any tissue, organ, or portion of the body that it may encounter, meaning that it must not carry a risk of toxicity, irritation, allergic response, immunogenicity, or any other complication that excessively outweighs its therapeutic benefits. Suitable excipients include water, saline, dextrose, glycerol, or the like and combinations thereof. In some embodiments, compositions comprising host cells as disclosed herein further comprise a suitable infusion media.

In some embodiments, the vaccine and/or immunogenic composition is formulated for intramuscular (IM) injection, intranasal (IN) instillation, intradermal injection, and/or scarification. In some embodiments, the vaccine and/or immunogenic composition is formulated for administration in a single dose. In some embodiments, the vaccine and/or immunogenic composition is formulated for administration in multiple doses. In some embodiments, the vaccine and/or immunogenic composition is formulated as a prime dose (series) and/or a booster dose. In some embodiments, the booster dose is in the same dosage as the prime dose. In some embodiments, the booster dose is in a lower dosage than the prime dose.

In some embodiments, COH04S1, a multi-antigenic poxvirus-vectored SARS-CoV-2 vaccine that co-expresses full-length S and N antigens, is provided. SEQ ID NO: 33 shows the full sequence of COH04S1. The DNA and protein sequences for the S antigen of COH04S1 are represented by SEQ ID NOs: 5 and 6, and the DNA and protein sequences for the N antigen of COH04S1 are represented by SEQ ID NOs: 7 and 8. Additional constructs (e.g., sMVA-S/N, sMVA-S, sMVA-N) are also disclosed in International Application Publication No. WO 2021/236550, which is hereby incorporated by reference as if fully disclosed herein. In some embodiments, the vaccines and/or immunogenic compositions comprise an sMVA vector, wherein the sMVA vector comprises or consists of a nucleotide sequence set forth in SEQ ID NO: 33, or a nucleotide sequence that is at least about 80% identical (e.g., at least about 80%, at least about 85%, at least about 90%, at least about 95%, at least about 96%, at least about 97%, at least about 98%, at least about 99%, or about 100% identical) to the nucleotide sequence set forth in SEQ ID NO: 33.

As demonstrated in the working examples, protection against SARS-CoV-2 by COH04S1 in animal models was achieved. For example, IM or IN vaccination of Syrian hamsters with COH04S1 stimulated robust Th1-biased S- and N-specific humoral immunity and cross-neutralizing antibodies (NAb) and protected against weight loss, lower respiratory tract infection, and lung injury following IN SARS-CoV-2 challenge. In addition, one- or two-dose vaccination of African green monkeys with COH04S1-induced robust antigen-specific binding antibodies, NAb, and Th1-biased T cells protected against both upper and lower respiratory tract infection following IN/intratracheal (IT) SARS-CoV-2 challenge, and triggered potent post-challenge anamnestic antiviral recall responses. These results demonstrate that COH04S1 stimulates protective immunity against SARS-CoV-2 in animal models through different vaccination routes and dose regimen, which complements ongoing investigation of this multi-antigen sMVA-vectored SARS-CoV-2 vaccine in clinical trials.

While NAb blocking S-mediated entry are considered the principal SARS-CoV-2 immune correlate of protection, humoral and cellular immune responses to multiple antigens have been implicated in the protection against SARS-CoV-2. Besides the S protein, the N protein is well recognized as a dominant target of antibody and T cell responses in SARS-CoV-2-infected individuals and therefore suggested as an additional immunogen to augment vaccine-mediated protective immunity. Its high conservation and universal cytoplasmic expression make the N protein an attractive complementary target antigen to elicit durable and broadly reactive T cells. Several recent studies highlight the benefits of N as a vaccine antigen in animal models.

Multi-antigenic SARS-CoV-2 vaccine candidates were previously constructed using a fully synthetic platform based on the well-characterized and clinically proven modified vaccinia Ankara (MVA) vector, which is marketed in the United States under the trade name Jynneos (Bavarian Nordic). Construction of SARS-CoV-2 vaccines is disclosed in International Application Publication No. WO 2021/236550, which is hereby incorporated by reference as if fully disclosed herein. MVA is a highly attenuated poxvirus vector that is widely used to develop vaccines for infectious diseases and cancer due to its excellent safety profile in animals and humans, versatile delivery and expression system, and ability to stimulate potent humoral and cellular immune responses to heterologous antigens. MVA has been used to develop vaccine candidates for preclinical testing in animal models of congenital cytomegalovirus disease while demonstrating vaccine efficacy in several clinical trials in solid tumor and stem cell transplant patients. Using the sMVA platform, sMVA vectors co-expressing full-length S and N antigen sequences were constructed and demonstrated potent immunogenicity in mice to stimulate SARS-CoV-2 antigen-specific humoral and cellular immune responses, including NAb. One of these sMVA constructs forms the basis of clinical vaccine candidate COH04S1, which has shown to be safe and immunogenic in a randomized, double-blind, placebo-controlled, single center Phase 1 trial in healthy adults (NCT04639466), and is currently evaluated in a randomized, double-blind, single center Phase 2 trial in hematology patients who have received cellular therapy (NCT04977024).

As demonstrated herein, COH04S1 stimulates protective immunity against SARS-CoV-2 in Syrian hamsters by IM and IN vaccination and in nonhuman primates (NHP) through two-dose (2D) and single-dose (1 D) vaccination regimen. These results complement the clinical evaluation of this multi-antigen sMVA-based SARS-CoV-2 vaccine.

Additionally, the emergence of several SARS-CoV-2 VOC with the capacity to evade S-specific NAb threatens the efficacy of approved COVID-19 vaccines, which primarily utilize a single antigen design based solely on the S protein. One way to avoid or minimize the risk for SARS-CoV-2 evasion of vaccine-induced immunity could be the stimulation of broadly functional humoral and cellular immunity beyond the induction of S-specific NAb. Particularly the stimulation of T cells by a combination of multiple immunodominant antigens could act as an additional countermeasure to confer long-term and broadly effective immunity against SARS-CoV-2 and its emerging VOC. Several recent studies indicate that SARS-CoV-2 VOC have the capacity to effectively escape humoral immunity, whereas they are unable to evade T cells elicited through natural infection and vaccination.

The multi-antigenic sMVA-vectored SARS-CoV-2 vaccine COH04S1 co-expressing full-length S and N antigens provides potent immunogenicity and protective efficacy in animal models. Using Syrian hamsters and NHP, it is shown that COH04S1 elicits robust antigen-specific humoral and cellular immune responses and protects against SARS-CoV-2 challenge through different vaccination routes and dose regimen. While these animal studies were not designed to assess the contribution of N in COH04S1-mediated protective immunity, these results warrant further evaluation of COH04S1 in ongoing and future clinical trials. COH04S1 represents a second-generation COVID-19 vaccine candidate that could be used alone or in combination with other existing vaccines in parenteral or mucosal prime-boost or single-shot vaccination strategies to augment vaccine-mediated protective immune responses against SARS-CoV-2.

Although several SARS-CoV-2 vaccines based on MVA have been developed and evaluated in animal models for immunogenicity and efficacy, there is currently no MVA-based SARS-CoV-2 vaccine approved for routine clinical use. COH04S1 and MVA-SARS-2-S, an MVA vector expressing S alone, are currently the only MVA-based SARS-CoV-2 vaccines that are clinically evaluated. In addition, COH04S1 and a recently developed adenovirus vector approach are currently the only clinically evaluated SARS-CoV-2 vaccines that utilize an antigen combination composed of S and N. These findings highlight the potential importance of COH04S1 as a second-generation multi-antigenic SARS-CoV-2 vaccine to contribute to the establishment of long-term protective immunity against COVID-19 disease. In addition, these findings highlight the potential of the sMVA platform and synthetic biology in poxvirus-vectored vaccine technology to rapidly generate protective and clinical-grade vaccine vectors for infectious disease prevention.

COH04S1 demonstrated potent immunogenicity in Syrian hamsters by IM and IN vaccination and in NHP by 2D and 1 D vaccination regimen to elicit robust SARS-CoV-2-specific humoral and cellular immune responses to both S and N antigens. This included high-titer S- and N-specific binding antibodies in addition to robust binding antibodies targeting the receptor binding domain (RBD), the primary target of NAb. Binding antibodies elicited by COH04S1 in hamsters as well as T cell responses induced by COH04S1 in NHP indicated Th1-biased immunity, which is thought to be the preferred antiviral adaptive immune response to avoid vaccine-associated enhanced respiratory disease. NAb elicited by COH04S1 in hamsters and NHP showed potent neutralizing activity against SARS-CoV-2 infectious virus, highlighting the potential of COH04S1 to induce antibody responses that are considered essential for protection against SARS-CoV-2. Notably, NAb titers stimulated by COH04S1 in NHP appeared similar to peak NAb titers stimulated in healthcare workers by two doses of the FDA-approved Pfizer/BioNTech mRNA vaccine. In addition, NAb stimulated by COH04S1 in hamsters showed neutralizing activity against PV variants based on several SARS-CoV-2 VOC, including Alpha, Beta, Gamma, and Delta variants, indicating the capacity of COH04S1 to stimulate cross-protective NAb against SARS-CoV-2 VOC.

Both IM and IN vaccination with COH04S1 provided potent efficacy to protect Syrian hamsters from progressive weight loss, lower respiratory tract infection, and lung injury upon IN challenge with SARS-CoV-2, highlighting the potential of COH04S1 to stimulate protective immunity against respiratory disease through parenteral and mucosal vaccination routes. In contrast, IM or IN vaccination of hamsters with COH04S1 appeared to provide only limited protection against upper respiratory tract infection following viral challenge, indicating that COH04S1-mediated parenteral or mucosal immune stimulation afforded only little protection in this small animal model at the site of viral inoculation, which may have been associated with the relatively aggressive sub-lethal viral challenge dose. While IM and IN vaccination with COH04S1 provided overall similar immunogenicity and protective efficacy against SARS-CoV-2 in hamsters, IN vaccination with COH04S1 appeared superior compared to IM vaccination to protect against initial minor weight loss at the early phase after SARS-CoV-2 challenge. On the other hand, hamsters vaccinated by IM route with COH04S1 were completely protected from lung injury following challenge, while hamsters vaccinated by IN route with COH04S1 showed minor lung pathology and inflammation at day 10 post-challenge, suggesting superior protection against viral- or immune-mediated lung pathology through IM vaccination compared to IN vaccination with COH04S1.

The immunogenicity and protective efficacy afforded by COH04S1 against SARS-CoV-2 in Syrian hamsters by IM and IN vaccination appears consistent with known properties of MVA-vectored vaccines. While MVA is well known to stimulate robust immunity by IM vaccination, MVA has also been shown to elicit potent immunity through IN vaccination strategies at mucosal surfaces. A recombinant MVA vector expressing the S protein of Middle East Respiratory Syndrome coronavirus (MERS-CoV), a close relative of SARS-CoV-2, has recently been shown to be safe and immunogenic following IM administration in a Phase 1 clinical trial. This MVA vaccine has also been shown to protect dromedary camels against MERS-CoV challenge by co-vaccination via IM and IN routes. In addition, several animal studies have shown that IN vaccination with MVA vaccines is a potent stimulator of bronchus-associated lymphoid tissue (BALT), a tertiary lymphoid tissue structure within the lung that is frequently present in children and adolescents and that serves as a general priming site for T cells. While the precise mechanism and levels of protection afforded by COH04S1 against SARS-CoV-2 in the Syrian hamster model by IM and IN vaccination remains unclear, especially at early phase after challenge, these findings support the use of COH04S1 to elicit SARS-CoV-2 protective immunity by mucosal vaccination.

In addition to the protection afforded by IM and IN vaccination with COH04S1 in hamsters, 2D and 1D vaccination with COH04S1 in NHP provided potent protection against lower and upper respiratory tract infection upon IN/IT challenge with SARS-CoV-2. These findings demonstrate protective efficacy of COH04S1 by different dose vaccination regimen in a larger animal model that is thought to be critical to assess preclinical vaccine efficacy. While 2D and 1D vaccination of NHP with COH04S1 stimulated similar S- and RBD-specific binding antibodies at the time of viral challenge, 2D vaccination appeared to be overall more effective than 1 D vaccination in stimulating humoral and cellular immune responses, including NAb. Despite these overall lower pre-challenge immune responses in 1D-vaccinated animals compared to 2D-vaccinated NHP, 1D and 2D vaccination of NHP with COH04S1 afforded similar protection against lower and upper respiratory tract infection following viral challenge, suggesting that a single shot with COH04S1 is sufficient to induce protective immunity to SARS-CoV-2. In addition, while 2D vaccination with COH04S1 appeared to elicit overall more potent pre-challenge responses than 1 D vaccination, overall more potent post-challenge anamnestic antiviral immune responses were observed in 1 D-vaccinated NHP compared to 2D-vaccinated NHP, suggesting that a single shot with COH04S1 is sufficient to effectively prime vaccine-mediated protective recall responses to SARS-CoV-2.

These results demonstrate that COH04S1 has the capacity to elicit potent and cross-reactive Th1-biased S- and N-specific humoral and cellular responses that protect hamsters and NHP from SARS-CoV-2 by different vaccination routes and dose regimen. This multi-antigen sMVA-vectored SARS-CoV-2 vaccine could be complementary to available vaccines to induce robust and long-lasting protective immunity against SARS-CoV-2 and its emerging VOC.

### Method of Treatment

In some aspects, provided are methods of preventing and/or treating a coronavirus infection, for example, SARS-CoV-2 infection, in a subject, comprising administering to the subject a prophylactically or therapeutically effective amount of the vaccine and/or immunogenic composition according to various embodiments of the present technology.

In some aspects, provided are methods of eliciting an immune response to a coronavirus, for example, SARS-CoV-2, in a subject comprising administering to the subject a prophylactically or therapeutically effective amount of the vaccine and/or immunogenic composition according to various embodiments of the present technology. In some embodiments, the subject is infected with SARS-CoV-2 or is at risk of being infected with SARS-CoV-2.

In some embodiments, the vaccines and/or immunogenic compositions are used for preventing or treating a coronavirus infection, for example, SARS-CoV-2 infection, caused by the ancestral Wuhan-Hu-1 reference strain or a VOC, including, but not limited to, B.1.1.7 (Alpha), B.1.351 (Beta), P.1 (Gamma), B.1.617.2 (Delta), B.1.621 (Mu), C.37 (Lambda), C.1.2, BA.1 (Omicron), BA.2 (Omicron), BA.2.12.1 (Omicron), BA.4 (Omicron), BA.5 (Omicron), BA.2.75 (Omicron), and newer Omicron variants such as BQ.1, BQ.1.1, and XBB among others that arise from time to time that are resistant to known antibody therapies such as Evushield and Bebtelovimab.

In some embodiments, the vaccine and/or immunogenic composition is administered to a subject by IM injection, IN injection, intradermal injection, instillation, and/or scarification. In some embodiments, the vaccine and/or immunogenic composition is administered to a subject in a single dose. In some embodiments, the vaccine and/or immunogenic composition is administered to a subject in a prime dose (series) followed by a booster dose. In some embodiments, the booster dose is in the same dosage as the prime dose. In some embodiments, the booster dose is in a lower dosage than the prime dose. In some embodiments, one or more additional doses are administered to the subject after administration of the prime (series) and booster doses. In some embodiments, the subject has previously received a different SARS-CoV-2 vaccine before administration of the vaccine and/or immunogenic composition disclosed herein.

The following examples are intended to illustrate various embodiments of the invention. As such, the specific embodiments discussed are not to be construed as limitations on the scope of the invention. It will be apparent to one skilled in the art that various equivalents, changes, and modifications may be made without departing from the scope of the invention, and it is understood that such equivalent embodiments are to be included herein. Further, all references cited in the disclosure are hereby incorporated by reference in their entirety, as if fully set forth herein.

### EXAMPLES

### Example 1: Materials and Methods

sMVA Vaccine Stocks

COH04S1 is a double-plaque purified virus isolate derived from the sMVA-N/S vector (NCBI Accession #MW036243), with N and S antigen sequences inserted into the MVA deletion sites 2 (Del2) and 3 (Del3), respectively. It was generated using the three-plasmid system of the sMVA platform and fowlpox virus TROVAC as a helper virus for virus reconstitution. COH04S1 co-expresses full-length, unmodified S and N antigen sequences based on the Wuhan-Hu-1 reference strain (NCBI Accession #NC_045512). Sequence identity of COH04S1 seed stock virus was assessed by PacBio long-read sequencing.

COH04S351 is a double-plaque purified virus isolate with analogous vaccine construction compared to the original COH04S1 vector and co-expresses modified S and N antigen sequences based on the B.1.351 Beta variant (Table 2).

COH04S529 is a non-plaque purified virus isolate with analogous vaccine construction compared to the original COH04S1 sMVA-N/S vaccine vector and co-expresses modified S and N antigen sequences based on the Omicron BA.1 variant (Table 2).

TABLE 2
S and N antigen-specific mutations in COH04S529
and COH04S351 vaccine sequences

| | | Substitutions/Deletions (Δ) to Wuhan-Hu-1 reference strain | |
|---|---|---|---|
| Vaccine | Strain | Spike (S) | Nucleocapsid (N) |
| COH04S529 | B.1.529 | A67V, Δ69-70 (HV), T95I, G142D, Δ143- | P13L, Δ31-33 |
| | | 145 (VYY), Δ211 (N), L212I, INS214 (EPE), | (ERS), R203K, |
| | | G339D, S371L, S373P, S375F, K417N, | G204R |
| | | N440K, G446S, S477N, T478K, E484A, | |
| | | Q493R, G496S, Q498R, N501Y, Y505H, | |
| | | T547K, D614G, H655Y, N679K, P681H, | |
| | | N764K, D796Y, N856K, Q954H, N969K, | |
| | | L981F | |
| COH04S351 | B.1.351 | L18F, D80A, D215G, Δ242-244, R246I, | T205I |
| | | N501Y, E484K, K417N, D614G, A701V | |

COH04S1, COH04S351, and COH04S529 were generated using the sMVA platform. Virus stocks of the vaccine vectors and sMVA control vector were produced using chicken embryo fibroblasts (CEF) and prepared by 36% sucrose cushion ultracentrifugation and virus resuspension in 1 mM Tris-HCl (pH 9). Virus stocks were stored at −80° C. and titrated on CEF by plaque immunostaining as described. Virus stocks were validated for antigen insertion and expression by PCR, sequencing, and immunoblot.

### Animals, Study Design, and Challenge

In-life portion of hamster and nonhuman primate (NHP) studies were carried out at Bioqual Inc. (Rockville, MD). Thirty female and male golden Syrian hamsters were randomly assigned to the groups, with 3 females and 3 males in each group. Hamsters were vaccinated by IM or IN route 4 weeks apart with 1×10⁸ plaque-forming units (PFU) of COH04S1 or sMVA vaccine stocks diluted in phosphate-buffered saline (PBS). Two weeks post-booster vaccination, animals were challenged intranasally (50 μl/nare) with 3×10⁴ PFU (or 1.99×10⁴ Median Tissue Culture Infectious Dose [TCID50]) of SARS-CoV-2 USA-WA1/2020 (BEI Resources; P4 animal challenge stock, NR-53780 lot no. 70038893). The stock was produced by infecting Vero E6-hACE2 cells (BEI Resources NR-53726) at low multiplicity of infection (MOI) with deposited passage 3 virus and resulted in a titer of 1.99×10⁶ TCID50/ml. Sequence identity was confirmed by next-generation sequencing. Body weight and temperature were recorded daily for 10 days. Hamsters were humanely euthanized for lung samples collection. A total of 24 African green monkeys (Chlorocebus aethiops; 20 females and 4 males) from St. Kitts weighing 3-6 kg were randomized by weight and sex to vaccine and control groups. For two-dose (2D) vaccination, NHP were either two times mock-vaccinated with PBS (N=3) at a 4-week interval or vaccinated twice, 4 weeks apart, with 2.5×10⁸ PFU of COH04S1 (N=6) or sMVA control vector (N=3) diluted in PBS. For single-dose (1 D) vaccination, NHP were either one time mock-vaccinated (N=3) with PBS or vaccinated once with 5×10⁸ PFU of COH04S1 (N=6) or sMVA control vector (N=3) diluted in PBS. At 6 weeks post-2D or post-1D vaccination, NHP were challenged with 1×10⁵ TCID50 of SARS-CoV-2 USA-WA1/2020 strain diluted in PBS via combined IT (1 ml)/IN (0.5 ml/nare) route. Necropsy was performed 7 days and 21 days following challenge and organs were collected for gross pathology and histopathology.

For the Omicron variant studies, 80 Syrian hamsters, 6 to 8 weeks old, were randomly assigned to the groups, with 5 females and 5 males in each challenge group. Hamsters were intramuscularly vaccinated 4 weeks apart with 1×10⁸ PFU of COH04S1, COH04S529, COH04S351, or sMVA virus stocks diluted in PBS or left unvaccinated. Blood samples were collected 2 weeks after the first vaccine dose and 2 weeks after the second dose. At this latter time point, animals were challenged intranasally (50 μl/nare) with 4.8×10⁴ TCID50 of SARS-CoV-2 BA.1 (BEI Resources NR-56486 LOT #: 70049695, titered by BEI Resources using Calu-3 cells) or with 5.16×10⁴ TCID50 of SARS-CoV-2 BA.2.12.1 (BEI Resources NR-56782 LOT #: 70052277, titered using Calu-3 cells). Body weight was recorded daily for 8 days. Hamsters were humanely euthanized for lung and turbinate samples collection at day 4 (n=5/group) and day 8 (n=5/group) post-challenge.

All animal studies were conducted in compliance with local, state, and federal regulations and were approved by Bioqual and City of Hope Institutional Animal Care and Use Committees (IACUC).

### ELISA Binding Antibody Detection

SARS-CoV-2-specific binding antibodies in hamsters and NHP samples were detected by indirect ELISA utilizing purified S, RBD, and N proteins (Sino Biological 40589-V08B1, 40592-V08H, 40588-V08B); Beta, Gamma, and Delta VOC-specific S proteins (Acro Biosystems SPN-C52Hk, SPN-C52Hg, SPN-C52He); or ancestral-specific, Beta-specific, Omicron BA.1-, BA.2-, and BA.4-specific S proteins (Sino Biological 40589-V08B1, 40588-V07E9, 40589-V08H26, 40589-V08H28, 40589-V08H32) and purified ancestral-specific, Beta-specific, Omicron BA.1-, BA.2-, and BA.4-specific N proteins (Sino Biological 40588-V08B, 40589-V08B7, 40588-V07E34, 40588-V07E35, 40588-V07E37). S and N mutations included in the antigens used for ELISA are indicated in Table 3. 96-well plates (Costar 3361) were coated with 100 ul/well of S, RBD, or N proteins at a concentration of 1 μg/ml in PBS and incubated overnight at 4° C. For binding antibody detection in hamster serum, plates were washed 5× with wash buffer (0.05% Tween-20/PBS), then blocked with 250 ul/well of blocking buffer (0.5% casein/154 mM NaCl/10 mM Tris-HCl [pH 7.6]) for 2 hours at room temperature. After washing, 3-fold diluted heat-inactivated serum in blocking buffer was added to the plates and incubated 2 hours at room temperature. After washing, anti-Hamster IgG HRP secondary antibodies measuring total IgG(H+L), IgG1, or IgG2/IgG3 (Southern Biotech 6061-05, 1940-05, 1935-05) were diluted 1:1000 in blocking buffer and added to the plates. After 1 hour incubation, plates were washed and developed with 1 Step TMB-Ultra (Thermo Fisher 34029). The reaction was stopped with 1 M H₂SO₄ and plates were immediately read on FilterMax F3 (Molecular Devices). For binding antibody detection in NHP serum, a similar protocol was used. Wash buffer was 0.1% Tween-20 in PBS, and blocking buffer was 1% casein/PBS for RBD- and N-antigen ELISA and 4% Normal Goat Serum/1% casein/PBS for S-antigen ELISA. For IgG quantification in NHP bronchoalveolar lavage (BAL) samples, 1% BSA/PBS was used as blocking and sample buffers. Goat anti-Monkey IgG (H+L) secondary antibody (Thermo Fisher Cat #PA1-84631) was diluted 1:10,000. Endpoint titers were calculated as the highest dilution to have an absorbance >0.100.

TABLE 3
Mutations in S and N antigens for ELISA testing

| Strain | VOC | Spike*^{#} | Nucleocapsid* | Notes |
|---|---|---|---|---|
| B.1.351 | Beta | L18F, D80A, D215G, Δ242-244 (LAL), R246L, | T205I | |
| | | K417N, E484K, N501Y, D614G, A701V | | |
| B.1.1.529 | Omicron | A67V, Δ69-70 (HV), T95I, G142D, Δ143-145 | P13L, Δ31-33 | |
| BA.1 | BA.1 | (VYY), Δ211 (N), L212I, INS214 (EPE), | (ERS), R203K, | |
| | | G339D, S371L, S373P, S375F, K417N, | G204R | |
| | | N440K, G446S, S477N, T478K, E484A, | | |
| | | Q493R, G496S, Q498R, N501Y, Y505H, | | |
| | | T547K, D614G, H655Y, N679K, P681H, | | |
| | | N764K, D796Y, (F817P), N856K, (A892P), | | |
| | | (A899P), (A942P), Q954H, N969K, L981F, | | |
| | | (K986P), (V987P) | | |
| B.1.1.529 | Omicron | T19I, L24S, Δ25-27 (PPA), G142D, V213G, | P13L, Δ31-33 | BA.2, |
| BA.2 | BA.2 | G339D, S371F, S373P, S375F, T376A, | (ERS), R203K, | BA.5, |
| | | D405N, R408S, K417N, N440K, S477N, | G204R, S413R | BA.2.12.1, |
| | | T478K, E484A, Q493R, Q498R, N501Y, | | BA.2.75 |
| | | Y505H, D614G, H655Y, N679K, P681H, | | share the |
| | | N764K, D796Y, (F817P), (A892P), (A899P), | | same N |
| | | (A942P), Q954H, N969K, (K986P), (V987P) | | mutations |
| B.1.1.529 | Omicron | T19I, L24S, Δ25-27 (PPA), Δ69-70 (HV), | P13L, Δ31-33 | BA.4 and |
| BA.4 | BA.4 | G142D, V213G, G339D, S371F, S373P, | (ERS), P151S, | BA.5 |
| | | S375F, T376A, D405N, R408S, K417N, | R203K, | share the |
| | | N440K, L452R, S477N, T478K, E484A, | G204R, S413R | same S |
| | | F486V, Q498R, N501Y, Y505H, D614G, | | mutations |
| | | H655Y, N679K, P681H, N764K, D796Y, | | |
| | | (F817P), (A892P), (A899P), (A942P), Q954H, | | |
| | | N969K, (K986P), (V987P) | | |

*Substitutions/Deletions (Δ)/Insertions (INS) to Wuhan-Hu-1 reference strain
^{#}in parenthesis mutations added to stabilize the trimer in pre-fusion conformation

### Neutralization Assay

NAb were measured by plaque reduction neutralization titer (PRNT) assay using SARS-CoV-2 USA-WA1/2020 strain (Lot #080420-900) or 1BA.1 variant. Ancestral stock was generated using Vero E6 cells infected with seed stock virus obtained from Kenneth Plante at UTMB (lot #TVP 23156). BA.1 stock (Bioqual Lot #122121-700) was originally received from Emory (B.1.1.529 PP3P1 hCoV19/EHC_C19_2811C 12/9/2021) and expanded in Calu-3 cells. Vero E6 cells (ATCC, CRL-1586) were seeded in 24-well plates at 175,000 cells/well in DMEM/10% FBS/Gentamicin. Serial 3-fold serum dilutions were incubated in 96-well plates with 30 PFU of SARS-CoV-2 USA-WA1/2020 strain (BEI Resources NR-53780 lot no. 70038893) for 1 hour at 37° C. The serum/virus mixture was transferred to Vero E6 cells and incubated for 1 hour at 37° C. After that, 1 ml of 0.5% methylcellulose media was added to each well and plates were incubated at 37° C. for 3 days. Plates were washed, and cells were fixed with methanol. Crystal violet staining was performed, and plaques were recorded. IC50 titers were calculated as the serum dilution that gave a 50% reduction in viral plaques in comparison to control wells. Serum samples collected from City of Hope healthcare workers (N=14) at day 60 post-Pfizer/BioNTech BNT162b2 mRNA vaccination were part of an IRB-approved observational study to establish durability of immunogenic properties of emergency use authorization (EUA) vaccines against COVID-19 (IRB20720).

### Pseudovirus Production

SARS-CoV-2 pseudovirus was produced using a plasmid lentiviral system based on pALD-gag-pol, pALD-rev, and pALD-GFP (Aldevron). Plasmid pALD-GFP was modified to express Firefly luciferase (pALD-Fluc). Plasmid pCMV3-S(Sino Biological VG40589-UT) was utilized and modified to express SARS-CoV-2 Wuhan-Hu-1 S with D614G modification. Customized gene sequences cloned into pTwist-CMV-BetaGlobin (Twist Biosciences) were used to express SARS-CoV-2 VOC-specific S variants (Table 4).

TABLE 4
SARS-CoV-2 VOC-specific S mutations included in pseudoviral particles

| | CDC | |
|---|---|---|
| VOC | Classification | Substitutions/Deletions (Δ) |
| B.1.1.7 | Alpha | Δ69/70, Δ144, N501Y, A570D, D614G, P681H, |
| | | T716I, S982A, D1118H |
| B.1351 | Beta | L18F, D80A, D215G, Δ242-244, R246I, K417N, |
| | | E484K, N501Y, D614G, A701V |
| P.1 | Gamma | L18F, T20N, P26S, D138Y, R190S, K417T, E484K, |
| | | N501Y, D614G, H655Y, T1027I, V1176F |
| B.1.617.2 | Delta | T19R, G142D, Δ156-157, R158G, A222V, L452R, |
| | | T478K, D614G, P681R, D950N |

All S antigen were expressed with C-terminal 19 amino acids deletion. A transfection mixture was prepared with 1 ml OptiMEM that contained 30 μl of TransIT-LT1 transfection reagent (Mirus MIR2300) and 6 μg pALD-Fluc, 6 μg pALD-gag-pol, 2.4 μg pALD-rev, and 6.6 μg S expression plasmid. The transfection mix was added to 5×10⁶ HEK293T/17 cells (ATCC CRL11268) seeded the day before in 10 cm dishes and the cells were incubated for 72 hours at 37° C. Supernatant-containing pseudovirus was harvested and frozen in aliquots at −80° C. Lentivirus was titrated using the Lenti-X™ p24 Rapid Titer Kit (Takara) according to the manufacturer's instructions.

### Pseudovirus Neutralization Assay

SARS-CoV-2 pseudoviruses were titrated in vitro to calculate the virus stock amount that equals 100,000-200,000 relative luciferase units. Flat-bottom 96-well plates were coated with 100 μL poly-L-lysine (0.01%). Serial 2-fold serum dilutions starting from 1:20 were prepared in 50 μL media and added to the plates in triplicates, followed by 50 μL of pseudovirus. Plates were incubated overnight at 4° C. The following day, 10,000 HEK293T-ACE2 cells were added to each well in the presence of 3 μg/ml polybrene and plates were incubated at 37° C. After 48 hours of incubation, luciferase lysis buffer (Promega E1531) was added and luminescence was quantified using SpectraMax L (Molecular Devices, 100 μL One-Glo (Promega E6110) luciferin/well, 10 seconds integration time). For each plate, positive (pseudovirus only) and negative (cells only) controls were added. The neutralization titer for each dilution was calculated as follows: NT=[1−(mean luminescence with immune sera/mean luminescence without immune sera)]×100. The titers that gave 50% neutralization (NT50) were calculated by determining the linear slope of the graph plotting NT versus serum dilution by using the next higher and lower NT using Microsoft Excel (v2019).

### ELISPOT T Cell Detection

Peripheral blood mononuclear cells (PBMC) were isolated from fresh blood using Ficoll and counted using Luna-FL cell counter (Logos Biosystems). Pre-immune samples were evaluated using Human IFNγ/IL-4 Double-Color FluoroSpot (ImmunoSpot); however, this kit only allowed assessment of NHP IFNγ but did not detect NHP IL-4. The remaining time points were evaluated using Monkey IFNγ/IL-4 FluoroSpot FLEX kit and Monkey IL-2 FluoroSpot FLEX kit (Mabtech, X-21A16B and X-22B) following manufacturer instructions. Briefly, 200,000 cells/well in CTL-test serum free media (ImmunoSpot CTLT-010) were added to duplicate wells and stimulated with peptide pools (15 mers, 11 aa overlap, >70% purity). Spike peptide library (GenScript) consisted of 316 peptides and was divided into 4 sub-pools spanning the S1 and S2 domains (181=1-86; 1S2=87-168; 2S1=169-242; 2S2=243-316; peptides 173 and 304-309 were not successfully synthesized and therefore were excluded from the pools). Nucleocapsid (GenScript) and Membrane (in-house synthesized) libraries consisted of 102 and 53 peptides, respectively. Each peptide pool (2 μg/ml) and αCD28 (0.1 μg/ml, Mabtech) were added to the cells and plates were incubated for 48 hours at 37° C. Control cells (25,000/well) were stimulated with PHA (10 μg/ml). After incubation, plates were washed and primary and secondary antibodies were added according to the manufacturer's protocol. Fluorescent spots were acquired using CTL S6 Fluorocore (ImmunoSpot). For each sample, spots in unstimulated DMSO-only control wells were subtracted from spots in stimulated wells. Total spike response was calculated as the sum of the response to each spike sub-pool.

Quantification of SARS-CoV-2 gRNA (Genomic RNA)

SARS-CoV-2 gRNA copies per ml nasal wash, BAL fluid or swab, or per gram of tissue were quantified by qRT-PCR assay (Bioqual, SOP BV-034) using primer/probe sequences binding to a conserved region of SARS-CoV-2 N gene. Viral RNA was isolated from BAL fluid or swabs using the Qiagen MinElute virus spin kit (57704). For tissues, viral RNA was extracted with RNA-STAT 60 (Tel-test B)/chloroform, precipitated and resuspended in RNAse-free water. The control RNA was prepared to contain 10⁶ to 10⁷ copies per 3 μl. Eight 10-fold serial dilutions of control RNA were prepared using RNAse-free water. Duplicate samples of each dilution were prepared as described. To generate a control for the amplification, RNA was isolated from SARS-CoV-2 virus stocks. RNA copies were determined from an O.D. reading at 260, using the estimate that 1.0 OD at A260 equals 40 μg/ml of RNA. A final dilution of 10⁸ copies per 3 μl was then divided into single-use aliquots of 10 μl. These were stored at −80° C. until needed. SensiFAST Probe Lo-ROX One-Step Kit (Bioline BIO-78005) was used following manufacturer instructions. For the master mix preparation, 2.5 ml of 2× buffer containing Taq-polymerase, obtained from the TaqMan RT-PCR kit (Bioline BIO-78005), was added to a 15 ml tube. From the kit, 50 μl of the RT and 100 μl of RNAse inhibitor were also added. The primer pair at 2 μM concentration was then added in a volume of 1.5 ml. Lastly, 0.5 ml of water and 350 μl of the probe at a concentration of 2 μM were added and the tube vortexed. For the reactions, 45 μl of the master mix and 5 μl of the sample RNA were added to the wells of a 96-well plate. All samples were tested in triplicate. The plates were sealed with a plastic sheet. For amplification, the plate was placed in an Applied Biosystems 7500 Sequence detector and amplified using the following program: 48° C. for 30 minutes, 95° C. for 10 minutes followed by 40 cycles of 95° C. for 15 seconds, and 55° C. for 1 minute. The number of copies of RNA per ml was calculated by extrapolation from the standard curve and multiplying by the reciprocal of 0.2 ml extraction volume. This gave a practical range of 50 to 5×10⁸ RNA copies per swabs or per ml BAL fluid. Primers/probe sequences: 5′-GAC CCC AAA ATC AGC GAA AT-3′ (SEQ ID NO: 34); 5′-TCT GGT TAC TGC CAG TTG AAT CTG-3′ (SEQ ID NO: 35); and 5′-FAM-ACC CCG CAT TAC GTT TGG TGG ACC-BHQ1-3′ (SEQ ID NO: 36).

Quantification of SARS-CoV-2 sgRNA (Subgenomic RNA)

SARS-CoV-2 sgRNA copies were assessed through quantification of N gene mRNA by qRT-PCR using primer/probes specifically designed to amplify and bind to a region of the N gene mRNA that is not packaged into virions. Briefly, SARS-CoV-2 RNA was extracted from tissues using TRIzol, precipitated and resuspended in RNAse-free water. The signal was compared to a known standard curve of plasmid containing a cDNA copy of the N gene mRNA target region to give copies per ml. For amplification, the plate was placed in an Applied Biosystems 7500 Sequence detector and amplified using the following program: 48° C. for 30 minutes, 95° C. for 10 minutes followed by 40 cycles of 95° C. for 15 seconds, and 55° C. for 1 minute. The number of copies of RNA per ml was calculated by extrapolation from the standard curve and multiplying by the reciprocal of 0.2 ml extraction volume. This gave a practical range of 50 to 5×10⁷ RNA copies per swab or ml BAL fluid. Primers/probe sequences: 5′-CGA TCT CTT GTA GAT CTG TTC TC-3′ (SEQ ID NO: 37); 5′-GGT GAA CCA AGA CGC AGT AT-3′ (SEQ ID NO: 38); 5′-FAM-TAA CCA GAA TGG AGA ACG CAG TGG G-BHQ-3′ (SEQ ID NO: 39).

### Quantification of SARS-CoV-2 Infectious Virus Titers

Vero TMPRSS2 cells (Vaccine Research Center-NIAID) were plated at 25,000 cells/well in DMEM/10% FBS/Gentamicin. Ten-fold dilutions of the sample starting from 20 ul of material were added to the cells in quadruplicate and incubated at 37° C. for 4 days. The cell monolayers were visually inspected, and presence of CPE noted. TCID50 values were calculated using the Read-Muench formula.

### Histopathology

Histopathological evaluation of hamsters and NHP lung sections were performed by Experimental Pathology Laboratories, Inc. (Sterling, VA), and Charles River (Wilmington, MA), respectively. At necropsy, organs were collected and placed in 10% neutral buffered formalin for histopathologic analysis. Tissues were processed through to paraffin blocks, sectioned once at approximately 5 microns thickness, and stained with H&E. Board-certified pathologists were blinded to the vaccine groups and mock controls were used as a comparator. Histopathological findings were assigned a severity score between 1 (minimal) and 5 (severe) (Table 5).

TABLE 5
Scoring parameters used to evaluate hamster lung histopathology

| Grade | Severity | Findings |
|---|---|---|
| 1 | Minimal | Histopathologic change ranging from inconspicuous to barely |
| | | noticeable but so minor, small, or infrequent as to warrant no more |
| | | than the least assignable grade. For multifocal or diffusely |
| | | distributed lesions, this grade was used for processes where less |
| | | than approximately 10% of the tissue in an average high-power field |
| | | was involved. For focal or diffuse hyperplastic/hypoplastic/atrophic |
| | | lesions, this grade was used when the affected structure or tissue |
| | | had undergone a less than approximately 10% increase or |
| | | decrease in volume. |
| 2 | Mild | Histopathologic change that is a noticeable but not a prominent |
| | | feature of the tissue. For multifocal or diffusely distributed lesions, |
| | | this grade was used for processes where between approximately |
| | | 10% and 25% of the tissue in an average high-power field was |
| | | involved. For focal or diffuse hyperplastic/hypoplastic/atrophic |
| | | lesions, this grade was used when the affected structure or tissue |
| | | had undergone between an approximately 10% to 25% increase or |
| | | decrease in volume. |
| 3 | Moderate | Histopathologic change that is a prominent but not a dominant |
| | | feature of the tissue. For multifocal or diffusely distributed lesions, |
| | | this grade was used for processes where between approximately |
| | | 25% and 50% of the tissue in an average high-power field was |
| | | involved. For focal or diffuse hyperplastic/hypoplastic/atrophic |
| | | lesions, this grade was used when the affected structure or tissue |
| | | had undergone between an approximately 25% to 50% increase or |
| | | decrease in volume. |
| 4 | Marked | Histopathologic change that is a dominant but not an overwhelming |
| | | feature of the tissue. For multifocal or diffusely distributed lesions, |
| | | this grade was used for processes where between approximately |
| | | 50% and 95% of the tissue in an average high-power field was |
| | | involved. For focal or diffuse hyperplastic/hypoplastic/atrophic |
| | | lesions, this grade was used when the affected structure or tissue |
| | | had undergone between an approximately 50% to 95% increase or |
| | | decrease in volume. |
| 5 | Severe | Histopathologic change that is an overwhelming feature of the |
| | | tissue. For multifocal or diffusely distributed lesions, this grade was |
| | | used for processes where greater than approximately 95% of the |
| | | tissue in an average high-power field was involved. For focal or |
| | | diffuse hyperplastic/hypoplastic/atrophic lesions, this grade was |
| | | used when the affected structure or tissue had undergone a greater |
| | | than approximately 95% increase or decrease in volume. |

### Statistical Analyses

Statistical analyses were performed using Prism 8 (GraphPad, v8.3.0). One-way ANOVA with Holm-Sidak's multiple comparison test, two-way ANOVA with Tukey's or Dunn's multiple comparison test, the Kruskal-Wallis test followed by Dunn's multiple comparison test, and Spearman correlation analysis were used. All tests were two-sided. The test applied for each analysis and the significance level is indicated in each figure legend. Prism 8 was used to derive correlation matrices.

### Example 2: COH04S1 Induces Robust Th1-Biased S and N Antigen-Specific Antibodies and Cross-NAb Responses Against SARS-CoV-2 in Hamsters Through IM and IN Vaccination

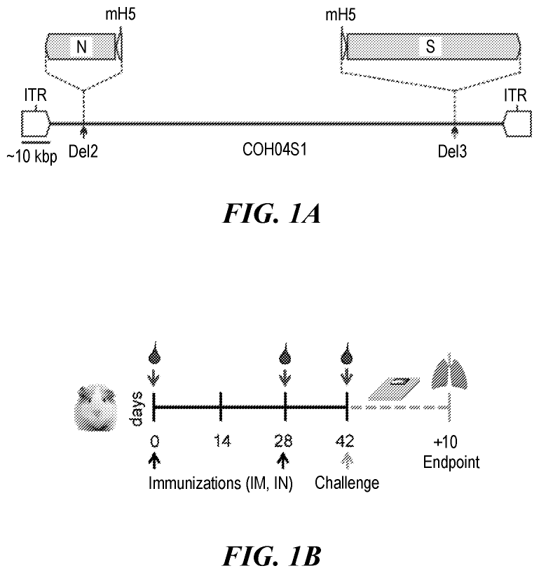

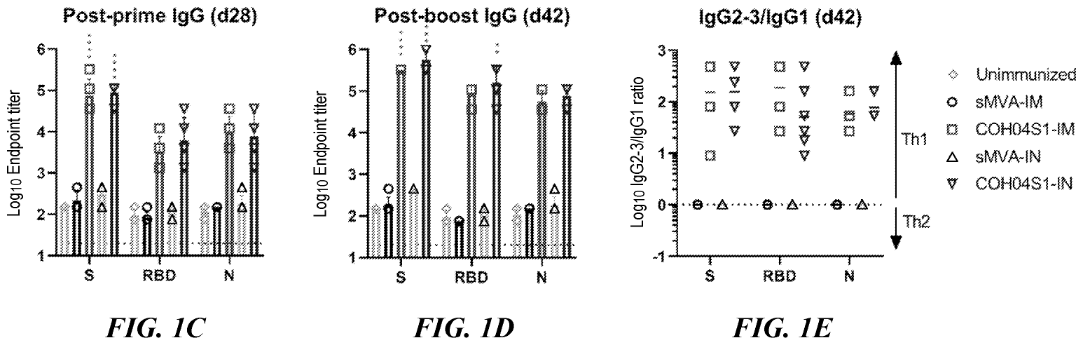

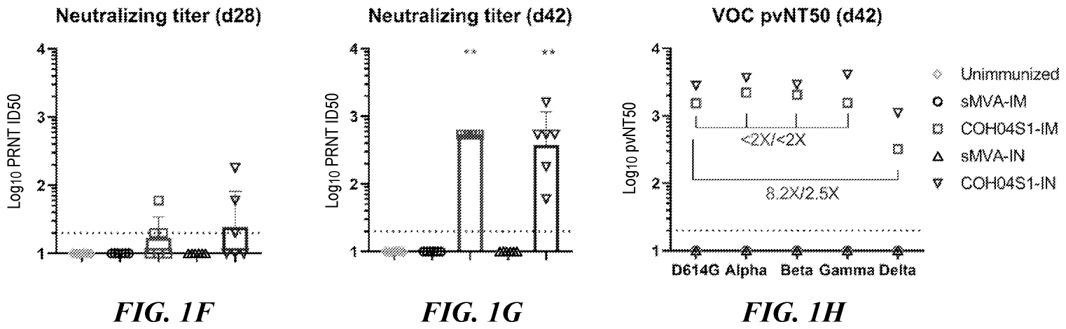

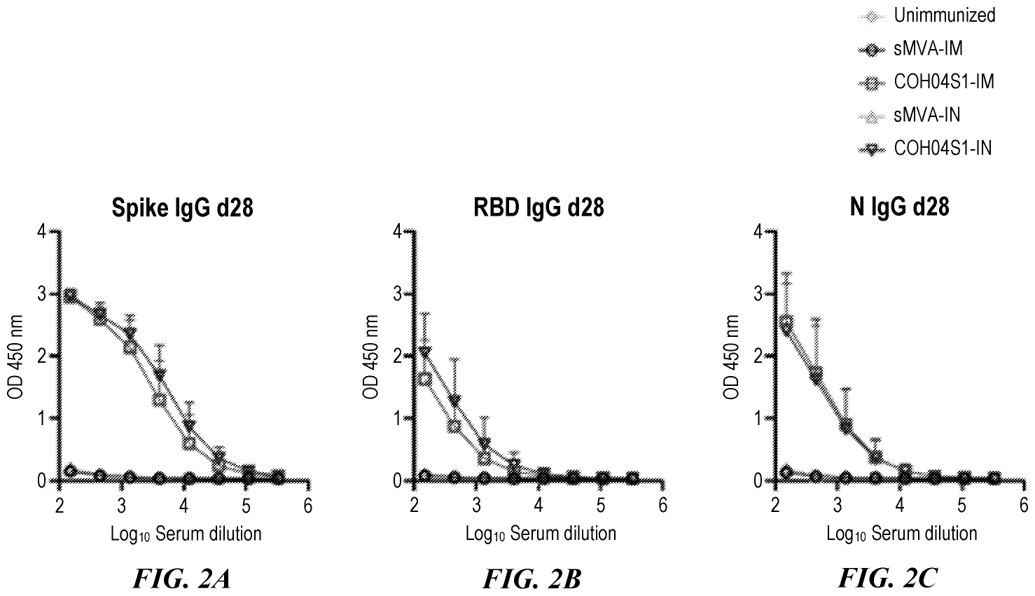

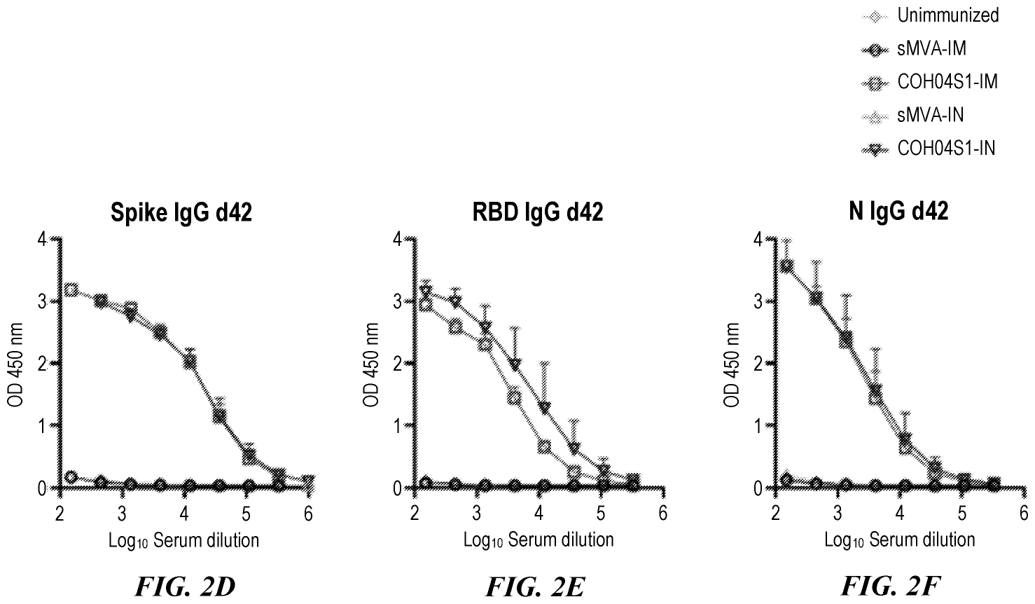

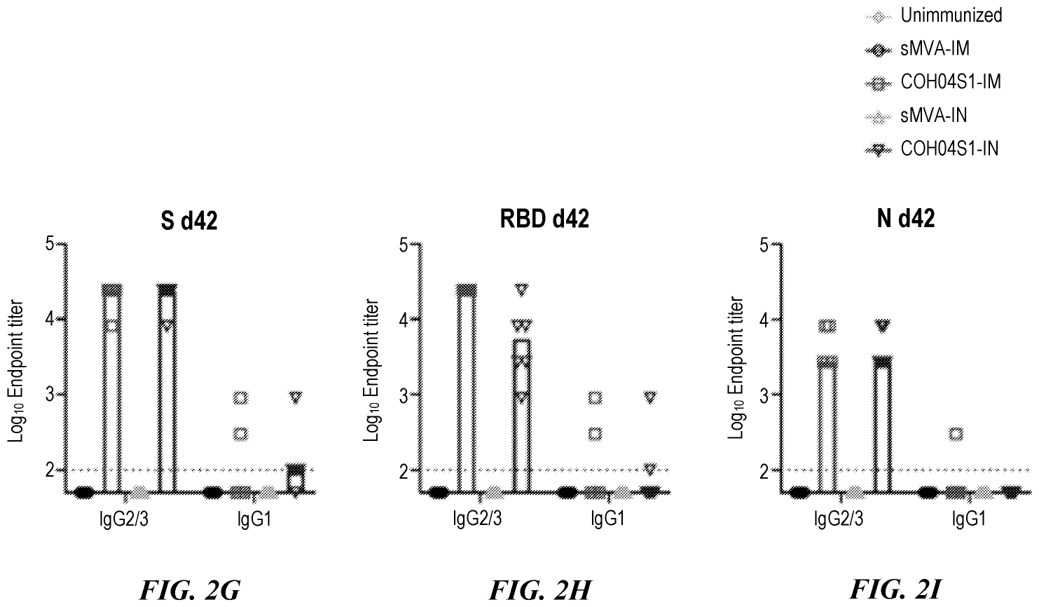

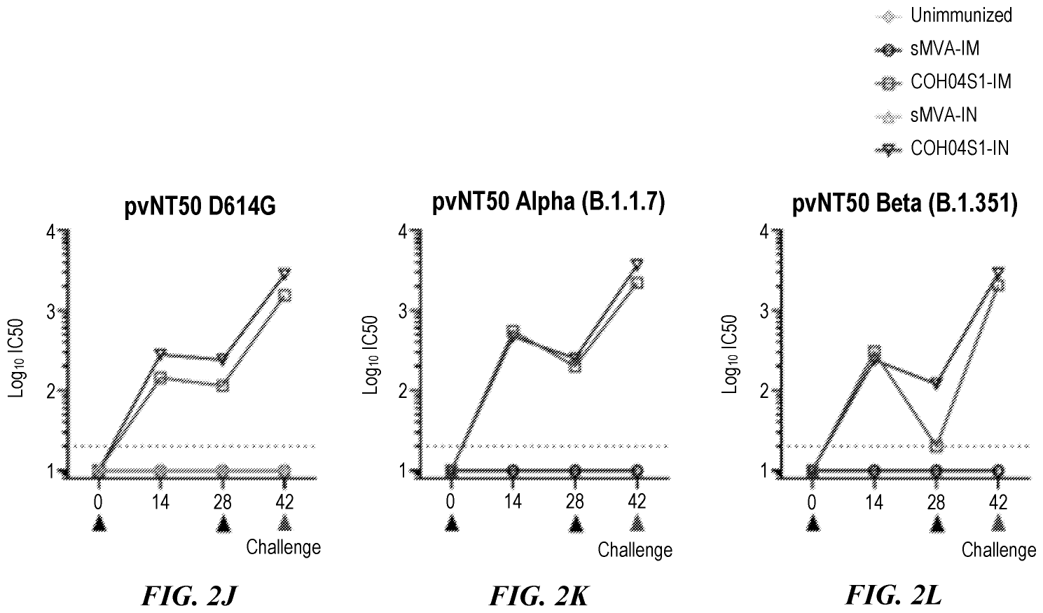

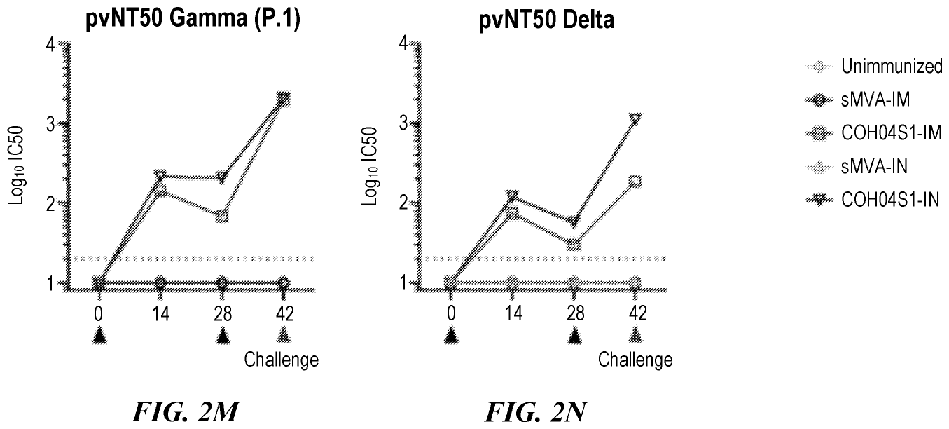

Syrian hamsters are widely used to evaluate vaccine protection against SARS-CoV-2 in a small animal model that mimics moderate-to-severe COVID-19 disease. Using this animal model, the efficacy of COH04S1 (FIG. 1A) to protect against SARS-CoV-2 challenge by either IM or IN vaccination was determined to assess vaccine protection via parenteral or mucosal immune stimulation. Hamsters were vaccinated twice in a 4-week interval with 1×10⁸ PFU of COH04S1 by IM or IN route, referred to as COH04S1-IM or COH04S1-IN, respectively (FIG. 1B). Unvaccinated animals and hamsters vaccinated by IM or IN route with empty sMVA vector were used as controls. COH04S1-IM and COH04S1-IN stimulated robust and comparable binding antibodies to both the S and N antigens, including antibodies to the RBD, the primary target of NAb. Binding antibodies to S, RBD, and N were detected at high levels in both the COH04S1-IM and COH04S1-IN vaccine groups after the first vaccination and boosted after the second vaccination (FIGS. 1C-1D, 2A-2N), whereby a particularly strong booster effect was observed for RBD-specific antibodies. Antigen-specific binding antibodies stimulated by COH04S1-IM and COH04S1-IN in hamsters were mainly composed of IgG2/3 isotypes and only to a minor extent of IgG1 isotype (FIGS. 1E, 2A-2N), indicating Th1-biased immune responses.

PRNT assay measuring neutralizing activity against SARS-CoV-2 infectious virus (USA-WA1/2020) demonstrated that potent and comparable NAb titers were stimulated by COH04S1-IM and COH04S1-IN after the booster vaccinations (FIGS. 1F-1G). Furthermore, pooled post-boost immune sera from COH04S1-IM- and COH04S1-IN-vaccinated animals demonstrated potent cross-reactive neutralizing activity against SARS-CoV-2 pseudoviruses (pv) with D614G S mutation or multiple S modifications based on several SARS-CoV-2 VOC (FIGS. 1H, 2A-2N; Table 3), including Alpha (B.1.1.7), Beta (B.1.351), Gamma (P.1), and Delta variants (B.1.617.2). Notably, while NAb titers measured with Alpha, Beta, and Gamma PV variants were generally similar to those determined with D614G PV, NAb titers measured with the Delta-matched PV variant were 2- to 8-fold reduced compared to those determined with D614G PV. In addition, NAb titers measured in COH04S1-IM immune sera using the different PV variants appeared lower overall than those measured in COH04S1-IN immune sera. These results demonstrate that IM and IN vaccination of Syrian hamsters with COH04S1 elicits robust Th1-biased S and N antigen-specific binding antibodies as well as potent NAb responses with cross-reactivity to prevent PV infection by several SARS-CoV-2 VOC.

### Example 3: IM and IN Vaccination with COH04S1 Protect Hamsters from Progressive Weight Loss, Lower Respiratory Tract Infection, and Lung Pathology Following SARS-CoV-2 Challenge

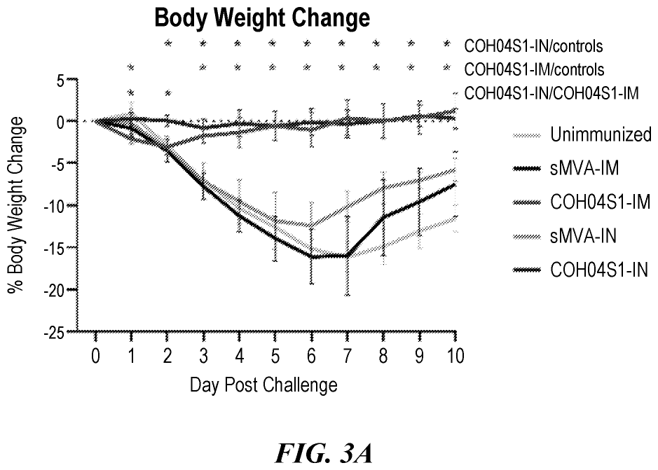

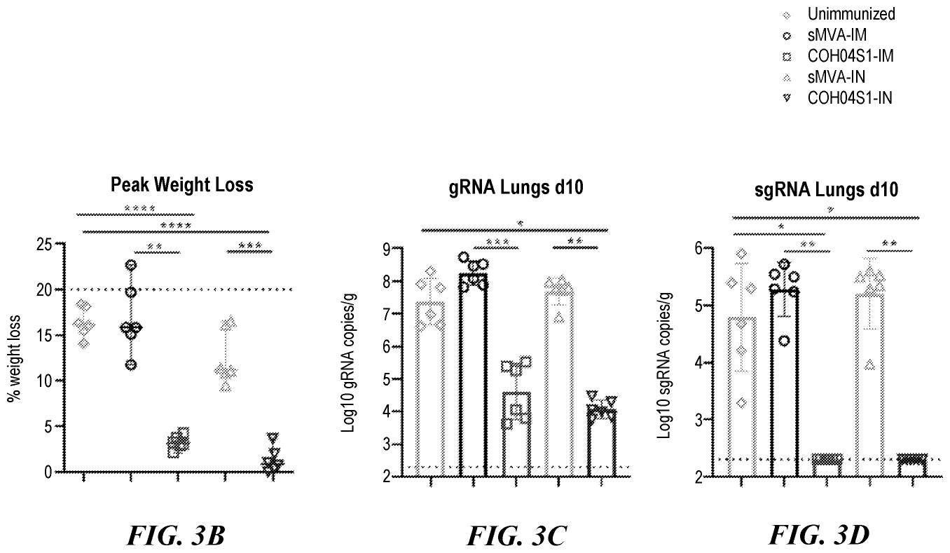

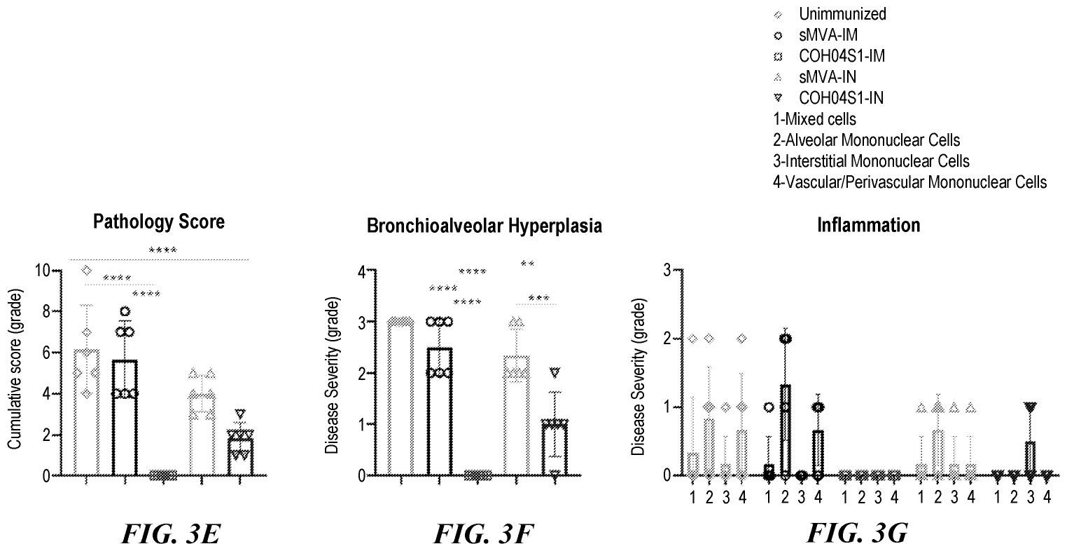

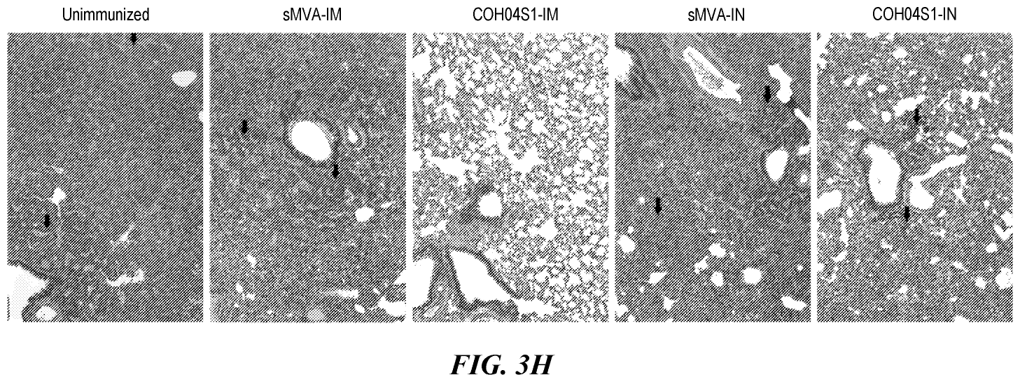

Two weeks after the booster vaccination, hamsters were challenged intranasally with 6×10⁴ PFU of SARS-CoV-2 reference strain USA-WA1/2020 and body weight changes were measured over a period of 10 days. While control animals showed rapid body weight loss post-challenge, hamsters vaccinated by IM or IN route with COH04S1 were protected from severe weight loss following challenge (FIGS. 3A-3B). Control animals showed rapid body weight loss for 6-7 days post-challenge, reaching maximum weight loss between 10% and 20%. In contrast, COH04S1-IM- and COH04S1-IN-vaccinated animals showed no or only very minor body weight decline post-challenge, with maximum body weight loss below 4% for all animals at any time point during the entire 10-day observation period (FIGS. 3A-3B). Notably, while minor weight loss was observed for COH04S1-IM-vaccinated animals at 1-2 days post-challenge, COH04S1-IN-vaccinated animals did not show body weight decline at these early time points post-challenge (FIG. 3A), suggesting improved protection from weight loss by COH04S1 through IN vaccination compared to IM vaccination at an early phase post-challenge.

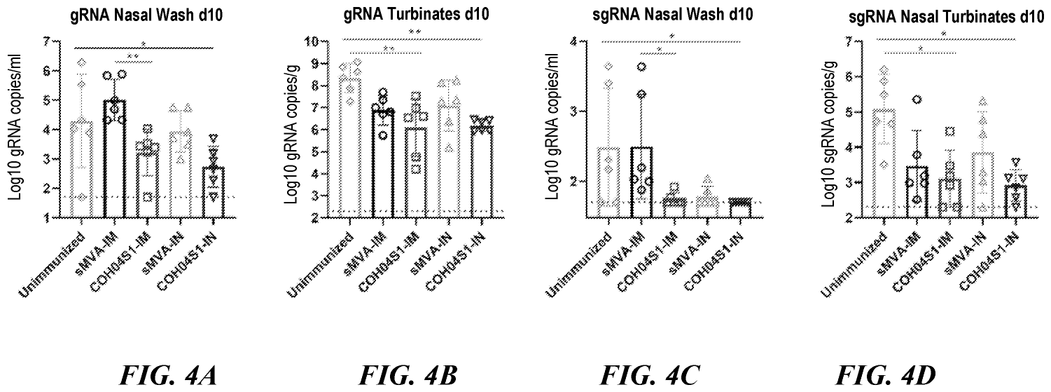

At day 10 post-challenge, hamsters were euthanized for viral load assessment and histopathology analysis. Viral load was measured in the lungs and nasal turbinates/wash by quantification of SARS-CoV-2 genomic RNA (gRNA) and subgenomic RNA (sgRNA) to gauge the magnitude of total and replicating virus at lower and upper respiratory tracts. Compared to lung viral loads of control animals, markedly reduced gRNA and sgRNA copies were observed in the lungs of COH04S1-IM- and COH04S1-IN-vaccinated animals (FIGS. 3C-3D), demonstrating potent vaccine protection against lower respiratory tract infection through IM and IN vaccination. SARS-CoV-2 gRNA copies in the lungs of COH04S1-IM- and COH04S1-IN-vaccinated animals were more than 3 to 4 orders of magnitude lower than in the lungs of control animals. Furthermore, while 103-10⁶ sgRNA copies were detected in the lungs of control animals, sgRNA was undetectable in the lungs of COH04S1-IM- and COH04S1-IN-vaccinated animals, indicating complete absence of replicating virus in lung tissue of vaccinated hamsters. Compared to nasal viral loads of controls, gRNA and sgRNA viral loads in nasal turbinates and wash of COH04S1-IM- and COH04S1-IN-vaccinated animals appeared only marginally reduced, indicating limited vaccine impact on upper respiratory tract infection by COH04S1 independent of vaccination route (FIGS. 4A-4D).

Histopathological findings in hematoxylin/eosin-stained lung sections of euthanized animals were assessed by a board-certified pathologist and scored in a blind manner on a scale from 1 to 5 based on the severity and diffusion of the lesions (Table 5).

Control animals demonstrated compromised lung structure characterized by moderate bronchioloalveolar hyperplasia with consolidation of lung tissue, minimal to mild mononuclear or mixed cell inflammation, and syncytial formation (FIGS. 3E-3H). In contrast, COH04S1-IM-vaccinated animals did not show lung pathology of any type and grade in 6/6 hamsters, demonstrating potent vaccine protection against SARS-CoV-2-mediated lung injury in hamsters by IM vaccination with COH04S1. While COH04S1-IN-vaccinated animals presented no severe histopathological findings and significantly reduced lung pathology compared to controls, COH04S1-IN-vaccinated hamsters consistently showed a mild form of bronchioloalveolar hyperplasia and grade 1 interstitial inflammation in a subset of animals, indicating that IN vaccination with COH04S1 mediated potent but incomplete vaccine protection against SARS-CoV-2-mediated lung damage in this model (FIGS. 3E-3H).

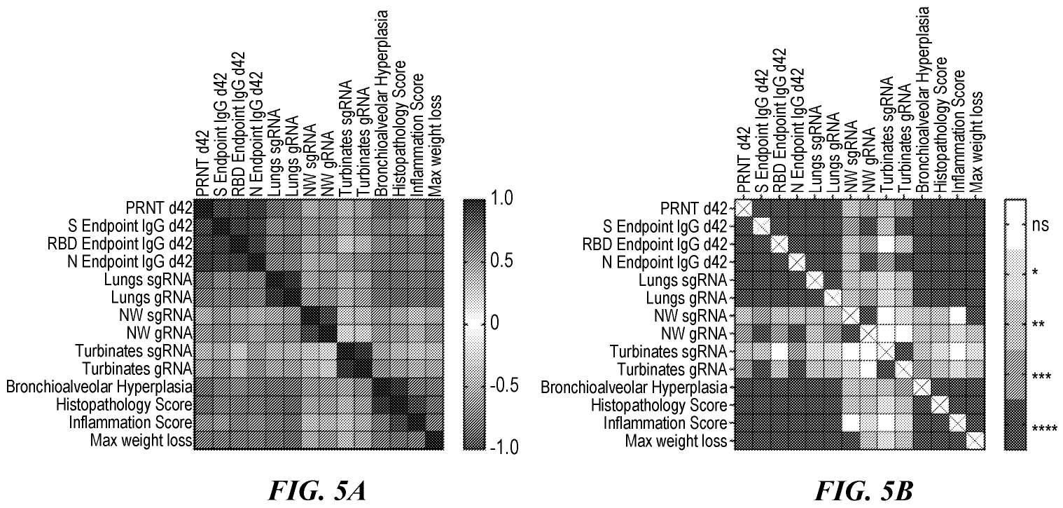

Correlative analysis of pre-challenge immunity and post-challenge outcome revealed that any of the evaluated COH04S1-induced responses, including S-, RBD-, and N-specific antibodies and NAb, correlated with protection from weight loss, lung infection, and lung pathology (FIGS. 5A-5B), confirming that the observed protection was vaccine-mediated. These results in sum demonstrate that hamsters vaccinated by IM and IN route with COH04S1 are protected from progressive weight loss, lower respiratory tract infection, and severe lung pathology following SARS-CoV-2 challenge.

### Example 4: 2D and 1 D Vaccination of NHP with COH04S1 Stimulates Robust Antigen-Specific Binding Antibodies, NAb Responses, and Antigen-Specific IFNγ- and IL-2-Expressing T Cells

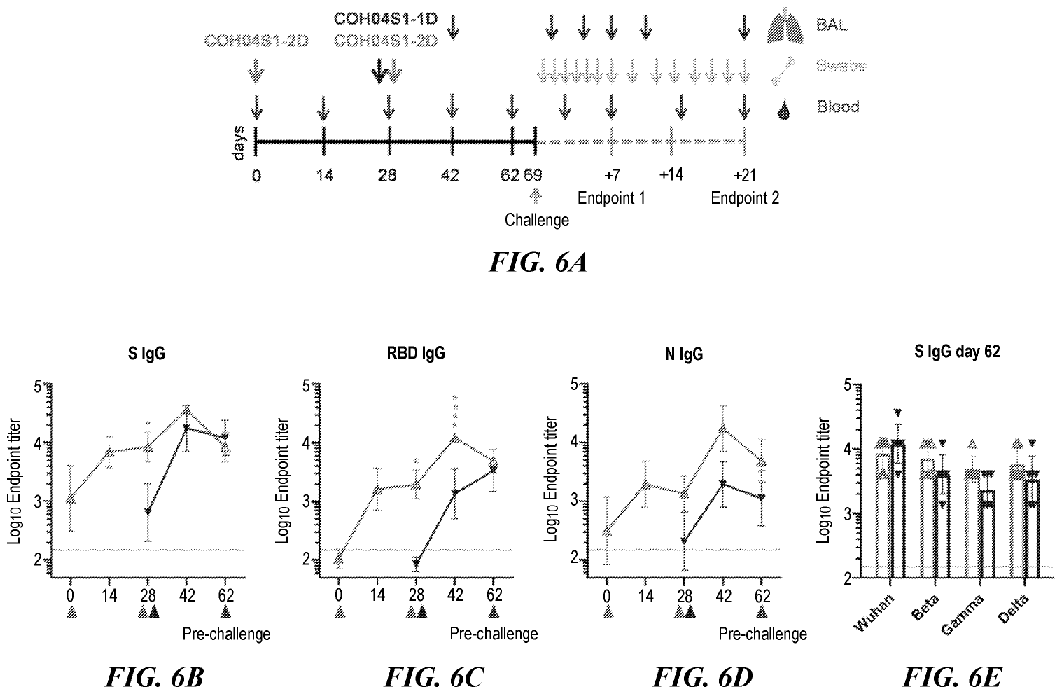

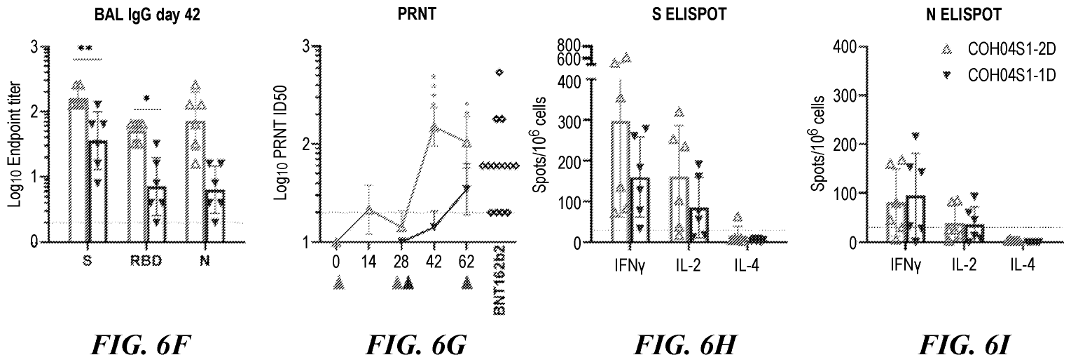

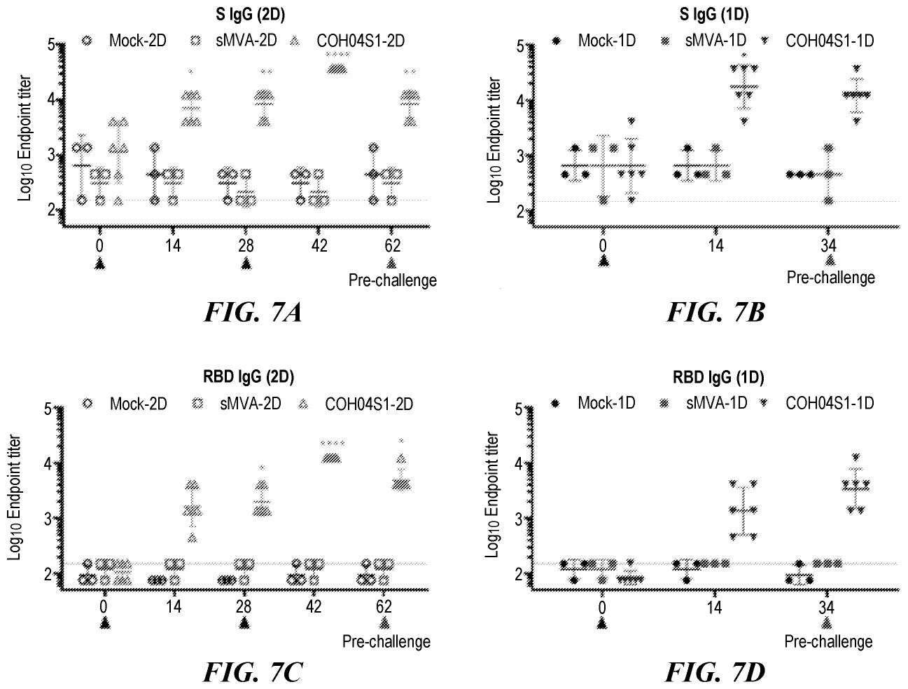

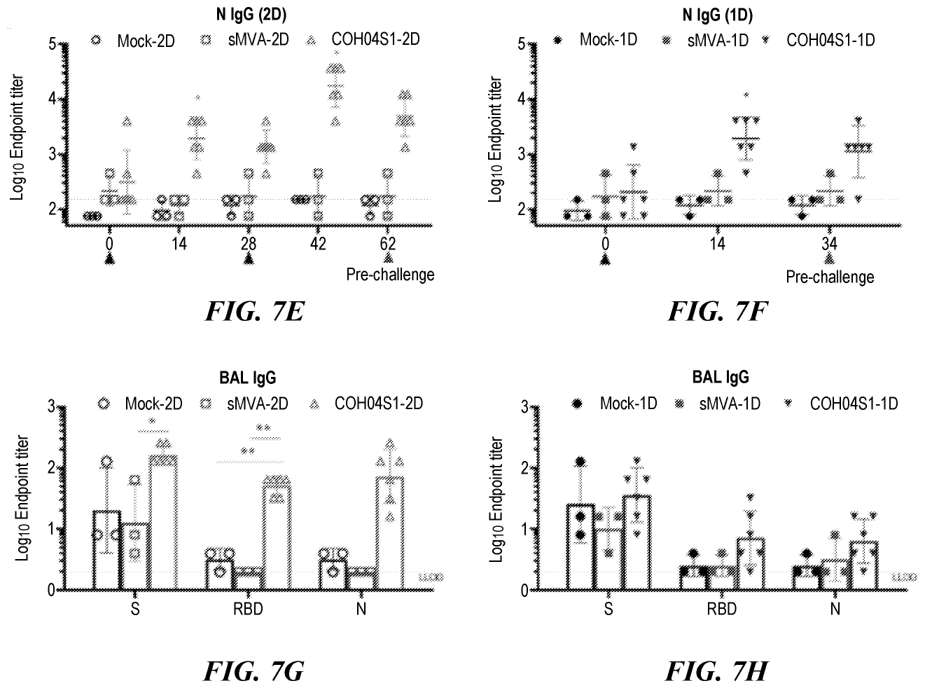

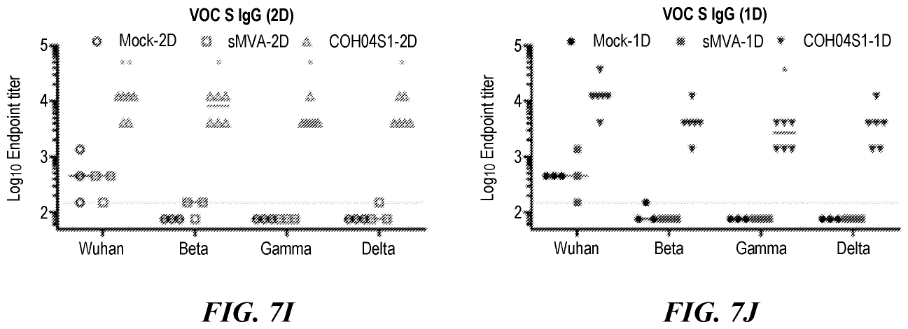

NHP represents a mild COVID-19 disease model that is widely used to bolster preclinical SARS-CoV-2 vaccine efficacy against upper and lower respiratory tract infection in an animal species that is more closely related to humans. The African green monkey NHP model was used to assess COH04S1 vaccine protection against SARS-CoV-2 by 2D and 1 D vaccination regimen, referred to as COH04S1-2D and COH04S1-1 D, respectively. For 2D vaccination, NHP were vaccinated twice in a 4-week interval with 2.5×10⁸ PFU of COH04S1. For 1D vaccination, monkeys were vaccinated once with 5×10⁸ PFU of COH04S1 (FIG. 6A). As controls, monkeys were either mock-vaccinated or vaccinated with empty sMVA vector via the same schedule and dose vaccination regimen. Robust serum binding antibodies to S, RBD, and N were stimulated in NHP by both COH04S1-2D and COH04S1-1 D, whereas binding antibodies in the 2D vaccine group were strongly boosted after the second dose. At the time of challenge, S- and RBD-specific antibody titers measured in the 2D and 1 D vaccine groups were comparable (FIGS. 6B-6D, 7A-7J), while N-specific titers appeared higher in the 2D vaccine group than in the 1 D vaccine group. Notably, similar S-specific antibody titers were measured in 2D- and 1 D-vaccinated NHP by S antigens based on the original Wuhan-Hu-1 reference strain and various VOC (Beta, Gamma, and Delta), whereas binding antibodies titers measured with VOC-specific S antigens tended to be lower than those measured with the Wuhan-Hu-1 S antigen (FIGS. 6E, 7A-7J). Antigen-specific binding antibodies of the IgG type were also measured in lung BAL samples 2 weeks pre-challenge. BAL IgG binding antibodies to S, RBD, and N were detected in both COH04S1-2D- and COH04S1-1D-vaccinated NHP, although BAL IgG antibody titers measured at this time point pre-challenge were higher in the 2D vaccine group than in the 1 D vaccine group (FIGS. 6F, 7A-7J).

NAb measurements based on PRNT assay revealed that both COH04S1-2D and COH04S1-1 D elicited NAb responses with efficacy to neutralize SARS-CoV-2 infectious virus (USA-WA1/2020). NAb responses measured in COH04S1-2D-vaccinated animals were boosted after the second dose and exceeded those measured in COH04S1-1 D-vaccinated NHP at the time of challenge (FIG. 6G). Notably, NAb measured by PRNT assay in COH04S1-2D- and COH04S1-1 D-vaccinated NHP were within the range of peak titers measured post-second dose in a cohort of healthcare workers that received the FDA-approved BNT162b2 mRNA vaccine from Pfizer/BioNTech.

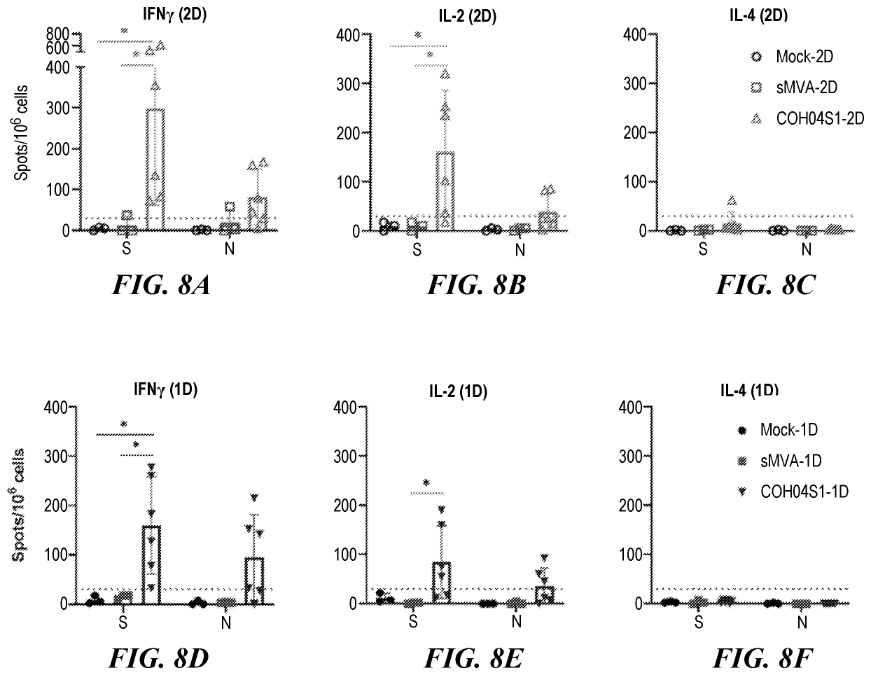

At 2 weeks pre-challenge, SARS-CoV-2 antigen-specific T cell responses in COH04S1-vaccinated NHP were also assessed. Both 2D and 1D vaccination with COH04S1 stimulated robust IFNγ- and IL-2-expressing S and N antigen-specific T cells, whereas no or only very low levels of IL-4-expressing S- and N-specific T cells were observed in COH04S1-2D- or COH04S1-1D-vaccinated animals, consistent with Th1-biased immunity (FIGS. 6H-6I, 8A-8F). S-specific T cells were generally detected at a higher frequency than N-specific T cells in both the COH04S1-2D and COH04S1-1D vaccine groups. In addition, S-specific T cells measured at this time point pre-challenge were detected at a higher frequency in 2D-vaccinated NHP than in 1 D-vaccinated NHP. These results demonstrate that COH04S1 elicits robust antigen-specific binding antibodies, NAb, and antigen-specific IFNγ- and IL-2-expressing T cells in NHP through 2D and 1D vaccination regimen.

### Example 5: 2D and 1 D Vaccination with COH04S1 Protects NHP from Lower and Upper Respiratory Tract Infection Following SARS-CoV-2 Challenge

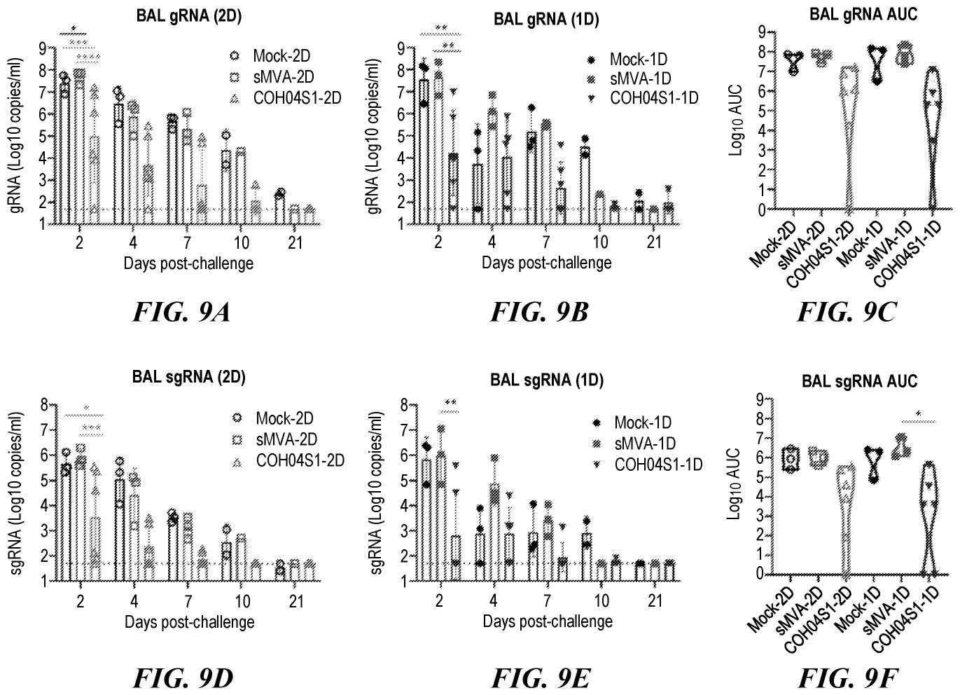

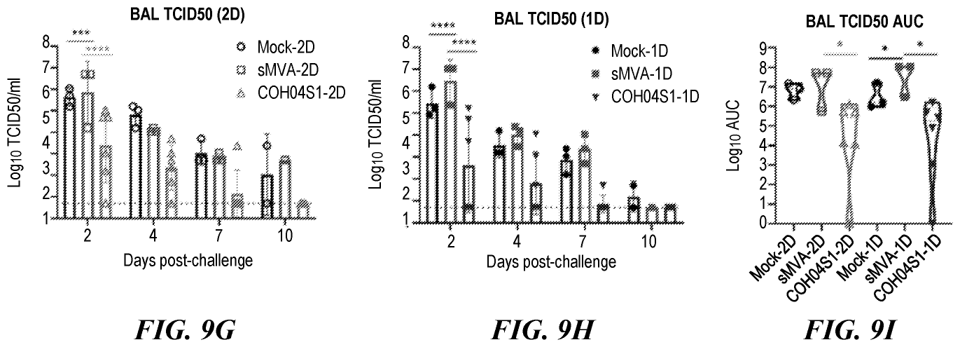

Six weeks after 2D or 1D vaccination with COH04S1, vaccinated NHP were challenged by IN/IT route with 1×10⁵ PFU of SARS-CoV-2 (USA-WA1/2020). SARS-CoV-2 viral loads in lower and upper respiratory tracts were assessed at several time points for 21 days post-challenge in BAL and nasal/oral swabs by quantification of gRNA and sgRNA and infectious virus titers (FIGS. 9A-9I, 10A-10I, 11A-11F). Progressively declining viral loads were overall measured over the 21 days post-challenge observation period in BAL and nasal/oral swabs of both COH04S1-vaccinated NHP and control animals. Compared to BAL viral loads of control animals, markedly reduced gRNA and sgRNA copies and infectious virus titers were measured in BAL of COH04S1-2D- and COH04S1-1 D-vaccinated NHP at almost all time points post-challenge (FIGS. 9A-9B, 9D-9E, 9G-9H), indicating a potent vaccine impact on lower respiratory tract infection. Notably, BAL gRNA and sgRNA copies and infectious titers measured in COH04S1-2D- and COH04S1-1 D-vaccinated NHP at day 2 immediately after challenge were on average 2 to 3 orders of magnitude lower than those measured in controls, confirming rapid vaccine efficacy. The marked reduction in BAL viral loads of COH04S1-vaccinated NHP compared to controls was confirmed when combining viral loads measured at all time points post-challenge by area under the curve (AUC; FIGS. 9C, 9F, 9I).

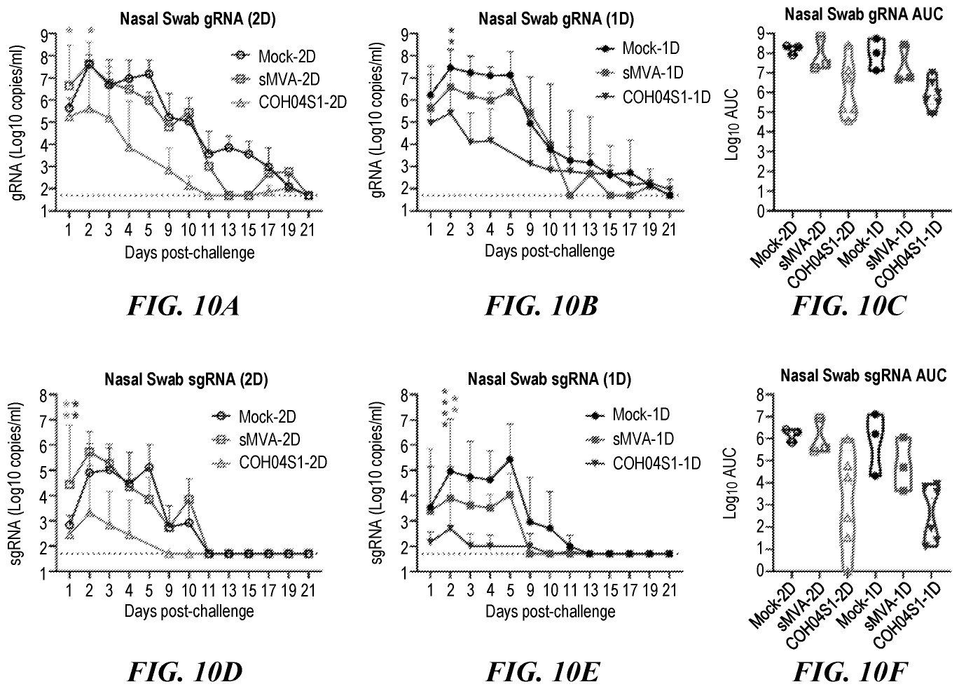

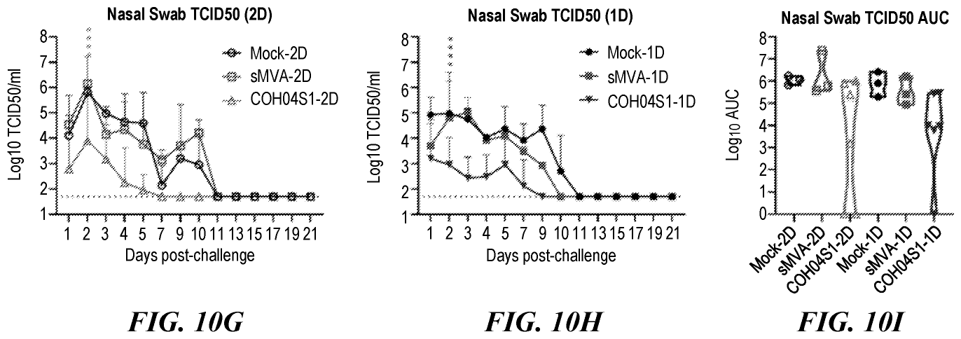

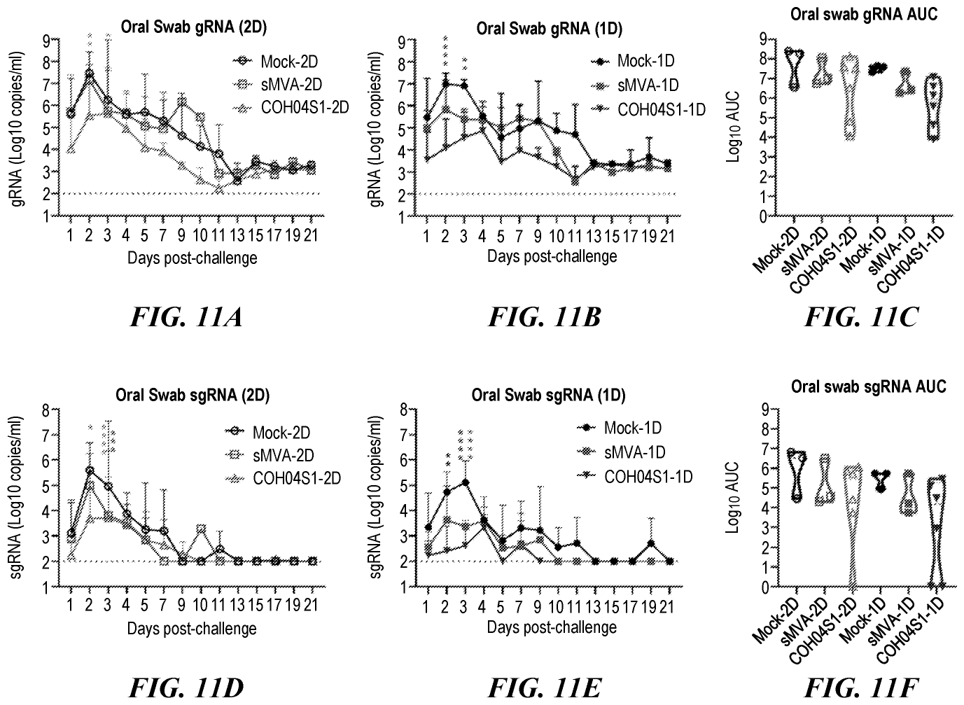

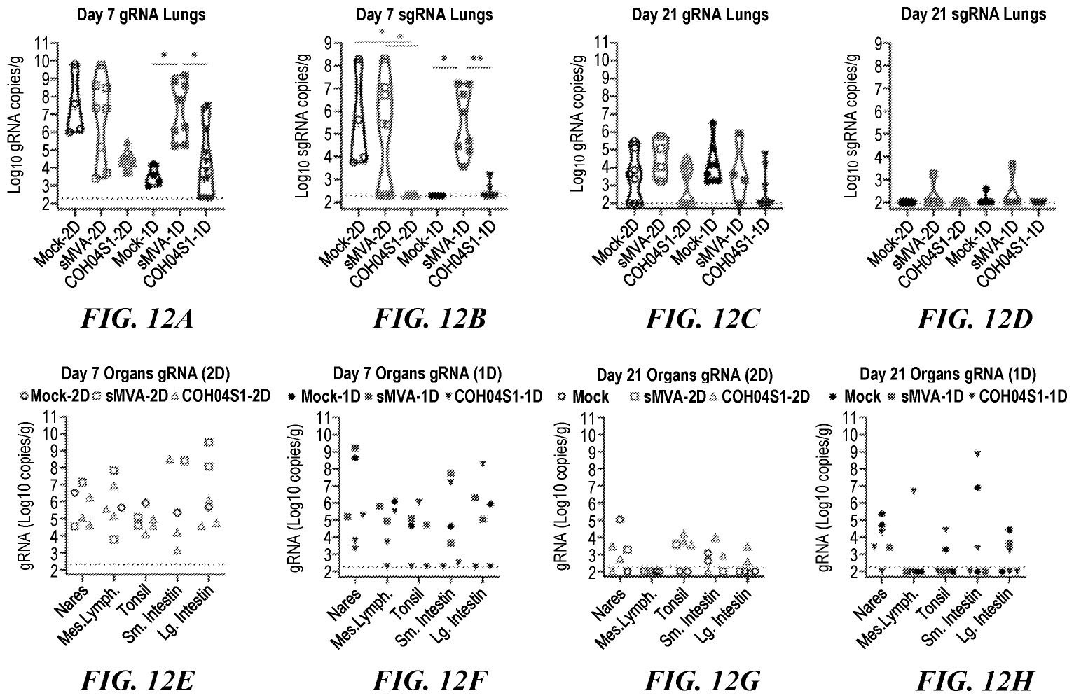

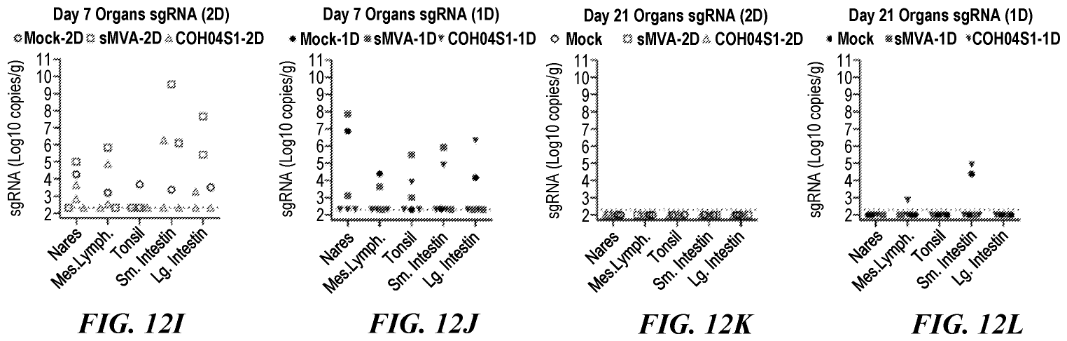

Similar to viral loads in BAL of COH04S1-vaccinated NHP, gRNA and sgRNA copies and infectious virus titers measured at the first 10 days post-challenge in nasal swabs of COH04S1-2D- or COH04S1-1 D-vaccinated NHP were consistently lower than those of control animals, demonstrating vaccine efficacy to prevent upper respiratory tract infection (FIGS. 10A-10I). In addition, gRNA and sgRNA in oral swabs of COH04S1-vaccinated NHP tended to be consistently lower than those in oral swabs of control animals (FIGS. 11A-11F). Notably, nasal and oral swab gRNA and sgRNA copies and nasal swab infectious titers measured at 1-3 days immediately after challenge in COH04S1-vaccinated NHP were significantly reduced compared to those of controls, indicating immediate vaccine protection. Overall reduced nasal and oral swab viral loads in COH04S1-vaccinated NHP compared to control animals were confirmed when evaluating the nasal and oral swab viral loads over time by AUC (FIGS. 10C, 10F, 10I, 11A-11F). No significant differences in viral loads were observed between COH04S1-2D and COH04S1-1 D-vaccinated animals, indicating similar protection against lower and upper respiratory tract infection by COH04S1 through 2D and 1 D vaccination. Viral loads measured at day 7 or day 21 post-challenge in lung tissue and several other organs of necropsied animals did not indicate evident differences between COH04S1-vaccinated NHP and control animals (FIGS. 12A-12L). While gRNA and sgRNA copies measured at day 7 post-challenge in lung samples of COH04S1-2D- and COH04S1-1 D-vaccinated NHP appeared reduced compared to those measured in lung samples of controls, these results were inconclusive due to low or undetectable gRNA and sgRNA levels in lung samples of the 1D mock-vaccinated control monkey (FIGS. 12A-12L). Histopathological findings assessed by a board-certified pathologist in a blind manner at days 7 and 21 post-challenge were generally only minor and comparable between COH04S1 vaccine and control groups, and no increase in inflammation was observed for COH04S1-vaccinated NHP compared to control animals (Table 6).

TABLE 6
NHP vaccine groups, necropsy schedule, and gross pathological findings

| | | Weight | | Nx | Nx | |
|---|---|---|---|---|---|---|
| Group | Study | (kg) | Sex | day +7 | day +21 | Gross pathology findings at Nx |
| Mock | 2D | 4.76 | F | | X | All lobes normal |
| | | 3.02 | F | X | | Right caudal lung, remaining lobes normal. Overall |
| | | | | | | hemorrhaging |
| | | 3.78 | F | | X | Left and right caudal lung, remaining lobes normal |
| | 1D | 3.56 | F | X | | Pathology and congestion in all lobes |
| | | 3.45 | F | | X | Pathology and congestion in all lobes |
| | | 6.79 | M | | X | Left and right caudal lung, remaining lobes normal |
| sMVA | 2D | 4.03 | F | X | | Right caudal lung, remaining lobes normal |
| | | 3.31 | F | X | | Gross congestion, possibly due to BALs. Lobes normal |
| | | 3.63 | F | | X | Right caudal lung, remaining lobes normal |
| | 1D | 3.41 | F | X | | Gross congestion, possibly due to BALs. Lobes normal |
| | | 3.73 | F | X | | Gross congestion, possibly due to BALs. Lobes normal |
| | | 6.05 | M | | X | All lobes normal |
| COH04S1 | 2D | 4.02 | F | X | | Gross congestion, possibly due to BALs. Lobes normal |
| | | 2.91 | F | X | | Right caudal lung, remaining lobes normal |
| | | 3.69 | F | | X | Gross congestion, possibly due to BALs. Lobes normal |
| | | 3.68 | F | | X | Right caudal lung, remaining lobes normal |
| | | 3.37 | F | X | | Right caudal lung, remaining lobes normal |
| | | 7.23 | M | | X | Right caudal lung, remaining lobes normal |
| | 1D | 3.79 | F | X | | Gross congestion, possibly due to BALs. Lobes normal |
| | | 3.83 | F | | X | Right caudal lung, remaining lobes normal |
| | | 2.95 | F | X | | Gross congestion, possibly due to BALs. Lobes normal |
| | | 3.86 | F | | X | Gross congestion, possibly due to BALs. Lobes normal |
| | | 3.36 | F | X | | Right caudal lung, remaining lobes normal |
| | | 5.63 | M | | X | All lobes normal |

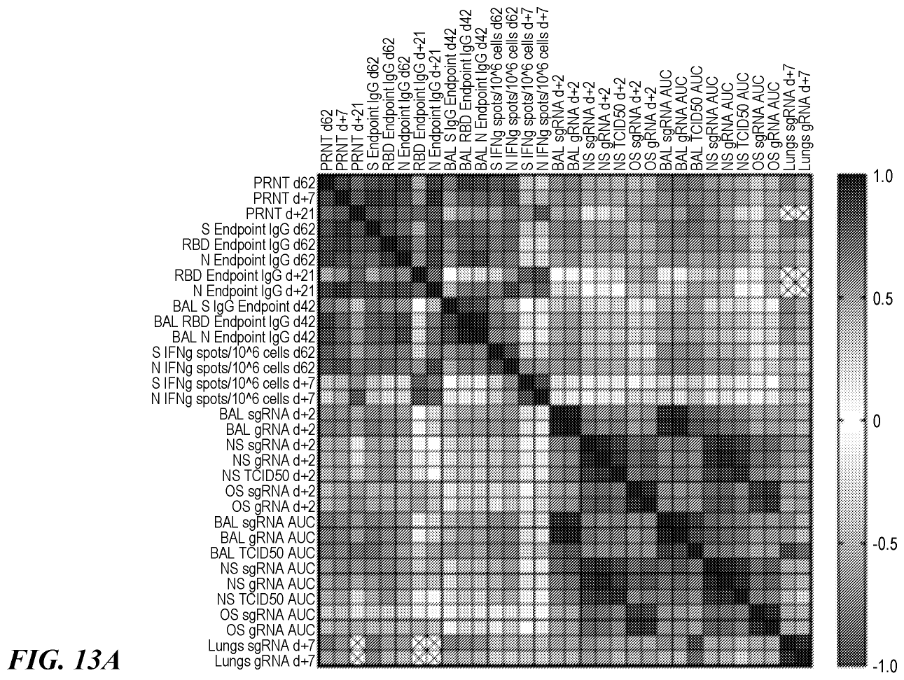

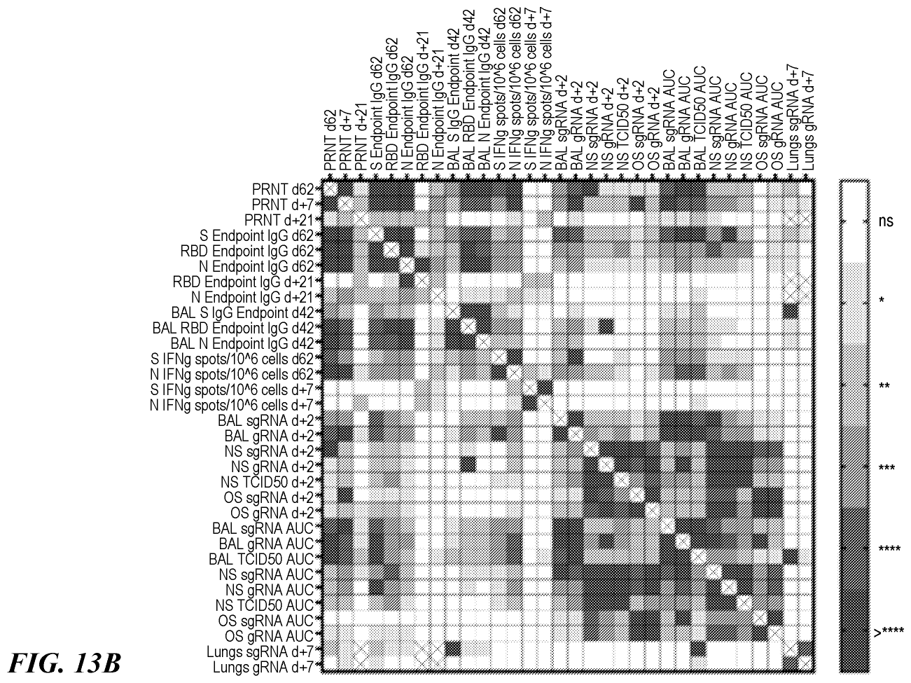

A strong inverse correlation could be assessed between vaccine-induced humoral and cellular immune responses, including S-, RBD-, and N-specific binding antibodies in serum and BAL, NAb, and S- and N-specific T cells, and SARS-CoV-2 viral loads in BAL and nasal swab samples (FIGS. 13A-13B). These results in sum demonstrate that 2D or 1 D vaccination regimen of COH04S1 protect NHP from lower and upper respiratory tract infection following INIT challenge with SARS-CoV-2.

### Example 6: NHP Vaccinated with 2D and 1 D Vaccination Regimen of COH04S1 Develop Robust Post-Challenge Anamnestic Immune Responses

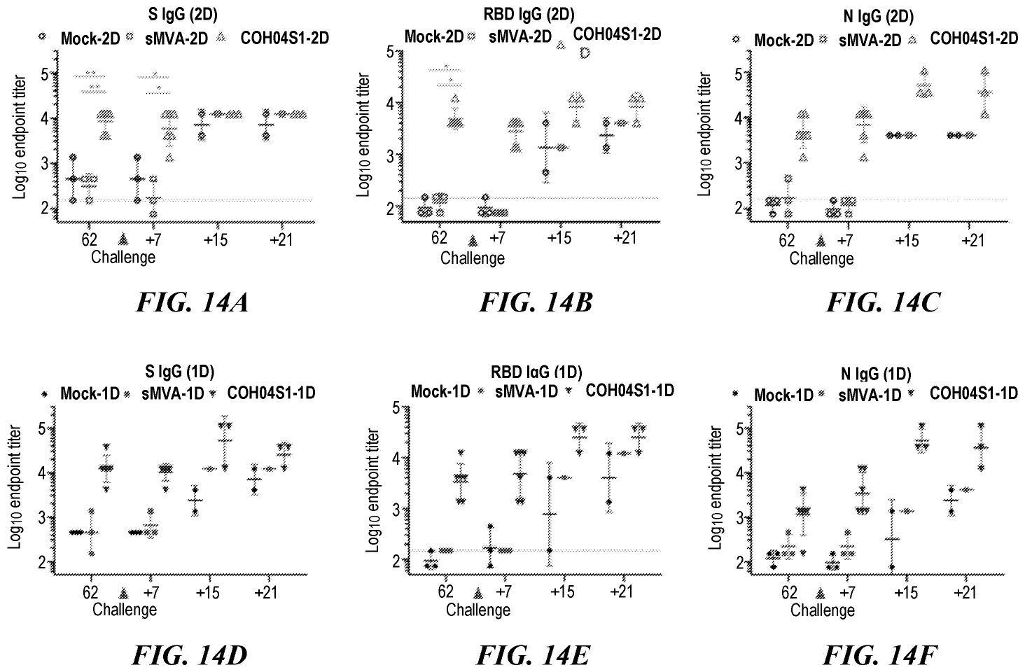

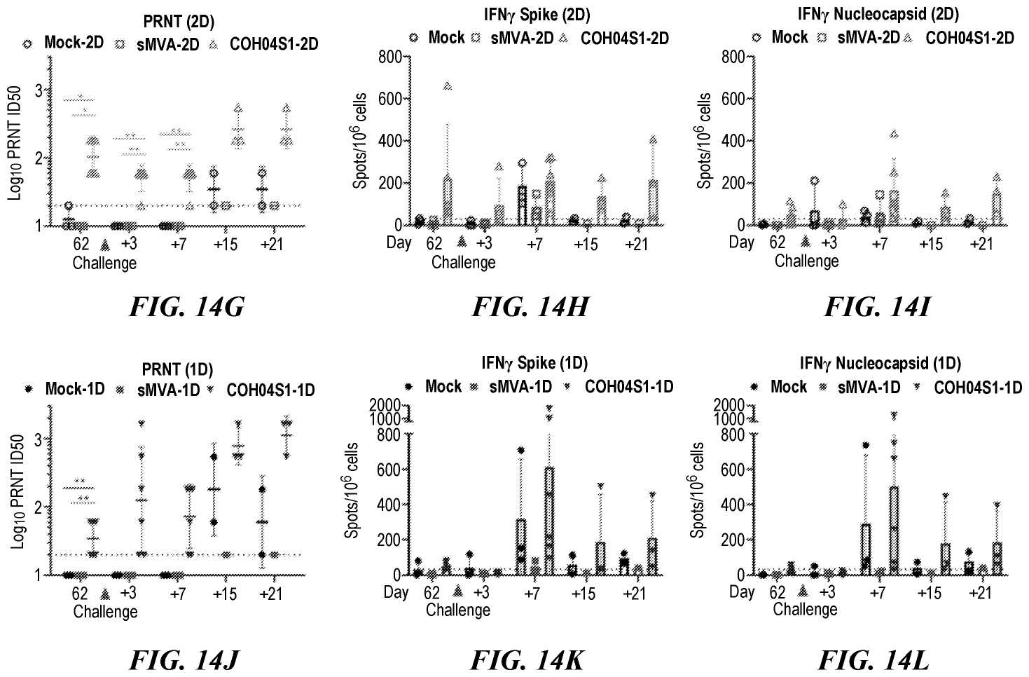

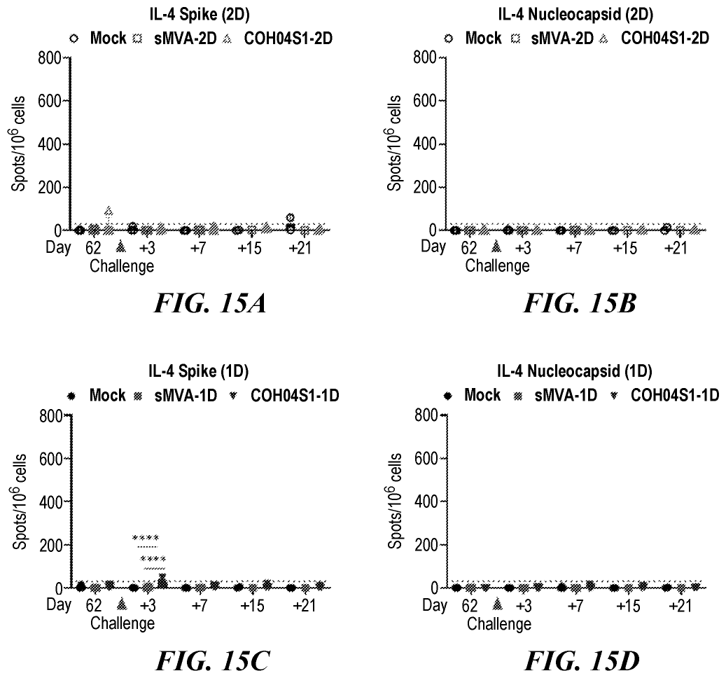

To assess the vaccine impact on post-challenge viral immunity, humoral and cellular responses were evaluated in COH04S1-vaccinated NHP and control animals at 1, 2, and 3 weeks post-challenge (FIGS. 14A-14L, 15A-15D). This analysis revealed that control animals developed robust binding antibodies to 8, RBD, and N at 15 or 21 days post-challenge (FIGS. 14A-14F), which indicated stimulation of potent humoral responses by the SARS-CoV-2 challenge virus in naïve NHP. In contrast, NAb titers measured post-challenge in control animals by PRNT assay against infectious virus were in general relatively low or at the limit of detection (FIGS. 14G, 14J), although elevated NAb responses were observed in 1D mock-vaccinated control animals at days 15 and 21 post-challenge (FIG. 14J). In addition, no or only low-level T cell responses were detected post-challenge in control animals, with the exception of elevated frequencies of S- and N-specific IFNγ-expressing T cell responses in mock-vaccinated control animals at day 7 post-challenge (FIGS. 14H-141, 14K-14L).

Compared to antibodies measured pre-challenge in COH04S1-vaccinated NHP, boosted titers of S, RBD, and N antigen-specific binding antibodies and PRNT-assayed NAb were measured in COH04S1-vaccinated animals at days 15 and 21 post-challenge (FIGS. 14A-14G, 14J), indicating induction of robust post-challenge anamnestic immune responses. This post-challenge booster effect on SARS-CoV-2-specific humoral immunity appeared more pronounced in COH04S1-1 D-vaccinated NHP than in COH04S1-2D-vaccinated NHP, which may be a result of the lower pre-challenge responses in the 1 D vaccine group compared to the 2D vaccine group. In addition, binding antibodies and NAb measured at days 15 and 21 post-challenge in COH04S1-vaccinated NHP generally exceeded those measured in control animals, indicating heightened vaccine-mediated humoral recall responses against SARS-CoV-2 through 2D or 1 D vaccination. While S and N antigen-specific IFNγ-expressing T cell levels measured in COH04S1-2D-vaccinated animals tended to increase only slightly after challenge, a marked increase in post-challenge S- and N-specific IFNγ-expressing T cells was observed in COH04S1-1 D-vaccinated NHP, indicating potent vaccine-mediated cellular recall responses following 1 D vaccination. S- and N-specific IL-4-expressing T cells were either only very low or undetectable in COH04S1-vaccinated NHP and control animals at any time point post-challenge, consistent with Th1-biased immunity following challenge (FIGS. 15A-15D). Correlation analysis did not unambiguously reveal a strong association between any of the post-challenge immunological parameters and post-challenge virological assessments (FIGS. 13A-13B). These results demonstrate that NHP vaccinated with 2D or 1D vaccination regimen of COH04S1 develop potent anamnestic antiviral post-challenge recall responses.

### Example 7: Vaccination with COH04S1 and COH04S351 Protects Hamsters from Weight Loss Following SARS-CoV-2 Delta Variant Challenge

Syrian hamsters (N=10) were intramuscularly vaccinated two times in a 28-day interval with COH04S1 or COH04S351. COH04S1 comprises S and N antigen sequences based on the original SARS-CoV-2 Wuhan isolate, while COH04S351 comprises S and N antigen sequences based on SARS-CoV-2 B.1.351 beta variant. The DNA and protein sequences for the S antigen of COH04S351 are represented by SEQ ID NOs: 21 and 22, respectively, and the DNA and protein sequences for the N antigen of COH04S351 are represented by SEQ ID NOs: 23 and 24, respectively.

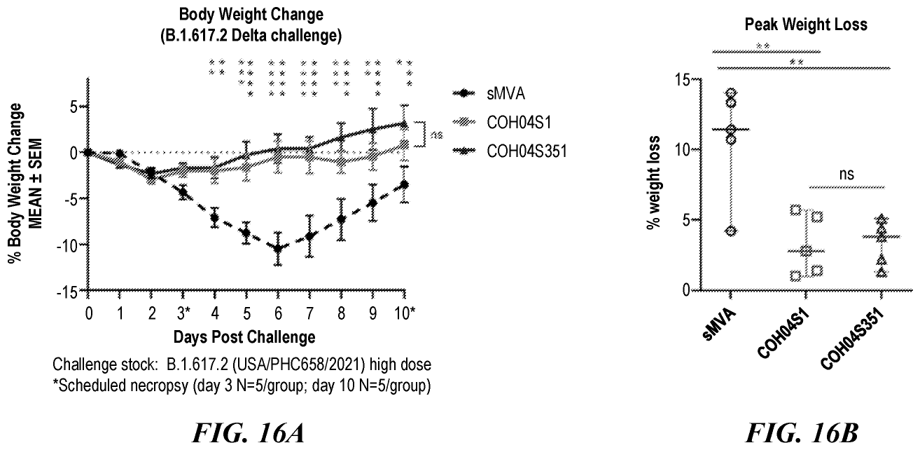

Control animals were vaccinated with sMVA control vector. All vaccines were administered at 1×10⁸ PFU. Two weeks post-boost, hamsters were challenged intranasally with a high dose (6.5×10⁴ TCID50 per hamster) of SARS-CoV-2 B.1.617.2 Delta variant (isolate USA/PHC658/2021, which has the following mutations in the S protein: T19R, K77T, deletion 157-158, L452R, T478K, D614G, P681R, and D950N). Body weight was measured daily for 10 days post-challenge. FIG. 16A shows that hamsters vaccinated with either COH04S1 or COH04S351 did not suffer from significant weight loss. FIG. 16B shows the maximal weight loss in single animals within vaccine and control groups (N=5).

As demonstrated herein, IM or IN vaccination of Syrian hamsters with COH04S1 stimulated robust Th1-biased S- and N-specific humoral immunity and cross-neutralizing antibodies (NAb) and protected against weight loss, lower respiratory tract infection, and lung injury following intranasal SARS-CoV-2 challenge. In addition, one- or two-dose vaccination of African green monkeys with COH04S1 induced robust antigen-specific binding antibodies, NAb, and Th1-biased T cells, protected against both upper and lower respiratory tract infection following IN/IT SARS-CoV-2 challenge, and triggered potent post-challenge anamnestic antiviral recall responses. These results demonstrate that COH04S1 stimulates protective immunity against SARS-CoV-2 in animal models through different vaccination routes and dose regimen, which complements ongoing investigation of this multi-antigen sMVA-vectored SARS-CoV-2 vaccine in clinical trials.

### Example 8: Comparison of COH04S1 and COH04S351 Immunogenicity in Mice

In this in vivo experiment, COH04S1 and COH04S351 immunogenicity was compared in balb/c mice.

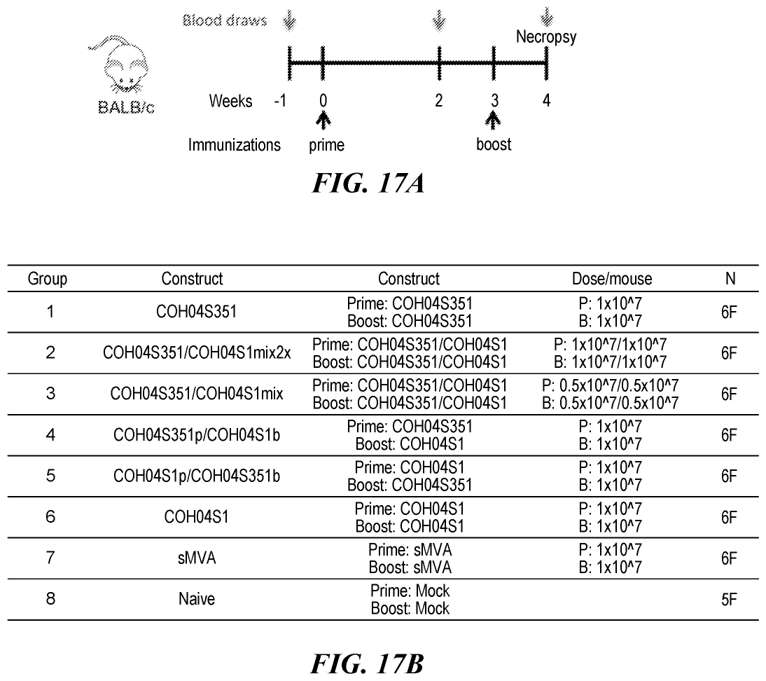

As shown in FIGS. 17A-17B, female balb/c mice (N=6) were intraperitoneally vaccinated two times in a 21-day interval with COH04S351, COH04S1, a combination of COH04S351/COH04S1 in two different amounts, or with the two vaccines used in a heterologous prime/boost schedule. Control animals were vaccinated with sMVA control vector (N=6) or PBS (N=5). Single vaccines were administered at 1×10⁷ PFU. The combination of COH04S351/COH04S1 was administered at either 1×10⁷ PFU (mix2×) or 0.5×10⁷ (mix) PFU/each vaccine. Serum samples were collected at baseline, week 2, and endpoint (week 4), when splenocytes were also collected for immunological analyses.

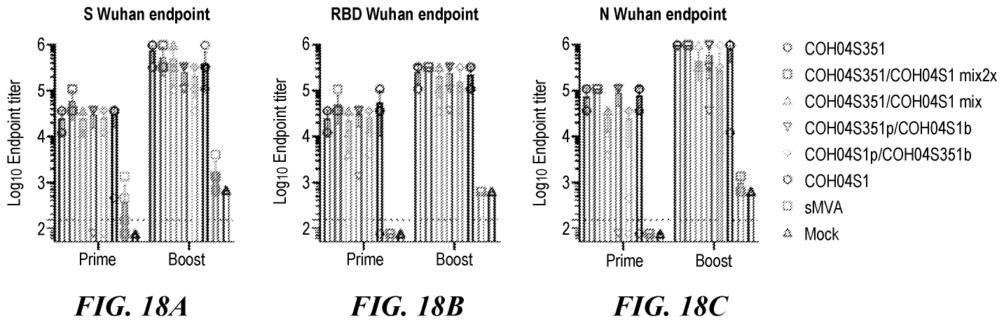

Serum binding antibodies recognizing S, RBD, and N antigens based on the sequence from the original Wuhan strain (the ancestral Wuhan strain) were measured post-prime and post-boost vaccination by ELISA. As shown in FIG. 18, similar levels of S-, RBD-, and N-specific binding antibodies were induced post-prime and post-boost in animals vaccinated with the different vaccines and vaccine combinations. Some variability in the antibody titers was observed within the groups, probably due to technical error.

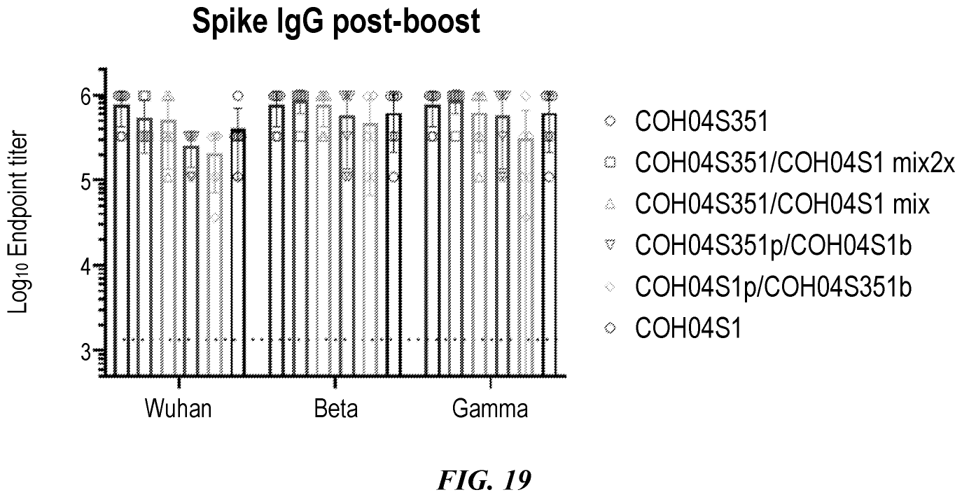

Binding antibodies recognizing S antigens from different variants of concern (VOC) were evaluated by ELISA. As shown in FIG. 19, both COH04S1 and COH04S351, and combinations of these vaccines, induced comparable S-specific binding antibodies recognizing S from the ancestral Wuhan strain, Beta, and Gamma (P.1) VOC.

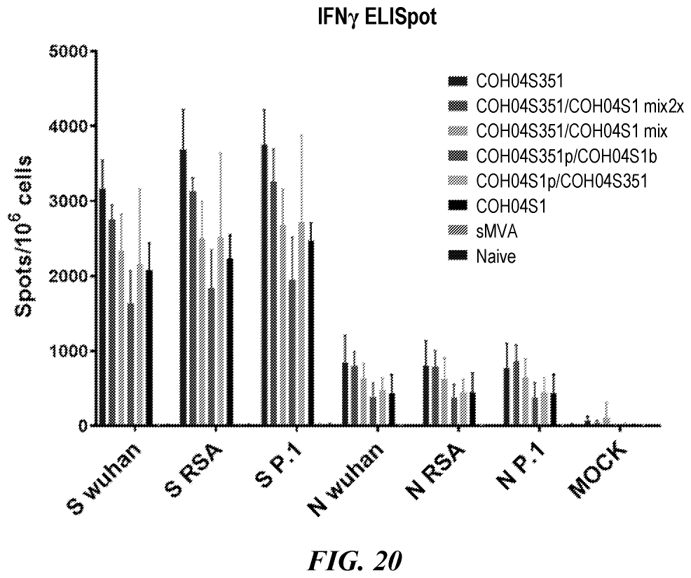

To evaluate T cell responses to S and N antigens from different VOC, peptide libraries were assembled with peptides covering S and N sequences from the ancestral Wuhan strain, Beta, and Gamma VOC. The peptides were used to stimulate mouse splenocytes collected post-boost in an ELISPOT assay. As shown in FIG. 20, all vaccine combinations induced robust T cell responses to S and N antigens. In particular, COH04S351 vaccination appears to have induced stronger T cell responses to ancestral and VOC antigens than COH04S1.

These results demonstrate that COH04S1 and COH04S351, and combinations of these antigens, are similarly immunogenic in balb/c mice.

### Example 9: Comparison of COH04S1 and C170 Immunogenicity in Mice

In this in vivo experiment, COH04S1 and C170 immunogenicity was compared in balb/c mice. C170 is a SARS-CoV-2 candidate vaccine encoding for S and N from the Gamma variant of concern, P.1, originally isolated in Brazil. The DNA and protein sequences for the S antigen of C170 are represented by SEQ ID NOs: 29 and 30, respectively, and the DNA and protein sequences for the N antigen of C170 are represented by SEQ ID NOs: 31 and 32, respectively.

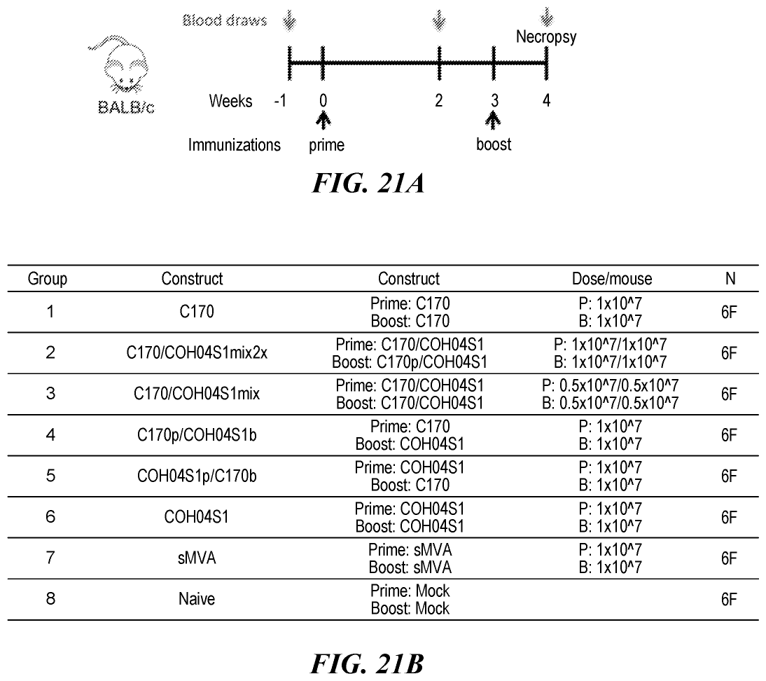

As shown in FIGS. 21A-21B, female balb/c mice (N=6) were intraperitoneally vaccinated two times in a 21-day interval with C170, COH04S1, a combination of C170/COH04S1 in two different amounts, or with the two vaccines used in a heterologous prime/boost schedule. Control animals were vaccinated with sMVA control vector or PBS (N=6). Single vaccines were administered at 1×10⁷ PFU. The combination of C170/COH04S1 was administered at either 1×10⁷ PFU (mix2×) or 0.5×10⁷ (mix) PFU/each vaccine. Serum samples were collected at baseline, week 2, and endpoint (week 4), when splenocytes were also collected for immunological analyses.

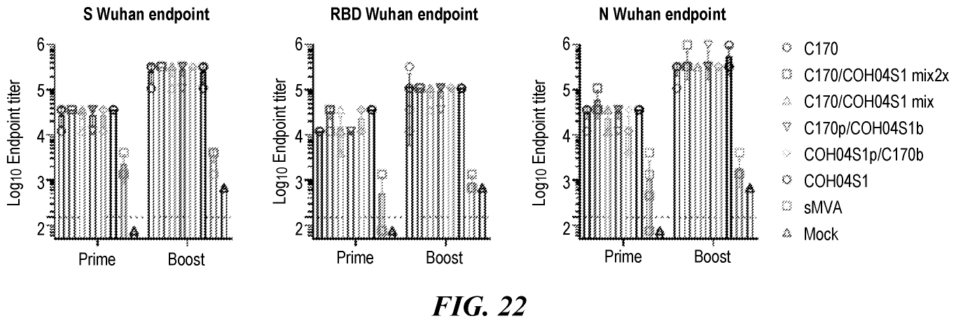

Serum binding antibodies recognizing S, RBD, and N antigens based on the sequence from the ancestral Wuhan strain were measured post-prime and post-boost vaccination by ELISA. As shown in FIG. 22, similar levels of S-, RBD-, and N-specific binding antibodies were induced post-prime and post-boost in animals vaccinated with the different vaccines and vaccine combinations.

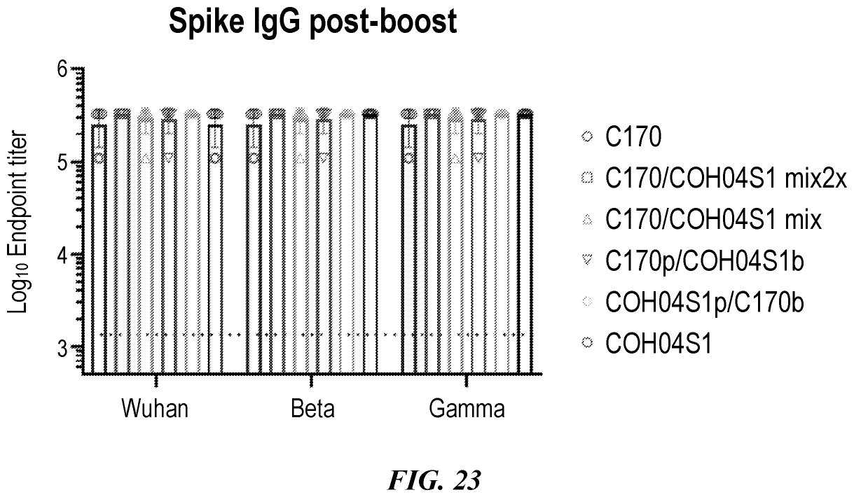

Binding antibodies recognizing S antigens from different VOC were evaluated by ELISA. As shown in FIG. 23, both COH04S1 and C170, and combinations of these vaccines, induced comparable S-specific binding antibodies recognizing S from ancestral Wuhan strain, Beta, and Gamma (P.1) VOC.

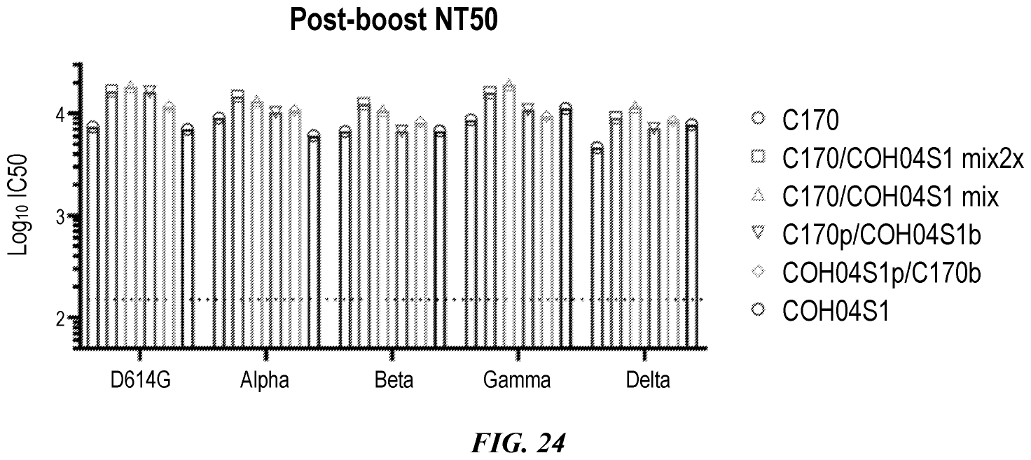

NAb titers were measured in pooled post-boost serum samples using S-pseudoviruses based on the ancestral Wuhan strain with D614G substitution, Alpha, Beta, Gamma, and Delta VOC. As shown in FIG. 24, comparable titers of NAb to ancestral strain and VOC were measured post-boost in pooled serum samples.

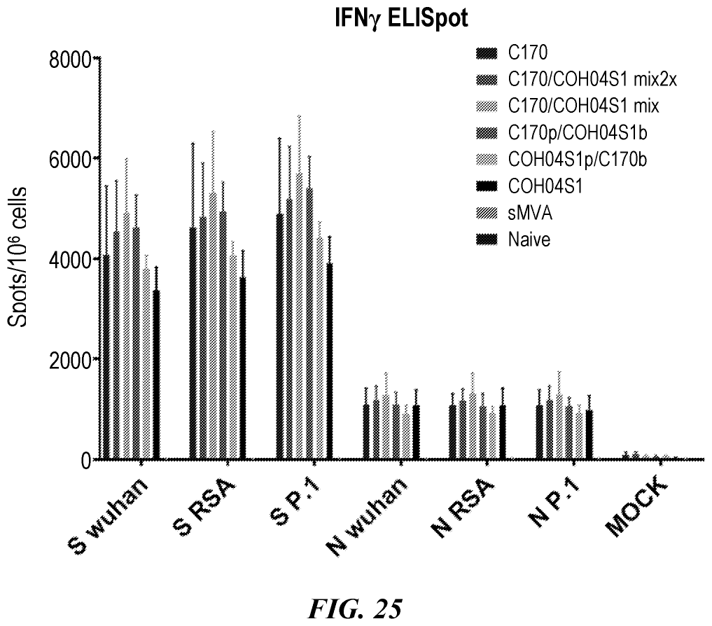

To evaluate T cell responses to S and N antigens from different VOC, peptide libraries were assembled with peptides covering S and N sequences from ancestral Wuhan strain, Beta, and Gamma VOC. The peptides were used to stimulate mouse splenocytes collected post-boost in an ELISPOT assay. As shown in FIG. 25, all vaccine combinations induced robust T cell responses to S and N antigens.

These results demonstrate that COH04S1 and C170, and combinations of these antigens, are similarly immunogenic in balb/c mice.

### Example 10: Evaluation of COH04S1 and COH04S351 Immunogenicity in Hamsters

This example shows an in vivo study in golden Syrian hamsters aimed at the evaluation of immunogenicity and protective efficacy of COH04S1 and COH04S351. In a previous study, COH04S1 has shown to be protective and significantly reduce viral loads in lungs of hamsters challenged with ancestral SARS-CoV-2 (Washington strain). COH04S1-mediated protection early after challenge with different SARS-CoV-2 strains is investigated in this study and compared to the protection conferred by COH04S351, a second-generation sMVA COVID-19 vaccine based on VOC S and N sequences.

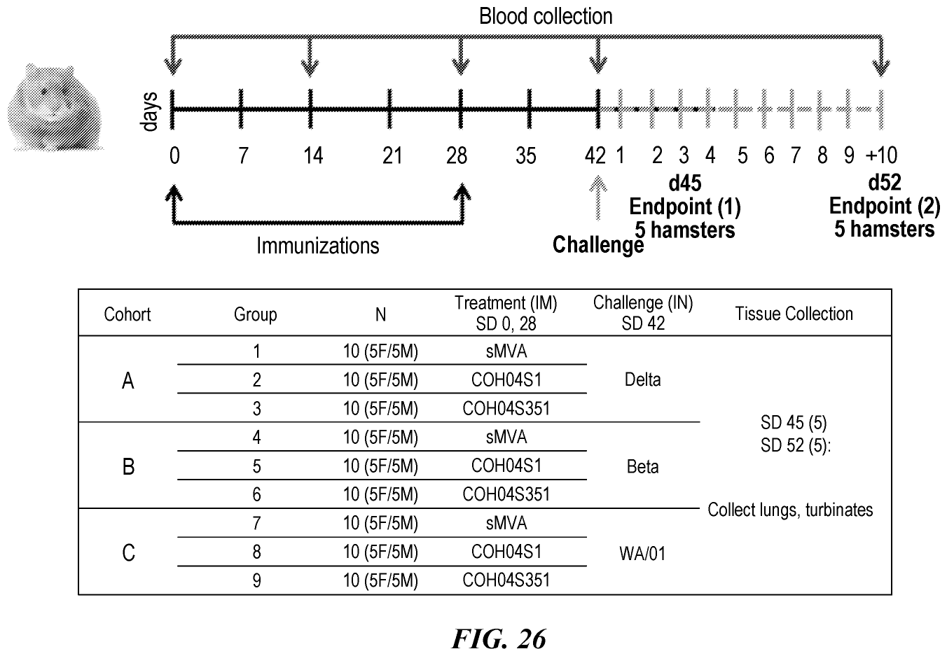

As shown in FIG. 26, Syrian hamsters (N=30) were intramuscularly vaccinated two times in a 28-day interval with COH04S1 or COH04S351. Control animals (N=30) were vaccinated with sMVA control vector. All vaccines were administered intramuscularly at 1×10⁸ PFU. Two weeks post-boost, hamsters were challenged intranasally with a high dose of SARS-CoV-2 Washington strain (WAS-Calu-3), B.1.351 Beta, or B.1.617.2 Delta variant (isolate USA/PHC658/2021). Body weight was measured daily for 10 days post-challenge. Because of the importance of assessing vaccine-mediated protective immunity at early and late time points post-challenge, 5 hamsters in each group were sacrificed at days 3 and 10 post-challenge for organ collection.

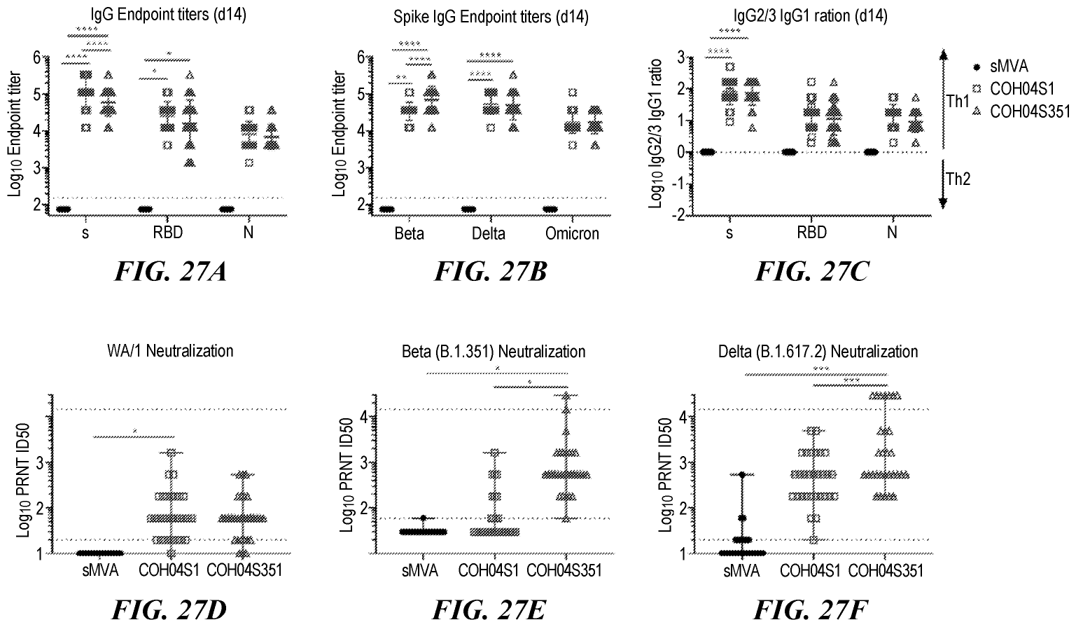

Serum samples collected 2 weeks post-prime were evaluated by ELISA for the presence of binding antibodies recognizing S, RBD, and N antigens based on ancestral SARS-CoV-2 sequences (FIG. 27A). High titers binding antibodies to S, RBD, and N were measured in all animals at 2 weeks post-prime vaccination. S-specific binding antibodies were higher in COH04S1-vaccinated animals than in COH04S351-vaccinated animals. RBD- and N-specific binding antibody titers were comparable between the groups. Both COH04S1- and COH04S351-vaccinated animals developed robust binding antibody responses to ancestral-specific S, RBD, and N antigens as well as S antigens based on Beta, Delta, and the currently dominating Omicron variant (FIGS. 27A-27B). The same samples were evaluated for the presence of binding antibodies of the IgG2/3 or IgG1 type, which indicate a Th1 or Th2 response, respectively (FIG. 27C). High titers IgG2/3 antibodies binding S, RBD, and N were measured in all animals, while IgG1 titers were low or absent. Consequently, IgG2/3-IgG1 ratio for both COH04S1- and COH04S351-vaccinated animals was skewed toward a Th1 response independently for the antigen analyzed.

NAb titers were evaluated in pre-challenge serum samples by PRNT assay using original Washington strain and Beta and Delta VOC. As shown in FIGS. 27D-27F, NAb responses against the ancestral virus were measured in both COH04S1- and COH04S351-vaccinated animals, with no significant differences between the two vaccine groups, although ancestral-specific NAb titers in COH04S351-vaccinated animals tended to be slightly lower than those in COH04S1-vaccinated animals. While ancestral-specific NAb titers were significantly elevated in COH04S1-vaccinated animals compared to controls, there was no statistical significance of ancestral-specific NAb titers in COH04S351-vaccinated animals compared to controls, indicating reduced neutralizing activity against the ancestral virus in COH04S351-vaccinated animals compared to COH04S1-vaccinated animals. In contrast, neutralizing titers measured against the Beta and Delta variants were significantly higher in COH04S351-vaccinated animals than in COH04S1-vaccinated animals. Both Beta- and Delta-specific neutralizing responses were significantly elevated in COH04S351-vaccinated animals compared to controls, whereas there was no statistical significance in Beta- and Delta-specific neutralizing titers when comparing COH04S1-vaccinated animals to controls, indicating reduced or relatively low levels of Beta- and Delta-specific NAb responses in COH04S1-vaccinated animals. In total, these results demonstrate that COH04S1 stimulated robust humoral immunity against the ancestral virus but reduced antibody levels against the Beta and Delta variants, whereas COH04S351 elicited significantly elevated humoral responses against the Beta and Delta variant but reduced antibody responses against the ancestral virus.

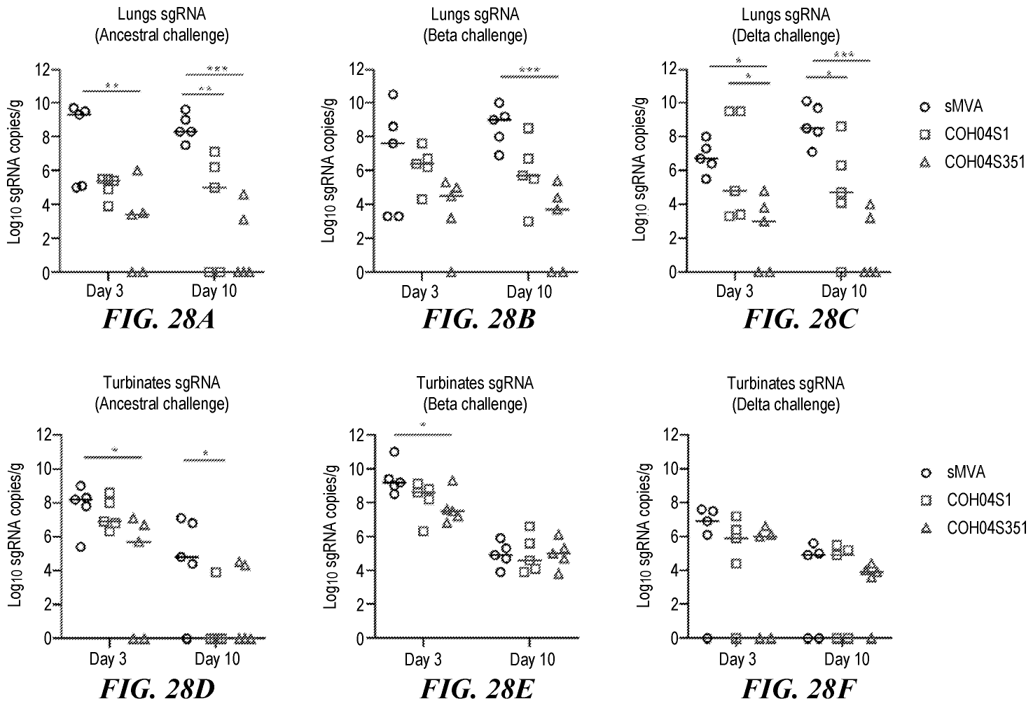

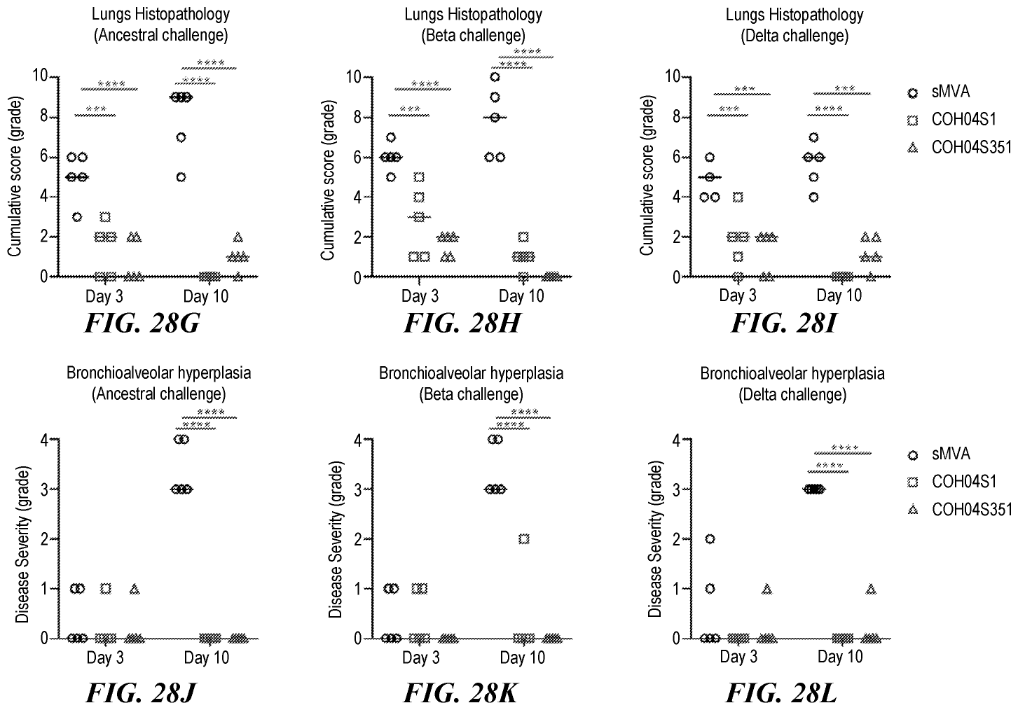

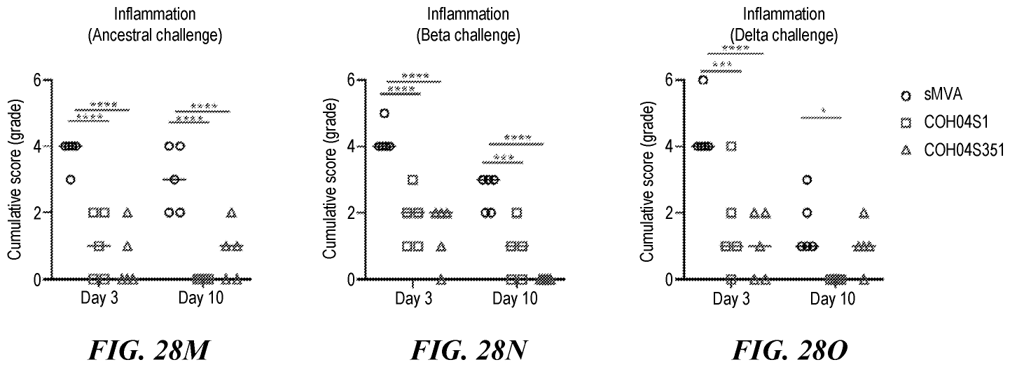

Vial loads were measured at day 3 and 10 post challenge in lung tissue and nasal turbinates by quantification of SARS-CoV-2 sgRNA to gauge the amount of replicating virus at lower and upper respiratory tracts. Viral sgRNA loads measured in lung tissue of COH04S1- and COH04S351-vaccinated animals at day 3 and 10 following challenge with the ancestral virus or the variant viruses were consistently lower than those of control animals, indicating efficacy of both vaccines to control lower respiratory infection by ancestral SARS-CoV-2 and SARS-CoV-2 Beta and Delta variants at early and late stages after viral challenge (FIGS. 28A-28C). Notably, lungs viral loads of COH04S351-vaccinated animals were consistently lower than those of COH04S1-vaccinated animals following challenge with either the ancestral virus or the two variant viruses, indicating improved efficacy of COH04S351 over COH04S1 to protect hamsters from lower respiratory tract infection by ancestral virus and SARS-CoV-2 VOC. In addition, while sgRNA was undetectable in the lungs of only one or two COH04S1-vaccinated hamsters at day 10 following challenge with the ancestral virus or the Delta variant, sgRNA was undetectable in the lungs of a proportion of COH04S351-vaccinated hamsters at day 3 and 10 following challenge with the ancestral virus or either of the two variant viruses. Viral loads measured in nasal turbinates of COH04S1- and COH04S351-vaccinated animals at day 3 or 10 following challenge with the ancestral virus or the variant viruses were either similar or only moderately reduced compared to those of controls, suggesting limited efficacy of COH04S1 and COH04S351 to control upper respiratory tract infection by ancestral SARS-CoV-2 or SARS-CoV-2 VOC (FIGS. 28D-28F). These results show that COH04S1 and COH04S351 protect hamsters from lower respiratory tract infection following challenge with SARS-CoV-2 ancestral virus and Beta and Delta variants.

Compared to controls, COH04S1- and COH04S351-vaccinated hamsters showed significantly reduced lung histopathology at day 3 and 10 following challenge with the ancestral virus or the two variant viruses (FIGS. 28G-281). Lung pathology in COH04S1- and COH04S351-vaccinated hamsters following challenge with the ancestral virus or the two variant viruses was in most cases either limited or undetectable, with no significant differences between the two vaccine groups. In addition, while all control animals showed high-grade bronchioalveolar hyperplasia (i.e., type II pneumocyte hyperplasia) at day 10 following challenge with the ancestral virus or the two variant viruses, COH04S1- and COH04S351-vaccinated animals showed no bronchioalveolar hyperplasia in almost all cases, regardless of the challenge virus (FIGS. 28J-28L). Furthermore, lung inflammation assessed at day 3 and 10 following challenge with the ancestral virus or the two variant viruses in COH04S1- and COH04S351-vaccinated hamsters was significantly reduced when compared to control animals, independent of the used challenge strain (FIGS. 28M-280). These results demonstrate that COH04S1 and COH04S351 afford potent efficacy to protect Syrian hamsters from lung pathology following challenge with ancestral SARS-CoV-2 and SARS-CoV-2 Beta and Delta variants.

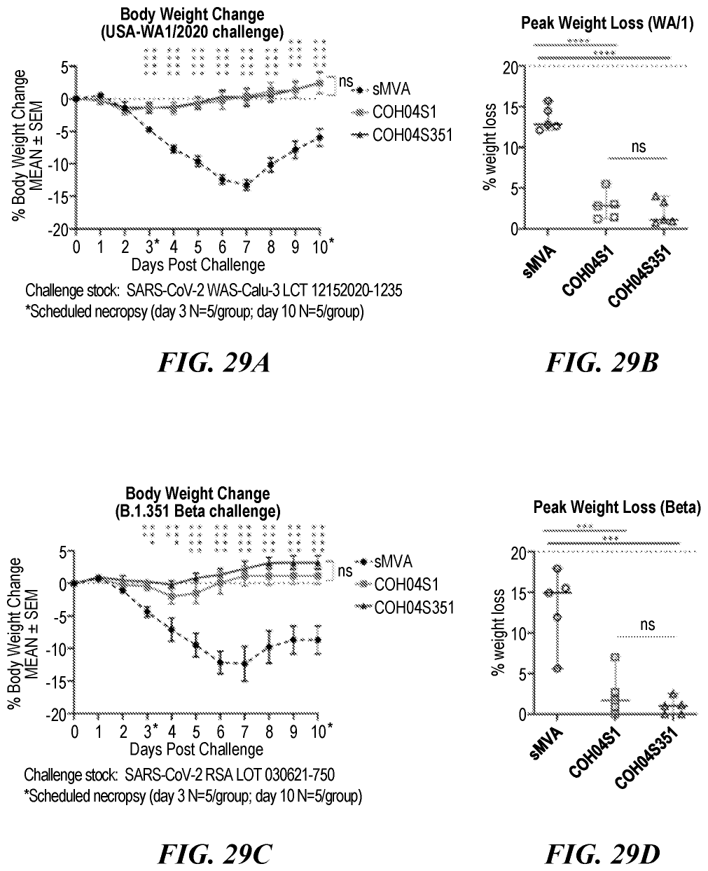

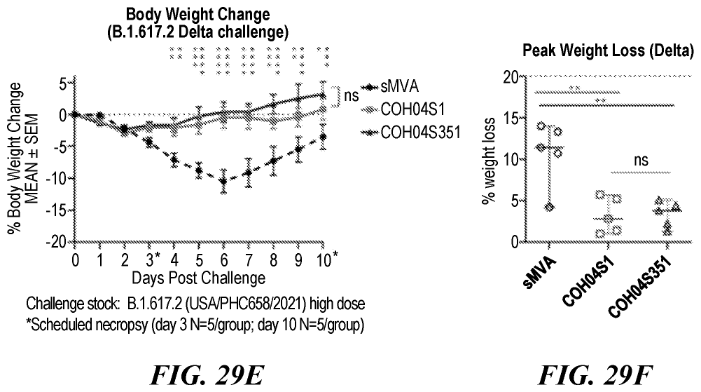

Two weeks post-boost, hamsters were challenged intranasally with a high inoculum of ancestral Wuhan SARS-CoV-2 or Beta or Delta VOC (FIGS. 29A-29F). In sMVA control animals, the animals' weight started decreasing from day 2 post-challenge and reached minimum levels between days 6 and 7, after which animals started gaining weight. By day 10 post-challenge, sMVA control animals had not returned to pre-challenge weight.

Hamsters vaccinated with COH04S1 and COH04S351 were protected against weight loss independently from the viral strain used to challenge the animals. Hamsters challenged with ancestral SARS-CoV-2 were equally protected by the two vaccines, which resulted in the animals starting to recover weight and showing significantly higher weight than sMVA control animals from day 3 post-challenge. Similar results were obtained in hamsters challenged with the beta and delta VOC with COH04S351-vaccinated hamsters presenting with a slightly reduced weight loss in comparison to COH04S1-vaccinated animals. Hamsters vaccinated with COH04S1 and COH04S351 and challenged with delta VOC had a significantly higher weight than sMVA controls starting from day 4 post-challenge, probably due to the overall milder weight loss induced by this delta variant challenge stock obtained from BEI Resources, which was later found to have a deleted orf7a gene possibly resulting in attenuated infectivity.

These results demonstrate that COH04S1 and COH04S351 are equally immunogenic and protective in hamsters against SARS-CoV-2 and its VOC.

### Example 11: Vaccine-Elicited Ancestral- and Variant-Specific Humoral Immune Responses

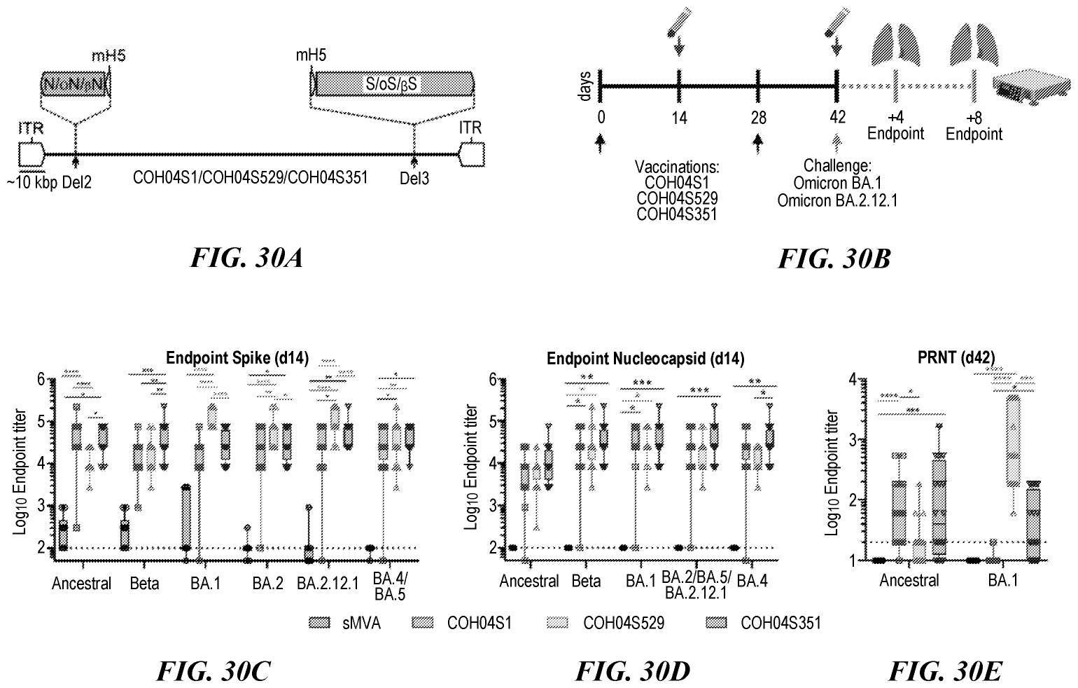

Syrian hamsters were vaccinated with COH04S1 or analogous vaccine constructs with Omicron BA.1 or Beta (B.1.351) sequence-specific S and N antigens, termed COH04S529 or COH04S351, respectively (FIGS. 30A-30B; Table 2). Hamsters were vaccinated twice in a 4-week interval by IM route. All three sMVA vaccines elicited potent post-prime binding antibody responses to S and N antigens of SARS-CoV-2 ancestral virus, Beta variant, and Omicron subvariants BA.1, BA.2, BA.2.12.1, and BA.4/5 (FIGS. 30C-30D). Consistent with the homologous antigen sequences, COH04S1 elicited higher IgG titers than the other vaccines to ancestral-specific S, while COH04S351 elicited higher IgG titers than the other vaccines to Beta (B.1.351)-specific S, and COH04S529 elicited higher IgG titers than the other vaccines to Omicron BA.1-specific S. While COH04S529 vaccination resulted in increased Omicron BA.2/BA.2.12.1-specific S titers compared to COH04S1 or COH04S351 vaccination, all three vaccines stimulated comparable IgG binding antibodies against Omicron BA.4/BA.5-specific S. Binding antibodies to ancestral- and variant-specific N antigens were generally comparable between the three vaccine groups, although BA.4-specific N titers induced by COH04S351 were significantly higher than those induced by COH04S529.

At 2 weeks post-vaccination prior to virus challenge, NAb responses were measured by PRNT assay against ancestral SARS-CoV-2 (USA-WA1/2020) and Omicron subvariant BA.1. NAb responses against ancestral SARS-CoV-2 were measured in all three vaccine groups, although ancestral-specific NAb titers in COH04S1- and COH04S351-vaccinated hamsters tended to be higher than those in COH04S529-vaccinated animals (FIG. 30E). Consistent with the exceptional Omicron immune evasion capacity, Omicron BA.1-specific NAb responses in COH04S1-vaccinated animals were either very low or remained undetectable. In contrast, hamsters vaccinated with COH04S529 or COH04S351 showed elevated Omicron BA.1-specific NAb titers, with highest BA.1-specific NAb titers in COH04S529-vaccinated animals, consistent with the BA.1-specific vaccine modification. These results demonstrate that COH04S1 and Omicron BA.1- and Beta-modified vaccines elicit strain-specific binding antibody and NAb responses against ancestral SARS-CoV-2, Beta variant, and Omicron subvariants.

### Example 12: Vaccine Protection Against Omicron BA.1 or BA.2.12.1 Virus-Induced Weight Loss

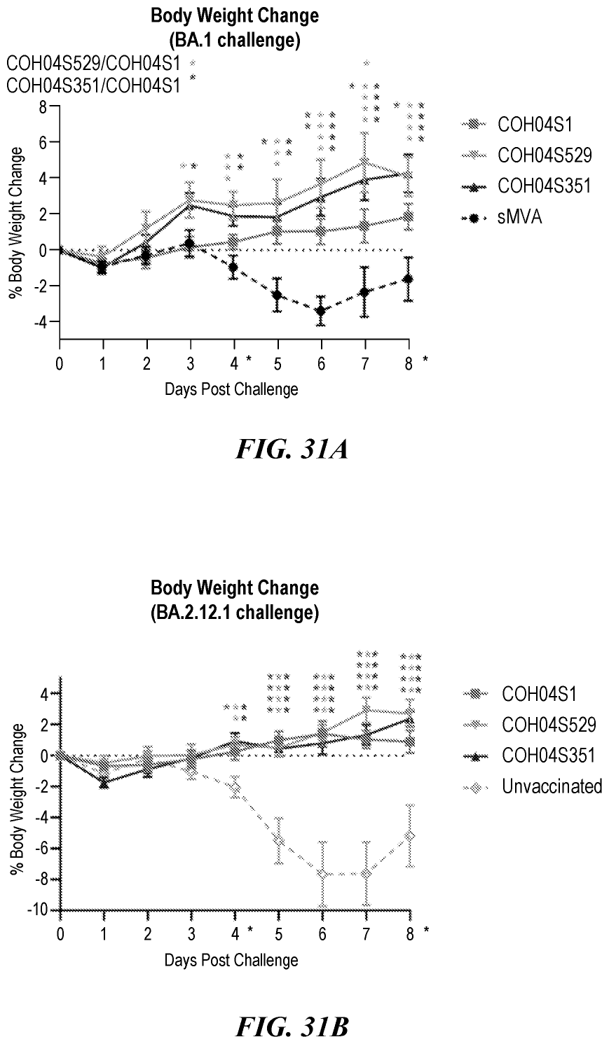

At 2 weeks post-vaccination, hamsters were challenged intranasally with either Omicron variant BA.1 or BA.2.12.1, and body weight was measured for 8 days. Hamsters vaccinated with sMVA without inserted antigens and unvaccinated hamsters were used as controls. Control animals showed progressive weight loss following challenge with BA.1 or BA.2.12.1, with maximum weight loss between 4% and 8% at day 6 post-vaccination. In contrast, body weight of hamsters vaccinated with COH04S1 or the variant-specific vaccines remained relatively stable or increased gradually over the 8-day observation period following challenge with either BA.1 or BA.2.12.1 (FIGS. 31A-31B). Notably, both COH04S529- and COH04S351-vaccinated animals showed consistently higher body weight than COH04S1-vaccinated hamsters following challenge with BA.1, suggesting improved vaccine efficacy through both the BA.1- and Beta-specific antigen modification to protect against BA.1-induced weight loss (FIG. 31A). In contrast, similar body weight changes were measured for all three vaccine groups following challenge with BA.2.12.1 (FIG. 31B). These results show that COH04S1 and Omicron BA.1 and Beta (B.1.351) sequence-modified vaccines protect hamsters from weight loss following challenge with Omicron BA.1 and BA.2.12.1.

### Example 13: Vaccine Protection Against Omicron BA.1 or BA.2.12.1 Respiratory Tract Infection

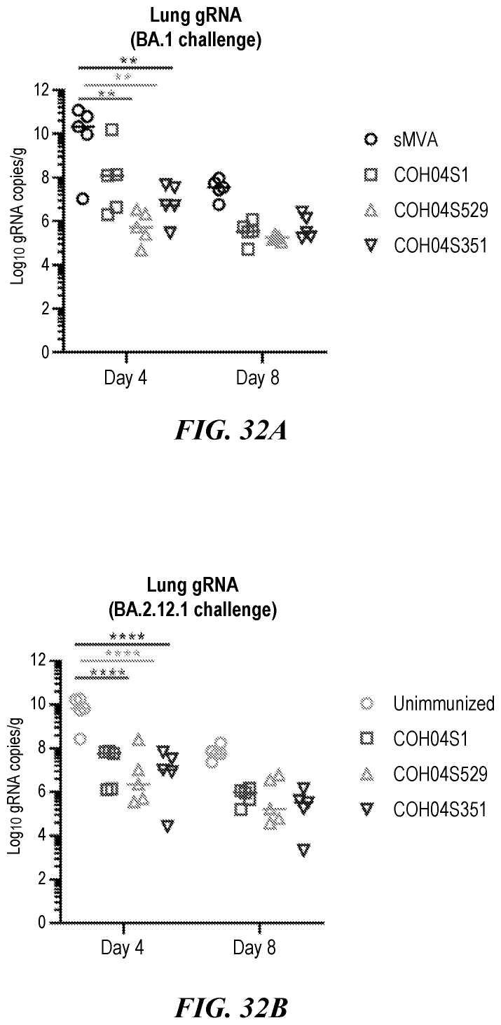

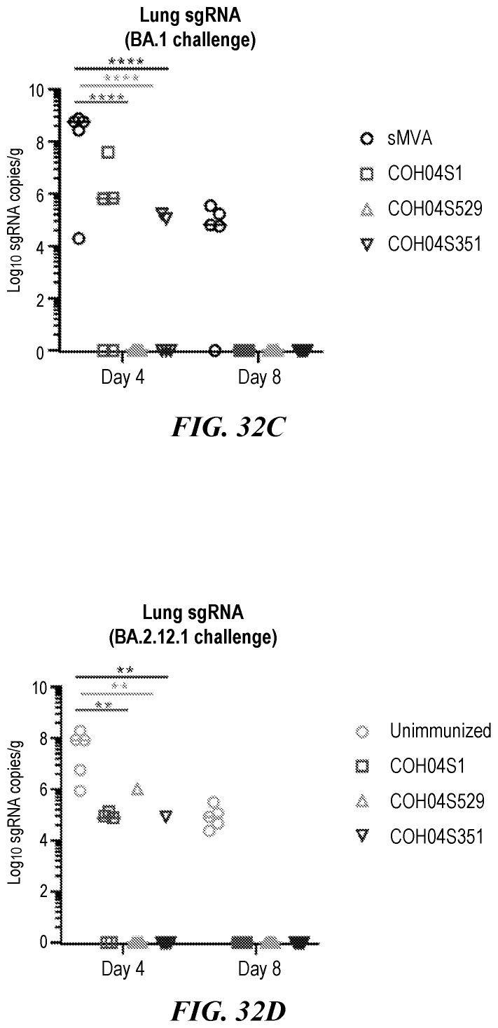

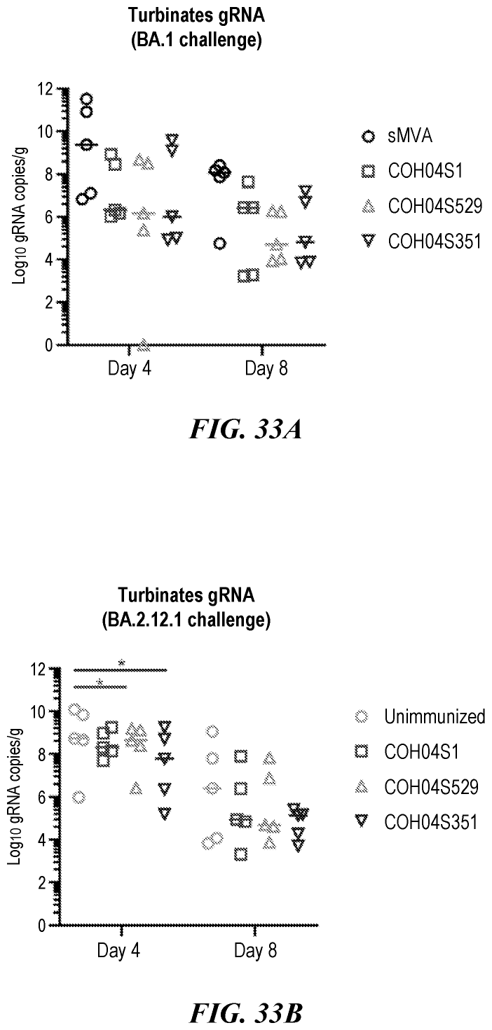

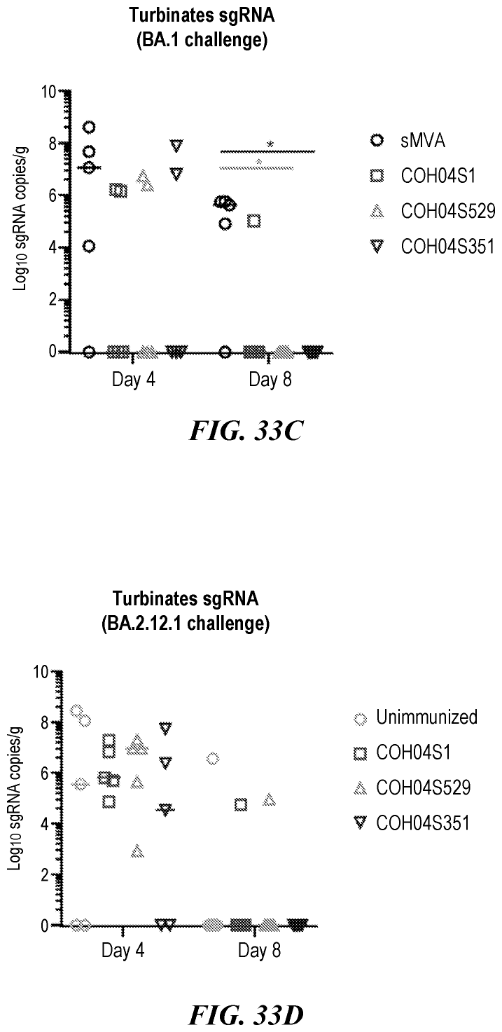

At days 4 and 8 post-challenge, viral loads were measured in lung tissue and nasal turbinates by quantification of SARS-CoV-2 genomic RNA (gRNA) and subgenomic RNA (sgRNA) to assess the magnitude of total and replicating virus at lower and upper respiratory tracts. Compared to the high lung viral load measured in control animals, significantly reduced gRNA and sgRNA levels were measured in the lungs of all three vaccine groups at day 4 following virus challenge with BA.1 or BA.2.12.1 (FIGS. 32A-32D), indicating potent efficacy of all three vaccines to control lower respiratory tract infection by BA.1 or BA.2.12.1 (FIGS. 32C-32D). At day 8 following challenge with BA.1 or BA.2.12.1, lung gRNA levels in animals from all three vaccine groups were substantially lower than those in control groups (FIGS. 32A-32B). In addition, in contrast to all or most control animals, all animals of the vaccine groups had undetectable lung sgRNA at day 8 following BA.1 or BA.2.12.1 virus challenge (FIGS. 32C-32D), indicating complete control of replicating virus in the lungs of all animals in the vaccine groups. While both COH04S529- and COH04S531-vaccinated animals had slightly reduced lung viral loads compared to COH04S1-vaccinated animals, these minor differences in lung viral loads between the three vaccine groups were not statistically significant. The strongest reduction in lung gRNA viral load following BA.1 or BA.2.12.1 challenge was observed for COH04S529-vaccinated animals (FIGS. 32A-32B). Moreover, in contrast to the animals in the COH04S1 and COH04S531 vaccine groups, all COH04S529-vaccinated animals had undetectable sgRNA at day 4 following BA.1 challenge (FIG. 32C). Compared to the potent vaccine efficacy to prevent Omicron infection in the lower respiratory tract, the vaccine efficacy to control Omicron infection in the upper respiratory tract was less evident (FIGS. 33A-33D). While gRNA and sgRNA viral loads measured in nasal turbinates of all vaccine groups following challenge with BA.1 were consistently lower than those of the control groups (FIGS. 33A-33B), significant differences were found only between nasal sgRNA viral loads of COH04S351- or COH04S529-vaccinated hamsters and controls at day 8 following BA.1 challenge (FIG. 33C). In addition, while all three vaccine groups tended to have reduced nasal gRNA levels compared to controls following virus challenge with BA.2.12.1, sgRNA viral loads in nasal turbinates of all vaccine groups following BA.2.12.1 virus challenge were comparable to those of the control groups (FIGS. 33B, 33D). These results show that COH04S1 and Omicron BA.1- and Beta (B.1.351) sequence-modified vaccines protect hamsters against lower respiratory tract infection by Omicron BA.1 and BA.2.12.1.

### Example 14: Vaccine Protection Against Omicron BA.1 or BA.2.12.1 Virus-Induced Lung Pathology

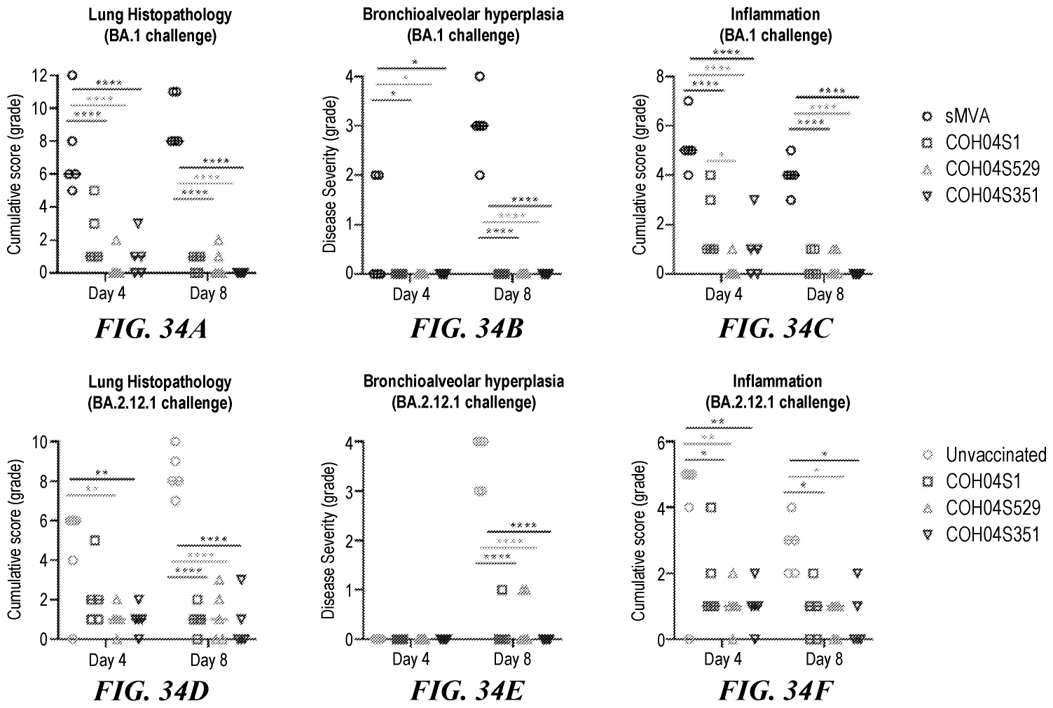

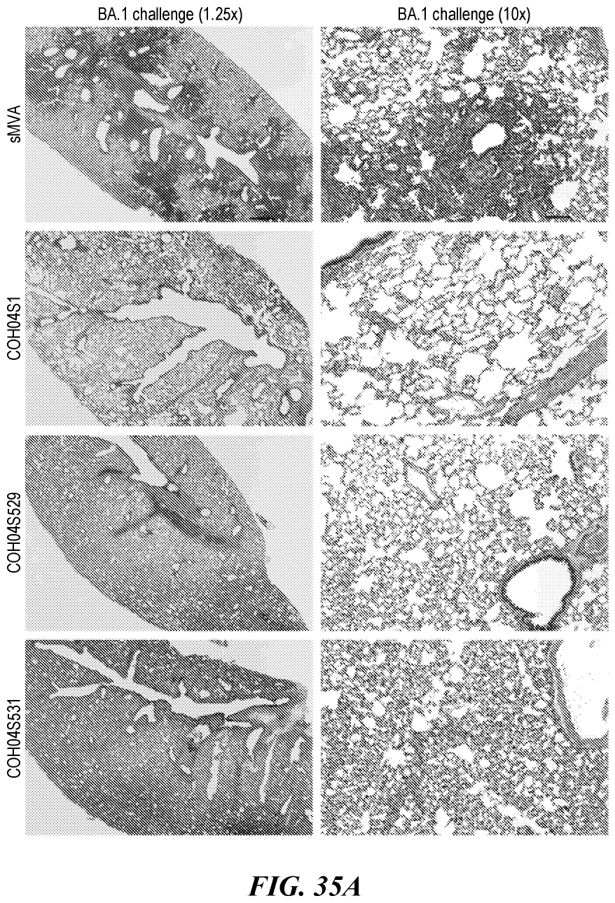

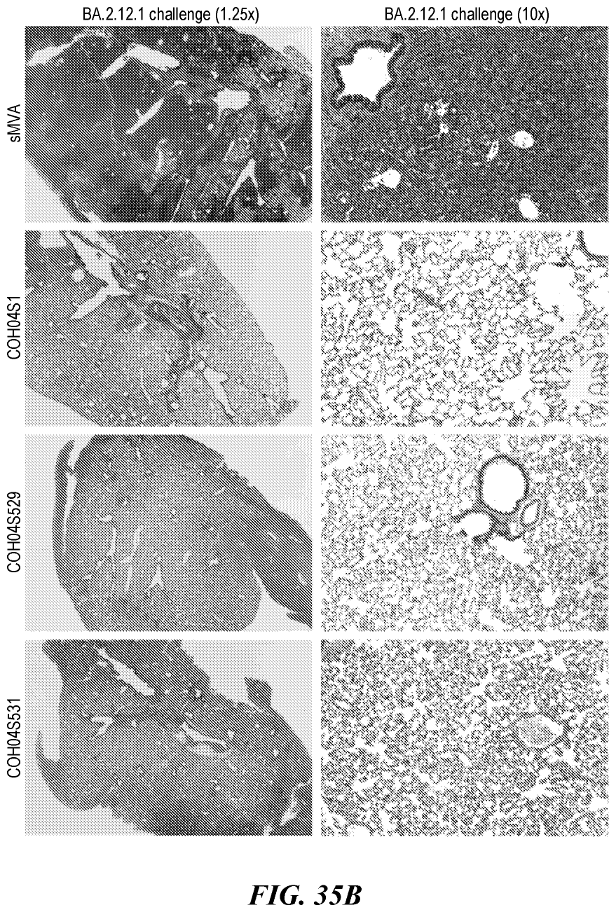

Lung pathology in all three vaccine groups following virus challenge with BA.1 or BA.2.12.1 was significantly reduced when compared to controls (FIGS. 34A-34F, 35A-35B), indicating efficacy of all three vaccines to protect hamsters from BA.1- and BA.2.12.1-induced lung injury. In addition, a subset of control hamsters had moderate bronchioalveolar hyperplasia (i.e., type II pneumocyte hyperplasia) at day 4 following BA.1 virus challenge, and all control animals had moderate to high-grade bronchioalveolar hyperplasia at day 8 following viral challenge with BA.1 or BA.2.12.1. In contrast, bronchioalveolar hyperplasia was undetectable in animals of all three vaccine groups at days 4 and 8 following viral challenge with BA.1 or BA.2.12.1, with only one or two exceptions in the COH04S1 and COH04S351 vaccine groups that showed low-grade bronchioalveolar hyperplasia (FIGS. 34B, 34E). Lung pathology in all three vaccine groups appeared mostly associated with inflammation, which was detectable at low levels in all vaccine groups at days 4 and 8 following virus challenge with BA.1 or BA.2.12.1, albeit at significantly reduced levels across all vaccine groups compared to controls (FIGS. 34C, 34F), confirming potent efficacy of all three vaccines to protect against BA.1- or BA.2.12.1-induced lung pathology. While both COH04S529- and COH04S351-vaccinated animals showed slightly reduced lung pathology compared to COH04S1-vaccinated animals following challenge with BA.2 or BA.2.12.1, these minor differences in lung pathology between the vaccine groups following challenge with BA.1 or BA.2.12.1 were not statistically significant (FIGS. 34A-34F). The only exception was found at day 4 following viral challenge with BA.1, where COH04S529-vaccinated hamsters showed significantly reduced lung inflammation compared to COH04S1-vaccinated animals (FIG. 34C). These results show that COH04S1 and Omicron BA.1 and Beta sequence-modified vaccines protect hamsters from lung pathology following virus challenge with Omicron subvariants BA.1 and BA.2.12.1.

### Example 15: Additional Examples

### Example 15-1

A reconstituted recombinant synthetic MVA (rsMVA) virus, comprising: a full-length synthetic MVA (sMVA) genome backbone comprising a nucleotide sequence identical or substantially identical to a parental MVA genome, wherein the full-length sMVA genome backbone is not intentionally modified as compared to a parental MVA genome; a heterologous DNA sequence encoding a coronavirus nucleocapsid (N) protein or immunogenic portion thereof inserted in the Del2 insertion site of the sMVA backbone, wherein the heterologous DNA sequence comprises any N protein sequence disclosed herein, in the appendices, or incorporated by reference; a heterologous DNA sequence encoding a coronavirus spike (S) protein or immunogenic portion thereof inserted in the Del3 insertion site of the sMVA backbone, wherein the heterologous DNA sequence comprises any S protein sequence disclosed herein, in the appendices, or incorporated by reference; wherein the N and S proteins are expressed by the reconstituted rsMVA virus.

### Example 15-2

A recombinant synthetic MVA (rsMVA) virus, comprising:
a full-length synthetic MVA (sMVA) genome backbone assembled upon homologous recombination of three chemically synthesized DNA fragments, F1, F2, and F3,
wherein each of F1, F2, and F3 contain a partial sequence of a full-length parental MVA genome,
wherein each of the partial sequences is not intentionally modified [[or identical or substantially identical]] as compared to the corresponding sequence of the parental MVA genome,
wherein F1 comprises (i) a first partial sequence of the parental MVA genome and (ii) a heterologous DNA sequence encoding a coronavirus nucleocapsid (N) protein or immunogenic portion thereof inserted in the Del2 insertion site of the first partial sequence, wherein the heterologous DNA sequence comprises any N protein sequence disclosed herein, in the appendices, or incorporated by reference,
wherein F2 comprises a second partial sequence of the parental MVA genome adjacent the first partial sequence, wherein the 5′ end of the second partial sequence overlaps with the 3′ end of the first partial sequence,
wherein F3 comprises (i) a third partial sequence of the parental MVA genome adjacent the second partial sequence, wherein the 5′ end of the second partial sequence overlaps with the 3′ end of the second partial sequence, and (ii) a heterologous DNA sequence encoding a coronavirus spike (S) protein or immunogenic portion thereof inserted in the Del3 insertion site of the first partial sequence, wherein the heterologous DNA sequence comprises any S protein sequence disclosed herein, in the appendices, or incorporated by reference.

### Example 15-3

The rsMVA virus of Example 15-1 or 15-2, wherein the N and S proteins are expressed by the reconstituted rsMVA virus.

### Example 15-4

The rsMVA virus of Example 15-1 or 15-2, wherein the full-length sMVA backbone is identical or substantially identical to the full-length parental MVA genome.

### Example 15-5

The rsMVA virus of Example 15-1 or 15-2, wherein the full-length parental MVA genome comprises a nucleotide sequence identical or substantially identical to NCBI Accession #U94848.

### Example 15-6

The rsMVA virus of Example 15-1 or 15-2, wherein the full-length sMVA backbone comprises one or more nucleotide alterations originating from the chemical synthesis of the first, second, or third partial sequences or from the cloning or propagation of F1, F2, or F3.

### Example 15-7

The rsMVA virus of Example 15-1 or 15-2, wherein the one or more nucleotide alterations comprise a single T to A nucleotide alteration located in the IGR at three base pairs downstream of open reading frame (ORF) 021L originating from the chemical synthesis of F1 or from the cloning or propagation of F1.

### Example 15-8

The rsMVA virus of Example 15-1 or 15-2, wherein the full-length sMVA genome backbone comprises the internal unique region (UR) of the full-length parental MVA genome.

### Example 15-9

The rsMVA virus of Example 15-1 or 15-2, wherein the full-length sMVA genome backbone further comprises inverted terminal repeat (ITR) regions of the full-length parental MVA genome flanking the UR.

### Example 15-10

The rsMVA virus of Example 15-1 or 15-2, wherein F1, F2, and F3 are each flanked by CR/HL/CR sequences derived from the full-length parental MVA genome.

### Example 15-11

The rsMVA virus of Example 15-1 or 15-2, wherein the first partial sequence comprises nucleotides 191-59743 of NCBI Accession #U94848; the second partial sequence comprises nucleotides 56744-119298 of NCBI Accession #U94848; and the third partial sequence comprises nucleotides 116299-177898 of NCBI Accession #U94848.

### REFERENCES

The references, patents, and published patent applications listed below, and all references cited in the specification above, are hereby incorporated by reference in their entirety, as if fully set forth herein.
1 Zhou, P. et al. A pneumonia outbreak associated with a new coronavirus of probable bat origin. Nature 579, 270-273, doi:10.1038/s41586-020-2012-7 (2020).
2 Zhu, N. et al. A Novel Coronavirus from Patients with Pneumonia in China, 2019. N Engl J Med 382, 727-733, doi:10.1056/NEJMoa2001017 (2020).
3 Baden, L. R. et al. Efficacy and Safety of the mRNA-1273 SARS-CoV-2 Vaccine. N Engl J Med 384, 403-416, doi:10.1056/NEJMoa2035389 (2021).
4 Polack, F. P. et al. Safety and Efficacy of the BNT162b2 mRNA Covid-19 Vaccine. N Engl J Med 383, 2603-2615, doi:10.1056/NEJMoa2034577 (2020).
Voysey, M. et al. Safety and efficacy of the ChAdOx1 nCoV-19 vaccine (AZD1222) against SARS-CoV-2: an interim analysis of four randomised controlled trials in Brazil, South Africa, and the UK. Lancet 397, 99-111, doi:10.1016/S0140-6736(20)32661-1 (2021).
6 Logunov, D. Y. et al. Safety and efficacy of an rAd26 and rAd5 vector-based heterologous prime-boost COVID-19 vaccine: an interim analysis of a randomised controlled phase 3 trial in Russia. Lancet 397, 671-681, doi:10.1016/S0140-6736(21)00234-8 (2021).
7 Sadoff, J. et al. Safety and Efficacy of Single-Dose Ad26.COV2.S Vaccine against Covid-19. N Engl J Med, doi:10.1056/NEJMoa2101544 (2021).
8 Krammer, F. SARS-CoV-2 vaccines in development. Nature 586, 516-527, doi:10.1038/s41586-020-2798-3 (2020).
9 Premkumar, L. et al. The receptor binding domain of the viral spike protein is an immunodominant and highly specific target of antibodies in SARS-CoV-2 patients. Sci Immunol 5, doi:10.1126/sciimmunol.abc8413 (2020).
McMahan, K. et al. Correlates of protection against SARS-CoV-2 in rhesus macaques. Nature 590, 630-634, doi:10.1038/s41586-020-03041-6 (2021).
11 Krammer, F. Correlates of protection from SARS-CoV-2 infection. Lancet 397, 1421-1423, doi:10.1016/S0140-6736(21)00782-0 (2021).
12 Heath, P. T. et al. Safety and Efficacy of NVX-CoV2373 Covid-19 Vaccine. N Engl J Med, doi:10.1056/NEJMoa2107659 (2021).
13 Tegally, H. et al. Detection of a SARS-CoV-2 variant of concern in South Africa. Nature 592, 438-443, doi:10.1038/s41586-021-03402-9 (2021).
14 Garcia-Beltran, W. F. et al. Multiple SARS-CoV-2 variants escape neutralization by vaccine-induced humoral immunity. Cell 184, 2523, doi:10.1016/j.cell.2021.04.006 (2021).
Zhou, D. et al. Evidence of escape of SARS-CoV-2 variant B.1.351 from natural and vaccine-induced sera. Cell 184, 2348-2361 e2346, doi:10.1016/j.cell.2021.02.037 (2021).
16 Faria, N. R. et al. Genomics and epidemiology of the P.1 SARS-CoV-2 lineage in Manaus, Brazil. Science, doi:10.1126/science.abh2644 (2021).
17 Dejnirattisai, W. et al. Antibody evasion by the P.1 strain of SARS-CoV-2. Cell, doi:10.1016/j.cell.2021.03.055 (2021).
18 Moore, J. P. & Offit, P. A. SARS-CoV-2 Vaccines and the Growing Threat of Viral Variants. JAMA 325, 821-822, doi:10.1001/jama.2021.1114 (2021).
19 Madhi, S. A. et al. Efficacy of the ChAdOx1 nCoV-19 Covid-19 Vaccine against the B.1.351 Variant. N Engl J Med, doi:10.1056/NEJMoa2102214 (2021).
20 Cox, R. J. & Brokstad, K. A. Not just antibodies: B cells and T cells mediate immunity to COVID-19. Nat Rev Immunol 20, 581-582, doi:10.1038/s41577-020-00436-4 (2020).
21 Grifoni, A. et al. Targets of T Cell Responses to SARS-CoV-2 Coronavirus in Humans with COVID-19 Disease and Unexposed Individuals. Cell 181, 1489-1501 e1415, doi:10.1016/j.cell.2020.05.015 (2020).
22 Ni, L. et al. Detection of SARS-CoV-2-Specific Humoral and Cellular Immunity in COVID-19 Convalescent Individuals. Immunity, doi:10.1016/j.immuni.2020.04.023 (2020).
23 Dutta, N. K., Mazumdar, K. & Gordy, J. T. The Nucleocapsid Protein of SARS-CoV-2: a Target for Vaccine Development. J Virol 94, doi:10.1128/JVI.00647-20 (2020).
24 Peng, Y. et al. Broad and strong memory CD4(+) and CD8(+) T cells induced by SARS-CoV-2 in UK convalescent individuals following COVID-19. Nat Immunol, doi:10.1038/s41590-020-0782-6 (2020).
Harris, P. E. et al. A Synthetic Peptide CTL Vaccine Targeting Nucleocapsid Confers Protection from SARS-CoV-2 Challenge in Rhesus Macaques. Vaccines (Basel) 9, doi:10.3390/vaccines9050520 (2021).
26 Dangi, T., Class, J., Palacio, N., Richner, J. M. & Penaloza MacMaster, P. Combining spike- and nucleocapsid-based vaccines improves distal control of SARS-CoV-2. Cell Rep, 109664, doi:10.1016/j.celrep.2021.109664 (2021).
27 Hong, S. H. et al. Immunization with RBD-P2 and N protects against SARS-CoV-2 in nonhuman primates. Sci Adv 7, doi:10.1126/sciadv.abg7156 (2021).
28 Matchett, W. E. et al. Nucleocapsid Vaccine Elicits Spike-Independent SARS-CoV-2 Protective Immunity. J Immunol, doi:10.4049/jimmunol.2100421 (2021).
29 Chiuppesi, F. et al. Development of a multi-antigenic SARS-CoV-2 vaccine candidate using a synthetic poxvirus platform. Nat Commun 11, 6121, doi:10.1038/s41467-020-19819-1 (2020).
30 Gilbert, S. C. Clinical development of Modified Vaccinia virus Ankara vaccines. Vaccine 31, 4241-4246, doi:10.1016/j.vaccine.2013.03.020 (2013).
31 Sutter, G. & Moss, B. Nonreplicating vaccinia vector efficiently expresses recombinant genes. Proc Natl Acad Sci USA 89, 10847-10851 (1992).
32 Volz, A. & Sutter, G. Modified Vaccinia Virus Ankara: History, Value in Basic Research, and Current Perspectives for Vaccine Development. Adv Virus Res 97, 187-243, doi:10.1016/bs.aivir.2016.07.001 (2017).
33 Pittman, P. R. et al. Phase 3 Efficacy Trial of Modified Vaccinia Ankara as a Vaccine against Smallpox. N Engl J Med 381, 1897-1908, doi:10.1056/NEJMoa1817307 (2019).
34 Wussow, F. et al. Human cytomegalovirus vaccine based on the envelope gH/gL pentamer complex. PLoS Pathog 10, e1004524, doi:10.1371/journal.ppat.1004524 (2014).
Wussow, F. et al. A vaccine based on the rhesus cytomegalovirus UL128 complex induces broadly neutralizing antibodies in rhesus macaques. J Virol 87, 1322-1332, doi:10.1128/JVI.01669-12 (2013).
36 Chiuppesi, F. et al. Multiantigenic Modified Vaccinia Virus Ankara Vaccine Vectors To Elicit Potent Humoral and Cellular Immune Reponses against Human Cytomegalovirus in Mice. J Virol 92, doi:10.1128/JVI.01012-18 (2018).
37 La Rosa, C. et al. MVA vaccine encoding CMV antigens safely induces durable expansion of CMV-specific T-cells in healthy adults. Blood, doi:10.1182/blood-2016-07-729756 (2016).
38 Aldoss, I. et al. Poxvirus Vectored Cytomegalovirus Vaccine to Prevent Cytomegalovirus Viremia in Transplant Recipients: A Phase 2, Randomized Clinical Trial. Ann Intern Med 172, 306-316, doi:10.7326/M19-2511 (2020).
39 Yuan, Y. et al. Complete regression of cutaneous metastases with systemic immune response in a patient with triple negative breast cancer receiving p53MVA vaccine with pembrolizumab. Oncoimmunology 6, e1363138, doi:10.1080/2162402X.2017.1363138 (2017).
Tostanoski, L. H. et al. Ad26 vaccine protects against SARS-CoV-2 severe clinical disease in hamsters. Nat Med 26, 1694-1700, doi:10.1038/s41591-020-1070-6 (2020).
41 Sanchez-Felipe, L. et al. A single-dose live-attenuated YF17D-vectored SARS-CoV-2 vaccine candidate. Nature 590, 320-325, doi:10.1038/s41586-020-3035-9 (2021).
42 Yahalom-Ronen, Y. et al. A single dose of recombinant VSV-G-spike vaccine provides protection against SARS-CoV-2 challenge. Nat Commun 11, 6402, doi:10.1038/s41467-020-20228-7 (2020).
43 Chan, J. F. et al. Simulation of the Clinical and Pathological Manifestations of Coronavirus Disease 2019 (COVID-19) in a Golden Syrian Hamster Model: Implications for Disease Pathogenesis and Transmissibility. Clin Infect Dis 71, 2428-2446, doi:10.1093/cid/ciaa325 (2020).
44 Sia, S. F. et al. Pathogenesis and transmission of SARS-CoV-2 in golden hamsters. Nature 583, 834-838, doi:10.1038/s41586-020-2342-5 (2020).
Imai, M. et al. Syrian hamsters as a small animal model for SARS-CoV-2 infection and countermeasure development. Proc Natl Acad Sci USA 117, 16587-16595, doi:10.1073/pnas.2009799117 (2020).
46 Hoffmann, M. et al. SARS-CoV-2 Cell Entry Depends on ACE2 and TMPRSS2 and Is Blocked by a Clinically Proven Protease Inhibitor. Cell 181, 271-280 e278, doi:10.1016/j.cell.2020.02.052 (2020).
47 Wrapp, D. et al. Cryo-EM structure of the 2019-nCoV spike in the prefusion conformation. Science 367, 1260-1263, doi:10.1126/science.abb2507 (2020).
48 Walls, A. C. et al. Structure, Function, and Antigenicity of the SARS-CoV-2 Spike Glycoprotein. Cell 181, 281-292 e286, doi:10.1016/j.cell.2020.02.058 (2020).
49 Woolsey, C. et al. Establishment of an African green monkey model for COVID-19 and protection against re-infection. Nat Immunol 22, 86-98, doi:10.1038/s41590-020-00835-8 (2021).
50 Munster, V. J. et al. Respiratory disease in rhesus macaques inoculated with SARS-CoV-2. Nature, doi:10.1038/s41586-020-2324-7 (2020).
51 Chandrashekar, A. et al. SARS-CoV-2 infection protects against rechallenge in rhesus macaques. Science 369, 812-817, doi:10.1126/science.abc4776 (2020).
52 Corbett, K. S. et al. Evaluation of the mRNA-1273 Vaccine against SARS-CoV-2 in Nonhuman Primates. N Engl J Med 383, 1544-1555, doi:10.1056/NEJMoa2024671 (2020).
53 Vogel, A. B. et al. BNT162b vaccines protect rhesus macaques from SARS-CoV-2. Nature 592, 283-289, doi:10.1038/s41586-021-03275-y (2021).
54 van Doremalen, N. et al. ChAdOx1 nCoV-19 vaccine prevents SARS-CoV-2 pneumonia in rhesus macaques. Nature 586, 578-582, doi:10.1038/s41586-020-2608-y (2020).
55 Yu, J. et al. DNA vaccine protection against SARS-CoV-2 in rhesus macaques. Science, doi:10.1126/science.abc6284 (2020).
56 Cross, R. W. et al. Intranasal exposure of African green monkeys to SARS-CoV-2 results in acute phase pneumonia with shedding and lung injury still present in the early convalescence phase. Virol J 17, 125, doi:10.1186/s12985-020-01396-w (2020).
57 Geers, D. eta. SARS-CoV-2 variants of concern partially escape humoral but not T-cell responses in COVID-19 convalescent donors and vaccines. Sci Immunol 6, doi:10.1126/sciimmunol.abj1750 (2021).
58 Routhu, N. K. et al. A modified vaccinia Ankara vector-based vaccine protects macaques from SARS-CoV-2 infection, immune pathology, and dysfunction in the lungs. Immunity 54, 542-556 e549, doi:10.1016/j.immuni.2021.02.001 (2021).
59 Liu, R. et al. One or two injections of MVA-vectored vaccine shields hACE2 transgenic mice from SARS-CoV-2 upper and lower respiratory tract infection. Proc Natl Acad Sci USA 118, doi:10.1073/pnas.2026785118 (2021).
60 Garcia-Arriaza, J. et al. COVID-19 vaccine candidates based on modified vaccinia virus Ankara expressing the SARS-CoV-2 spike induce robust T- and B-cell immune responses and full efficacy in mice. J Virol, doi:10.1128/JVI.02260-20 (2021).
61 Tscherne, A. et al. Immunogenicity and efficacy of the COVID-19 candidate vector vaccine MVA-SARS-2-S in preclinical vaccination. Proc Natl Acad Sci USA 118, doi:10.1073/pnas.2026207118 (2021).
62 Rice, A. et al. Intranasal plus subcutaneous prime vaccination with a dual antigen COVID-19 vaccine elicits T-cell and antibody responses in mice. Sci Rep 11, 14917, doi:10.1038/s41598-021-94364-5 (2021).
63 Iwasaki, A. & Yang, Y. The potential danger of suboptimal antibody responses in COVID-19. Nat Rev Immunol 20, 339-341, doi:10.1038/s41577-020-0321-6 (2020).
64 Graham, B. S. Rapid COVID-19 vaccine development. Science 368, 945-946, doi:10.1126/science.abb8923 (2020).
65 Koch, T. et al. Safety and immunogenicity of a modified vaccinia virus Ankara vector vaccine candidate for Middle East respiratory syndrome: an open-label, phase 1 trial. Lancet Infect Dis 20, 827-838, doi:10.1016/S1473-3099(20)30248-6 (2020).
66 Haagmans, B. L. et al. An orthopoxvirus-based vaccine reduces virus excretion after MERS-CoV infection in dromedary camels. Science 351, 77-81, doi:10.1126/science.aad1283 (2016).
67 Halle, S. et al. Induced bronchus-associated lymphoid tissue serves as a general priming site for T cells and is maintained by dendritic cells. J Exp Med 206, 2593-2601, doi:10.1084/jem.20091472 (2009).
68 Crawford, K. H. D. et al. Protocol and Reagents for Pseudotyping Lentiviral Particles with SARS-CoV-2 Spike Protein for Neutralization Assays. Viruses 12, doi:10.3390/v12050513 (2020).

1. A vaccine and/or immunogenic composition for preventing or treating SARS-CoV-2 infection in a subject comprising:
(i) a single synthetic DNA fragment comprising the entire genome of a modified vaccinia Ankara (MVA), or two or more synthetic DNA fragments each comprising a partial sequence of the genome of the MVA such that the two or more DNA fragments, when transferred into a host cell, are assembled sequentially and comprise the full-length sequence of the MVA genome, and
(ii) one or more DNA sequences encoding the spike (S) protein and the nucleocapsid (N) protein of SARS-CoV-2, subunits, or fragments thereof, inserted into one or more insertion sites of the MVA, wherein the S protein and the N protein are expressed in the host cell upon transfection of the one or more MVA DNA fragments.

2. The vaccine and/or immunogenic composition of claim 1, wherein the one or more insertion sites comprise Del2, IGR69/70, and Del3.

3. The vaccine and/or immunogenic composition of claim 1, wherein the one or more DNA sequences encoding the S protein and the N protein are under the control of an mH5 promoter.

4. The vaccine and/or immunogenic composition of claim 1, wherein the DNA sequence encoding the S protein is based on the Wuhan-Hu-1 reference strain or is derived from a variant of concern (VOC).

5. The vaccine and/or immunogenic composition of claim 1, wherein the DNA sequence encoding the N protein is based on the Wuhan-Hu-1 reference strain or is derived from a variant of concern (VOC).

6. The vaccine and/or immunogenic composition of claim 4, wherein the VOC is selected from the group consisting of B.1.1.7 (Alpha), B.1.351 (Beta), P.1 (Gamma), B.1.617.2 (Delta), B.1.621 (Mu), C.37 (Lambda), C.1.2, BA.1 (Omicron), BA.2 (Omicron), BA.2.12.1 (Omicron), BA.4 (Omicron), BA.5 (Omicron), BA.2.75 (Omicron), BQ.1 (Omicron), BQ.1.1 (Omicron), and XBB (Omicron).

7. The vaccine and/or immunogenic composition of claim 1, wherein the DNA sequence encoding the S protein comprises a nucleotide sequence that is at least 80% identical to any one of SEQ ID NOs: 5, 9, 11, 13, 15, 21, 25, and 29.

8. The vaccine and/or immunogenic composition of claim 1, wherein the DNA sequence encoding the N protein comprises a nucleotide sequence that is at least 80% identical to any one of SEQ ID NOs: 7, 17, 19, 23, 27, and 31.

9. A vaccine and/or immunogenic composition for preventing or treating SARS-CoV-2 infection in a subject comprising a synthetic modified vaccinia Ankara (sMVA) vector comprising a nucleotide sequence that is at least 80% identical to SEQ ID NO: 33.

10. The vaccine and/or immunogenic composition of claim 9, further comprising a pharmaceutically acceptable carrier, adjuvant, additive, or combination thereof.

11. A method of preventing, treating SARS-CoV-2 infection, or eliciting an immune response to SARS-CoV-2 in a subject comprising administering a prophylactically or therapeutically effective amount of a vaccine and/or immunogenic composition to the subject, wherein the vaccine and/or immunogenic composition comprises:
(i) a single synthetic DNA fragment comprising the entire genome of a modified vaccinia Ankara (MVA), or two or more synthetic DNA fragments each comprising a partial sequence of the genome of the MVA such that the two or more DNA fragments, when transferred into a host cell, are assembled sequentially and comprise the full-length sequence of the MVA genome, and
(ii) one or more DNA sequences encoding the spike (S) protein and the nucleocapsid (N) protein of SARS-CoV-2, subunits, or fragments thereof, inserted into one or more insertion sites of the MVA, wherein the S protein and the N protein are expressed in the host cell upon transfection of the one or more MVA DNA fragments.

12. The method of claim 11, wherein the subject is infected with SARS-CoV-2 or is at risk of being infected with SARS-CoV-2.

13. The method of claim 11, wherein the SARS-CoV-2 comprises the Wuhan-Hu-1 reference strain or a VOC.

14. The method of claim 13, wherein the VOC is selected from the group consisting of B.1.1.7 (Alpha), B.1.351 (Beta), P.1 (Gamma), B.1.617.2 (Delta), B.1.621 (Mu), C.37 (Lambda), C.1.2, BA.1 (Omicron), BA.2 (Omicron), BA.2.12.1 (Omicron), BA.4 (Omicron), BA.5 (Omicron), BA.2.75 (Omicron), BQ.1 (Omicron), BQ.1.1 (Omicron), and XBB (Omicron).

15. The method of claim 11, wherein the vaccine and/or immunogenic composition is administered by intramuscular injection, intranasal instillation, intradermal injection, and/or scarification.

16. The method of claim 11, wherein a single dose of the vaccine composition is administered.

17. The method of claim 11, wherein two doses of the vaccine composition are administered.

18. The method of claim 11, wherein three or more doses of the vaccine composition are administered.

19. The method of claim 11, wherein one booster dose of the vaccine and/or immunogenic composition is administered.

20. The method of claim 11, wherein two or more booster doses of the vaccine and/or immunogenic composition are administered.
